SEQUENCE LISTING (775 sequences)

SEQ ID NO: 1 - protein, 9 aa, Homo sapiens
NTDNNLAVY

SEQ ID NO: 2 - protein, 9 aa, Homo sapiens
ETDNNIVVY

SEQ ID NO: 3 - protein, 9 aa, Homo sapiens
YTDNWLAVY

SEQ ID NO: 4 - protein, 9 aa, Homo sapiens
GTDNWLAQY

SEQ ID NO: 5 - protein, 9 aa, Homo sapiens
PTDENLARY

SEQ ID NO: 6 - protein, 9 aa, Homo sapiens
NTDNLLTEY

SEQ ID NO: 7 - protein, 5 aa, synthetic construct
GGGGS

SEQ ID NO: 8 - protein, 5 aa, synthetic construct
DYNIH

SEQ ID NO: 9 - protein, 17 aa, synthetic construct
WINPNSGGTNYAQKFQG

SEQ ID NO: 10 - protein, 7 aa, synthetic construct
DKVGLDY

SEQ ID NO: 11 - protein, 11 aa, synthetic construct
RASQGINNWLA

SEQ ID NO: 12 - protein, 7 aa, synthetic construct
AASSLQA

SEQ ID NO: 13 - protein, 9 aa, synthetic construct
QQSYLTPYT

SEQ ID NO: 14 - protein, 116 aa, synthetic construct
QVQLVQSGAEVKKPGASVKVSCKASGYTVTDYNIHWVRQAPGQGLEWMGWINPNSGGTNYAQKFQGRVTMTRDTSTSTVYMELSSLRSEDTAVYYCARDKVGLDYWGQGTLVTVSS

SEQ ID NO: 15 - protein, 107 aa, synthetic construct
DIQMTQSPSSLSASVGDRVTITCRASQGINNWLAWYQQKPGKAPKLLIYAASSLQAGVPSRFSGSGSGTDFTLTISSLQPEDFATYYCQQSYLTPYTFGQGTKLEIK

SEQ ID NO: 16 - protein, 5 aa, synthetic construct
DYNIN

SEQ ID NO: 17 - protein, 17 aa, synthetic construct
WINPNSGGTNYAQKLQG

SEQ ID NO: 18 - protein, 6 aa, synthetic construct
ANWLDY

SEQ ID NO: 19 - protein, 11 aa, synthetic construct
RASQDINRWLA

SEQ ID NO: 20 - protein, 7 aa, synthetic construct
AASTLQS

SEQ ID NO: 21 - protein, 9 aa, synthetic construct
QQSYGSPYT

SEQ ID NO: 22 - protein, 5 aa, synthetic construct
DYYLH

SEQ ID NO: 23 - protein, 17 aa, synthetic construct
WMNPNSGGTNYAQKFQG

SEQ ID NO: 24 - protein, 6 aa, synthetic construct
GEWFDP

SEQ ID NO: 25 - protein, 11 aa, synthetic construct
RASQSISRWLA

SEQ ID NO: 26 - protein, 9 aa, synthetic construct
QQSYSTPYT

SEQ ID NO: 27 - protein, 5 aa, synthetic construct
DYYMH

SEQ ID NO: 28 - protein, 17 aa, synthetic construct
WIFPNSGGTNYAQKFQG

SEQ ID NO: 29 - protein, 6 aa, synthetic construct
EDWCXP

SEQ ID NO: 30 - protein, 11 aa, synthetic construct
RASQDISRWLA

SEQ ID NO: 31 - protein, 7 aa, synthetic construct
AASXLQS

SEQ ID NO: 32 - protein, 9 aa, synthetic construct
QQSYSSPYT

SEQ ID NO: 33 - protein, 5 aa, synthetic construct
GKYMH

SEQ ID NO: 34 - protein, 17 aa, synthetic construct
WMNPNSGNTGYAQKFQG

SEQ ID NO: 35 - protein, 14 aa, synthetic construct
GGYYGSGSWTYFDL

SEQ ID NO: 36 - protein, 11 aa, synthetic construct
RASQSISSYLN

SEQ ID NO: 37 - protein, 7 aa, synthetic construct
DASNLET

SEQ ID NO: 38 - protein, 9 aa, synthetic construct
QQTDSIPIT

SEQ ID NO: 39 - protein, 5 aa, synthetic construct
GYLMH

SEQ ID NO: 40 - protein, 17 aa, synthetic construct
WINPNSGGTNYAPKFQG

SEQ ID NO: 41 - protein, 7 aa, synthetic construct
ESSGWGN

SEQ ID NO: 42 - protein, 11 aa, synthetic construct
RASQGIGRWLA

SEQ ID NO: 43 - protein, 7 aa, synthetic construct
GASSLQS

SEQ ID NO: 44 - protein, 9 aa, synthetic construct
QQSYITPYT

SEQ ID NO: 45 - protein, 5 aa, synthetic construct
GYNMH

SEQ ID NO: 46 - protein, 17 aa, synthetic construct
WINPNTGGTSYAQKFQG

SEQ ID NO: 47 - protein, 11 aa, synthetic construct
RAXQXIXRWLA

SEQ ID NO: 48 - protein, 9 aa, synthetic construct
QQSYSIPIT

SEQ ID NO: 49 - protein, 11 aa, synthetic construct
RASQSIGRWLA

SEQ ID NO: 50 - protein, 7 aa, synthetic construct
AASRLQS

SEQ ID NO: 51 - protein, 8 aa, synthetic construct
QQSYGVPT

SEQ ID NO: 52 - protein, 11 aa, synthetic construct
RASQSINRWLA

SEQ ID NO: 53 - protein, 5 aa, synthetic construct
GYQMH

SEQ ID NO: 54 - protein, 8 aa, synthetic construct
DGGSYLDY

SEQ ID NO: 55 - protein, 11 aa, synthetic construct
RASQSINSWLA

SEQ ID NO: 56 - protein, 9 aa, synthetic construct
QQSYSIPYN

SEQ ID NO: 57 - protein, 5 aa, synthetic construct
GYYMH

SEQ ID NO: 58 - protein, 17 aa, synthetic construct
RIFPNSGGTDYAQKFQG

SEQ ID NO: 59 - protein, 6 aa, synthetic construct
GDWFDP

SEQ ID NO: 60 - protein, 5 aa, synthetic construct
HYGMS

SEQ ID NO: 61 - protein, 7 aa, synthetic construct
DLGXYDY

SEQ ID NO: 62 - protein, 11 aa, synthetic construct
RASQSISSWLA

SEQ ID NO: 63 - protein, 7 aa, synthetic construct
AASSLQS

SEQ ID NO: 64 - protein, 8 aa, synthetic construct
QQSYSYPT

SEQ ID NO: 65 - protein, 5 aa, synthetic construct
KYDIA

SEQ ID NO: 66 - protein, 17 aa, synthetic construct
WIYPNSGGTNYAQKFQG

SEQ ID NO: 67 - protein, 11 aa, synthetic construct
RASQSVSSWLA

SEQ ID NO: 68 - protein, 7 aa, synthetic construct
AASTLHS

SEQ ID NO: 69 - protein, 5 aa, synthetic construct
NFGIS

SEQ ID NO: 70 - protein, 5 aa, synthetic construct
NFPMH

SEQ ID NO: 71 - protein, 6 aa, synthetic construct
GEWLGY

SEQ ID NO: 72 - protein, 11 aa, synthetic construct
RASQSVSNWLA

SEQ ID NO: 73 - protein, 5 aa, synthetic construct
NFPMX

SEQ ID NO: 74 - protein, 17 aa, synthetic construct
WINPNSGGTNYAQNFQG

SEQ ID NO: 75 - protein, 11 aa, synthetic construct
RASQSVSXWLA

SEQ ID NO: 76 - protein, 5 aa, synthetic construct
NFVVS

SEQ ID NO: 77 - protein, 7 aa, synthetic construct
SVGWFDP

SEQ ID NO: 78 - protein, 11 aa, synthetic construct
RASESIGRWLA

SEQ ID NO: 79 - protein, 9 aa, synthetic construct
QQSYISPYT

SEQ ID NO: 80 - protein, 5 aa, synthetic construct
NHNMH

SEQ ID NO: 81 - protein, 11 aa, synthetic construct
RASQSIDRWLA

SEQ ID NO: 82 - protein, 9 aa, synthetic construct
QQSYSIPYT

SEQ ID NO: 83 - protein, 5 aa, synthetic construct
NSAIH

SEQ ID NO: 84 - protein, 17 aa, synthetic construct
YISGSGDTMFYADSVKG

SEQ ID NO: 85 - protein, 13 aa, synthetic construct
DPYYYGSGSYFDY

SEQ ID NO: 86 - protein, 7 aa, synthetic construct
YASQSIS

SEQ ID NO: 87 - protein, 5 aa, synthetic construct
NSVIS

SEQ ID NO: 88 - protein, 17 aa, synthetic construct
WISVYNGETNYAQKLQG

SEQ ID NO: 89 - protein, 8 aa, synthetic construct
DRIDGMDV

SEQ ID NO: 90 - protein, 11 aa, synthetic construct
RASQRISYWVA

SEQ ID NO: 91 - protein, 7 aa, synthetic construct
DASTLQS

SEQ ID NO: 92 - protein, 9 aa, synthetic construct
QQSYSTPLT

SEQ ID NO: 93 - protein, 5 aa, synthetic construct
NYAFS

SEQ ID NO: 94 - protein, 6 aa, synthetic construct
GDWLDY

SEQ ID NO: 95 - protein, 9 aa, synthetic construct
QQSYNTPYT

SEQ ID NO: 96 - protein, 5 aa, synthetic construct
NYAIS

SEQ ID NO: 97 - protein, 11 aa, synthetic construct
RASQNINRWLA

SEQ ID NO: 98 - protein, 9 aa, synthetic construct
QQSYSPPYT

SEQ ID NO: 99 - protein, 5 aa, synthetic construct
NYDIN

SEQ ID NO: 100 - protein, 6 aa, synthetic construct
GEWLSV

SEQ ID NO: 101 - protein, 11 aa, synthetic construct
RASQEIRRWLA

SEQ ID NO: 102 - protein, 9 aa, synthetic construct
QQSYTTPYT

SEQ ID NO: 103 - protein, 5 aa, synthetic construct
NYGMS

SEQ ID NO: 104 - protein, 6 aa, synthetic construct
GDFLDV

SEQ ID NO: 105 - protein, 11 aa, synthetic construct
RASQGISSWLA

SEQ ID NO: 106 - protein, 7 aa, synthetic construct
GASRLQS

SEQ ID NO: 107 - protein, 9 aa, synthetic construct
QQSYLPPYT

SEQ ID NO: 108 - protein, 5 aa, synthetic construct
NYPIT

SEQ ID NO: 109 - protein, 11 aa, synthetic construct
RASQSIGTWLA

SEQ ID NO: 110 - protein, 9 aa, synthetic construct
QQSYSPPYA

SEQ ID NO: 111 - protein, 5 aa, synthetic construct
NYTIS

SEQ ID NO: 112 - protein, 7 aa, synthetic construct
AASSLHS

SEQ ID NO: 113 - protein, 5 aa, synthetic construct
NYTVT

SEQ ID NO: 114 - protein, 5 aa, synthetic construct
NYVIS

SEQ ID NO: 115 - protein, 17 aa, synthetic construct
WINPNSGGTISAPNFQG

SEQ ID NO: 116 - protein, 7 aa, synthetic construct
AASTVQS

SEQ ID NO: 117 - protein, 5 aa, synthetic construct
RHLLH

SEQ ID NO: 118 - protein, 11 aa, synthetic construct
RASQNIGSWLA

SEQ ID NO: 119 - protein, 5 aa, synthetic construct
RYIMH

SEQ ID NO: 120 - protein, 8 aa, synthetic construct
DTQSGMDV

SEQ ID NO: 121 - protein, 11 aa, synthetic construct
RASQTITRYLN

SEQ ID NO: 122 - protein, 7 aa, synthetic construct
GASTLQS

SEQ ID NO: 123 - protein, 9 aa, synthetic construct
QQSFSTPLT

SEQ ID NO: 124 - protein, 5 aa, synthetic construct
RYNMH

SEQ ID NO: 125 - protein, 8 aa, synthetic construct
DGYSGLDV

SEQ ID NO: 126 - protein, 9 aa, synthetic construct
QQSYSVPYS

SEQ ID NO: 127 - protein, 5 aa, synthetic construct
RYVMN

SEQ ID NO: 128 - protein, 11 aa, synthetic construct
RASQSIDSWLA

SEQ ID NO: 129 - protein, 5 aa, synthetic construct
SHLIH

SEQ ID NO: 130 - protein, 6 aa, synthetic construct
DYGMDV

SEQ ID NO: 131 - protein, 11 aa, synthetic construct
RASQNVNRWLA

SEQ ID NO: 132 - protein, 5 aa, synthetic construct
SYAIS

SEQ ID NO: 133 - protein, 17 aa, synthetic construct
WINPNSGGTRYAQKFQG

SEQ ID NO: 134 - protein, 11 aa, synthetic construct
ARGMTTGYMDV

SEQ ID NO: 135 - protein, 11 aa, synthetic construct
RASQDIGTWLA

SEQ ID NO: 136 - protein, 9 aa, synthetic construct
QQSYRIPYT

SEQ ID NO: 137 - protein, 17 aa, synthetic construct
WIKPDSGGTIYAQKFQG

SEQ ID NO: 138 - protein, 7 aa, synthetic construct
DGNWFDP

SEQ ID NO: 139 - protein, 11 aa, synthetic construct
RASQAISRWLA

SEQ ID NO: 140 - protein, 9 aa, synthetic construct
QESYIPPYT

SEQ ID NO: 141 - protein, 11 aa, synthetic construct
RASQSIRRWLA

SEQ ID NO: 142 - protein, 6 aa, synthetic construct
DEWLQH

SEQ ID NO: 143 - protein, 7 aa, synthetic construct
AASRLHS

SEQ ID NO: 144 - protein, 17 aa, synthetic construct
WISPNSGGTNYAQKFQG

SEQ ID NO: 145 - protein, 9 aa, synthetic construct
QQSYTSPYR

SEQ ID NO: 146 - protein, 5 aa, synthetic construct
SYDIN

SEQ ID NO: 147 - protein, 17 aa, synthetic construct
RINPYNDETHYAQKFQG

SEQ ID NO: 148 - protein, 10 aa, synthetic construct
DTSGDGRFHP

SEQ ID NO: 149 - protein, 11 aa, synthetic construct
RASQSVSRYLN

SEQ ID NO: 150 - protein, 7 aa, synthetic construct
AASSLSN

SEQ ID NO: 151 - protein, 9 aa, synthetic construct
QHNYIRPPT

SEQ ID NO: 152 - protein, 5 aa, synthetic construct
SYEIH

SEQ ID NO: 153 - protein, 8 aa, synthetic construct
EGGTKLDV

SEQ ID NO: 154 - protein, 11 aa, synthetic construct
RASQGISNYLA

SEQ ID NO: 155 - protein, 9 aa, synthetic construct
QQSYIIPLT

SEQ ID NO: 156 - protein, 5 aa, synthetic construct
SYGIS

SEQ ID NO: 157 - protein, 7 aa, synthetic construct
GSGWTDN

SEQ ID NO: 158 - protein, 11 aa, synthetic construct
RASQSVDRWLA

SEQ ID NO: 159 - protein, 7 aa, synthetic construct
AASNLQS

SEQ ID NO: 160 - protein, 17 aa, synthetic construct
WINPNSGGTNYARKFQG

SEQ ID NO: 161 - protein, 7 aa, synthetic construct
EMGFLDY

SEQ ID NO: 162 - protein, 11 aa, synthetic construct
RASQSISTWLA

SEQ ID NO: 163 - protein, 5 aa, synthetic construct
SYGIT

SEQ ID NO: 164 - protein, 5 aa, synthetic construct
SYGMH

SEQ ID NO: 165 - protein, 17 aa, synthetic construct
VISYDGSNQYYADSVKG

SEQ ID NO: 166 - protein, 14 aa, synthetic construct
TYSYGNYYYYGMDV

SEQ ID NO: 167 - protein, 11 aa, synthetic construct
RASESISTYLN

SEQ ID NO: 168 - protein, 9 aa, synthetic construct
QQSYSTPIT

SEQ ID NO: 169 - protein, 5 aa, synthetic construct
SYNMH

SEQ ID NO: 170 - protein, 8 aa, synthetic construct
EGAVAVDY

SEQ ID NO: 171 - protein, 9 aa, synthetic construct
QQGYSTPYT

SEQ ID NO: 172 - protein, 5 aa, synthetic construct
SYTLS

SEQ ID NO: 173 - protein, 8 aa, synthetic construct
DGGGGLDY

SEQ ID NO: 174 - protein, 11 aa, synthetic construct
RASQSVGRWLA

SEQ ID NO: 175 - protein, 5 aa, synthetic construct
SYVIN

SEQ ID NO: 176 - protein, 8 aa, synthetic construct
ERGGAYDY

SEQ ID NO: 177 - protein, 11 aa, synthetic construct
RASQSIGSWLA

SEQ ID NO: 178 - protein, 9 aa, synthetic construct
QQTYSPPYT

SEQ ID NO: 179 - protein, 5 aa, synthetic construct
SYVIS

SEQ ID NO: 180 - protein, 17 aa, synthetic construct
WISPDSGGTKYAQKFQG

SEQ ID NO: 181 - protein, 7 aa, synthetic construct
DSYGMDV

SEQ ID NO: 182 - protein, 5 aa, synthetic construct
SYYMH

SEQ ID NO: 183 - protein, 6 aa, synthetic construct
GEWLDY

SEQ ID NO: 184 - protein, 11 aa, synthetic construct
RASQTISSWLA

SEQ ID NO: 185 - protein, 5 aa, synthetic construct
THTIT

SEQ ID NO: 186 - protein, 17 aa, synthetic construct
WINPDSGGTNYAQKFQG

SEQ ID NO: 187 - protein, 8 aa, synthetic construct
EGYSGLDY

SEQ ID NO: 188 - protein, 11 aa, synthetic construct
RASQSISNWLA

SEQ ID NO: 189 - protein, 7 aa, synthetic construct
GASSLHS

SEQ ID NO: 190 - protein, 9 aa, synthetic construct
QQSYSVPYT

SEQ ID NO: 191 - protein, 5 aa, synthetic construct
TYLIH

SEQ ID NO: 192 - protein, 8 aa, synthetic construct
ERASGYDI

SEQ ID NO: 193 - protein, 7 aa, synthetic construct
GASVLQS

SEQ ID NO: 194 - protein, 9 aa, synthetic construct
QQSYHVPYT

SEQ ID NO: 195 - protein, 5 aa, synthetic construct
TYPLS

SEQ ID NO: 196 - protein, 11 aa, synthetic construct
RASQRIGSWLA

SEQ ID NO: 197 - protein, 9 aa, synthetic construct
QQSYSAPYS

SEQ ID NO: 198 - protein, 5 aa, synthetic construct
TYPMX

SEQ ID NO: 199 - protein, 11 aa, synthetic construct
RASQGIDRWLA

SEQ ID NO: 200 - protein, 5 aa, synthetic construct
TYYMH

SEQ ID NO: 201 - protein, 7 aa, synthetic construct
AASKLQG

SEQ ID NO: 202 - protein, 5 aa, synthetic construct
AYHVH

SEQ ID NO: 203 - protein, 5 aa, synthetic construct
AYNMH

SEQ ID NO: 204 - protein, 5 aa, synthetic construct
DYGLS

SEQ ID NO: 205 - protein, 11 aa, synthetic construct
RASQTIGPWLA

SEQ ID NO: 206 - protein, 5 aa, synthetic construct
DYHLH

SEQ ID NO: 207 - protein, 17 aa, synthetic construct
WINPNSGGTNFAQKFQG

SEQ ID NO: 208 - protein, 5 aa, synthetic construct
DYLMH

SEQ ID NO: 209 - protein, 17 aa, synthetic construct
WINPNSGGTNYAQKXQG

SEQ ID NO: 210 - skipped

SEQ ID NO: 211 - protein, 7 aa, synthetic construct
AASKLET

SEQ ID NO: 212 - protein, 7 aa, synthetic construct
AASTLQT

SEQ ID NO: 213 - protein, 5 aa, synthetic construct
DYQIH

SEQ ID NO: 214 - protein, 17 aa, synthetic construct
WISPYSGGTNYAQKFQG

SEQ ID NO: 215 - protein, 7 aa, synthetic construct
DRVGLDY

SEQ ID NO: 216 - protein, 5 aa, synthetic construct
DYTIH

SEQ ID NO: 217 - protein, 17 aa, synthetic construct
WINPNSGGTNYAQQFQG

SEQ ID NO: 218 - protein, 11 aa, synthetic construct
RASQNIRRWLA

SEQ ID NO: 219 - protein, 7 aa, synthetic construct
AASSLRS

SEQ ID NO: 220 - protein, 5 aa, synthetic construct
ENEMH

SEQ ID NO: 221 - protein, 17 aa, synthetic construct
WINPNSGGTSYAPNFQG

SEQ ID NO: 222 - protein, 6 aa, synthetic construct
ANWLDF

SEQ ID NO: 223 - protein, 5 aa, synthetic construct
FSTIH

SEQ ID NO: 224 - protein, 17 aa, synthetic construct
WISPHSGGTNYAQEFQG

SEQ ID NO: 225 - protein, 8 aa, synthetic construct
DSGSYLDY

SEQ ID NO: 226 - protein, 11 aa, synthetic construct
QASQDINNYLN

SEQ ID NO: 227 - protein, 5 aa, synthetic construct
GYAIS

SEQ ID NO: 228 - protein, 5 aa, synthetic construct
GYYLH

SEQ ID NO: 229 - protein, 17 aa, synthetic construct
WINPKSGGTNFAQKFQG

SEQ ID NO: 230 - protein, 13 aa, synthetic construct
ERGATGYYYGMDV

SEQ ID NO: 231 - protein, 11 aa, synthetic construct
RASQRVDSWLA

SEQ ID NO: 232 - protein, 7 aa, synthetic construct
KASILES

SEQ ID NO: 233 - protein, 9 aa, synthetic construct
QQTYSTPWT

SEQ ID NO: 234 - protein, 17 aa, synthetic construct
WIYPNSGGTNYAQNLQG

SEQ ID NO: 235 - protein, 5 aa, synthetic construct
DDFDY

SEQ ID NO: 236 - protein, 11 aa, synthetic construct
QASQDISNYLN

SEQ ID NO: 237 - protein, 7 aa, synthetic construct
VASTLQS

SEQ ID NO: 238 - protein, 5 aa, synthetic construct
KYGIS

SEQ ID NO: 239 - protein, 17 aa, synthetic construct
WINPYSGGTNYAQNFQG

SEQ ID NO: 240 - protein, 7 aa, synthetic construct
GDGWYDP

SEQ ID NO: 241 - protein, 5 aa, synthetic construct
NFLIN

SEQ ID NO: 242 - protein, 8 aa, synthetic construct
ERELPFDI

SEQ ID NO: 243 - protein, 11 aa, synthetic construct
RASQSISSWVA

SEQ ID NO: 244 - protein, 7 aa, synthetic construct
GASNLES

SEQ ID NO: 245 - protein, 9 aa, synthetic construct
QQTYNPPYS

SEQ ID NO: 246 - protein, 5 aa, synthetic construct
NHIMH

SEQ ID NO: 247 - protein, 17 aa, synthetic construct
WIYPDSGGTKYAQKFQG

SEQ ID NO: 248 - protein, 7 aa, synthetic construct
ATGFMDV

SEQ ID NO: 249 - protein, 9 aa, synthetic construct
QQSYNTPFT

SEQ ID NO: 250 - protein, 5 aa, synthetic construct
NYAFN

SEQ ID NO: 251 - protein, 17 aa, synthetic construct
WIYPNTGGTHYAPKFQG

SEQ ID NO: 252 - protein, 7 aa, synthetic construct
GESWFDP

SEQ ID NO: 253 - protein, 7 aa, synthetic construct
AASNLES

SEQ ID NO: 254 - protein, 17 aa, synthetic construct
WINPNTGGTNYAQSLQG

SEQ ID NO: 255 - protein, 7 aa, synthetic construct
SSGWYDY

SEQ ID NO: 256 - protein, 9 aa, synthetic construct
QQSYSPPYS

SEQ ID NO: 257 - protein, 5 aa, synthetic construct
NYGIS

SEQ ID NO: 258 - protein, 17 aa, synthetic construct
WISPDSGGTIYAQRFQG

SEQ ID NO: 259 - protein, 17 aa, synthetic construct
WINPYSGGTNYAQKFQG

SEQ ID NO: 260 - protein, 9 aa, synthetic construct
QQSYSIPYS

SEQ ID NO: 261 - protein, 5 aa, synthetic construct
NYHMH

SEQ ID NO: 262 - protein, 6 aa, synthetic construct
GAWFDP

SEQ ID NO: 263 - protein, 7 aa, synthetic construct
GASNLQS

SEQ ID NO: 264 - protein, 5 aa, synthetic construct
NYNIA

SEQ ID NO: 265 - protein, 5 aa, synthetic construct
NYPIH

SEQ ID NO: 266 - protein, 8 aa, synthetic construct
DQGAGWDY

SEQ ID NO: 267 - protein, 5 aa, synthetic construct
NYQIH

SEQ ID NO: 268 - protein, 17 aa, synthetic construct
WINPNSGGTIYAQKFQG

SEQ ID NO: 269 - protein, 17 aa, synthetic construct
WIYPNTGGTNYAQKFQG

SEQ ID NO: 270 - protein, 8 aa, synthetic construct
DRGYGMDV

SEQ ID NO: 271 - protein, 11 aa, synthetic construct
RASQDISTWLA

SEQ ID NO: 272 - protein, 8 aa, synthetic construct
QQSYSSWT

SEQ ID NO: 273 - protein, 5 aa, synthetic construct
PYTFS

SEQ ID NO: 274 - protein, 17 aa, synthetic construct
WINPSSGGAQYAQKLQG

SEQ ID NO: 275 - protein, 11 aa, synthetic construct
RASQSIRSWLA

SEQ ID NO: 276 - protein, 5 aa, synthetic construct
RHPMH

SEQ ID NO: 277 - protein, 17 aa, synthetic construct
WIHPDSGGTNYSQKFQG

SEQ ID NO: 278 - protein, 9 aa, synthetic construct
QQSYSTPYS

SEQ ID NO: 279 - protein, 5 aa, synthetic construct
SHGIT

SEQ ID NO: 280 - protein, 17 aa, synthetic construct
WINPNSGGTNYAQNVQG

SEQ ID NO: 281 - protein, 6 aa, synthetic construct
GPWLDY

SEQ ID NO: 282 - protein, 6 aa, synthetic construct
GIFEDY

SEQ ID NO: 283 - protein, 7 aa, synthetic construct
ATSRLQS

SEQ ID NO: 284 - protein, 9 aa, synthetic construct
QQSYGTPYT

SEQ ID NO: 285 - protein, 11 aa, synthetic construct
RASQSIDRWLT

SEQ ID NO: 286 - protein, 9 aa, synthetic construct
QQSYRTPYT

SEQ ID NO: 287 - protein, 17 aa, synthetic construct
WINPNSGGTKYAPKFQG

SEQ ID NO: 288 - protein, 6 aa, synthetic construct
GDWYDY

SEQ ID NO: 289 - protein, 17 aa, synthetic construct
WINPNSGGTNYAQSFQG

SEQ ID NO: 290 - protein, 9 aa, synthetic construct
QQSYSAPLT

SEQ ID NO: 291 - protein, 5 aa, synthetic construct
SYAMH

SEQ ID NO: 292 - protein, 17 aa, synthetic construct
GISGNGGSTNYADSVKG

SEQ ID NO: 293 - protein, 13 aa, synthetic construct
DAYYYGSGSYFDY

SEQ ID NO: 294 - protein, 7 aa, synthetic construct
DYSWFDP

SEQ ID NO: 295 - protein, 11 aa, synthetic construct
RASQDISNWLA

SEQ ID NO: 296 - protein, 7 aa, synthetic construct
ATSSLQS

SEQ ID NO: 297 - protein, 17 aa, synthetic construct
WINPNSGGTNYAQRLQG

SEQ ID NO: 298 - protein, 7 aa, synthetic construct
DFSWFDP

SEQ ID NO: 299 - protein, 7 aa, synthetic construct
GASNLQP

SEQ ID NO: 300 - protein, 7 aa, synthetic construct
GYNWFDP

SEQ ID NO: 301 - protein, 7 aa, synthetic construct
SINWFDP

SEQ ID NO: 302 - protein, 11 aa, synthetic construct
RASQSVGRWMA

SEQ ID NO: 303 - protein, 17 aa, synthetic construct
WINPHSGGTFYAQKFQG

SEQ ID NO: 304 - protein, 5 aa, synthetic construct
SYLMH

SEQ ID NO: 305 - protein, 8 aa, synthetic construct
DRGSGYDY

SEQ ID NO: 306 - protein, 11 aa, synthetic construct
RASQTIGNWLA

SEQ ID NO: 307 - protein, 9 aa, synthetic construct
QQSYSPPLT

SEQ ID NO: 308 - protein, 5 aa, synthetic construct
SYPIH

SEQ ID NO: 309 - protein, 5 aa, synthetic construct
SYPIS

SEQ ID NO: 310 - protein, 17 aa, synthetic construct
WINPSNGGTNYAQKFQG

SEQ ID NO: 311 - protein, 6 aa, synthetic construct
SNWLDY

SEQ ID NO: 312 - protein, 5 aa, synthetic construct
SYTIS

SEQ ID NO: 313 - protein, 11 aa, synthetic construct
RTSQGIGTWLA

SEQ ID NO: 314 - protein, 5 aa, synthetic construct
TFPMH

SEQ ID NO: 315 - protein, 9 aa, synthetic construct
QQSYNPPYT

SEQ ID NO: 316 - protein, 5 aa, synthetic construct
TYGIS

SEQ ID NO: 317 - protein, 11 aa, synthetic construct
RASQDISRWSA

SEQ ID NO: 318 - protein, 5 aa, synthetic construct
TYNIH

SEQ ID NO: 319 - protein, 7 aa, synthetic construct
AASNLET

SEQ ID NO: 320 - protein, 5 aa, synthetic construct
TYTVS

SEQ ID NO: 321 - protein, 11 aa, synthetic construct
RASQGISRWLA

SEQ ID NO: 322 - protein, 9 aa, synthetic construct
QQSYETPYT

SEQ ID NO: 323 - protein, 5 aa, synthetic construct
AYYMH

SEQ ID NO: 324 - protein, 6 aa, synthetic construct
GDWNDW

SEQ ID NO: 325 - protein, 17 aa, synthetic construct
WINPNSGGTNYAQMFQG

SEQ ID NO: 326 - protein, 5 aa, synthetic construct
DYIVH

SEQ ID NO: 327 - protein, 17 aa, synthetic construct
WINPYSGGTKYAQKFQG

SEQ ID NO: 328 - protein, 8 aa, synthetic construct
VLQEGMDV

SEQ ID NO: 329 - protein, 7 aa, synthetic construct
GVSSLQS

SEQ ID NO: 330 - protein, 8 aa, synthetic construct
QQSYSNPT

SEQ ID NO: 331 - protein, 5 aa, synthetic construct
DYLIH

SEQ ID NO: 332 - protein, 17 aa, synthetic construct
WINPASGGTNYAQKFQG

SEQ ID NO: 333 - protein, 8 aa, synthetic construct
GYRWLHDY

SEQ ID NO: 334 - protein, 17 aa, synthetic construct
WINPNSGGTNYAQRFQG

SEQ ID NO: 335 - protein, 11 aa, synthetic construct
RASQDISLWLA

SEQ ID NO: 336 - protein, 9 aa, synthetic construct
QQSYMTPRT

SEQ ID NO: 337 - protein, 5 aa, synthetic construct
DYQMF

SEQ ID NO: 338 - protein, 9 aa, synthetic construct
GGGGYGMDV

SEQ ID NO: 339 - protein, 9 aa, synthetic construct
QQSYSDQWT

SEQ ID NO: 340 - protein, 5 aa, synthetic construct
FYAIS

SEQ ID NO: 341 - protein, 17 aa, synthetic construct
WMNPNSGGTTYAQKFQG

SEQ ID NO: 342 - protein, 10 aa, synthetic construct
GVQDYYGSDY

SEQ ID NO: 343 - protein, 9 aa, synthetic construct
QQGYSSPYT

SEQ ID NO: 344 - protein, 5 aa, synthetic construct
GYNIH

SEQ ID NO: 345 - protein, 8 aa, synthetic construct
DEAGYFDL

SEQ ID NO: 346 - protein, 7 aa, synthetic construct
AASTLES

SEQ ID NO: 347 - protein, 9 aa, synthetic construct
QQSFTIPWT

SEQ ID NO: 348 - protein, 5 aa, synthetic construct
GYNVH

SEQ ID NO: 349 - protein, 8 aa, synthetic construct
DHSSGYDY

SEQ ID NO: 350 - protein, 5 aa, synthetic construct
GYYIH

SEQ ID NO: 351 - protein, 8 aa, synthetic construct
ENYFDXXX

SEQ ID NO: 352 - protein, 11 aa, synthetic construct
RASQSISRWLT

SEQ ID NO: 353 - protein, 9 aa, synthetic construct
QQSYSPPXT

SEQ ID NO: 354 - protein, 5 aa, synthetic construct
GYYIN

SEQ ID NO: 355 - protein, 9 aa, synthetic construct
QQSMIAPYT

SEQ ID NO: 356 - protein, 17 aa, synthetic construct
GSEIIDYDSSGLGAFDI

SEQ ID NO: 357 - protein, 11 aa, synthetic construct
QASQSIYNYLN

SEQ ID NO: 358 - protein, 9 aa, synthetic construct
QQSYSVPHT

SEQ ID NO: 359 - protein, 5 aa, synthetic construct
NSIIN

SEQ ID NO: 360 - protein, 8 aa, synthetic construct
RWMDAFDI

SEQ ID NO: 361 - protein, 7 aa, synthetic construct
AASTLQG

SEQ ID NO: 362 - protein, 9 aa, synthetic construct
QQTYSVPLT

SEQ ID NO: 363 - protein, 5 aa, synthetic construct
NYGIT

SEQ ID NO: 364 - protein, 17 aa, synthetic construct
WINANSGGTNYAQKFQG

SEQ ID NO: 365 - protein, 9 aa, synthetic construct
QQSYSAPYT

SEQ ID NO: 366 - protein, 5 aa, synthetic construct
NYILS

SEQ ID NO: 367 - protein, 7 aa, synthetic construct
GSYGMDV

SEQ ID NO: 368 - protein, 5 aa, synthetic construct
NYNIH

SEQ ID NO: 369 - protein, 8 aa, synthetic construct
EGLAALDY

SEQ ID NO: 370 - protein, 5 aa, synthetic construct
NYPMT

SEQ ID NO: 371 - protein, 8 aa, synthetic construct
DAAAGFDP

SEQ ID NO: 372 - protein, 7 aa, synthetic construct
AASKLQS

SEQ ID NO: 373 - protein, 8 aa, synthetic construct
EGRSSYDY

SEQ ID NO: 374 - protein, 7 aa, synthetic construct
GASSVQS

SEQ ID NO: 375 - protein, 5 aa, synthetic construct
NYYMH

SEQ ID NO: 376 - protein, 17 aa, synthetic construct
WMNPNSGHTSYAQKFQG

SEQ ID NO: 377 - protein, 12 aa, synthetic construct
VGYSSGWLGMDV

SEQ ID NO: 378 - protein, 11 aa, synthetic construct
QASQDIRKYLN

SEQ ID NO: 379 - protein, 9 aa, synthetic construct
QQSYTIPYT

SEQ ID NO: 380 - protein, 17 aa, synthetic construct
WINPNTGGTNYAQKFQG

SEQ ID NO: 381 - protein, 5 aa, synthetic construct
SSDIN

SEQ ID NO: 382 - protein, 17 aa, synthetic construct
GIIPIFGTANYAQKFQG

SEQ ID NO: 383 - protein, 15 aa, synthetic construct
ADTAMGHYYYYGMDV

SEQ ID NO: 384 - protein, 16 aa, synthetic construct
RSSQSLLNKNGNNYID

SEQ ID NO: 385 - protein, 7 aa, synthetic construct
QGSNRAS

SEQ ID NO: 386 - protein, 9 aa, synthetic construct
MQALQTPPT

SEQ ID NO: 387 - protein, 5 aa, synthetic construct
SYAFS

SEQ ID NO: 388 - protein, 17 aa, synthetic construct
VIYPSGGTTGYAQKFQG

SEQ ID NO: 389 - protein, 11 aa, synthetic construct
GLHQLPNWFDP

SEQ ID NO: 390 - protein, 11 aa, synthetic construct
DLVGGVFAFDI

SEQ ID NO: 391 - protein, 11 aa, synthetic construct
RASQNITTYLN

SEQ ID NO: 392 - protein, 9 aa, synthetic construct
QQSYSMPYS

SEQ ID NO: 393 - protein, 17 aa, synthetic construct
GIIPIFGAPVYAQDFQG

SEQ ID NO: 394 - protein, 9 aa, synthetic construct
DRGADAFDI

SEQ ID NO: 395 - protein, 17 aa, synthetic construct
KSSQSVLYSSNNKNYLA

SEQ ID NO: 396 - protein, 7 aa, synthetic construct
WASTRAS

SEQ ID NO: 397 - protein, 9 aa, synthetic construct
QQYERTPLT

SEQ ID NO: 398 - protein, 17 aa, synthetic construct
WINPNSGGTKYAQKFQG

SEQ ID NO: 399 - protein, 7 aa, synthetic construct
EISFLDY

SEQ ID NO: 400 - protein, 5 aa, synthetic construct
SYAVS

SEQ ID NO: 401 - protein, 17 aa, synthetic construct
WIIPDSGGTNYAQKLQG

SEQ ID NO: 402 - protein, 7 aa, synthetic construct
GSGWYDY

SEQ ID NO: 403 - protein, 8 aa, synthetic construct
QQSYSKPT

SEQ ID NO: 404 - protein, 5 aa, synthetic construct
SYGIF

SEQ ID NO: 405 - protein, 11 aa, synthetic construct
RASQDIGSWLA

SEQ ID NO: 406 - protein, 8 aa, synthetic construct
QQSYSMIT

SEQ ID NO: 407 - protein, 7 aa, synthetic construct
DFDAFDI

SEQ ID NO: 408 - protein, 11 aa, synthetic construct
RASEAIGTWLA

SEQ ID NO: 409 - protein, 9 aa, synthetic construct
STDYYYMDV

SEQ ID NO: 410 - protein, 11 aa, synthetic construct
RASQNIRGYLN

SEQ ID NO: 411 - protein, 9 aa, synthetic construct
QQSYSNPWT

SEQ ID NO: 412 - protein, 7 aa, synthetic construct
NYDSYDY

SEQ ID NO: 413 - protein, 11 aa, synthetic construct
RASQSVGSWLA

SEQ ID NO: 414 - protein, 7 aa, synthetic construct
GASTLQD

SEQ ID NO: 415 - protein, 17 aa, synthetic construct
WLNPNSGGTNYAQQFQG

SEQ ID NO: 416 - protein, 11 aa, synthetic construct
RASQRIGNWLA

SEQ ID NO: 417 - protein, 9 aa, synthetic construct
LQDYNYPYT

SEQ ID NO: 418 - protein, 7 aa, synthetic construct
DYGALDY

SEQ ID NO: 419 - protein, 11 aa, synthetic construct
RASQAIGSWLA

SEQ ID NO: 420 - protein, 17 aa, synthetic construct
WIIPNSGGTNYAQKFQG

SEQ ID NO: 421 - protein, 7 aa, synthetic construct
GSGWSDY

SEQ ID NO: 422 - protein, 11 aa, synthetic construct
RASQNIGRWLA

SEQ ID NO: 423 - protein, 9 aa, synthetic construct
RQSYSPPYT

SEQ ID NO: 424 - protein, 7 aa, synthetic construct
GASNLQT

SEQ ID NO: 425 - protein, 7 aa, synthetic construct
ATSTLQS

SEQ ID NO: 426 - protein, 9 aa, synthetic construct
QQSYSIPYA

SEQ ID NO: 427 - protein, 11 aa, synthetic construct
RASQTISRWLA

SEQ ID NO: 428 - protein, 17 aa, synthetic construct
WINPNNGGTNYAQKFQG

SEQ ID NO: 429 - protein, 8 aa, synthetic construct
DSGVGMDV

SEQ ID NO: 430 - protein, 11 aa, synthetic construct
RASQSISKWLA

SEQ ID NO: 431 - protein, 7 aa, synthetic construct
DSAGMDV

SEQ ID NO: 432 - protein, 5 aa, synthetic construct
SYVIT

SEQ ID NO: 433 - protein, 17 aa, synthetic construct
WINPNSGGTNYAQEFQG

SEQ ID NO: 434 - protein, 7 aa, synthetic construct
GHMALDY

SEQ ID NO: 435 - protein, 11 aa, synthetic construct
RASQNINTWLA

SEQ ID NO: 436 - protein, 9 aa, synthetic construct
QQSYIAPYT

SEQ ID NO: 437 - protein, 5 aa, synthetic construct
SYVMN

SEQ ID NO: 438 - protein, 17 aa, synthetic construct
WINPNSGGTSYAQKFQG

SEQ ID NO: 439 - protein, 5 aa, synthetic construct
SYYLH

SEQ ID NO: 440 - protein, 17 aa, synthetic construct
IINPSGGSTSYAQKFQG

SEQ ID NO: 441 - protein, 16 aa, synthetic construct
DTEGYGSGSRPYGMDV

SEQ ID NO: 442 - protein, 9 aa, synthetic construct
QQAYSFPYT

SEQ ID NO: 443 - protein, 5 aa, synthetic construct
TYDIS

SEQ ID NO: 444 - protein, 17 aa, synthetic construct
WMSPNSGGTNYAQNFQG

SEQ ID NO: 445 - protein, 11 aa, synthetic construct
SLHPYSNYWVY

SEQ ID NO: 446 - protein, 7 aa, synthetic construct
AASSLRG

SEQ ID NO: 447 - protein, 9 aa, synthetic construct
QQSYNTPYS

SEQ ID NO: 448 - protein, 5 aa, synthetic construct
TYTIS

SEQ ID NO: 449 - protein, 6 aa, synthetic construct
GDWQDI

SEQ ID NO: 450 - protein, 5 aa, synthetic construct
TYTIT

SEQ ID NO: 451 - protein, 17 aa, synthetic construct
WINPKSGGTNYAQKFQG

SEQ ID NO: 452 - protein, 9 aa, synthetic construct
QQSYGTPWT

SEQ ID NO: 453 - protein, 5 aa, synthetic construct
TYYVH

SEQ ID NO: 454 - protein, 17 aa, synthetic construct
RISPNSGGTNYAQKFQG

SEQ ID NO: 455 - protein, 6 aa, synthetic construct
DWSMDV

SEQ ID NO: 456 - protein, 8 aa, synthetic construct
QQSYSTWT

SEQ ID NO: 457 - protein, 5 aa, synthetic construct
XHLIH

SEQ ID NO: 458 - protein, 11 aa, synthetic construct
RASQXVNRWLA

SEQ ID NO: 459 - protein, 115 aa, synthetic construct
QVQLVQSGAEVKKPGASVKVSCKASGYTFIDYNINWVRQAPGQGLEWMGWINPNSGGTNYAQKLQGRVTMTRDTSTSTVYMELSSLRSEDTAVYYCARANWLDYWGQGTLVTVSS

SEQ ID NO: 460 - protein, 107 aa, synthetic construct
DIQMTQSPSSLSASVGDRVTITCRASQDINRWLAWYQQKPGKAPKLLIYAASTLQSGVPSRFSGSGSGTDFTLTISSLQPEDFATYYCQQSYGSPYTFGQGTKVEIK

SEQ ID NO: 461 - protein, 115 aa, synthetic construct
QVQLVQSGAEVKKPGASVKVSCKASGYTFTDYYLHWVRQAPGQGLEWMGWMNPNSGGTNYAQKFQGRVTMTLDTSTSTVYMELSSLRSEDTAVYYCARGEWFDPWGQGTLVTVSS

SEQ ID NO: 462 - protein, 107 aa, synthetic construct
DIQMTQSPSSLSASVGDRVTITCRASQSISRWLAWYQQKPGKAPKLLIYAASTLQSGVPSRFSGSGSGTDFTLTISSLQPEDFATYYCQQSYSTPYTFGQGTRLEIK

SEQ ID NO: 463 - protein, 115 aa, synthetic construct
QVQLVQSGAEVKKPGASVKVSCKASGYTFTDYYMHWVRQAPGQGLEWMGWIFPNSGGTNYAQKFQGRVTMTRDTSTNTVYMELSSLRSEDTAVYYCAREDWCXPWGRGTLVTVSS

SEQ ID NO: 464 - protein, 107 aa, synthetic construct
DIQMTQXPSSLXASVGDRVTITCRASQDISRWLAWYQQKPGKAXKLLXYAASXLQSGVPSRFSGSGXGTDFTLTISSLQPEDFATYYCQQSYSSPYTFGQGTKVEIK

SEQ ID NO: 465 - protein, 123 aa, synthetic construct
QVQLVQSGAEVKKPGASVKVSCKASGYSFTGKYMHWVRQAPGQGLEWMGWMNPNSGNTGYAQKFQGRVTMTRDTSTSTVYMELSSLRSEDTAVYYCARGGYYGSGSWTYFDLWGRGTLVTVSS

SEQ ID NO: 466 - protein, 107 aa, synthetic construct
DIQMTQSPSSLSASVGDRVTITCRASQSISSYLNWYQQKPGKAPKLLIYDASNLETGVPSRFSGSGSGTDFTLTISSLQPEDFATYYCQQTDSIPITFGQGTKVEIK

SEQ ID NO: 467 - protein, 116 aa, synthetic construct
QVQLVQSGAEVKKPGSSVKVSCKASGYTFTGYLMHWVRQAPGQGLEWMGWINPNSGGTNYAPKFQGRVTMTRDTSTSTVYMELSSLRSEDTAVYYCALESSGWGNWGQGTLVTVSS

SEQ ID NO: 468 - protein, 107 aa, synthetic construct
DIQMTQSPSSLSASVGDRVTITCRASQGIGRWLAWYQQKPGKAPKLLIYGASSLQSGVPSRFSGSGSGTDFTLTISSLQPEDFATYYCQQSYITPYTFGQGTKLEIK

SEQ ID NO: 469 - protein, 115 aa, synthetic construct
QVQLVQSGAEVKKPGASVKVSCKASGYTFIGYNMHWVRQAPGQGLEWMGWINPNTGGTSYAQKFQGRVTMTRDTSTSTVYMELSSLRSEDTAVYYCARANWLDYWGQGTLVTVSS

SEQ ID NO: 470 - protein, 107 aa, synthetic construct
DIQMTQSPSSLSASVGDRVTITCRAXQXIXRWLAWYQQKPGKAPKLLIYAASTLQSGVPSRFSGSGSGTDFTLTISSLQPEDFATYYCQQSYSIPITFGQGTKVEIK

SEQ ID NO: 471 - protein, 115 aa, synthetic construct
QVQLVQSGAEVKKPGASVKVSCKASGYTFTGYNMHWVRQAPGQGLEWMGWINPNSGGTNYAQKFQGRVTMTRDTSTSTVYMELSSLRSEDTAVYYCARANWLDYWGQGTLVTVSS

SEQ ID NO: 472 - protein, 106 aa, synthetic construct
DIQMTQSPSSLSASVGDRVTITCRASQSIGRWLAWYQQKPGKAPKLLIYAASRLQSGVPSRFSGSGSGTDFTLTISSLQPEDFATYYCQQSYGVPTFGQGTKVEIK

SEQ ID NO: 473 - protein, 107 aa, synthetic construct
DIQMTQSPSSLSASVGDRVTITCRASQSINRWLAWYQQKPGKAPKLLIYAASTLQSGVPSRFSGSGSGTDFTLTISSLQPEDFATYYCQQSYITPYTFGQGTKLEIK

SEQ ID NO: 474 - protein, 117 aa, synthetic construct
EVQLVQSGAEVKKPGSSVKVSCKASGYTFTGYQMHWVRQAPGQGLEWMGWINPNSGGTNYAQKFQGRVTMTRDTSTSTVYMEVSSLRSEDTAVYYCARDGGSYLDYWGQGTLVTVSS

SEQ ID NO: 475 - protein, 107 aa, synthetic construct
DIQMTQSPSSLSASVGDRVTITCRASQSINSWLAWYQQKPGKAPKLLIYAASTLQSGVPSRFSGSGSGTDFTLTISSLQPEDFATYYCQQSYSIPYNFGQGTKLEIK

SEQ ID NO: 476 - protein, 115 aa, synthetic construct
QVQLVQSGAEVKKPGASVKVSCKASGYTFTGYYMHWVRQAPGQGLEWMGRIFPNSGGTDYAQKFQGRVTMTRDTSTNTVYMELSSLRSEDTAVYYCARGDWFDPWGQGTLVTVSS

SEQ ID NO: 477 - protein, 107 aa, synthetic construct
DIQMTQSPSSLSASVGDRVTITCRASQDISRWLAWYQQKPGKAPKLLIYAASTLQSGVPSRFSGSGSGTDFTLTISSLQPEDFATYYCQQSYSSPYTFGQGTKVEIK

SEQ ID NO: 478 - protein, 116 aa, synthetic construct
QVQLVQSGAEVKKPGASVKVSCKASGYTLTHYGMSWVRQAPGQGLEWMGWINPNSGGTNYAQKFQGRVTMTRDTSTSTVYMELSSLRSEDTAVYYCARDLGXYDYWGQGTLVTVSS

SEQ ID NO: 479 - protein, 106 aa, synthetic construct
DIQMTQSPSSLSASVGDRVTITCRASQSISSWLAWYQQKPGKAPKLLIYAASSLQSGVPSRFSGSGSGTDFTLTISSLQPEDFATYYCQQSYSYPTFGQGTKVDIK

SEQ ID NO: 480 - protein, 115 aa, synthetic construct
QVQLVQSGAEVKKPGASVKVSCKASGYNFNKYDIAWVRQAPGQGLEWMGWIYPNSGGTNYAQKFQGRVTMTRDTSTSTVYMELSSLRSEDTAVYYCARANWLDYWGQGTLVTVSS

SEQ ID NO: 481 - protein, 107 aa, synthetic construct
DIQMTQSPSSLSASVGDRVTITCRASQSVSSWLAWYQQKPGKAPKLLIYAASTLHSGVPSRFSGSGSGTDFTLTISSLQPEDFATYYCQQSYSTPYTFGQGTKLEIK

SEQ ID NO: 482 - protein, 115 aa, synthetic construct
EVQLLESGAEVKKPGSSVKVSCKASGYTFKNFGISWVRQAPGQGLEWMGWINPNSGGTNYAQKFQGRVTMTRDTSTSTVYMELSSLRSEDTAVYYCARANWLDYWGQGTLVTVSS

SEQ ID NO: 483 - protein, 107 aa, synthetic construct
DIQMTQSPSSLSASVGDRVTITCRASQSISRWLAWYQQKPGKAXKLXIYAASSLQSGVPSRFSGSGSGTDFTLTISSLQPEDFATYYCQQSYSTPYTFGQGTKLEIK

SEQ ID NO: 484 - protein, 115 aa, synthetic construct
QVQLVQSGAEVKKPGASVKVSCKASGYTFTNFPMHWVRQAPGQGLEWMGWINPNSGGTNYAQKFQGRVTMTRDTSTSTVYMELSSLRSEDTAVYYCARGEWLGYWGQGTLVTVSS

SEQ ID NO: 485 - protein, 107 aa, synthetic construct
DIQMTQSPSSLSASVGDRVTITCRASQSVSNWLAWYQQKPGKAPKLLIYAASSLQSGVPSRFSGSGSGTDFTLTISSLQPEDFATYYCQQSYSTPYTFGQGTKVEIK

SEQ ID NO: 486 - protein, 115 aa, synthetic construct
QVQLVQSGAEVKKPGASVKVSCKASGYTFTNFPMXWVRQAPGQGLEWMGWINPNSGGTNYAQNFQGRVTMTRDTSTSTVYMELSSLRSEDTAVYYCARGEWLGYWGRGTLXTVSS

SEQ ID NO: 487 - protein, 107 aa, synthetic construct
DIQMTQSPSSLSASVGDRVTITCRASQSVSXWLAWYQQKPGKAPKLLIYAASSLQSGVPSRFSGSGSGTDFTLTISSLQPEDFATYYCQQSYSTPYTFGQGTKVEIK

SEQ ID NO: 488 - protein, 116 aa, synthetic construct
QVQLVQSGAEVKKPGASVKVSCKASGGTFGNFVVSWVRQAPGQGLEWMGWINPNSGGTNYAQKFQGRVTMTRDTSTSTVYMELSSLRSEDTAVYYCANSVGWFDPWGQGTLVTVSS

SEQ ID NO: 489 - protein, 107 aa, synthetic construct
DIQMTQSPSSLSASVGDRVTITCRASESIGRWLAWYQQKPGKAPKLLIYAASTLQSGVPSRFSGSGSGTDFTLTISSLQPEDFATYYCQQSYISPYTFGQGTKLEIK

SEQ ID NO: 490 - protein, 115 aa, synthetic construct
EVQLLESGAEVKKPGASVKVSCKASGFTFTNHNMHWVRQAPGQGLEWMGWINPNSGGTNYAQKFQGRVTMTRDTSTSTVYMELSSLRSEDTAVYYCARANWLDYWGQGTLVTVSS

SEQ ID NO: 491 - protein, 107 aa, synthetic construct
DIQMTQSPSSLSASVGDRVTITCRASQSIDRWLAWYQQKPGKAPKLLIYAASSLQSGVPSRFSGSGSGTDFTLTISSLQPEDFATYYCQQSYSIPYTFGQGTKLEIK

SEQ ID NO: 492 - protein, 122 aa, synthetic construct
EVQLLESGGGLVQPGGSLRLSCAASGFTFSNSAIHWVRQAPGKGLEWVSYISGSGDTMFYADSVKGRFTISRDNSKNTLYLQMNSLRAEDTAVYYCARDPYYYGSGSYFDYWGQGTLVTVSS

SEQ ID NO: 493 - protein, 107 aa, synthetic construct
DIQMTQSPSSLSASVGDRVTITCRASQSISSYLNWYQQKPGKAPKLLIKYASQSISGVPSRFSGSGSGTDFTLTISSLQPEDFATYYCQQSYSTPYTFGQGTKVEIK

SEQ ID NO: 494 - protein, 117 aa, synthetic construct
QVQLVQSGAEVKKPGASVKVSCKASGGTFSNSVISWVRQAPGQGLEWMGWISVYNGETNYAQKLQGRVTMTRDTSTSTVYMELSSLRSEDTAVYYCARDRIDGMDVWGQGTTVTVSS

SEQ ID NO: 495 - protein, 107 aa, synthetic construct
DIQMTQSPSSLSASVGDRVTITCRASQRISYWVAWYQQKPGKAPKLLIYDASTLQSGVPSRFSGSGSGTDFTLTISSLQPEDFATYYCQQSYSTPLTFGGGTKVEIK

SEQ ID NO: 496 - protein, 115 aa, synthetic construct
QVQLVQSGAEVKKPGASVKVSCKASGGTFSNYAFSWVRQAPGQGLEWMGWINPNSGGTNYAQKFQGRVTMTRDTSTSTVYMELSSLRSEDTAVYYCARGDWLDYWGQGTLVTVSS

SEQ ID NO: 497 - protein, 107 aa, synthetic construct
DIQMTQSPSSLSASVGDRVTITCRASQSISSWLAWYQQKPGKAPKLLIYAASSLQSGVPSRFSGSGSGTDFTLTISSLQPEDFATYYCQQSYNTPYTFGQGTKLEIK

SEQ ID NO: 498 - protein, 115 aa, synthetic construct
QVQLVQSGAEVKKPGASVKVSCKASGGTFSNYAISWVRQAPGQGLEWMGWINPNSGGTNYAQKFQGRVTMTRDTSTSTVYMELSSLRSEDTAVYYCARANWLDYWGQGTLVTVSS

SEQ ID NO: 499 - protein, 107 aa, synthetic construct
DIQMTQSPSSLSASVGDRVTITCRASQNINRWLAWYQQKPGKAPKLLIYAASTLQSGVPSRFSGSGSGTDFTLTISSLQPEDFATYYCQQSYSPPYTFGQGTKLEIK

SEQ ID NO: 500 - protein, 115 aa, synthetic construct
QVQLVQSGAEVKKPGASVKVSCKASGYTFTNYDINWVRQAPGQGLEWMGWINPNSGGTNYAQKFQGRVTMTRDTSTSTVYMELSSLRSEDTAVYYCARGEWLSVWGQGTLVTVSS

SEQ ID NO: 501 - protein, 107 aa, synthetic construct
DIQMTQSPSSLSASVGDRVTITCRASQEIRRWLAWYQQKPGKAPKLLIYAASSLQSGVPSRFSGSGSGTDFTLTISSLQPEDFATYYCQQSYTTPYTFGQGTKLEIK

SEQ ID NO: 502 - protein, 115 aa, synthetic construct
QVQLVQSGAEVKKPGASVKVSCKASGGTFSNYGMSWVRQAPGQGLEWMGWINPNSGGTNYAQKFQGRVTMTRDTSTSTVYMELSSLRSEDTAVYYCALGDFLDVWGQGTTVTVSS

SEQ ID NO: 503 - protein, 107 aa, synthetic construct
DIQMTQSPSSLSASVGDRVTITCRASQGISSWLAWYQQKPGKAPKLLIYGASRLQSGVPSRFSGSGSGTDFTLTISSLQPEDFATYYCQQSYLPPYTFGQGTKLEIK

SEQ ID NO: 504 - protein, 115 aa, synthetic construct
QVQLVQSGAEVKKPGASVKVSCKASGGTFSNYPITWVRQAPGQGLEWMGWINPNSGGTNYAQKFQGRVTMTRDTSTSTVYMELSSLRSEDTAVYYCARANWLDYWGQGTLVTVSS

SEQ ID NO: 505 - protein, 107 aa, synthetic construct
DIQMTQSPSSLSASVGDRVTITCRASQSIGTWLAWYQQKPGKAPKLLIYAASSLQSGVPSRFSGSGSGTDFTLTISSLQPEDFATYYCQQSYSPPYAFGQGTKLEIK

SEQ ID NO: 506 - protein, 115 aa, synthetic construct
QVQLVQSGAEVKKPGSSVKVSCKASGGTFSNYTISWVRQAPGQGLEWMGWINPNSGGTNYAQKFQGRVTMTRDTSTSTVYMELSSLRSEDTAVYYCARANWLDYWGQGTLVTVSS

SEQ ID NO: 507 - protein, 107 aa, synthetic construct
DIQMTQSPSSLSASVGDRVTITCRASQSISSWLAWYQQKPGKAPKLLIYAASSLHSGVPSRFSGSGSGTDFTLTISSLQPEDFATYYCQQSYSPPYTFGQGTKLEIK

SEQ ID NO: 508 - protein, 115 aa, synthetic construct
QVQLVQSGAEVKKPGASVKVSCKASGGTFNNYTVTWVRQAPGQGLEWMGWINPNSGGTNYAQKLQGRVTMTRDTSTSTVYMELSSLRSEDTAVYYCARANWLDYWGQGTLVTVSS

SEQ ID NO: 509 - protein, 107 aa, synthetic construct
DIQMTQSPSSLSASVGDRVTITCRASQSISRWLAWYQQKPGKAPKLLIYAASTLQSGVPSRFSGSGSGTDFTLTISSLQPEDFATYYCQQSYITPYTFGQGTKLEIK

SEQ ID NO: 510 - protein, 115 aa, synthetic construct
QVQLVQSGAEVKKPGASVKVSCKASGGTFSNYVISWVRQAPGQGLEWMGWINPNSGGTISAPNFQGRVTMTRDTSTSTVYMELSSLRSEDTAVYYCARANWLDYWGQGTLVTVSS

SEQ ID NO: 511 - protein, 107 aa, synthetic construct
DIQMTQSPSSLSASVGDRVTITCRASQSISRWLAWYQQKPGKAPKLLIYAASTVQSGVPSRFSGSGSGTDFTLTISSLQPEDFATYYCQQSYSTPYTFGQGTKLEIK

SEQ ID NO: 512 - protein, 115 aa, synthetic construct
EVQLLESGAEVKKPGASVKVSCKASGYTFTRHLLHWVRQAPGQGLEWMGWINPNSGGTNYAQKFQGRVTMTRDTSTSTVYMELSSLRSEDTAVYYCARANWLDYWGQGTLVTVSS

SEQ ID NO: 513 - protein, 107 aa, synthetic construct
DIQMTQSPSSLSASVGDRVTITCRASQNIGSWLAWYQQKPGKAPKLLXYAASSLQSGVPSRFSGSGSGTDFTLTISSLQPEDFATYYCQQSYSTPYTFGQGTKVEIK

SEQ ID NO: 514 - protein, 117 aa, synthetic construct
QVQLVQSGAEVKKPGASVKVSCKASGYTLTRYIMHWVRQAPGQGLEWMGWINPNSGGTNYAQKFQGRVTMTRDTSTSTVYMELSSLRSEDTAVYYCAKDTQSGMDVWGQGTTVTVSS

SEQ ID NO: 515 - protein, 107 aa, synthetic construct
DIQMTQSPSSLSASVGDRVTITCRASQTITRYLNWYQQKPGKAPKLLIYGASTLQSGVPSRFSGSGSGTDFTLTISSLQPEDFATYYCQQSFSTPLTFGQGTKVEIK

SEQ ID NO: 516 - protein, 117 aa, synthetic construct
QVQLVQSGAEVKKPGASVKVSCKASGYSFTRYNMHWVRQAPGQGLEWMGWINPNSGGTNYAQKFQGRVTMTRDTSTSTVYMELSSLRSEDTAVYYCARDGYSGLDVWGKGTTVTVSS

SEQ ID NO: 517 - protein, 107 aa, synthetic construct
DIQMTQSPSSLSASVGDRVTITCRASQSISSWLAWYQQKPGKAPKLLIYGASSLQSGVPSRFSGSGSGTDFTLTISSLQPEDFATYYCQQSYSVPYSFGQGTKLEIK

SEQ ID NO: 518 - protein, 115 aa, synthetic construct
QVQLVQSGAEVKKPGASVKVSCKASGDTFNRYVMNWVRQAPGQGLEWMGWINPNSGGTNYAQKFQGRVTMTRDTSTSTVYMELSSLRSEDTAVYYCARANWLDYWGQGTLVTVSS

SEQ ID NO: 519 - protein, 107 aa, synthetic construct
DIQMTQSPSSLSASVGDRVTITCRASQSIDSWLAWYQQKPGKAPKLLIYAASTLQSGVPSRFSGSGSGTDFTLTISSLQPEDFATYYCQQSYSTPYTFGQGTKLEIK

SEQ ID NO: 520 - protein, 115 aa, synthetic construct
QVQLVQSGAEVKKPGASVKVSCKASGYTLTSHLIHWVRQAPGQGLEWMGWINPNSGGTNYAQKLQGRVTMTRDTSTSTVYMELSSLRSEDTAVYYCARDYGMDVWGQGTTVTVSS

SEQ ID NO: 521 - protein, 107 aa, synthetic construct
DIQMTQSPSSLSASVGDRVTITCRASQNVNRWLAWYQQKPGKAPKLLIYAASSLQSGVPSRFSGSGSGTDFTLTISSLQPEDFATYYCQQSYITPYTFGQGTKLEIK

SEQ ID NO: 522 - protein, 120 aa, synthetic construct
QVQLVQSGAEVKKPGASVKVSCKASGGTFSSYAISWVRQAPGQGLEWMGWINPNSGGTRYAQKFQGRVTMTRDTSTSTVYMELSSLRSEDTAVYYCARARGMTTGYMDVWGKGTTVTVSS

SEQ ID NO: 523 - protein, 107 aa, synthetic construct
DIQMTQSPSSLSASVGDRVTITCRASQDIGTWLAWYQQKPGKAPKLLIYDASTLQSGVPSRFSGSGSGTDFTLTISSLQPEDFATYYCQQSYRIPYTFGQGTKLEIK

SEQ ID NO: 524 - protein, 116 aa, synthetic construct
QVQLVQSGAEVKKPGASVKVSCKASGGTFSSYAISWVRQAPGQGLEWMGWIKPDSGGTIYAQKFQGRVTMTRDTSTSTVYMELSSLRSEDTAVYYCARDGNWFDPWGQGTLVTVSS

SEQ ID NO: 525 - protein, 107 aa, synthetic construct
DIQMTQSPSSLSASVGDRVTITCRASQAISRWLAWYQQKPGKAPKLLIYAASTLQSGVPSRFSGSGSGTDFTLTISSLQPEDFATYYCQESYIPPYTFGQGTKVEIK

SEQ ID NO: 526 - protein, 115 aa, synthetic construct
QVQLVQSGAEVKKPGASVKVSCKASGYTFSSYAISWVRQAPGQGLEWMGWINPNSGGTNYAQKFQGRVTMTRDTSTSTVYMELSSLRSEDTAVYYCARANWLDYWGQGTLVTVSS

SEQ ID NO: 527 - protein, 107 aa, synthetic construct
DIQMTQSPSSLSASVGDRVTITCRASQSIRRWLAWYQQKPGKAPKLLIYAASSLQSGVPSRFSGSGSGTDFTLTISSLQPEDFATYYCQQSYSTPYTFGQGTKLEIK

SEQ ID NO: 528 - protein, 115 aa, synthetic construct
QVQLVQSGAEVKKPGASVKVSCKASGGTFSSYAISWVRQAPGQGLEWMGWINPNSGGTNYAQKFQGRVTMTRDTSTSTVYMELSSLRSEDTAVYYCARDEWLQHWGQGTLVTVSS

SEQ ID NO: 529 - protein, 107 aa, synthetic construct
DIQMTQSPSSLSASVGDRVTITCRASQSISRWLAWYQQKPGKAPKLLIYAASTLQSGVPSRFSGSGSGTDFTLTISSLQPEDFATYYCQQSYSTPYTFGQGTKLEIK

SEQ ID NO: 530 - protein, 115 aa, synthetic construct
QVQLVQSGAEVKKPGASVKVSCKASGGTFSSYAISWVRQAPGQGLEWMGWINPNSGGTNYAQKFQGRVTMTRDTSTSTVYMELSSLRSEDTAVYYCARANWLDYWGQGTLVTVSS

SEQ ID NO: 531 - protein, 107 aa, synthetic construct
DIQMTQSPSSLSASVGDRVTITCRASQSISRWLAWYQQKPGKAPKLLIYAASRLHSGVPSRFSGSGSGTDFTLTISSLQPEDFATYYCQQSYSPPYTFGQGTKVEIK

SEQ ID NO: 532 - protein, 115 aa, synthetic construct
QVQLVQSGAEVKKPGASVKVSCKASGGTFSSYAISWVRQAPGQGLEWMGWISPNSGGTNYAQKFQGRVTMTRDTSTSTVYMELSSLRSEDTAVYYCARANWLDYWGQGTLVTVSS

SEQ ID NO: 533 - protein, 107 aa, synthetic construct
DIQMTQSPSSLSASVGDRVTITCRASQSIGRWLAWYQQKPGKAPKLLIYAASSLQSGVPSRFSGSGSGTDFTLTISSLQPEDFATYYCQQSYTSPYRFGQGTKLEIK

SEQ ID NO: 534 - protein, 119 aa, synthetic construct
QVQLVQSGAEVKKPGASVKVSCKASGYTFISYDINWVRQAPGQGLEWMGRINPYNDETHYAQKFQGRVTMTRDTSTSTVYMELSSLRSEDTAVYYCARDTSGDGRFHPWGQGTLVTVSS

SEQ ID NO: 535 - protein, 107 aa, synthetic construct
DIQMTQSPSSLSASVGDRVTITCRASQSVSRYLNWYQQKPGKAPKLLIYAASSLSNGVPSRFSGSGSGTDFTLTISSLQPEDFATYYCQHNYIRPPTFGRGTKVEIK

SEQ ID NO: 536 - protein, 117 aa, synthetic construct
QVQLVQSGAEVKKPGASVKVSCKASGYSFTSYEIHWVRQAPGQGLEWMGWINPNSGGTNYAQKFQGRVTMTRDTSTSTVYMELSSLRSEDTAVYYCAREGGTKLDVWGQGTTVTVSS

SEQ ID NO: 537 - protein, 107 aa, synthetic construct
DIQMTQSPSSLSASVGDRVTITCRASQGISNYLAWYQQKPGKAPKLLIYGASSLQSGVPSRFSGSGSGTDFTLTISSLQPEDFATYYCQQSYIIPLTFGQGTKVEIK

SEQ ID NO: 538 - protein, 116 aa, synthetic construct
QVQLVQSGAEVKKPGASVKVSCKASGYTFTSYGISWVRQAPGQGLEWMGWINPNSGGTNYAQKFQGRVTMTRDTSTSTVYMELSSLRSEDTAVYYCTRGSGWTDNWGQGTLVTVSS

SEQ ID NO: 539 - protein, 107 aa, synthetic construct
DIQMTQSPSSLSASVGDRVTITCRASQSVDRWLAWYQQKPGKAPKLLIYAASNLQSGVPSRFSGSGSGTDFTLTISSLQPEDFATYYCQQSYSTPYTFGQGTKLEIK

SEQ ID NO: 540 - protein, 116 aa, synthetic construct
QVQLVQSGAEVKKPGASVKVSCKASGYTFTSYGISWVRQAPGQGLEWMGWINPNSGGTNYARKFQGRVTMTRDTSTSTVYMELSSLRSEDTAVYYCAKEMGFLDYWGQGTTVTVSS

SEQ ID NO: 541 - protein, 107 aa, synthetic construct
DIQMTQSPSSLSASVGDRVTITCRASQSISTWLAWYQQKPGKAPKLLIYAASSLQSGVPSRFSGSGSGTDFTLTISSLQPEDFATYYCQQSYSTPYTFGQGTKLEIK

SEQ ID NO: 542 - protein, 115 aa, synthetic construct
QVQLVQSGAEVKKPGASVKVSCKASGYTFSSYGITWVRQAPGQGLEWMGWINPNSGGTNYAQKFQGRVTMTRDTSTSTVYMELSSLRSEDTAVYYCARANWLDYWGQGTLVTVSS

SEQ ID NO: 543 - protein, 107 aa, synthetic construct
DIQMTQSPSSLSASVGDRVTITCRASQSINRWLAWYQQKPGKAPKLLIYAASTLQSGVPSRFSGSGSGTDFTLTISSLQPEDFATYYCQQSYTTPYTFGQGTKLEIK

SEQ ID NO: 544 - protein, 123 aa, synthetic construct
EVQLLESGGGLVQPGGSLRLSCAASGFTFSSYGMHWVRQAPGKGLEWVAVISYDGSNQYYADSVKGRFTISRDNSKNTLYLQMNSLRAEDTAVYYCARTYSYGNYYYYGMDVWGQGTTVTVSS

SEQ ID NO: 545 - protein, 107 aa, synthetic construct
DIQMTQSPSSLSASVGDRVTITCRASESISTYLNWYQQKPGKAPKLLIYAASSLQSGVPSRFSGSGSGTDFTLTISSLQPEDFATYYCQQSYSTPITFGQGTRLEIK

SEQ ID NO: 546 - protein, 117 aa, synthetic construct
QVQLVQSGAEVKKPGASVKVSCKASGYTFTSYNMHWVRQAPGQGLEWMGWINPNSGGTNYAQKFQGRVTMTRDTSTSTVYMELSSLRSEDTAVYYCAREGAVAVDYWGRGTLVTVSS

SEQ ID NO: 547 - protein, 107 aa, synthetic construct
DIQMTQSPSSLSASVGDRVTITCRASQSISRWLAWYQQKPGKAPKLLIYAASTLQSGVPSRFSGSGSGTDFTLTISSLQPEDFATYYCQQGYSTPYTFGQGTKVEIK

SEQ ID NO: 548 - protein, 117 aa, synthetic construct
QVQLVQSGAEVKKPGASVKVSCKASGGTLSSYTLSWVRQAPGQGLEWMGWINPNSGGTNYAQKFQGRVTMTRDTSTSTVYMELSSLRSEDTAVYYCARDGGGGLDYWGQGTLVTVSS

SEQ ID NO: 549 - protein, 107 aa, synthetic construct
DIQMTQSPSSLSASVGDRVTITCRASQSVGRWLAWYQQKPGKAPKLLIYAASSLQSGVPSRFSGSGSGTDFTLTISSLQPEDFATYYCQQSYSTPYTFGQGTKLEIK

SEQ ID NO: 550 - protein, 117 aa, synthetic construct
QVQLVQSGAEVKKPGASVKVSCKASGYTFTSYVINWVRQAPGQGLEWMGWINPNSGGTNYAQKLQGRVTMTRDTSTSTVYMELSSLRSEDTAVYYCARERGGAYDYWGQGTLVTVSS

SEQ ID NO: 551 - protein, 107 aa, synthetic construct
DIQMTQSPSSLSASVGDRVTITCRASQSIGSWLAWYQQKPGKAPKLLIYAASSLQSGVPSRFSGSGSGTDFTLTISSLQPEDFATYYCQQTYSPPYTFGQGTKLEIK

SEQ ID NO: 552 - protein, 116 aa, synthetic construct
QVQLVQSGAEVKKPGASVKVSCKASGGTFRSYVISWVRQAPGQGLEWMGWISPDSGGTKYAQKFQGRVTMTRDTSTSTVYMELSSLRSEDTAVYYCARDSYGMDVWGQGTTVTVSS

SEQ ID NO: 553 - protein, 107 aa, synthetic construct
EIVMTQSPSSLSASVGDRVTITCRASQSISSWLAWYQQKPGKAPKLLIYAASSLQSGVPSRFSGSGSGTDFTLTISSLQPEDFATYYCQQSYSPPYTFGQGTKLEIK

SEQ ID NO: 554 - protein, 115 aa, synthetic construct
QVQLVQSGAEVKKPGASVKVSCKASGYTFTSYYMHWVRQAPGQGLEWMGWINPNSGGTNYAQKFQGRVTMTRDTSTSTVYMELSSLRSEDTAVYYCARGEWLDYWGQGTLVTVSS

SEQ ID NO: 555 - protein, 107 aa, synthetic construct
DIQMTQSPSSLSASVGDRVTITCRASQTISSWLAWYQQKPGKAPKLLIYAASSLQSGVPSRFSGSGSGTDFTLTISSLQPEDFATYYCQQSYSTPYTFGQGTKLEIK

SEQ ID NO: 556 - protein, 117 aa, synthetic construct
QVQLVQSGAEVKKPGASVKVSCKASGGTFSTHTITWVRQAPGQGLEWMGWINPDSGGTNYAQKFQGRVTMTRDTSTSTVYMELSSLRSEDTAVYYCAREGYSGLDYWGQGTLVTVSS

SEQ ID NO: 557 - protein, 107 aa, synthetic construct
DIQMTQSPSSLSASVGDRVTITCRASQSISNWLAWYQQKPGKAPKLLIYGASSLHSGVPSRFSGSGSGTDFTLTISSLQPEDFATYYCQQSYSVPYTFGQGTKVEIK

SEQ ID NO: 558 - protein, 117 aa, synthetic construct
QVQLVQSGAEVKKPGASVKVSCKASGYTFTTYLIHWVRQAPGQGLEWMGWINPNSGGTNYAQKFQGRVTMTRDTSTSTVYMELSSLRSEDTAVYYCARERASGYDIWGRGTLVTVSS

SEQ ID NO: 559 - protein, 107 aa, synthetic construct
DIQMTQSPSSLSASVGDRVTITCRASQSISSWLAWYQQKPGKAPKLLIYGASVLQSGVPSRFSGSGSGTDFTLTISSLQPEDFATYYCQQSYHVPYTFGQGTKLEIK

SEQ ID NO: 560 - protein, 115 aa, synthetic construct
QVQLVQSGAEVKKPGASVKVSCKASGYTFTTYPLSWVRQAPGQGLEWMGWINPNSGGTNYAQKFQGRVTMTRDTSTSTVYMELSSLRSEDTAVYYCARANWLDYWGQGTLVTVSS

SEQ ID NO: 561 - protein, 107 aa, synthetic construct
DIQMTQSPSSLSASVGDRVTITCRASQRIGSWLAWYQQKPGKAPKLLIYAASTLQSGVPSRFSGSGSGTDFTLTISSLQPEDFATYYCQQSYSAPYSFGQGTKLEIK

SEQ ID NO: 562 - protein, 115 aa, synthetic construct
QVQLVQSGAEVKKPGSSVKVSCKASGYTFTTYPMXWVRQAPGQGLEWMGWINPNSGGTNYAQKFQGRVTMTRDTSTSTVYMELSSLRSEDTAVYYCAXANWLDYWGQGTLVTVSS

SEQ ID NO: 563 - protein, 107 aa, synthetic construct
DIQMTQSPSSLSASVGDRVTITCRASQGIDRWLAWYQQKPGKAPKLLIYAASSLQSGVPSRFSGSGSGTDFTLTISSLQPEDFATYYCQQSYSPPYTFGQGTKVEIK

SEQ ID NO: 564 - protein, 115 aa, synthetic construct
QVQLVQSGAEVKKPGASVKVSCKASGYPFSTYYMHWVRQAPGQGLEWMGWINPNSGGTNYAQKFQGRVTMTRDTSTSTVYMELSSLRSEDTAVYYCARGEWLDYWGQGTLVTVSS

SEQ ID NO: 565 - protein, 107 aa, synthetic construct
DIQMTQSPSSLSASVGDRVTITCRASQGISSWLAWYQQKPGKAPKLLIYAASKLQGGVPSRFSGSGSGTDFTLTISSLQPEDFATYYCQQSYTTPYTFGQGTKLEIK

SEQ ID NO: 566 - protein, 115 aa, synthetic construct
QVQLVQSGAEVKKPGASVKVSCKASGYSFTAYHVHWVRQAPGQGLEWMGWINPNSGGTNYAQKFQGRVTMTRDTSTSTVYMELSSLRSEDTAVYYCARANWLDYWGQGTLVTVSS

SEQ ID NO: 567 - protein, 115 aa, synthetic construct
QVQLVQSGAEVKKPGASVKVSCKASGYTFTAYNMHWVRQAPGQGLEYMGWINPNSGGTNYAQKFQGRVTMTRDTSTSTVYMELSSLRSEDTAVYYCARANWLDYWGQGTLVTVSS

SEQ ID NO: 568 - protein, 107 aa, synthetic construct
DIQMTQSPSSLSASVGDRVTITCRASQSISRWLAWYQQKPGKAPKLLIYAASSLQSGVPSRFSGSGSGTDFTLTISSLQPEDFATYYCQQSYSTPYTFGQGTKLEIK

SEQ ID NO: 569 - protein, 115 aa, synthetic construct
EVQLLESGAEVKKPGASVKVSCKASGYTFTDYGLSWVRQAPGQGLEWMGWINPNSGGTNYAQKFQGRVTMTRDTSTSTVYMELSSLRSEDTAVYYCARANWLDYWGQGTLVTVSS

SEQ ID NO: 570 - protein, 107 aa, synthetic construct
DIQMTQSPSSLSASVGDRVTITCRASQTIGPWLAWYQQKPGKAPKLLIYAASTLQSGVPSRFSGSGSGTDFTLTISSLQPEDFATYYCQQSYSTPYTFGQGTKLEIK

SEQ ID NO: 571 - protein, 115 aa, synthetic construct
QVQLVQSGAEVKKPGSSVKVSCKASGYTFTDYHLHWVRQAPGQGLEWVGWINPNSGGTNFAQKFQGRVTMTRDTSTSTVYMELSSLRSEDTAVYYCARGEWLDYWGQGTLVTVSS

SEQ ID NO: 572 - protein, 107 aa, synthetic construct
DIQMTQSPSSLSASVGDRVTITCRASQSISSWLAWYQQKPGKAPKLLIYAASSLQSGVPSRFSGSGSGTDFTLTISSLQPEDFATYYCQQSYSTPYTFGQGTKLEIK

SEQ ID NO: 573 - protein, 115 aa, synthetic construct
QVQLVQSGAEVKKPGASVKVSCKASGYTFTDYLMHWVRQAPGQGLEWMGWINPNSGGTNYAQKXQGRVTMTRDTSTSTVYMELXSLRSEDTAVYYCARXNXXDYWGXGXLXXVSS

SEQ ID NO: 574 - protein, 107 aa, synthetic construct
DIQMTQSPSSLSASVGDRVTITCRASQSINRWLAWYQQKPGKAPKLLIYAASKLETGVPSRFSGSGSGTDFTLTISSLQPEDFATYYCQQSYNTPYTFGQGTKLEIK

SEQ ID NO: 575 - protein, 115 aa, synthetic construct
QVQLVQSGAEVKKPGASVKVSCKASGFTFTDYNIHWVRQAPGQGLEWMGWINPNSGGTNYAQKFQGRVTMTRDTSTSTVYMELSSLRSEDTAVYYCARANWLDYWGRGTLVTVSS

SEQ ID NO: 576 - protein, 107 aa, synthetic construct
DIQMTQSPSSLSASVGDRVTITCRASQSISRWLAWYQQKPGKAPKLLIYAASTLQTGVPSRFSGSGSGTDFTLTISSLQPEDFATYYCQQSYSTPYTFGQGTKLEIK

SEQ ID NO: 577 - protein, 116 aa, synthetic construct
QVQLVQSGAEVKKPGASVKVSCKASGYTFTDYQIHWVRQAPGQGLEWMGWISPYSGGTNYAQKFQGRVTMTRDTSTSTVYMELSSLRSEDTAVYYCARDRVGLDYWGQGTLVTVSS

SEQ ID NO: 578 - protein, 107 aa, synthetic construct
DIQMTQSPSSLSASVGDRVTITCRASQSISTWLAWYQQKPGKAPKLLIYAASSLQSGVPSRFSGSGSGTDFTLTISSLQPEDFATYYCQQSYSIPYTFGQGTKVEIK

SEQ ID NO: 579 - protein, 115 aa, synthetic construct
EVQLLESGAEVKKPGASVKVSCKASGYTFTDYTIHWVRQAPGQGLEWMGWINPNSGGTNYAQQFQGRVTMTRDTSTSTVYMELSSLRSEDTAVYYCARANWLDYWGQGTLVTVSS

SEQ ID NO: 580 - protein, 107 aa, synthetic construct
DIQMTQSPSSLSASVGDRVTITCRASQNIRRWLAWYQQKPGKAPKLLIYAASSLRSGVPSRFSGSGSGTDFTLTISSLQPEDFATYYCQQSYSTPYTFGQGTKVEIK

SEQ ID NO: 581 - protein, 115 aa, synthetic construct
QVQLVQSGAEVKKPGASVKVSCKASGYTFTENEMHWVRQAPGQGLEWMGWINPNSGGTSYAPNFQGRVTMTRDTSTSTVYMELSSLRSEDTAVYYCARANWLDFWGQGTLVTVSS

SEQ ID NO: 582 - protein, 107 aa, synthetic construct
DIQMTQSPSSLSASVGDRVTITCRASQEIRRWLAWYQQKPGKAPKLLIYAASSLQSGVPSRFSGSGSGTDFTLTISSLQPEDFATYYCQQSYSPPYTFGQGTKLEIK

SEQ ID NO: 583 - protein, 117 aa, synthetic construct
QVQLVQSGAEVKKPGASVKVSCKASGYAFTFSTIHWVRQAPGQGLEWMGWISPHSGGTNYAQEFQGRVTMTRDTSTSTVYMELSSLRSEDTAVYYCARDSGSYLDYWGQGTLVTVSS

SEQ ID NO: 584 - protein, 107 aa, synthetic construct
DIQMTQSPSSLSASVGDRVTITCQASQDINNYLNWYQQKPGKAPKLLIYAASSLQSGVPSRFSGSGSGTDFTLTISSLQPEDFATYYCQQSYSTPYTFGQGTKLEIK

SEQ ID NO: 585 - protein, 115 aa, synthetic construct
QVQLVQSGAEVKKPGASVKVSCKASGGTFSGYAISWVRQAPGQGLEWMGWINPNSGGTNYAQKFQGRVTMTHDTSTSTVYMDLSSLRSEDTAVYYCARGDWFDPWGQGTLVTVSS

SEQ ID NO: 586 - protein, 107 aa, synthetic construct
DIQMTQSPSSLSASVGDRVTITCRASQEIRRWLAWYQQKPGKAPKLLIYAASSLQSGVPSRFSGSGSGTDFTLTISSLQPEDFATYYCQQSYSTPYTFGQGTKLEIK

SEQ ID NO: 587 - protein, 115 aa, synthetic construct
EVQLLESGAEVKKPGASVKVSCKASGYTFTGYNMHWVRQAPGQGLEWMGWINPNSGGTNYAQKFQGRVTMTRDTSTSTVYMELSSLRSEDTAVYYCARANWLDYWGQGTLVTVSS

SEQ ID NO: 588 - protein, 107 aa, synthetic construct
DIQMTQSPSSLSASVGNRVTITCRASQSINRWLAWYQQKPGKAPKLLIYAASTLQSGVPSRFSGSGSGTDFTLTISSLQPEDFATYYCQQSYITPYTFGQGTKLEIK

SEQ ID NO: 589 - protein, 122 aa, synthetic construct
QVQLVQSGAEVKKPGASVKVSCKASGYTFTGYYLHWVRQAPGQGLEWMGWINPKSGGTNFAQKFQGRVTMTRDTSTSTVYMELSSLRSEDTAVYYCARERGATGYYYGMDVWGQGTTVTVSS

SEQ ID NO: 590 - protein, 107 aa, synthetic construct
DIQMTQSPSSLSASVGDRVTITCRASQRVDSWLAWYQQKPGKAPKLLIYKASILESGVPSRFSGSGSGTDFTLTISSLQPEDFATYYCQQTYSTPWTFGQGTKVEIK

SEQ ID NO: 591 - protein, 114 aa, synthetic construct
QVQLVQSGAEVKKPGASVKVSCKASGYTFTGYYMHWVRQAPGQGLEWMGWIYPNSGGTNYAQNLQGRVTMTRDTSTSTVYMELSSLRSEDTAVYYCARDDFDYWGQGTLVTVSS

SEQ ID NO: 592 - protein, 107 aa, synthetic construct
DIQMTQSPSSLSASVGDRVTITCQASQDISNYLNWYQQKPGKAPKLLISVASTLQSGVPSRFSGSGSGTDFTLTISSLQPEDFATYYCQQSYNTPYTFGQGTKLEIK

SEQ ID NO: 593 - protein, 116 aa, synthetic construct
QVQLVQSGAEVKKPGASVKVSCKASGYTFTKYGISWVRQAPGQGLEWMGWINPYSGGTNYAQNFQGRVTMTRDTSTSTVYMELSSLRSEDTAVYYCARGDGWYDPWGQGTLVTVSS

SEQ ID NO: 594 - protein, 107 aa, synthetic construct
DIQMTQSPSSLSASVGDRVTITCRASQSISNWLAWYQQKPGKAPKLLIYAASSLQSGVPSRFSGSGSGTDFTLTISSLQPEDFATYYCQQSYSPPYTFGQGTKLEIK

SEQ ID NO: 595 - protein, 117 aa, synthetic construct
QVQLVQSGAEVKKPGASVKVSCKASGYTFSNFLINWVRQAPGQGLEWMGWINPNSGGTNYAQKFQGRVTMTRDTSTSTVYMELSSLRSEDTAVYYCASERELPFDIWGQGTMVTVSS

SEQ ID NO: 596 - protein, 107 aa, synthetic construct
DIQMTQSPSSLSASVGDRVTITCRASQSISSWVAWYQQKPGKAPKLLIYGASNLESGVPSRFSGSGSGTDFTLTISSLQPEDFATYYCQQTYNPPYSFGQGTKLEIK

SEQ ID NO: 597 - protein, 116 aa, synthetic construct
QVQLVQSGAEVKKPGASVKVSCKASGYTFSNHIMHWVRQAPGQGLEWMGWIYPDSGGTKYAQKFQGRVTMTRDTSTSTVYMELSSLRSEDTAVYYCARATGFMDVWGQGTTVTVSS

SEQ ID NO: 598 - protein, 107 aa, synthetic construct
DIQMTQSPSSLSASVGDRVTITCRASQSISNWLAWYQQKPGKAPKLLIYAASSLQSGVPSRFSGSGSGTDFTLTISSLQPEDFATYYCQQSYNTPFTFGPGTKVDIK

SEQ ID NO: 599 - protein, 115 aa, synthetic construct
QVQLVQSGAEVKKPGASVKVSCKASGFTFTNHNMHWVRQAPGQGLEWMGWINPNSGGTNYAQKFQGRVTMTRDTSTSTVYMELSSLRSEDTAVYYCARANWLDYWGQGTLVTVSS

SEQ ID NO: 600 - protein, 115 aa, synthetic construct
QVQLVQSGAEVKKPGSSVKVSCKASGGTFSNYAFNWVRQAPGQGLEWMGWIYPNTGGTHYAPKFQGRVTMTRDTSTSTVYMELSSLRSEDTAVYYCARANWLDYWGQGTLVTVSS

SEQ ID NO: 601 - protein, 107 aa, synthetic construct
DIQMTQSPSSLSASVGDRVTITCRASQSIDRWLAWYQQKPGKAPKLLIYAASTLQSGVPSRFSGSGSGTDFTLTISSLQPEDFATYYCQQSYITPYTFGQGTKLEIK

SEQ ID NO: 602 - protein, 116 aa, synthetic construct
QVQLVQSGAEVKKPGASVKVSCKASGGTFSNYAFSWVRQAPGQGLEWMGWIYPNSGGTNYAQKFQGRVTMTRDTSTSTVYMELSSLRSEDTAVYYCARGESWFDPWGQGTLVTVSS

SEQ ID NO: 603 - protein, 107 aa, synthetic construct
DIQMTQSPSSLSASVGDRVTITCRASQSISRWLAWYQQKPGKAPKLLIYAASNLESGVPSRFSGSGSGTDFTLTISSLQPEDFATYYCQQSYSTPYTFGQGTKLEIK

SEQ ID NO: 604 - protein, 116 aa, synthetic construct
QVQLVQSGAEVKKPGASVKVSCKASGGTFSNYAISWVRQAPGQGLEWMGWINPNTGGTNYAQSLQGRVTMTRDTSTSTVYMELSSLRSEDTAVYYCARSSGWYDYWGQGTLVTVSS

SEQ ID NO: 605 - protein, 107 aa, synthetic construct
DIQMTQSPSSLSASVGDRVTITCRASQEIRRWLAWYQQKPGKAPKLLIYAASSLQSGVPSRFSGSGSGTDFTLTISSLQPEDFATYYCQQSYSPPYSFGQGTKLEIK

SEQ ID NO: 606 - protein, 115 aa, synthetic construct
QVQLVQSGAEVKKPGASVKVSCKASGGTFSNYGISWVRQAPGQGLEWMGWISPDSGGTIYAQRFQGRVTMTRDTSTSTVYMELSSLRSEDTAVYYCARANWLDYWGQGTLVTVSS

SEQ ID NO: 607 - protein, 107 aa, synthetic construct
DIQMTQSPSSLSASVGDRVTITCRASQSISRWLAWYQQKPGKAPKLLIYAASSLQSGVPSRFSGSGSGTDFTLTISSLQPEDFATYYCQQSYSTPYTFGQGTKVEIK

SEQ ID NO: 608 - protein, 115 aa, synthetic construct
EVQLLESGAEVKKPGASVKVSCKASGYTFTNYGISWVRQAPGQGLEWMGWINPYSGGTNYAQKFQGRVTMTRDTSTSTVYMELSSLRSEDTAVYYCARANWLDYWGQGTLVTVSS

SEQ ID NO: 609 - protein, 107 aa, synthetic construct
DIQMTQSPSSLSASVGDRVTITCRASQSVGRWLAWYQQKPGKAPKLLIYAASSLQSGVPSRFSGSGSGTDFTLTISSLQPEDFATYYCQQSYSIPYSFGQGTKLEIK

SEQ ID NO: 610 - protein, 115 aa, synthetic construct
QVQLVQSGAEVKKPGASVKVSCKASGYSFTNYHMHWVRQAPGQGLEWMGWIYPNSGGTNYAQKFQGRVTMTRDTSTSTVYMELSSLRSEDTAVYYCARGAWFDPWGQGTLVTVSS

SEQ ID NO: 611 - protein, 107 aa, synthetic construct
DIQMTQSPSSLSASVGDRVTITCRASQSINSWLAWYQQKPGKAPKLLIYGASNLQSGVPSRFSGSGSGTDFTLTISSLQPEDFATYYCQQSYSTPYTFGQGTKLEIK

SEQ ID NO: 612 - protein, 115 aa, synthetic construct
EVQLLESGAEVKKPGASVKVSCKASGYNFINYNIAWVRQAPGQGLEWMGWINPNSGGTNYAQKFQGRVTMTRDTSTSTVYMELSSLRSEDTAVYYCARANWLDYWGQGTLVTVSS

SEQ ID NO: 613 - protein, 107 aa, synthetic construct
DIQMTQSPSSLSASVGDRVTITCRASQGISSWLAWYQQKPGKAPKLLIYAASTLQSGVPSRFSGSGSGTDFTLTISSLQPEDFATYYCQQSYSTPYTFGQGTKLEIK

SEQ ID NO: 614 - protein, 117 aa, synthetic construct
QVQLVQSGAEVKKPGASVKVSCKASGYTFTNYPIHWVRQAPGQGLEWMGWINPNSGGTNYAQKLQGRVTMTRDTSTSTVYMELSSLRSEDTAVYYCARDQGAGWDYWGQGTLVTVSS

SEQ ID NO: 615 - protein, 107 aa, synthetic construct
DIQMTQSPSSLSASVGDRVTITCRASQEIRRWLAWYQQKPGKAPKLLIYAASNLQSGVPSRFSGSGSGTDFTLTISSLQPEDFATYYCQQSYSTPYTFGQGTKLEIK

SEQ ID NO: 616 - protein, 115 aa, synthetic construct
QVQLVQSGAEVKKPGASVKVSCKASGYTFTNYQIHWVRQAPGQGLEWMGWINPNSGGTISAPNFQGRVTMTRDTSTSTVYMELSSLRSEDTAVYYCARANWLDYWGQGTLVTVSS

SEQ ID NO: 617 - protein, 115 aa, synthetic construct
QVQLVQSGAEVKKPGASVKVSCKASGGTFSNYVISWVRQAPGQGLEWMGWINPNSGGTIYAQKFQGRVTMTRDTSTSTVYMELSSLRSEDTAVYYCARGEWFDPWGQGTLVTVSS

SEQ ID NO: 618 - protein, 107 aa, synthetic construct
DIQMTQSPSSLSASVGDRVTITCRASQSINSWLAWYQQKPGKAPKLLIYGASSLQSGVPSRFSGSGSGTDFTLTISSLQPEDFATYYCQQSYSTPYTFGQGTKLEIK

SEQ ID NO: 619 - protein, 117 aa, synthetic construct
QVQLVQSGAEVKKPGASVKVSCKASGGTFSNYVISWVRQAPGQGLEWMGWIYPNTGGTNYAQKFQGRVTMTRDTSTSTVYMELSSLRSEDTAVYYCARDRGYGMDVWGQGTKVTVSS

SEQ ID NO: 620 - protein, 106 aa, synthetic construct
DIQMTQSPSSLSASVGDRVTITCRASQDISTWLAWYQQKPGKAPKLLIYAASSLQSGVPSRFSGSGSGTDFTLTISSLQPEDFATYYCQQSYSSWTFGQGTKVEIK

SEQ ID NO: 621 - protein, 115 aa, synthetic construct
QVQLVQSGAEVKKPGASVKVSCKASGGNFTPYTFSWVRQAPGQGLEWMGWINPSSGGAQYAQKLQGRVTMTRDTSTSTVYMELSSLRSEDTAVYYCARGEWLDYWGQGTLVTVSS

SEQ ID NO: 622 - protein, 107 aa, synthetic construct
DIQMTQSPSSLSASVGDRVTITCRASQSIRSWLAWYQQKPGKAPKLLIYAASSLQSGVPSRFSGSGSGTDFTLTISSLQPEDFATYYCQQSYITPYTFGQGTKLEIK

SEQ ID NO: 623 - protein, 115 aa, synthetic construct
QVQLVQSGAEVKKPGASVKFSCKASGYTFTRHPMHWVRQAPGQGLEWMGWIHPDSGGTNYSQKFQGRVTMTRDTSTSTVYMELSSLRSEDTAVYYCARANWLDYWGQGTLVTVSS

SEQ ID NO: 624 - protein, 107 aa, synthetic construct
DIQMTQSPSSLSASVGDRVTITCRASQSIDRWLAWYQQKPGKAPKLLIYAASTLQSGVPSRFSGSGSGTDFTLTISSLQPEDFATYYCQQSYSTPYSFGQGTKVEIK

SEQ ID NO: 625 - protein, 115 aa, synthetic construct
QVQLVQSGAEVKKHGASVKVSCKASGYTFPSHGITWVRQAPGQGLEWMGWINPNSGGTNYAQNVQGRVTMTRDTSTSTVYMELSSLRSEDTAVYYCARGPWLDYWGQGTLVTVSS

SEQ ID NO: 626 - protein, 115 aa, synthetic construct
QVQLVQSGAXVXKPGASLKVSCRASGYTLTSHLIHWVRQAPGQGLEWMGWINPNSGGTNYAQKFQGRVTMTRDTSTSTXYMELSSLRSEDTAVYYCARGIFEDYWGQGTLVTGSS

SEQ ID NO: 627 - protein, 107 aa, synthetic construct
DIQMTQSPSSLSASVGDRVTITCRASQGIGRWLAWYQQKPGKAPKLLIYATSRLQSGVPSRFSGSGSGTDFTLTISSLQPEDFATYYCQQSYGTPYTFGQGTKVEIK

SEQ ID NO: 628 - protein, 115 aa, synthetic construct
EVQLLESGAEVKKPGASVKVSCKASGYTFTSHLIHWVRQAPGQGLEWMGWINPNSGGTNYAQKFQGRVTMTRDTSTSTVYMELSSLRSEDTAVYYCARANWLDYWGQGTLVTVSS

SEQ ID NO: 629 - protein, 107 aa, synthetic construct
DIQMTQSPSSLSASVGDRVTITCRASQSIDRWLTWYQQKPGKAPKLLIYAASRLQSGVPSRFSGSGSGTDFTLTISSLQPEDFATYYCQQSYRTPYTFGQGTKVEIK

SEQ ID NO: 630 - protein, 115 aa, synthetic construct
QVQLVQSGVEVKKPGASVKVSCKASGYTLTSHLIHWVRQAPGQGLEWMGWINPNSGGTKYAPKFQGRVTMTRDTSTSTVYMEVSSLRSEDTAVYYCARGDWYDYWGQGTLVTVSS

SEQ ID NO: 631 - protein, 107 aa, synthetic construct
DIQMTQSPSSLSASVGDRVTITCRASQSIGRWLAWYQQKPGKAPKLLIYAASTLQSGVPSRFSGSGSGTDFTLTISSLQPEDFATYYCQQSYSSPYTFGQGTKVEIK

SEQ ID NO: 632 - protein, 115 aa, synthetic construct
QVQLVQSGAEVKKPGASVKVSCKASGYTLTSHLIHWVRQAPGQGLEWMGWINPNSGGTNYAQSFQGRVTMTRDTSTSTVYMELSSLRSEDTAVYYCARANWLDYWGQGTLVTVSS

SEQ ID NO: 633 - protein, 107 aa, synthetic construct
DIQMTQSPSSLSASVGDRVTITCRASQSISRWLAWYQQKPGKAPKLLIYAASSLQSGVPSRFSGSGSGTDFTLTISSLQPEDFATYYCQQSYSAPLTFGGGTKVEIK

SEQ ID NO: 634 - protein, 115 aa, synthetic construct
EVQLVQSGAEVKKPGASVKVSCKASGGTFSSYAISWVRQAPGQGLEWMGWINPNSGGTNYAQKFQGRVTMTRDTSTSTVYMELSSLRSEDTAVYYCARANWLDYWGQGTLVTVSS

SEQ ID NO: 635 - protein, 122 aa, synthetic construct
EVQLLESGGGLVQPGGSLRLSCAASGFTFSSYAMHWVRQAPGKGLEWVSGISGNGGSTNYADSVKGRFTISRDNSKNTLYLQMNSLRAEDTAVYYCAKDAYYYGSGSYFDYWGQGTLVTVSS

SEQ ID NO: 636 - protein, 107 aa, synthetic construct
DIQMTQSPSSLSASVGDRVTITCRASQSISSYLNWYQQKPGKAPKLLIYAASTLQTGVPSRFSGSGSGTDFTLTISSLQPEDFATYYCQQSYTTPYTFGQGTKLEIK

SEQ ID NO: 637 - protein, 115 aa, synthetic construct
QVQLVQSGAEVKKPGASVKVSCKASGYTFTSYDINWVRQAPGQGLEWMGWINPNSGGTNYAQKFQGRVTMTRDTSTSTVYMELSSLRSEDTAVYYCARGEWLDYWGQGTLVTVSS

SEQ ID NO: 638 - protein, 107 aa, synthetic construct
DIQMTQSPSSLSASVGDRVTITCRASQSIRSWLAWYQQKPGKAPKLLIYAASSLQSGVPSRFSGSGSGTDFTLTISSLQPEDFATYYCQQSYSTPYTFGQGTKLEIK

SEQ ID NO: 639 - protein, 116 aa, synthetic construct
QVQLVQSGAEVKKPGASVKVSCKASGYTFSSYGISWVRQAPGQGLEWMGWINPNSGGTNYAQKLQGRVTMTRDTSTSTVYMELSSLRSEDTAVYYCTGDYSWFDPWGQGTLVTVSS

SEQ ID NO: 640 - protein, 107 aa, synthetic construct
DIQMTQSPSSLSASVGDRVTITCRASQDISNWLAWYQQKPGKAPKLLIYGASTLQSGVPSRFSGSGSGTDFTLTISSLQPEDFATYYCQQSYSTPYTFGQGTKLEIK

SEQ ID NO: 641 - protein, 115 aa, synthetic construct
EVQLLESGAEVKKPGASVKVSCKASGYTFTSYGISWVRQAPGQGLEWMGWINPNSGGTNYAQKFQGRVTMTRDTSTSTVYMELSSLRSEDTAVYYCARANWLDYWGQGTLVTVSS

SEQ ID NO: 642 - protein, 107 aa, synthetic construct
DIQMTQSPSSLSASVGDRVTITCRASQSIGRWLAWYQQKPGKAPKLLIYATSSLQSGVPSRFSGSGSGTDFTLTISSLQPEDFATYYCQQSYSTPYTFGQGTKLEIK

SEQ ID NO: 643 - protein, 116 aa, synthetic construct
QVQLVQSGAEVKKPGASVKVSCKASGYTFTSYGISWVRQAPGQGLEWMGWINPNSGGTNYAQRLQGRVTMTRDTSTSTVYMELSSLRSEDTAVYYCARDFSWFDPWGQGTLVTVSS

SEQ ID NO: 644 - protein, 107 aa, synthetic construct
DIQMTQSPSSLSASVGDRVTITCRASQSISNWLAWYQQKPGKAPKLLIYGASNLQPGVPSRFSGSGSGTDFTLTISSLQPEDFATYYCQQSYSTPYSFGQGTKLEIK

SEQ ID NO: 645 - protein, 116 aa, synthetic construct
QVQLVQSGAEVKKPGASVKVSCKASGYTFTSYGISWVRQAPGQGLEWMGWINPNSGGTNYAQKFQGRVTMTRDTSTSTVYMELSSLRSEDTAVYYCARGYNWFDPWGQGTLVTVSS

SEQ ID NO: 646 - protein, 107 aa, synthetic construct
DIQMTQSPSSLSASVGDRVTITCRASQSISNWLAWYQQKPGKAPKLLIYAASNLQSGVPSRFSGSGSGTDFTLTISSLQPEDFATYYCQQSYNTPFTFGPGTKVDIK

SEQ ID NO: 647 - protein, 107 aa, synthetic construct
DIQMTQSPSSLSASVGDRVTITCRASQDISRWLAWYQQKPGKAPKLLIYAASSLQSGVPSRFSGSGSGTDFTLTISSLQPEDFATYYCQQSYNTPYTFGQGTKLEIK

SEQ ID NO: 648 - protein, 116 aa, synthetic construct
QVQLVQSGAEVKKPGASVKVSCKASGYTFTSYGISWVRQAPGQGLEWMGWINPNSGGTNYAQKFQGRVTMTRDTSTSTVYMELSSLRSEDTAVYYCARSINWFDPWGQGTLVTVSS

SEQ ID NO: 649 - protein, 107 aa, synthetic construct
DIQMTQSPSSLSASVGDRVTITCRASQSVGRWMAWYQQKPGKAPKLLIYAASTLQTGVPSRFSGSGSGTDFTLTISSLQPEDFATYYCQQSYSPPYTFSQGTKLEIK

SEQ ID NO: 650 - protein, 115 aa, synthetic construct
EVQLLESGAEVKKPGASVKVSCKASGYTFTSYGISWVRQAPGQGLEWMGWINPHSGGTFYAQKFQGRVTMTRDTSTSTVYMELSSLRSEDTAVYYCARANWLDYWGQGTLVTVSS

SEQ ID NO: 651 - protein, 107 aa, synthetic construct
DIQMTQSPSSLSASVGDRVTITCRASQSISSWLAWYQQKPGKAPKLLIYAASRLQSGVPSRFSGSGSGTDFTLTISSLQPEDFATYYCQQSYSPPYTFGQGTKLEIK

SEQ ID NO: 652 - protein, 117 aa, synthetic construct
QVQLVQSGAEVKKPGASVKVSCKASGYTLTSYLMHWVRQAPGQGLEWMGWINPYSGGTNYAQKFQGRVTMTRDTSTSTVYMELSSRRSEDTAVYYCARDRGSGYDYWGQGTLVTVSS

SEQ ID NO: 653 - protein, 107 aa, synthetic construct
DIQMTQSPSSLSASVGDRVTITCRASQTIGNWLAWYQQKPGKAPKLLIYGASSLQSGVPSRFSGSGSGTDFTLTISSLQPEDFATYYCQQSYSPPLTFGPGTKVDIK

SEQ ID NO: 654 - protein, 115 aa, synthetic construct
QVQLVQSGAEVKKPGASVKVSCKASGYTFTSYNMHWVRQAPGQGLEWMGWINPNSGGTNYAQKLQGRVTMTRDTSTSTVYMELSSLRSEDTAVYYCARANWLDYWGQGTLVTVSS

SEQ ID NO: 655 - protein, 115 aa, synthetic construct
EVQLLESGAEVKKPGASVKVSCKASGYIFTSYPIHWVRQAPGQGLEWMGWINPNSGGTNYAQKFQGRVTMTRDTSTSTVYMELSSLRSEDTAVYYCARANWLDYWGQGTLVTVSS

SEQ ID NO: 656 - protein, 107 aa, synthetic construct
DIQMTQSPSSLSASVGDRVTITCRASQSISRWLAWYQQKPGKAPKLLIYAASNLQSGVPSRFSGSGSGTDFTLTISSLQPEDFATYYCQQSYSPPYTFGQGTKLEIK

SEQ ID NO: 657 - protein, 115 aa, synthetic construct
QVQLVQSGAEVKKPGASVKVSCKASGGTFTSYPISWVRQAPGQGLEWMGWINPSNGGTNYAQKFQGRVTMTRETSTSTAYMELSSLRSEDTAVYYCARSNWLDYWGQGTLVTVSS

SEQ ID NO: 658 - protein, 107 aa, synthetic construct
DIQMTQSPSSLSASVGDRVTITCRASQSIGRWLAWYQQKPGKAPKLLIYAASTLQSGVPSRFSGSGSGTDFTLTISSLQPEDFATYYCQQSYSTPYTFGQGTKLEIK

SEQ ID NO: 659 - protein, 115 aa, synthetic construct
EVQLVQSGAEVKKPGASVKVSCKASGGTFSSYTISWVRQAPGQGLEWMGWISPNSGGTNYAQKFQGRVTMTRDTSTSTVYMELSSLRSEDTAVYYCARANWLDYWGQGTLVTVSS

SEQ ID NO: 660 - protein, 107 aa, synthetic construct
DIQMTQSPSSLSASVGDRVTITCRTSQGIGTWLAWYQQKPGKAPKLLIYAASSLQSGVPSRFSGSGSGTDFTLTISSLQPEDFATYYCQQSYSTPYTFGQGTKLEIK

SEQ ID NO: 661 - protein, 115 aa, synthetic construct
QVQLVQSGAEVKKPGASVKVSCKASGGTFSTFPMHWVRQAPGQGLEWMGWINPNSGGTNYAQKFQGRVTMTRDTSTSTVYMELSSLRSEDTAVYYCARGEWLGYWGQGTLVTVSS

SEQ ID NO: 662 - protein, 107 aa, synthetic construct
DIQMTQSPSSLSASVGDRVTITCRASQSISSWLAWYQQKPGKAPKLLIYAASTLQSGVPSRFSGSGSGTDFTLTISSLQPEDFATYYCQQSYNPPYTFGQGTKLEIK

SEQ ID NO: 663 - protein, 115 aa, synthetic construct
EVQLLESGAEVKKPGASVKVSCKASGYSFTTYGISWVRQAPGQGLEWMGWINPNSGGTNYAQKFQGRVTMTRDTSTSTVYMELSSLRSEDTAVYYCARANWLDYWGQGTLVTVSS

SEQ ID NO: 664 - protein, 107 aa, synthetic construct
DIQMTQSPSSLSASVGDRVTITCRASQDISRWSAWYQQKPGKAPKLLIYGASSLQSGVPSRFSGSGSGTDFTLTISSLQPEDFATYYCQQSYSPPYTFGQGTKLEIK

SEQ ID NO: 665 - protein, 115 aa, synthetic construct
QVQLVQSGAEVKKPGASVKVSCKASGYTFTTYNIHWVRQAPGQGLEWMGWINPNSGGTNYAQKFQGRVTMTRDTSTSTVYMELSSLRSEDTAVYYCARANWLDYWGQGTLVTVSS

SEQ ID NO: 666 - protein, 107 aa, synthetic construct
DIQMTQSPSSLSASVGDRVTITCRASQEIRRWLAWYQQKPGKAPKLLIYAASNLETGVPSRFSGSGSGTDFTLTISSLQPEDFATYYCQQSYSIPYTFGQGTKLEIK

SEQ ID NO: 667 - protein, 115 aa, synthetic construct
QVQLVQSGAEVKKPGASVKVSCKASGGTFSTYTVSWVRQAPGQGLEWMGWINPNSGGTNYAQKFQGRVTMTRDTSTSTVYMELSSLRSEDTAVYYCARGDWFDPWGQGTLVTVSS

SEQ ID NO: 668 - protein, 107 aa, synthetic construct
DIQMTQSPSSLSASVGDRVTITCRASQGISRWLAWYQQKPGKAPKLLIYAASTLQSGVPSRFSGSGSGTDFTLTISSLQPEDFATYYCQQSYETPYTFGQGTKVEIK

SEQ ID NO: 669 - protein, 115 aa, synthetic construct
QVQLVQSGAEVKKPGASVKVSCKASGYTFTAYYMHWVRQAPGQGLEWMGWISPNSGGTNYAQKFQGRVTMTRDTSTSTVYMELSSLRSEDTAVYYCARGDWNDWWGQGTLVTVSS

SEQ ID NO: 670 - protein, 107 aa, synthetic construct
DIQMTQSPSSLSASVGDRVTITCRASQDISRWLAWYQQKPGKAPKLLIYAASTLQSGVPSRFSGSGSGTDFTLTISSLQPEDFATYYCQQGYSTPYTFGQGTKLEIK

SEQ ID NO: 671 - protein, 115 aa, synthetic construct
QVQLVQSGAEVKKPGASVKVSCKASGYTFTDYHLHWVRQAPGQGLEWMGWINPNSGGTNYAQMFQGRVTMTRDTSTSTVYMELSSLRSEDTAVYYCARGEWFDPWGQGTLVTVSS

SEQ ID NO: 672 - protein, 107 aa, synthetic construct
DIQMTQSPSSLSASVGDRVTITCRASQSISSWLAWYQQKPGKAPKLLIYAASTLQSGVPSRFSGSGSGTDFTLTISSLQPEDFATYYCQQSYSTPYTFGQGTKLEIK

SEQ ID NO: 673 - protein, 117 aa, synthetic construct
QVQLVQSGAEVKKPGASVKVSCKASGYTFTDYIVHWVRQAPGQGLEWMGWINPYSGGTKYAQKFQGRVTMTRDTSTSTVYMELSSLRSEDTAVYYCARVLQEGMDVWGQGTLVTVSS

SEQ ID NO: 674 - protein, 106 aa, synthetic construct
DIQMTQSPSSLSASVGDRVTITCRASQSISSYLNWYQQKPGKAPKLLIYGVSSLQSGVPSRFSGSGSGTDFTLTISSLQPEDFATYYCQQSYSNPTFGQGTKVEIK

SEQ ID NO: 675 - protein, 115 aa, synthetic construct
EVQLLESGAEVKKPGASVKVSCKASGYTFTDYIVHWLRQAPGQGLEWMGWINPNSGGTNYAQNFQGRVTMTRDTSTSTVYMELSSLRSEDTAVYYCARANWLDYWGQGTLVTVSS

SEQ ID NO: 676 - protein, 107 aa, synthetic construct
DIQMTQSPSSLSASVGDRVTITCRASQSISRWLAWYQQKPGKAPKLLIYGASSLQSGVPSRFSGSGSGTDFTLTISSLQPEDFATYYCQQSYITPYTFGQGTKVEIK

SEQ ID NO: 677 - protein, 117 aa, synthetic construct
QVQLVQSGAEVKKPGASVKVSCKASGYTFTDYLIHWVRQAPGQGLEWMGWINPASGGTNYAQKFQGRVTMTRDTSTSTVYMELSSLRSEDTAVYYCAGGYRWLHDYWGQGTLVTVSS

SEQ ID NO: 678 - protein, 107 aa, synthetic construct
DIQMTQSPSSLSASVGDRVTITCRASQSISRWLAWYQQKPGKAPKLLIYAASTLQSGVPSRFSGSGSGTDFTLTISSLQPEDFATYYCQQSYSPPYTFGQGTKLEIK

SEQ ID NO: 679 - protein, 115 aa, synthetic construct
EVQLLESGAEVKKPGSSVKVSCKASGYTFTDYNIHWVRQAPGQGLEWMGWINPNSGGTNYAQRFQGRVTMTRDTSTSTVYMELSSLRSEDTAVYYCARANWLDYWGQGTLVTVSS

SEQ ID NO: 680 - protein, 107 aa, synthetic construct
DIQMTQSPSSLSASVGDRVTITCRASQDISLWLAWYQQKPGKAPKLLIYAASTLQSGVPSRFSGSGSGTDFTLTISSLQPEDFATYYCQQSYMTPRTFGPGTKVDIK

SEQ ID NO: 681 - protein, 118 aa, synthetic construct
QVQLVQSGAEVKKPGASVKVSCKASGYTFTDYQMFWVRQAPGQGLEWMGWINPNSGGTNYAQKFQGRVTMTRDTSTSTVYMELSSLRSEDTAVYYCAKGGGGYGMDVWGQGTTVTVSS

SEQ ID NO: 682 - protein, 107 aa, synthetic construct
DIQMTQSPSSLSASVGDRVTITCRASQGISNYLAWYQQKPGKAPKLLIYAASSLQSGVPSRFSGSGSGTDFTLTISSLQPEDFATYYCQQSYSDQWTFGQGTKVEIK

SEQ ID NO: 683 - protein, 119 aa, synthetic construct
QVQLVQSGAEVKKPGASVKVSCKASGDTFSFYAISWVRQAPGQGLEWMGWMNPNSGGTTYAQKFQGRVTMTRDTSTSTVYMELSSLRSEDTAVYYCARGVQDYYGSDYWGQGTLXTVSS

SEQ ID NO: 684 - protein, 107 aa, synthetic construct
DIQMTQSPSSLSASVGDRVTITCRASQSIGRWLAWYQQKPGKAPKLLIYAASTLQTGVPSRFSGSGSGTDFTLTISSLQPEDFATYYCQQGYSSPYTFGQGTKVEIK

SEQ ID NO: 685 - protein, 117 aa, synthetic construct
QVQLVQSGAEVKKPGASVQVSCKASGYTFTGYNIHWVRQAPGQGLEWMGWINPNSGGTNYAQKFQGRVTMTRDTSTSTVYMELSSLRSEDTAVYYCARDEAGYFDLWGRGTLVTVSS

SEQ ID NO: 686 - protein, 107 aa, synthetic construct
DIQMTQSPSSLSASVGDRVTITCQASQDISNYLNWYQQKPGKAPKLLIYAASTLESGVPSRFSGSGSGTDFTLTISSLQPEDFATYYCQQSFTIPWTFGQGTKLEIK

SEQ ID NO: 687 - protein, 107 aa, synthetic construct
DIQMXQSPSSLSASVGDRVTITCRASQSINRWLAWYQQKPGKAPKLLIYAASTLQSGVPSRFSGSGSGTDFTLTISSLQPEDFATYYCQQSYITPYTFGQGTKLEIK

SEQ ID NO: 688 - protein, 115 aa, synthetic construct
QVQLVQSGAEVKKPGASVKVSCKASGYTFTGYNMHWVRQAPGQGLEWMGWINPNSGGTNYAQKFQGRVTMTRDTSTSTVYMELSSLRSEDTAVYYCARGDWFDPWGQGTLVTVSS

SEQ ID NO: 689 - protein, 107 aa, synthetic construct
DIQMTQSPSSLSASVGDRVTITCRASQSISSWLAWYQQKPGKAPKLLIYAASSLQSGVPSRFSGSGSGTDFTLTISSLQPEDFATYYCQQSYSPPYTFGQGTKVDIK

SEQ ID NO: 690 - protein, 117 aa, synthetic construct
QVQLVQSGAEVKKPGASVKVSCKASGYMFTGYNVHWVRQAPGQGLEWMGWINPNSGGTNYAQKFQGRVTMTRDTSTSTVYMELSSLRSEDTAVYYCARDHSSGYDYWGQGTLVTVSS

SEQ ID NO: 691 - protein, 107 aa, synthetic construct
DIQMTQSPSSLSASVGDRVTITCRASQGISSWLAWYQQKPGKAPKLLIYAASSLQSGVPSRFSGSGSGTDFTLTISSLQPEDFATYYCQQSYSTPYTFGQGTKLEIK

SEQ ID NO: 692 - protein, 117 aa, synthetic construct
QVQLVQSGAEVKKPGASVKVSCKASGYTLTGYYIHWVRQAPGQGLEWMGWINPNSGGTNYAQKFQGRVTMTRDTSTSTVYMELSSLRSEDTAVYYCARENYFDXXXWGXXTTVTASS

SEQ ID NO: 693 - protein, 107 aa, synthetic construct
DIQMTQSPSSLSASVGDRVTITCRASQSISRWLTWYQQKPGKAPKLLIYAASSLQSGVPSRFSGSGSGTDFTLTISSLQPEDFATYYCQQSYSPPXTFGRGTKVEIK

SEQ ID NO: 694 - protein, 115 aa, synthetic construct
QVQLVQSGAEVKKPGASVKVSCKASGYSFTGYYINWVRQAPGQGLEWMGWINPNSGGTNYAQKFQGRVTMTRDTSTSTVYMELSSLRSEDTAVYYCAAGEWFDPWGQGTLVTVSS

SEQ ID NO: 695 - protein, 107 aa, synthetic construct
DIQMTQSPSSLSASVGDRVTITCRASQDISNWLAWYQQKPGKAPKLLIYGASTLQSGVPSRFSGSGSGTDFTLTISSLQPEDFATYYCQQSMIAPYTFGQGTKVEIK

SEQ ID NO: 696 - protein, 126 aa, synthetic construct
QVQLVQSGAEVKKPGASVKVSCKASGYTFTGYYMHWVRQAPGQGLEWMGWMNPNSGNTGYAQKFQGRVTMTRDTSTSTVYMELSSLRSEDTAVYYCARGSEIIDYDSSGLGAFDIWGQGTMVTVSS

SEQ ID NO: 697 - protein, 107 aa, synthetic construct
DIQMTQSPSSLSASVGDRVTITCQASQSIYNYLNWYQQKPGKAPKLLIYAASSLQSGVPSRFSGSGSGTDFTLTISSLQPEDFATYYCQQSYSVPHTFGQGTKLEIK

SEQ ID NO: 698 - protein, 117 aa, synthetic construct
QVQLVQSGAEVKKPGASVKVSCKASGASFSNSIINWVRQAPGQGLEWMGWIYPNSGGTNYAQKFQGRVTMTRDTSTSTVYMELSSLRSEDTAVYYCARRWMDAFDIWGQGTTVTVSS

SEQ ID NO: 699 - protein, 107 aa, synthetic construct
DIQMTQSPSSLSASVGDRVTITCRASQGISNYLAWYQQKPGKAPKLLIYAASTLQGGVPSRFSGSGSGTDFTLTISSLQPEDFATYYCQQTYSVPLTFGGGTKVEIK

SEQ ID NO: 700 - protein, 115 aa, synthetic construct
EVQLLESGAEVKKPGASVKVSCKASGYTFINYGITWVRQAPGQGLEWMGWINANSGGTNYAQKFQGRVTMTRDTSTSTVYMELSSLRSEDTAVYYCARANWLDYWGQGTLVTVSS

SEQ ID NO: 701 - protein, 107 aa, synthetic construct
DIQMTQSPSSLSASVGDRVTITCRASQSIGSWLAWYQQKPGKAPKLLIYAASSLQSGVPSRFSGSGSGTDFTLTISSLQPEDFATYYCQQSYSAPYTFGQGTKLEIK

SEQ ID NO: 702 - protein, 116 aa, synthetic construct
QVQLVQSGAEVKKPGASVKVSCKASGGTFSNYILSWVRQAPGQGLEWMGWINPDSGGTNYAQKFQGRVTMTRDTSTSTVYMELSSLRSEDTAVYYCARGSYGMDVWGQGTTVTVSS

SEQ ID NO: 703 - protein, 117 aa, synthetic construct
QVQLVQSGAEVKKPGASVKVSCKASGYTFTNYNIHWVRQAPGQGLEWMGWINPNSGGTNYAQRFQGRVTMTRDTSTSTVYMELSSLRSEDTAVYYCAREGLAALDYWGQGTLVTVSS

SEQ ID NO: 704 - protein, 107 aa, synthetic construct
DIQMTQSPSSLSASVGDRVTITCRASQSISNWLAWYQQKPGKAPKLLIYAASTLQSGVPSRFSGSGSGTDFTLTISSLQPEDFATYYCQQSYISPYTFGPGTKVDIK

SEQ ID NO: 705 - protein, 117 aa, synthetic construct
QVQLVQSGAEVKKPGASVKVSCKASGYMFTNYPMTWVRQAPGQGLEWMGWINPNSGGTNYAQKFQGRVTMTRDTSTSTVYMELSSLRSEDTAVYYCARDAAAGFDPWGQGTLVTVSS

SEQ ID NO: 706 - protein, 107 aa, synthetic construct
DIQMTQSPSSLSASVGDRVTITCRASQDISNWLAWYQQKPGKAPKLLIYAASKLQSGVPSRFSGSGSGTDFTLTISSLQPEDFATYYCQQSYSPPYSFGQGTKLEIK

SEQ ID NO: 707 - protein, 117 aa, synthetic construct
QVQLVQSGAEVKKPGASVKVSCKASGGTFSNYVISWVRQAPGQGLEWMGWINPNSGGTNFAQKFQGRVTMTRDTSTSTVYMELSSLRSEDTAVYYCAREGRSSYDYWGQGTLVTVSS

SEQ ID NO: 708 - protein, 107 aa, synthetic construct
DIQMTQSPSSLSASVGDRVTITCRASQSIGRWLAWYQQKPGKAPKLLIYGASSVQSGVPSRFSGSGSGTDFTLTISSLQPEDFATYYCQQSYSSPYTFGQGTKLEIK

SEQ ID NO: 709 - protein, 121 aa, synthetic construct
QVQLVQSGAEVKKPGASVKVSCKASGYTFTNYYMHWVRQAPGQGLEWMGWMNPNSGHTSYAQKFQGRVTMTRDTSTSTVYMELSSLRSEDTAVYYCARVGYSSGWLGMDVWGQGTTVTVSS

SEQ ID NO: 710 - protein, 107 aa, synthetic construct
DIQMTQSPSSLSASVGDRVTITCQASQDIRKYLNWYQQKPGKAPKLLIYAASSLQSGVPSRFSGSGSGTDFTLTISSLQPEDFATYYCQQSYTIPYTFGQGTKLEIK

SEQ ID NO: 711 - protein, 115 aa, synthetic construct
EVQLLESGAEVKKPGASVKVSCKASGYTLTSHLIHWVRQAPGQGLEWVGWINPNTGGTNYAQKFQGRVTMTRDTSTSTVYMELSSLRSEDTAVYYCARANWLDYWGQGTLVTVSS

SEQ ID NO: 712 - protein, 107 aa, synthetic construct
DIQMTQSPSSLSASVGDRVTITCRASQDISRWLAWYQQKPGKAPKLLIYAASSLQSGVPSRFSGSGSGTDFTLTISSLQPEDFATYYCQQSYTTPYTFGQGTKLEIK

SEQ ID NO: 713 - protein, 124 aa, synthetic construct
QVQLVQSGAEVKKPGSSVKVSCKASGYTFSSSDINWVRQAPGQGLEWMGGIIPIFGTANYAQKFQGRVTITADESTSTAYMELSSLRSEDTAVYYCARADTAMGHYYYYGMDVWGQGTTVTVSS

SEQ ID NO: 714 - protein, 112 aa, synthetic construct
DIVMTQSPLSLPVTPGEPASISCRSSQSLLNKNGNNYIDWYLQKPGQSPQLLIYQGSNRASGVPDRFSGSGSGTDFTLKISRVEAEDVGVYYCMQALQTPPTFGGGTKVEIK

SEQ ID NO: 715 - protein, 120 aa, synthetic construct
QVQLVQSGAEVKKPGASVKVSCKASGGTFSSYAFSWVRQAPGQGLEWMGVIYPSGGTTGYAQKFQGRVTMTRDTSTSTVYMELSSLRSEDTAVYYCARGLHQLPNWFDPWGQGTLVTVSS

SEQ ID NO: 716 - protein, 107 aa, synthetic construct
DIQMTQSPSSLSASVGDRVTITCRASQSISRWLAWYQQKPGKAPKLLIYAASSLQSGVPSRFSGSGSGTDFTLTISSLQPEDFATYYCQQSYSSPYTFGQGTKVEIK

SEQ ID NO: 717 - protein, 120 aa, synthetic construct
QVQLVQSGAEVKKPGASVKVSCKASGGTFSSYAISWVRQAPGQGLEWMGWINPNSGGTNYAQKFQGRVTMTRDTSTSTVYMELSSLRSEDTAVYYCARDLVGGVFAFDIWGQGTTVTVSS

SEQ ID NO: 718 - protein, 107 aa, synthetic construct
DIQMTQSPSSLSASVGDRVTITCRASQNITTYLNWYQQKPGKAPKLLIYAASTLQSGVPSRFSGSGSGTDFTLTISSLQPEDFATYYCQQSYSMPYSFGQGTKLEIK

SEQ ID NO: 719 - protein, 118 aa, synthetic construct
QVQLVQSGAEVKKPGASVKVSCKASGGTFSSYAISWVRQAPGQGLEWMGGIIPIFGAPVYAQDFQGRVTITADESTSTAYMELSSLRSEDTAVYYCARDRGADAFDIWGQGTTVTVSS

SEQ ID NO: 720 - protein, 113 aa, synthetic construct
DIVMTQSPDSLAVSLGERATINCKSSQSVLYSSNNKNYLAWYQQKPGQPPKLLIYWASTRASGVPDRFSGSGSGTDFTLTISSLQAEDVAVYYCQQYERTPLTFGGGTKVEIK

SEQ ID NO: 721 - protein, 115 aa, synthetic construct
EVQLVQSGAEVKKPGASVKVSCKASGGTFSSYAISWVRQAPGQGLEWIGWINPNSGGTNYAQKFQGRVTMTRDTSTSTVYMELSSLRSEDTAVYYCARANWLDYWGQGTLVTVSS

SEQ ID NO: 722 - protein, 107 aa, synthetic construct
DIQMTQSPSSLSASVGDRVTITCRASQSISRWLAWYQQKPGKAPKLLIYAASSLQSGVPSRFSGSGSGTDFTLTISSLQPEDFATYYCQQSYSPPYTFGQGTKLEIK

SEQ ID NO: 723 - protein, 116 aa, synthetic construct
QVQLVQSGAEVKKPGASVKVSCKASGGTFSSYAISWVRQAPGQGLEWMGWINPNSGGTKYAQKFQGRVTMTRDTSTSTVYMELSSLRSEDTAVYYCAREISFLDYWGQGTLVTVSS

SEQ ID NO: 724 - protein, 107 aa, synthetic construct
DIQMTQSPSSLSASVGDRVTITCRASQSISNWLAWYQQKPGKAPKLLIYGASSLQSGVPSRFSGSGSGTDFTLTISSLQPEDFATYYCQQSYTTPYTFGQGTKLEIK

SEQ ID NO: 725 - protein, 116 aa, synthetic construct
QVQLVQSGAEVKKPGASVKVSCKASGGTFSSYAVSWVRQAPGQGLEWMGWIIPDSGGTNYAQKLQGRVTMTTDTSTGTAYMELRSLRSDDTAVYYCTRGSGWYDYWGQGTLVTVSS

SEQ ID NO: 726 - protein, 106 aa, synthetic construct
DIQMTQSPSSLSASVGDRVTITCRASQSISNWLAWYQQKPGKAPKLLIYGASTLQSGVPSRFSGSGSGTDFTLTISSLQPEDFATYYCQQSYSKPTFGQGTKVEIK

SEQ ID NO: 727 - protein, 115 aa, synthetic construct
QVQLVQSGAEVKKPGASVKVSCKASGYTFTSYDINWVRQAPGQGLEWMGWINPNSGGTNYAQKFQGRVTMTRDTSTSTVYMELSSLRSEDTAVYYCARANWLDYWGQGTLVTISS

SEQ ID NO: 728 - protein, 107 aa, synthetic construct
DIQMTQSPSSLSASVGDRVTITCRASQNINRWLAWYQQKPGKAPKLLIYAASSLQSGVPSRFSGSGSGTDFTLTISSLQPEDFATYYCQQSYSPPYTFGQGTKVEIK

SEQ ID NO: 729 - protein, 107 aa, synthetic construct
DIQMTQSPSSLSASVGDRVTITCRASQSIRSWLAWYQQKPGKAPKLLIYAASSLQSGVPSRFSGSGSGTDFTLTISSLQPEDFATYYCQQSYSTPYTFGQGTKVEIK

SEQ ID NO: 730 - protein, 115 aa, synthetic construct
EVQLLESGAEVKKPGASVKVSCKASGYTFSSYGIFWVRQAPGQGLEWMGWINPNSGGTNYAQKFQGRVTMTRDTSTSTVYMELSSLRSEDTAVYYCARANWLDYWGQGTLVTVSS

SEQ ID NO: 731 - protein, 106 aa, synthetic construct
DIQMTQSPSSLSASVGDRVTITCRASQDIGSWLAWYQQKPGKAPKLLIYAASSLQSGVPSRFSGSGSGTDFTLTISSLQPEDFATYYCQQSYSMITFGQGTKVEIK

SEQ ID NO: 732 - protein, 116 aa, synthetic construct
QVQLVQSGAEVKKPGASVKVSCKASGYTFTSYGISWVRQAPGQGLEWMGWINPNSGGTNYAQKFQGRVTMTRDTSTSTVYMELSSLRSEDTAVYYCARDFDAFDIWGQGTMVTVSS

SEQ ID NO: 733 - protein, 107 aa, synthetic construct
DIQMTQSPSSLSASVGDRVTITCRASEAIGTWLAWYQQKPGKAPKLLIYAASSLQSGVPSRFSGSGSGTDFTLTISSLQPEDFATYYCQQSYSPPYTFGQGTKLEIK

SEQ ID NO: 734 - protein, 118 aa, synthetic construct
QVQLVQSGAEVKKPGASVKVSCKASGYTFTSYGISWVRQAPGQGLEWMGWINPNSGGTNYAQKFQGRVTMTRDTSTSTVYMELSSLRSEDTAVYYCARSTDYYYMDVWGKGTTVTVSS

SEQ ID NO: 735 - protein, 107 aa, synthetic construct
DIQMTQSPSSLSASVGDRVTITCRASQNIRGYLNWYQQKPGKAPKLLIYAASSLQSGVPSRFSGSGSGTDFTLTISSLQPEDFATYYCQQSYSNPWTFGQGTKVEIK

SEQ ID NO: 736 - protein, 116 aa, synthetic construct
QVQLVQSGAEVKKPGASVKVSCKASGYTFTSYGISWVRQAPGQGLEWMGWINPNSGGTNYAQNFQGRVTMTRDTSTSTVYMELSSLRSEDTAVYYCARNYDSYDYWGQGTLVTVSS

SEQ ID NO: 737 - protein, 107 aa, synthetic construct
DIQMTQSPSSLXASVGDRVTITCRASQSVGSWLAWYQQKPGKAPKLLIYGASTLQDGVPSRFSGSGSGTDFTLTISSLQPEDFATYYCQQSYNTPYTFGRGTKVEIK

SEQ ID NO: 738 - protein, 115 aa, synthetic construct
EVQLLESGAEVKKPGASVKVSCKASGYTFTSYGISWVRQAPGQGLEWMGWLNPNSGGTNYAQQFQGRVTMTRDTSTSTVYMELSSLRSEDTAVYYCARANWLDYWGQGTLVTVSS

SEQ ID NO: 739 - protein, 107 aa, synthetic construct
DIQMTQSPSSLSASVGDRVTITCRASQRIGNWLAWYQQKPGKAPKLLIYAASSLQSGVPSRFSGSGSGTDFTLTISSLQPEDFATYYCLQDYNYPYTFGQGTKLEIK

SEQ ID NO: 740 - protein, 116 aa, synthetic construct
QVQLVQSGAEVKKPGASVKVSCKASGYTFTSYGISWVRQAPGQGLEWMGWINPNSGGTNYAQKLQGRVTMTRDTSTSTVYMELSSLRSEDTAVYYCARDYGALDYWGQGTLVTVSS

SEQ ID NO: 741 - protein, 107 aa, synthetic construct
DIQMTQSPSSLSASVGDRVTITCRASQAIGSWLAWYQQKPGKAPKLLIYAASSLQSGVPSRFSGSGSGTDFTLTISSLQPEDFATYYCQQSYSAPYTFGPGTKVDIK

SEQ ID NO: 742 - protein, 116 aa, synthetic construct
QVQLVQSGAEVKKPGASVKVSCKASGDTFTSYGISWVRQAPGQGLEWMGWIIPNSGGTNYAQKFQGRVTMTRDTSTSTVYMELSSLRSEDTAVYYCARGSGWSDYWGQGTLVTVSS

SEQ ID NO: 743 - protein, 107 aa, synthetic construct
DIQMTQSPSSLSASVGDRVTITCRASQSISSWLAWYQQKPGKAPKLLIYAASSLQSGVPSRFSGSGSGTDFTLTISSLQPEDFATYYCQQGYSTPYTFGQGTKLEIK

SEQ ID NO: 744 - protein, 115 aa, synthetic construct
EVQLLESGAEVKKPGASVKVSCKASGYTLTSYGISWVRQAPGQGLEWMGWINPNSGGTNYAQNFQGRVTMTRDTSTSTVYMELSSLRSEDTAVYYCARANWLDYWGQGTLVTVSS

SEQ ID NO: 745 - protein, 107 aa, synthetic construct
DIQMTQSPSSLSASVGDRVTITCRASQNIGRWLAWYQQKPGKAPKLLIYAASSLQSGVPSRFSGSGSGTDFTLTISSLQPEDFATYYCRQSYSPPYTFGQGTKLEIK

SEQ ID NO: 746 - protein, 115 aa, synthetic construct
EVQLLESGAEVKKPGSSVKVSCKASGYTFSSYNMHWVRQAPGQGLEWMGWINPNSGGTNYAQKFQGRVTMTRDTSTSTVYMELSSLRSEDTAVYYCARANWLDYWGQGTLVTVSS

SEQ ID NO: 747 - protein, 107 aa, synthetic construct
DIQMTQSPSSLSASVGDRVTITCRASQSISSWLAWYQQKPGKAPKLLIYGASNLQTGVPSRFSGSGSGTDFTLTISSLQPEDFATYYCQQSYSPPYTFGQGTKVEIK

SEQ ID NO: 748 - protein, 115 aa, synthetic construct
EVQLLESGAEVKKPGSSVKVSCKASGGTFSSYPISWVRQAPGQGLEWMGWINPNSGGTNYAQNFQGRVTITADESTSTAYMELSSLRSEDTAVYYCARANWLDYWGQGTLVTVSS

SEQ ID NO: 749 - protein, 107 aa, synthetic construct
DIQMTQSPSSLSASVGDRVTITCRASQSISRWLAWYQQKPGKAPKLLIYATSTLQSGVPSRFSGSGSGTDFTLTISSLQPEDFATYYCQQSYSIPYAFGQGTKVEIK

SEQ ID NO: 750 - protein, 115 aa, synthetic construct
EVQLLESGAEVKKPGASVKVSCKASGYTFSSYTISWVRQAPGQGLEWMGWINPNSGGTNYAQKFQGRVTMTRDTSTSTVYMELSSLRSEDTAVYYCARANWLDYWGQGTLVTVSS

SEQ ID NO: 751 - protein, 107 aa, synthetic construct
DIQMTQSPSSLSASVGDRVTITCRASQTISRWLAWYQQKPGKAPKLLIYAASTLQSGVPSRFSGSGSGTDFTLTISSLQPEDFATYYCQQSYSTPYTFGQGTKLEIK

SEQ ID NO: 752 - protein, 117 aa, synthetic construct
QVQLVQSGAEVKKPGASVKVSCKASGGTFSSYVISWVRQAPGQGLEWMGWINPNNGGTNYAQKFQGRVTMTRDTSTSTVYMELSSLRSEDTAVYYCARDSGVGMDVWGQGTTVTVSS

SEQ ID NO: 753 - protein, 107 aa, synthetic construct
DIQMTQSPSSLSASVGDRVTITCRASQSISKWLAWYQQKPGKAPKLLIYAASSLQSGVPSRFSGSGSGTDFTLTISSLQPEDFATYYCQQSYSAPYTFGQGTKVEIK

SEQ ID NO: 754 - protein, 116 aa, synthetic construct
QVQLVQSGAEVKKPGASVKVSCKASGGTFSSYVISWVRQAPGQGLEWMGWINPNSGGTNYAQKFQGRVTMTRDTSTSTVYMELSSLRSEDTAVYYCARDSAGMDVWGQGTTVTVSS

SEQ ID NO: 755 - protein, 107 aa, synthetic construct
DIQMTQSPSSLSASVGDRVTITCRASQSISSWLAWYQQKPGKAPKLLIYAASSLQSGVPSRFSGSGSGTDFTLTISSLQPEDFATYYCQQSYSSPYTFGQGTKVEIK

SEQ ID NO: 756 - protein, 116 aa, synthetic construct
QVQLVQSGAEVKKPGASVKVSCKASGGTFSSYVITWVRQAPGQGLEWMGWINPNSGGTNYAQEFQGRVTMTRDTSTSTVYMELSSLRSEDTAVYYCARGHMALDYWGQGTLVTVSS

SEQ ID NO: 757 - protein, 107 aa, synthetic construct
DIQMTQSPSSLSASVGDRVTITCRASQNINTWLAWYQQKPGKAPKLLIYAASTLQSGVPSRFSGSGSGTDFTLTISSLQPEDFATYYCQQSYIAPYTFGQGTKLEIK

SEQ ID NO: 758 - protein, 115 aa, synthetic construct
EVQLLESGAEVKKPGASVKVSCKASGYTFTSYVMNWVRQAPGQGLEWMGWINPNSGGTSYAQKFQGRVTMTRDTSTSTVYMELSSLRSEDTAVYYCARANWLDYWGQGTTVTVSS

SEQ ID NO: 759 - protein, 107 aa, synthetic construct
DIQMTQSPSSLSASVGDRVTITCRASQGISSWLAWYQQKPGKAPKLLIYAASSLQSGVPSRFSGSGSGTDFTLTISSLQPEDFATYYCQQSYSPPYTFGQGTKLEIK

SEQ ID NO: 760 - protein, 125 aa, synthetic construct
QVQLVQSGAEVKKPGASVKVSCKASGYTFTSYYLHWVRQAPGQGLEWMGIINPSGGSTSYAQKFQGRVTMTRDTSTSTVYMELSSLRSEDTAVYYCAKDTEGYGSGSRPYGMDVWGQGTLVTVSS

SEQ ID NO: 761 - protein, 107 aa, synthetic construct
DIQMTQSPSSLSASVGDRVTITCRASQGISSWLAWYQQKPGKAPKLLIYAASSLQSGVPSRFSGSGSGTDFTLTISSLQPEDFATYYCQQAYSFPYTFGQGTKVEIK

SEQ ID NO: 762 - protein, 115 aa, synthetic construct
QVQLVQSGAEVKKPGASVKVSCKASGYTFTSYYMHWVRQAPGQGLEWMGWINPNSGGTNYAQKFQGRVTMTRDTSTSTVYMELSSLRSEDTAVYYCARGEWFDPWGQGTLVTVSS

SEQ ID NO: 763 - protein, 107 aa, synthetic construct
DIQMTQSPSSLSASVGDRVTITCRASQSIGTWLAWYQQKPGKAPKLLIYAASTLQSGVPSRFSGSGSGTDFTLTISSLQPEDFATYYCQQSYSTPYTFGQGTKLEIK

SEQ ID NO: 764 - protein, 107 aa, synthetic construct
DIQMTQSPSSLSASVGDRVTITCRASQSISSWLAWYQQKPGKAPKLLIYAASSLQSGVPSRFSGSGSGTDFTLTISSLQPEDFATYYCQQSYRTPYTFGQGTKLEIK

SEQ ID NO: 765 - protein, 120 aa, synthetic construct
QVQLVQSGAEVKKPGASVKVSCKASGYTFTTYDISWVRQAPGQGLEWMGWMSPNSGGTNYAQNFQGRVTMTRDTSTSTVYMELSSLRSEDTAVYYCARSLHPYSNYWVYWGPGTLVTVSS

SEQ ID NO: 766 - protein, 107 aa, synthetic construct
DIQMTQSPSSLSASVGDRVTITCQASQDISNYLNWYQQKPGKAPKLLIYAASSLRGGVPSRFSGSGSGTDFTLTISSLQPEDFATYYCQQSYNTPYSFGQGTKLEIK

SEQ ID NO: 767 - protein, 115 aa, synthetic construct
QVQLVQSGAEVKKPGASVKVSCKASGYSFTTYLIHWVRQAPGQGLEWMGWINPNSGGTNYAQKFQGRVTMTRDTSTSTVYMELSSLRSEDTAVYYCARANWLDYWGQGTLVTVSS

SEQ ID NO: 768 - protein, 107 aa, synthetic construct
DIQMTQSPSSLSASVGDRVTITCRASQSISTWLAWYQQKPGKAPKLLIYAASSLQSGVPSRFSGSGSGTDFTLTISSLQPEDFATYYCQQSYSAPYTFGQGTKLEIK

SEQ ID NO: 769 - protein, 115 aa, synthetic construct
QVQLVQSGAEVKKPGASVKVSCKASGGTLSTYTISWVRQAPGQGLEWMGWMNPNSGGTNYAQKFQGRVTMTRDTSTSTVYMELSSLRSEDTAVYYCARGDWQDIWGQGTTVTVSS

SEQ ID NO: 770 - protein, 115 aa, synthetic construct
QVQLVQSGAEVKKPGASVKVSCKASGYTFSTYTITWVRQAPGQGLEWMGWINPKSGGTNYAQKFQGRVTMTRDTSTSTVYMELSSLRSEDTAVYYCARGEWLDYWGQGTLVTVSS

SEQ ID NO: 771 - protein, 107 aa, synthetic construct
DIQMTQSPSSLSASVGDRVTITCRASQSIGTWLAWYQQKPGKAPKLLIYAASTLQSGVPSRFSGSGSGTDFTLTISSLQPEDFATYYCQQSYGTPWTFGQGTKVEIK

SEQ ID NO: 772 - protein, 115 aa, synthetic construct
QVQLVQSGAEVKKPGASVKVSCKASGYTFTTYYVHWVRQAPGQGLEWMGRISPNSGGTNYAQKFQGRVTMTRDTSTSTVYMELSSLRSEDTAVYYCARDWSMDVWGQGTLVTVSS

SEQ ID NO: 773 - protein, 106 aa, synthetic construct
DIQMTQSPSSLSASVGDRVTITCQASQDISNYLNWYQQKPGKAPKLMIYAASTLQSGVPSRFSGSGSGTDFTLTISSLQPEDFATYYCQQSYSTWTFGQGTKVEIK

SEQ ID NO: 774 - protein, 115 aa, synthetic construct
QVQLVQSGAEVKKPGASVKVSCKASGYTLTXHLIHWVRQAPGQGLEWMGWINPNSGGTNYAQKLQGRVTMTRDTSTSTVYMELSSLRSEDTAVYYCARDYGMDVWGXGTTXTVSS

SEQ ID NO: 775 - protein, 107 aa, synthetic construct
DIQMTQSPSSLSASVGDRVTITCRASQXVNRWLAWYQQKPGKAPKLLIYAASSLQSGVPSRFSGSGSGTDFTLTISSLQPEDFATYYCQQSYITPYTFGQGTKLEIK

(19) United States
(12) Patent Application Publication
(10) Pub. No.: US 2023/0287128 A1
(43) Pub. Date: Sep. 14, 2023

(54) ANTIGEN-BINDING PROTEINS TARGETING KKLC-1 SHARED ANTIGEN

(71) Applicant: Gritstone bio, Inc., Emeryville, CA (US)

(72) Inventors: Godfrey Jonah Anderson Rainey, San Diego, CA (US); Karin Jooss, San Diego, CA (US); Manankumar Anilkumar Shah, Emeryville, CA (US)

(21) Appl. No.: 17/820,434

(22) Filed: Aug. 17, 2022

Related U.S. Application Data
(63) Continuation of application No. PCT/US2021/018912, filed on Feb. 19, 2021.
(60) Provisional application No. 62/979,271, filed on Feb. 20, 2020.

(51) Int. Cl. C07K 16/28; C07K 14/725; A61K 39/00

(52) U.S. Cl. CPC C07K 16/2833; C07K 14/7051; A61K 39/4611; A61K 39/4631; C07K 2317/92; C07K 2317/622

(57) ABSTRACT

Provided herein are antigen binding proteins that selectively bind a particular KKLC-1 shared antigen, as well as related methods, kits, and compositions.

### CROSS REFERENCE TO RELATED APPLICATIONS

This application is a continuation of International Application No. PCT/US2021/018912, filed Feb. 19, 2021, which claims priority to and the benefit of U.S. Provisional Patent Application No. 62/979,271, filed on Feb. 20, 2020, each of which is hereby incorporated in its entirety by reference for all purposes.

### REFERENCE TO A SEQUENCE LISTING XML

The instant application contains a Sequence Listing which has been filed electronically in XML format. The Sequence Listing XML is hereby incorporated by reference in its entirety. Said XML file, created on Aug. 13, 2022, is named GSO-087WOC1_SL.xml and is 704,931 bytes in size.

### BACKGROUND

The immune system employs two types of adaptive immune responses to provide antigen specific protection from pathogens; humoral immune responses, and cellular immune responses, which involve specific recognition of pathogen antigens via B lymphocytes and T lymphocytes, respectively.

T lymphocytes, by virtue of being the antigen specific effectors of cellular immunity, play a central role in the body's defense against diseases mediated by intracellular pathogens, such as viruses, intracellular bacteria, mycoplasmas, and intracellular parasites, and against cancer cells by directly cytolysing the affected cells. The specificity of T lymphocyte responses is conferred by, and activated through T-cell receptors (TCRs) binding to (major histocompatibility complex) MHC molecules on the surface of affected cells. T-cell receptors are antigen specific receptors clonally distributed on individual T lymphocytes whose repertoire of antigenic specificity is generated via somatic gene rearrangement mechanisms analogous to those involved in generating the antibody gene repertoire. T-cell receptors include a heterodimer of transmembrane molecules, the main type being composed of an alpha-beta polypeptide dimer and a smaller subset of a gamma-delta polypeptide dimer. T lymphocyte receptor subunits comprise a variable and constant region similar to immunoglobulins in the extracellular domain, a short hinge region with cysteine that promotes alpha and beta chain pairing, a transmembrane and a short cytoplasmic region. Signal transduction triggered by TCRs is indirectly mediated via CD3-zeta, an associated multi-subunit complex comprising signal transducing subunits.

T lymphocyte receptors do not generally recognize native antigens but rather recognize cell-surface displayed complexes comprising an intracellularly processed fragment of an antigen in association with a major histocompatibility complex (MHC) for presentation of peptide antigens. Major histocompatibility complex genes are highly polymorphic across species populations, comprising multiple common alleles for each individual gene. In humans, MHC is referred to as human leukocyte antigen (HLA).

Major histocompatibility complex class I molecules are expressed on the surface of virtually all nucleated cells in the body and are dimeric molecules comprising a transmembrane heavy chain, comprising the peptide antigen binding cleft, and a smaller extracellular chain termed beta2-microglobulin. MHC class I molecules present peptides derived from the degradation of cytosolic proteins by the proteasome, a multi-unit structure in the cytoplasm, (Niedermann G, 2002. Curr Top Microbiol Immunol. 268:91-136; for processing of bacterial antigens, refer to Wick M J, and Ljunggren H G., 1999. Immunol Rev. 172:153-62). Cleaved peptides are transported into the lumen of the endoplasmic reticulum (ER) by the transporter associated with antigen processing (TAP) where they are bound to the groove of the assembled class I molecule, and the resultant MHC/peptide complex is transported to the cell membrane to enable antigen presentation to T lymphocytes (Yewdell J W., 2001. Trends Cell Biol. 11:294-7; Yewdell J W. and Bennink J R., 2001. Curr Opin Immunol. 13:13-8). Alternatively, cleaved peptides can be loaded onto MHC class I molecules in a TAP-independent manner and can also present extracellularly-derived proteins through a process of cross-presentation. As such, a given MHC/peptide complex presents a novel protein structure on the cell surface that can be targeted by a novel antigen-binding protein (e.g., antibodies or TCRs) once the identity of the complex's structure (peptide sequence and MHC subtype) is determined.

Tumor cells can express antigens and may display such antigens on the surface of the tumor cell. Such tumor-associated antigens can be used for development of novel immunotherapeutic reagents for the specific targeting of tumor cells. For example, tumor-associated antigens can be used to identify therapeutic antigen binding proteins, e.g., antibodies or antigen-binding fragments thereof.

Normal cells also display restricted peptides on their surface. In some cases, restricted peptides displayed by normal cells can have sequence overlap to the tumor-specific antigens. Such sequence-overlapping restricted peptides therefore represent potential off-target liabilities for therapeutic cancer immunotherapy.

Therefore, there exists a need for antigen-binding proteins that selectively bind tumor-specific antigens displayed on the surface of tumor cells, with minimal or no off-target liability.

### SUMMARY

Provided herein is an isolated antigen binding protein (ABP) that specifically binds to a human leukocyte antigen (HLA)-PEPTIDE target, wherein the HLA-PEPTIDE target comprises an HLA-restricted peptide complexed with an HLA Class I molecule, wherein the HLA-restricted peptide is located in the peptide binding groove of an α1/α2 heterodimer portion of the HLA Class I molecule, wherein the HLA Class I molecule is HLA subtype HLA-A*01:01 and the HLA-restricted peptide comprises the sequence NTDNNLAVY (SEQ ID NO: 1), and wherein the ABP binds the HLA-PEPTIDE target with greater affinity as compared to an off-target HLA-PEPTIDE comprising an off-target restricted peptide complexed with an HLA Class I molecule, wherein the off-target restricted peptide is located in the peptide binding groove of an α1/α2 heterodimer portion of the HLA Class I molecule.

In some embodiments, the HLA Class I molecule of the off-target HLA-PEPTIDE is HLA subtype A*01:01.

In some embodiments, the restricted peptide of the off-target HLA-PEPTIDE comprises a sequence that has no more than 5 amino acid mismatches from the G2 target restricted peptide NTDNNLAVY (SEQ ID NO: 1).

In some embodiments, the restricted peptide of the off-target HLA-PEPTIDE is 5-14 amino acids in length. In some embodiments, the restricted peptide of the off-target HLA-PEPTIDE is 7-12 amino acids in length. In some embodiments, the restricted peptide of the off-target HLA-PEPTIDE is 8-10 amino acids in length. In some embodiments, the restricted peptide of the off-target HLA-PEPTIDE is 9 amino acids in length.

In some embodiments, the restricted peptide of the off-target HLA-PEPTIDE is expressed in normal human tissue as indicated by the public GTEx database.

In some embodiments, the restricted peptide of the off-target HLA-PEPTIDE is derived from the gene product PTS, DSG3, DSG4, KDM7A, or ICE1. In some embodiments, the restricted peptide of the off-target HLA-PEPTIDE is derived from the gene product PTS.

In some embodiments, the restricted peptide of the off-target HLA-PEPTIDE comprises the sequence ETDNNIVVY (SEQ ID NO: 2), YTDNWLAVY (SEQ ID NO: 3), GTDNWLAQY (SEQ ID NO: 4), PTDENLARY (SEQ ID NO: 5), or NTDNLLTEY (SEQ ID NO: 6). In some embodiments, the restricted peptide of the off-target HLA-PEPTIDE consists essentially of the sequence ETDNNIVVY (SEQ ID NO: 2), YTDNWLAVY (SEQ ID NO: 3), GTDNWLAQY (SEQ ID NO: 4), PTDENLARY (SEQ ID NO: 5), or NTDNLLTEY (SEQ ID NO: 6). In some embodiments, the restricted peptide of the off-target HLA-PEPTIDE consists of the sequence ETDNNIVVY (SEQ ID NO: 2), YTDNWLAVY (SEQ ID NO: 3), GTDNWLAQY (SEQ ID NO: 4), PTDENLARY (SEQ ID NO: 5), or NTDNLLTEY (SEQ ID NO: 6).

Also provided herein is an isolated antigen binding protein (ABP) that specifically binds to a human leukocyte antigen (HLA)-PEPTIDE target, wherein the HLA-PEPTIDE target comprises an HLA-restricted peptide complexed with an HLA Class I molecule, wherein the HLA-restricted peptide is located in the peptide binding groove of an α1/α2 heterodimer portion of the HLA Class I molecule, wherein the HLA Class I molecule is HLA subtype HLA-A*01:01 and the HLA-restricted peptide comprises the sequence NTDNNLAVY (SEQ ID NO: 1), and wherein the ABP binds the HLA-PEPTIDE target with greater affinity as compared to an off-target HLA-PEPTIDE comprising an off-target restricted peptide complexed with an HLA Class I molecule, wherein the off-target restricted peptide is located in the peptide binding groove of an α1/α2 heterodimer portion of the HLA Class I molecule, and wherein the off-target HLA-PEPTIDE is selected from HLA-A*01:01_ETDNNIVVY (SEQ ID NO: 2), HLA-A*01:01_YTDNWLAVY (SEQ ID NO: 3), HLA-A*01:01_GTDNWLAQY (SEQ ID NO: 4), HLA-A*01:01_PTDENLARY (SEQ ID NO: 5), and HLA-A*01:01_NTDNLLTEY (SEQ ID NO: 6).

In some embodiments, the ABP binds to the HLA-PEPTIDE target with 100-10,000 stronger affinity as compared to the off-target HLA-PEPTIDE, or for which binding to off-target HLA-PEPTIDE is not detectable.

In some embodiments, the ABP binds to the HLA-PEPTIDE target with 100-10,000 stronger affinity as compared to the off-target HLA-PEPTIDE A*01:01_ETDNNIVVY (SEQ ID NO: 2), or for which binding to HLA-PEPTIDE A*01:01_ETDNNIVVY (SEQ ID NO: 2) is not detectable

In some embodiments, the ABP exhibits little or weak binding to the off-target HLA-PEPTIDE.

In some embodiments, the ABP binds to the off-target HLA-PEPTIDE with a Kd that is at least 1 uM or higher, or for which binding is undetectable.

In some embodiments, the ABP binds to the off-target HLA-PEPTIDE A*01:01_ETDNNIVVY (SEQ ID NO: 2) with a Kd that is at least 1 uM or higher, or for which binding is undetectable.

In some embodiments, the ABP does not bind to the off-target HLA-PEPTIDE.

In some embodiments, the ABP comprises at least one complementarity-determining region (CDR) from the clone designated 1C08, 1A03, 1A07, 1B04, 1C11, 1H06, 1D11, 1G01, 1H04, 1G06, 1B05, 1A08, 1F11, 1A04, 1F08, 1G09, 1F02, 1F04, 1C10, 1E04, 1B07, 1E01, 1F07, 1A10, 1E10, 1D10, 1D07, 1E05, 1F01, 1E08, 1C06, 1H03, 1H08, 1C04, 1D02, 1D05, 1E11, 1F05, 1G11, 1A02, 1A06, 1C05, 1D04, 1E03, 1A11, 1E09, 1C01, 1D09, 1B03, 1H02, 1D03, 1B02, 1F03, 1C09, 1C07, 2G03, 2H05, 2C03, 2F05, 2F04, 2G04, 2C01, 2E01, 2A10, 2G06, 2H07, 2G07, 2B01, 2B10, 2D11, 2G02, 2H08, 2G01, 2E06, 2B02, 2D04, 2A07, 2H04, 2B11, 2F11, 2D08, 2F03, 2A05, 2D07, 2E09, 2G09, 2A04, 2H03, 2E04, 2A09, 2D09, 2B08, 2G11, 2C08, 2C05, 2C07, 2F01, 2H01, 2B07, 2C06, 2A11, 2E10, 2A06, 2C04, 2D02, 2E05, 2F02, 2F07, 2G10, 2E03, 2H06, 2F09, 2E08, 2F08, 2C09, 2B06, 2F10, 2E07, 3F02, 3H02, 3A09, 3G05, 3F07, 3A06, 3G01, 3D02, 3F01, 3B07, 3F06, 3A10, 3C02, 3E02, 3D11, 3A05, 3A08, 3F11, 3E11, 3A03, 3D03, 3E03, 3D06, 3G08, 3A01, 3B09, 3E04, 3B06, 3C04, 3D01, 3D09, 3E08, 3E09, 3B05, 3D04, 3D07, 3F04, 3B02, 3B04, 3C01, 3C06, 3F03, 3G04, G307, 3E01, 3F08, 3C09, 3A02, 3F05, 3E07, 3E05, 3F09, 3C07, 3C08, 3G10, 3H01, 3G03, 3D10, 3B10, 3D08, or 3B11.

In some embodiments, the ABP comprises the heavy chain CDR3 (HCDR3) and the light chain CDR3 (LCDR3) from the clone designated 1C08, 1A03, 1A07, 1B04, 1C11, 1H06, 1D11, 1G01, 1H04, 1G06, 1B05, 1A08, 1F11, 1A04, 1F08, 1G09, 1F02, 1F04, 1C10, 1E04, 1B07, 1E01, 1F07, 1A10, 1E10, 1D10, 1D07, 1E05, 1F01, 1E08, 1C06, 1H03, 1H08, 1C04, 1D02, 1D05, 1E11, 1F05, 1G11, 1A02, 1A06, 1C05, 1D04, 1E03, 1A11, 1E09, 1C01, 1D09, 1B03, 1H02, 1D03, 1B02, 1F03, 1C09, 1C07, 2G03, 2H05, 2C03, 2F05, 2F04, 2G04, 2C01, 2E01, 2A10, 2G06, 2H07, 2G07, 2B01, 2B10, 2D11, 2G02, 2H08, 2G01, 2E06, 2B02, 2D04, 2A07, 2H04, 2B11, 2F11, 2D08, 2F03, 2A05, 2D07, 2E09, 2G09, 2A04, 2H03, 2E04, 2A09, 2D09, 2B08, 2G11, 2C08, 2C05, 2C07, 2F01, 2H01, 2B07, 2C06, 2A11, 2E10, 2A06, 2C04, 2D02, 2E05, 2F02, 2F07, 2G10, 2E03, 2H06, 2F09, 2E08, 2F08, 2C09, 2B06, 2F10, 2E07, 3F02, 3H02, 3A09, 3G05, 3F07, 3A06, 3G01, 3D02, 3F01, 3B07, 3F06, 3A10, 3C02, 3E02, 3D11, 3A05, 3A08, 3F11, 3E11, 3A03, 3D03, 3E03, 3D06, 3G08, 3A01, 3B09, 3E04, 3B06, 3C04, 3D01, 3D09, 3E08, 3E09, 3B05, 3D04, 3D07, 3F04, 3B02, 3B04, 3C01, 3C06, 3F03, 3G04, G307, 3E01, 3F08, 3C09, 3A02, 3F05, 3E07, 3E05, 3F09, 3C07, 3C08, 3G10, 3H01, 3G03, 3D10, 3B10, 3D08, or 3B11.

In some embodiments, the ABP comprises all three heavy chain CDRs (HCDR1, HCDR2, HCDR3) and all three light chain CDRs (LCDR1, LCDR2, LCDR3) from the clone designated 1C08, 1A03, 1A07, 1B04, 1C11, 1H06, 1D11, 1G01, 1H04, 1G06, 1B05, 1A08, 1F11, 1A04, 1F08, 1G09, 1F02, 1F04, 1C10, 1E04, 1B07, 1E01, 1F07, 1A10, 1E10, 1D10, 1D07, 1E05, 1F01, 1E08, 1C06, 1H03, 1H08, 1C04, 1D02, 1D05, 1E11, 1F05, 1G11, 1A02, 1A06, 1C05, 1D04, 1E03, 1A11, 1E09, 1C01, 1D09, 1B03, 1H02, 1D03, 1B02, 1F03, 1C09, 1C07, 2G03, 2H05, 2C03, 2F05, 2F04, 2G04, 2C01, 2E01, 2A10, 2G06, 2H07, 2G07, 2B01, 2B10, 2D11, 2G02, 2H08, 2G01, 2E06, 2B02, 2D04, 2A07, 2H04, 2B11, 2F11, 2D08, 2F03, 2A05, 2D07, 2E09, 2G09, 2A04, 2H03, 2E04, 2A09, 2D09, 2B08, 2G11, 2C08, 2C05, 2C07, 2F01, 2H01, 2B07, 2C06, 2A11, 2E10, 2A06, 2C04, 2D02, 2E05, 2F02, 2F07, 2G10, 2E03, 2H06, 2F09, 2E08, 2F08, 2C09, 2B06, 2F10, 2E07, 3F02, 3H02, 3A09, 3G05, 3F07, 3A06, 3G01, 3D02, 3F01, 3B07, 3F06, 3A10, 3C02, 3E02, 3D11, 3A05, 3A08, 3F11, 3E11, 3A03, 3D03, 3E03, 3D06, 3G08, 3A01, 3B09, 3E04, 3B06, 3C04, 3D01, 3D09, 3E08, 3E09, 3B05, 3D04, 3D07, 3F04, 3B02, 3B04, 3C01, 3C06, 3F03, 3G04, G307, 3E01, 3F08, 3C09, 3A02, 3F05, 3E07, 3E05, 3F09, 3C07, 3C08, 3G10, 3H01, 3G03, 3D10, 3B10, 3D08, or 3B11.

In some embodiments, the ABP comprises a variable heavy chain (VH) sequence from the clone designated 1C08, 1A03, 1A07, 1B04, 1C11, 1H06, 1D11, 1G01, 1H04, 1G06, 1B05, 1A08, 1F11, 1A04, 1F08, 1G09, 1F02, 1F04, 1C10, 1E04, 1B07, 1E01, 1F07, 1A10, 1E10, 1D10, 1D07, 1E05, 1F01, 1E08, 1C06, 1H03, 1H08, 1C04, 1D02, 1D05, 1E11, 1F05, 1G11, 1A02, 1A06, 1C05, 1D04, 1E03, 1A11, 1E09, 1C01, 1D09, 1B03, 1H02, 1D03, 1B02, 1F03, 1C09, 1C07, 2G03, 2H05, 2C03, 2F05, 2F04, 2G04, 2C01, 2E01, 2A10, 2G06, 2H07, 2G07, 2B01, 2B10, 2D11, 2G02, 2H08, 2G01, 2E06, 2B02, 2D04, 2A07, 2H04, 2B11, 2F11, 2D08, 2F03, 2A05, 2D07, 2E09, 2G09, 2A04, 2H03, 2E04, 2A09, 2D09, 2B08, 2G11, 2C08, 2C05, 2C07, 2F01, 2H01, 2B07, 2C06, 2A11, 2E10, 2A06, 2C04, 2D02, 2E05, 2F02, 2F07, 2G10, 2E03, 2H06, 2F09, 2E08, 2F08, 2C09, 2B06, 2F10, 2E07, 3F02, 3H02, 3A09, 3G05, 3F07, 3A06, 3G01, 3D02, 3F01, 3B07, 3F06, 3A10, 3C02, 3E02, 3D11, 3A05, 3A08, 3F11, 3E11, 3A03, 3D03, 3E03, 3D06, 3G08, 3A01, 3B09, 3E04, 3B06, 3C04, 3D01, 3D09, 3E08, 3E09, 3B05, 3D04, 3D07, 3F04, 3B02, 3B04, 3C01, 3C06, 3F03, 3G04, G307, 3E01, 3F08, 3C09, 3A02, 3F05, 3E07, 3E05, 3F09, 3C07, 3C08, 3G10, 3H01, 3G03, 3D10, 3B10, 3D08, or 3B11.

In some embodiments, the ABP comprises a variable light chain (VL) sequence from the clone designated 1C08, 1A03, 1A07, 1B04, 1C11, 1H06, 1D11, 1G01, 1H04, 1G06, 1B05, 1A08, 1F11, 1A04, 1F08, 1G09, 1F02, 1F04, 1C10, 1E04, 1B07, 1E01, 1F07, 1A10, 1E10, 1D10, 1D07, 1E05, 1F01, 1E08, 1C06, 1H03, 1H08, 1C04, 1D02, 1D05, 1E11, 1F05, 1G11, 1A02, 1A06, 1C05, 1D04, 1E03, 1A11, 1E09, 1C01, 1D09, 1B03, 1H02, 1D03, 1B02, 1F03, 1C09, 1C07, 2G03, 2H05, 2C03, 2F05, 2F04, 2G04, 2C01, 2E01, 2A10, 2G06, 2H07, 2G07, 2B01, 2B10, 2D11, 2G02, 2H08, 2G01, 2E06, 2B02, 2D04, 2A07, 2H04, 2B11, 2F11, 2D08, 2F03, 2A05, 2D07, 2E09, 2G09, 2A04, 2H03, 2E04, 2A09, 2D09, 2B08, 2G11, 2C08, 2C05, 2C07, 2F01, 2H01, 2B07, 2C06, 2A11, 2E10, 2A06, 2C04, 2D02, 2E05, 2F02, 2F07, 2G10, 2E03, 2H06, 2F09, 2E08, 2F08, 2C09, 2B06, 2F10, 2E07, 3F02, 3H02, 3A09, 3G05, 3F07, 3A06, 3G01, 3D02, 3F01, 3B07, 3F06, 3A10, 3C02, 3E02, 3D11, 3A05, 3A08, 3F11, 3E11, 3A03, 3D03, 3E03, 3D06, 3G08, 3A01, 3B09, 3E04, 3B06, 3C04, 3D01, 3D09, 3E08, 3E09, 3B05, 3D04, 3D07, 3F04, 3B02, 3B04, 3C01, 3C06, 3F03, 3G04, G307, 3E01, 3F08, 3C09, 3A02, 3F05, 3E07, 3E05, 3F09, 3C07, 3C08, 3G10, 3H01, 3G03, 3D10, 3B10, 3D08, or 3B11.

In some embodiments, the ABP comprises the VH sequence and the VL sequence from the clone designated 1C08, 1A03, 1A07, 1B04, 1C11, 1H06, 1D11, 1G01, 1H04, 1G06, 1B05, 1A08, 1F11, 1A04, 1F08, 1G09, 1F02, 1F04, 1C10, 1E04, 1B07, 1E01, 1F07, 1A10, 1E10, 1D10, 1D07, 1E05, 1F01, 1E08, 1C06, 1H03, 1H08, 1C04, 1D02, 1D05, 1E11, 1F05, 1G11, 1A02, 1A06, 1C05, 1D04, 1E03, 1A11, 1E09, 1C01, 1D09, 1B03, 1H02, 1D03, 1B02, 1F03, 1C09, 1C07, 2G03, 2H05, 2C03, 2F05, 2F04, 2G04, 2C01, 2E01, 2A10, 2G06, 2H07, 2G07, 2B01, 2B10, 2D11, 2G02, 2H08, 2G01, 2E06, 2B02, 2D04, 2A07, 2H04, 2B11, 2F11, 2D08, 2F03, 2A05, 2D07, 2E09, 2G09, 2A04, 2H03, 2E04, 2A09, 2D09, 2B08, 2G11, 2C08, 2C05, 2C07, 2F01, 2H01, 2B07, 2C06, 2A11, 2E10, 2A06, 2C04, 2D02, 2E05, 2F02, 2F07, 2G10, 2E03, 2H06, 2F09, 2E08, 2F08, 2C09, 2B06, 2F10, 2E07, 3F02, 3H02, 3A09, 3G05, 3F07, 3A06, 3G01, 3D02, 3F01, 3B07, 3F06, 3A10, 3C02, 3E02, 3D11, 3A05, 3A08, 3F11, 3E11, 3A03, 3D03, 3E03, 3D06, 3G08, 3A01, 3B09, 3E04, 3B06, 3C04, 3D01, 3D09, 3E08, 3E09, 3B05, 3D04, 3D07, 3F04, 3B02, 3B04, 3C01, 3C06, 3F03, 3G04, G307, 3E01, 3F08, 3C09, 3A02, 3F05, 3E07, 3E05, 3F09, 3C07, 3C08, 3G10, 3H01, 3G03, 3D10, 3B10, 3D08, or 3B11.

In some embodiments, the ABP comprises an antibody or antigen-binding fragment thereof.

In some embodiments, the antigen binding protein is linked to a scaffold, optionally wherein the scaffold comprises serum albumin or Fc, optionally wherein Fc is human Fc and is an IgG (IgG1, IgG2, IgG3, IgG4), an IgA (IgA1, IgA2), an IgD, an IgE, or an IgM.

In some embodiments, the antigen binding protein is linked to a scaffold via a linker, optionally wherein the linker is a peptide linker, optionally wherein the peptide linker is a hinge region of a human antibody.

In some embodiments, the antigen binding protein comprises an Fv fragment, a Fab fragment, a F(ab′)₂ fragment, a Fab′ fragment, an scFv fragment, an scFv-Fc fragment, and/or a single-domain antibody or antigen binding fragment thereof.

In some embodiments, the antigen binding protein comprises an scFv fragment.

In some embodiments, the antigen binding protein comprises one or more antibody complementarity determining regions (CDRs), optionally six antibody CDRs.

In some embodiments, the antigen binding protein comprises an antibody.

In some embodiments, the antigen binding protein is a monoclonal antibody.

In some embodiments, the antigen binding protein is a humanized, human, or chimeric antibody.

In some embodiments, the antigen binding protein is multispecific, optionally bispecific.

In some embodiments, the antigen binding protein binds greater than one antigen or greater than one epitope on a single antigen.

In some embodiments, the antigen binding protein comprises a heavy chain constant region of a class selected from IgG, IgA, IgD, IgE, and IgM.

In some embodiments, the antigen binding protein comprises a heavy chain constant region of the class human IgG and a subclass selected from IgG1, IgG4, IgG2, and IgG3.

In some embodiments, the antigen binding protein comprises a modified Fc, optionally wherein the modified Fc comprises one or more mutations that extend half-life, optionally wherein the one or more mutations that extend half-life is YTE.

In some embodiments, the antigen binding protein is multispecific, optionally bispecific.

In some embodiments, a sequence comprising the CH2-CH3 domains of the first polypeptide is distinct from a sequence comprising the CH2-CH3 domains of the second polypeptide.

In some embodiments, the ABP comprises a variant Fc region.

In some embodiments, the variant Fc region comprises a modification that alters an affinity of the ABP for an Fc receptor as compared to a multispecific ABP with a non-variant Fc region.

In some embodiments, the variant Fc region comprises a set of mutations that renders homodimerization electrostatically unfavorable but heterodimerization favorable.

In some embodiments, the antigen binding protein is a portion of a chimeric antigen receptor (CAR) comprising: an extracellular portion comprising the antigen binding protein; and an intracellular signaling domain.

Also provided herein is an isolated polynucleotide or set of polynucleotides encoding an antigen binding protein described herein or an antigen-binding portion thereof.

Also provided herein is a vector or set of vectors comprising the polynucleotide or set of polynucleotides described herein.

Also provided herein is a host cell comprising the polynucleotide or set of polynucleotides described herein or the vector or set of vectors described herein, optionally wherein the host cell is CHO or HEK293, or optionally wherein the host cell is a T cell.

Also provided herein is a method of producing an antigen binding protein comprising expressing the antigen binding protein with a host cell described herein and isolating the expressed antigen binding protein.

Also provided herein is a pharmaceutical composition comprising the antigen binding protein described herein and a pharmaceutically acceptable excipient.

Also provided herein is a method of increasing an immune response in a subject, comprising administering to the subject the ABP described herein or a pharmaceutical composition described herein, optionally wherein the subject has cancer, optionally wherein the cancer is selected from a solid tumor and a hematological tumor.

Also provided herein is a method of treating cancer in a subject, comprising administering to the subject an effective amount of the antigen binding protein described herein or a pharmaceutical composition described herein, optionally wherein the cancer is selected from a solid tumor and a hematological tumor.

In some embodiments, the cancer expresses or is predicted to express the HLA-PEPTIDE target.

In some embodiments, the method comprises, prior to the administering, determining or having determined the presence of any one or more of the HLA-PEPTIDE target, the restricted peptide of the HLA-PEPTIDE target, and the HLA molecule of the HLA-PEPTIDE target in a biological sample obtained from the subject.

In some embodiments, the biological sample is a blood sample or a tumor sample. In some embodiments, the blood sample is a plasma or serum sample.

In some embodiments, after having determined the presence of the HLA-PEPTIDE target, restricted peptide, or HLA in the biological sample obtained from the subject, the method comprises administering to the subject an ABP that selectively binds to the HLA-PEPTIDE antigen.

Also provided herein is a kit comprising the antigen binding protein described herein or a pharmaceutical composition described herein and instructions for use.

Also provided herein is a method of identifying an antigen binding protein described herein, comprising (a) binding an antigen binding protein to an HLA-PEPTIDE target comprising an HLA-restricted peptide complexed with an HLA Class I molecule, wherein the HLA-restricted peptide is located in the peptide binding groove of an α1/α2 heterodimer portion of the HLA Class I molecule, wherein the HLA Class I molecule is HLA subtype HLA-A*01:01 and the HLA-restricted peptide comprises the sequence NTDNNLAVY (SEQ ID NO: 1); (b) contacting the antigen binding protein with one or more off-target HLA-PEPTIDEs described herein; and (c) identifying the antigen binding protein if the antigen binding protein does not bind to the one or more off-target HLA-PEPTIDEs.

### BRIEF DESCRIPTION OF THE DRAWINGS

These and other features, aspects, and advantages of the present invention will become better understood with regard to the following description, and accompanying drawings, where:

FIG. 1 depicts exemplary extracted ion chromatogram for MS2 transitions for peptide ETDNNIVVY (SEQ ID NO: 2). The figure contains the peptide fragmentation information as well as relative peak area abundances of each transition for both heavy (standard) and light (sample) forms of the peptide.

FIG. 2 depicts exemplary extracted ion chromatogram for MS2 transitions for peptide NTDNLLTEY (SEQ ID NO: 6). The figure contains the peptide fragmentation information as well as relative peak area abundances of each transition for both heavy (standard) and light (sample) forms of the peptide.

FIG. 3 depicts exemplary extracted ion chromatogram for MS2 transitions for peptide PTDENLARY (SEQ ID NO: 5). The figure contains the peptide fragmentation information as well as relative peak area abundances of each transition for both heavy (standard) and light (sample) forms of the peptide.

FIG. 4 depicts exemplary extracted ion chromatogram for MS2 transitions for peptide YTDNWLAVY (SEQ ID NO: 3). The figure contains the peptide fragmentation information as well as relative peak area abundances of each transition for both heavy (standard) and light (sample) forms of the peptide.

FIG. 5 depicts MSD results for each scFv PPE selected via panning Strategy 1 (deselection against “ETD” peptide only) and panning Strategy 2 (deselection against all five OTLA peptides), plotted as binding intensity for the “ETD” peptide (Y axis) vs. binding intensity for the “NTD” peptide (X axis).

FIG. 6 depicts MSD results for each scFv PPE selected via panning Strategy 1 (deselection against “ETD” peptide only) and panning Strategy 2 (deselection against all five OTLA peptides), plotted as Target/OTLA intensity ratio (Y axis) vs. ETD binding intensity (X axis).

### DETAILED DESCRIPTION

Unless otherwise defined, all terms of art, notations and other scientific terminology used herein are intended to have the meanings commonly understood by those of skill in the art. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, N.Y. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise. The terms “include,” “such as,” and the like are intended to convey inclusion without limitation, unless otherwise specifically indicated.

As used herein, the term “comprising” also specifically includes embodiments “consisting of” and “consisting essentially of” the recited elements, unless specifically indicated otherwise. For example, a multispecific ABP “comprising a diabody” includes a multispecific ABP “consisting of a diabody” and a multispecific ABP “consisting essentially of a diabody.”

The term “about” indicates and encompasses an indicated value and a range above and below that value. In certain embodiments, the term “about” indicates the designated value ±10%, ±5%, or ±1%. In certain embodiments, where applicable, the term “about” indicates the designated value(s) ±one standard deviation of that value(s).

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, Pa. Briefly, each heavy chain typically comprises a heavy chain variable region (V_{H}) and a heavy chain constant region (C_{H}). The heavy chain constant region typically comprises three domains, abbreviated C_{H1}, C_{H2}, and C_{H3}. Each light chain typically comprises a light chain variable region (V_{L}) and a light chain constant region. The light chain constant region typically comprises one domain, abbreviated C_{L}.

The term “antigen binding protein” or “ABP” is used herein in its broadest sense and includes certain types of molecules comprising one or more antigen-binding domains that specifically bind to an antigen or epitope.

In some embodiments, the ABP comprises an antibody. In some embodiments, the ABP consists of an antibody. In some embodiments, the ABP consists essentially of an antibody. An ABP specifically includes intact antibodies (e.g., intact immunoglobulins), antibody fragments, ABP fragments, and multispecific antibodies. In some embodiments, the ABP comprises an alternative scaffold. In some embodiments, the ABP consists of an alternative scaffold. In some embodiments, the ABP consists essentially of an alternative scaffold. In some embodiments, the ABP comprises an antibody fragment. In some embodiments, the ABP consists of an antibody fragment. In some embodiments, the ABP consists essentially of an antibody fragment. In some embodiments, a CAR comprises an ABP provided herein. An “HLA-PEPTIDE ABP,” “anti-HLA-PEPTIDE ABP,” or “HLA-PEPTIDE-specific ABP” is an ABP, as provided herein, which specifically binds to the antigen HLA-PEPTIDE. An ABP includes proteins comprising one or more antigen-binding domains that specifically bind to an antigen or epitope via a variable region, such as a variable region derived from a B cell (e.g., antibody).

The term “antibody” herein is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, variable heavy chain (VH) regions capable of specifically binding the antigen, single chain antibody fragments, including single chain variable fragments (scFv), and single domain antibodies (e.g., sdAb, sdFv, nanobody, camelid VHH, engineered or evolved human VH that does not require pairing to VL for solubility or activity) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

As used herein, “variable region” refers to a variable nucleotide sequence that arises from a recombination event, for example, it can include a V, J, and/or D region of an immunoglobulin.

The term “antigen-binding domain” means the portion of an ABP that is capable of specifically binding to an antigen or epitope. One example of an antigen-binding domain is an antigen-binding domain formed by an antibody V_{H}-V_{L} dimer of an ABP. Another example of an antigen-binding domain is an antigen-binding domain formed by diversification of certain loops from the tenth fibronectin type III domain of an Adnectin. An antigen-binding domain can include antibody CDRs 1, 2, and 3 from a heavy chain in that order; and antibody CDRs 1, 2, and 3 from a light chain in that order.

The antibody V_{H} and V_{L} regions may be further subdivided into regions of hypervariability (“hypervariable regions (HVRs);” also called “complementarity determining regions” (CDRs)) interspersed with regions that are more conserved. The more conserved regions are called framework regions (FRs). Each V_{H} and V_{L} generally comprises three antibody CDRs and four FRs, arranged in the following order (from N-terminus to C-terminus): FR1-CDR1-FR2-CDR2-FR3-CDR3-FR4. The antibody CDRs are involved in antigen binding, and influence antigen specificity and binding affinity of the ABP. See Kabat et al., Sequences of Proteins of Immunological Interest 5th ed. (1991) Public Health Service, National Institutes of Health, Bethesda, Md., incorporated by reference in its entirety.

The light chain from any vertebrate species can be assigned to one of two types, called kappa (κ) and lambda (λ), based on the sequence of its constant domain

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

The amino acid sequence boundaries of an antibody CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Plückthun, J. Mol. Biol., 2001, 309:657-70 (“AHo” numbering scheme); each of which is incorporated by reference in its entirety.

Table 1 provides the positions of antibody LCDR1, LCDR2, LCDR3,

HCDR1, HCDR2, and HCDR3 as identified by the Kabat and Chothia schemes. For HCDR1, residue numbering is provided using both the Kabat and Chothia numbering schemes.

Antibody CDRs may be assigned, for example, using ABP numbering software, such as Abnum, available at www.bioinf.org.uk/abs/abnum/, and described in Abhinandan and Martin, Immunology, 2008, 45:3832-3839, incorporated by reference in its entirety.

TABLE 1
Residues in CDRs according to Kabat
and Chothia numbering schemes

| CDR | Kabat | Chothia |
|---|---|---|
| L1 | L24-L34 | L24-L34 |
| L2 | L50-L56 | L50-L56 |
| L3 | L89-L97 | L89-L97 |
| H1 (Kabat Numbering) | H31-H35B | H26-H32 or H34* |
| H1 (Chothia Numbering) | H31-H35 | H26-H32 |
| H2 | H50-H65 | H52-H56 |
| H3 | H95-H102 | H95-H102 |

*The C-terminus of CDR-H1, when numbered using the Kabat numbering convention, varies between H32 and H34, depending on the length of the CDR.

The “EU numbering scheme” is generally used when referring to a residue in an ABP heavy chain constant region (e.g., as reported in Kabat et al., supra). Unless stated otherwise, the EU numbering scheme is used to refer to residues in ABP heavy chain constant regions described herein.

The terms “full length antibody,” “intact antibody,” and “whole antibody” are used herein interchangeably to refer to an antibody having a structure substantially similar to a naturally occurring antibody structure and having heavy chains that comprise an Fc region. For example, when used to refer to an IgG molecule, a “full length antibody” is an antibody that comprises two heavy chains and two light chains.

An “ABP fragment” comprises a portion of an intact ABP, such as the antigen-binding or variable region of an intact ABP. ABP fragments include, for example, Fv fragments, Fab fragments, F(ab′)₂ fragments, Fab′ fragments, scFv (sFv) fragments, and scFv-Fc fragments. ABP fragments include antibody fragments. Antibody fragments can include Fv fragments, Fab fragments, F(ab′)₂ fragments, Fab′ fragments, scFv (sFv) fragments, and scFv-Fc fragments

“Fv” fragments comprise a non-covalently-linked dimer of one heavy chain variable domain and one light chain variable domain

“Fab” fragments comprise, in addition to the heavy and light chain variable domains, the constant domain of the light chain and the first constant domain (Cm) of the heavy chain. Fab fragments may be generated, for example, by recombinant methods or by papain digestion of a full-length ABP.

“F(ab′)₂” fragments contain two Fab′ fragments joined, near the hinge region, by disulfide bonds. F(ab′)₂ fragments may be generated, for example, by recombinant methods or by pepsin digestion of an intact ABP. The F(ab′) fragments can be dissociated, for example, by treatment with ß-mercaptoethanol.

“Single-chain Fv” or “sFv” or “scFv” fragments comprise a V_{H} domain and a V_{L} domain in a single polypeptide chain. The V_{H} and V_{L} are generally linked by a peptide linker. See Plückthun A. (1994). Any suitable linker may be used. In some embodiments, the linker is a (GGGGS)ₙ (SEQ ID NO: 7 when n=1). In some embodiments, n=1, 2, 3, 4, 5, or 6. See ABPs from Escherichia coli. In Rosenberg M. & Moore G. P. (Eds.), The Pharmacology of Monoclonal ABPs vol. 113 (pp. 269-315). Springer-Verlag, New York, incorporated by reference in its entirety.

“scFv-Fc” fragments comprise an scFv attached to an Fc domain. For example, an Fc domain may be attached to the C-terminal of the scFv. The Fc domain may follow the V_{H} or V_{L}, depending on the orientation of the variable domains in the scFv (i.e., V_{H}-V_{L} or V_{L}-V_{H}). Any suitable Fc domain known in the art or described herein may be used. In some cases, the Fc domain comprises an IgG4 Fc domain

The term “single domain antibody” refers to a molecule in which one variable domain of an ABP specifically binds to an antigen without the presence of the other variable domain. Single domain ABPs, and fragments thereof, are described in Arabi Ghahroudi et al., FEBS Letters, 1998, 414:521-526 and Muyldermans et al., Trends in Biochem. Sci., 2001, 26:230-245, each of which is incorporated by reference in its entirety. Single domain ABPs are also known as sdAbs or nanobodies.

The term “Fc region” or “Fc” means the C-terminal region of an immunoglobulin heavy chain that, in naturally occurring antibodies, interacts with Fc receptors and certain proteins of the complement system. The structures of the Fc regions of various immunoglobulins, and the glycosylation sites contained therein, are known in the art. See Schroeder and Cavacini, J. Allergy Clin. Immunol., 2010, 125:S41-52, incorporated by reference in its entirety. The Fc region may be a naturally occurring Fc region, or an Fc region modified as described in the art or elsewhere in this disclosure.

The term “alternative scaffold” refers to a molecule in which one or more regions may be diversified to produce one or more antigen-binding domains that specifically bind to an antigen or epitope. In some embodiments, the antigen-binding domain binds the antigen or epitope with specificity and affinity similar to that of an ABP. Exemplary alternative scaffolds include those derived from fibronectin (e.g., Adnectins™), the β-sandwich (e.g., iMab), lipocalin (e.g., Anticalins®), EETI-II/AGRP, BPTI/LACI-D1/ITI-D2 (e.g., Kunitz domains), thioredoxin peptide aptamers, protein A (e.g., Affibody®), ankyrin repeats (e.g., DARPins), gamma-B-crystallin/ubiquitin (e.g., Affilins), CTLD₃ (e.g., Tetranectins), Fynomers, and (LDLR-A module) (e.g., Avimers). Additional information on alternative scaffolds is provided in Binz et al., Nat. Biotechnol., 2005 23:1257-1268; Skerra, Current Opin. in Biotech., 2007 18:295-304; and Silacci et al., J. Biol. Chem., 2014, 289:14392-14398; each of which is incorporated by reference in its entirety. An alternative scaffold is one type of ABP.

A “multispecific ABP” is an ABP that comprises two or more different antigen-binding domains that collectively specifically bind two or more different epitopes. The two or more different epitopes may be epitopes on the same antigen (e.g., a single HLA-PEPTIDE molecule expressed by a cell) or on different antigens (e.g., different HLA-PEPTIDE molecules expressed by the same cell, or a HLA-PEPTIDE molecule and a non-HLA-PEPTIDE molecule). In some aspects, a multispecific ABP binds two different epitopes (i.e., a “bispecific ABP”). In some aspects, a multispecific ABP binds three different epitopes (i.e., a “trispecific ABP”).

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “chimeric antibody” refers to an antibody in which a portion of the heavy and/or light chain is derived from a particular source or species, while the remainder of the heavy and/or light chain is derived from a different source or species.

“Humanized” forms of non-human antibodies are chimeric antibodies that contain minimal sequence derived from the non-human antibody. A humanized antibody is generally a human antibody (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. Such modifications may be made to further refine antibody function. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

A “human antibody” is one which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

“Affinity” refers to the strength of the sum total of non-covalent interactions between a single binding site of a molecule (e.g., an ABP) and its binding partner (e.g., an antigen or epitope). Unless indicated otherwise, as used herein, “affinity” refers to intrinsic binding affinity, which reflects a 1:1 interaction between members of a binding pair (e.g., ABP and antigen or epitope). The affinity of a molecule X for its partner Y can be represented by the dissociation equilibrium constant (K_{D}). The kinetic components that contribute to the dissociation equilibrium constant are described in more detail below. Affinity can be measured by common methods known in the art, including those described herein, such as surface plasmon resonance (SPR) technology (e.g., BIACORE®) or biolayer interferometry (e.g., FORTEBIO®).

With regard to the binding of an ABP to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). Specific binding can be measured, for example, by measuring binding to a target molecule and comparing it to binding to a non-target molecule. Specific binding can also be determined by competition with a control molecule that mimics the epitope recognized on the target molecule. In that case, specific binding is indicated if the binding of the ABP to the target molecule is competitively inhibited by the control molecule. In some aspects, the affinity of a HLA-PEPTIDE ABP for a non-target molecule is less than about 50% of the affinity for HLA-PEPTIDE. In some aspects, the affinity of a HLA-PEPTIDE ABP for a non-target molecule is less than about 40% of the affinity for HLA-PEPTIDE. In some aspects, the affinity of a HLA-PEPTIDE ABP for a non-target molecule is less than about 30% of the affinity for HLA-PEPTIDE. In some aspects, the affinity of a HLA-PEPTIDE ABP for a non-target molecule is less than about 20% of the affinity for HLA-PEPTIDE. In some aspects, the affinity of a HLA-PEPTIDE ABP for a non-target molecule is less than about 10% of the affinity for HLA-PEPTIDE. In some aspects, the affinity of a HLA-PEPTIDE ABP for a non-target molecule is less than about 1% of the affinity for HLA-PEPTIDE. In some aspects, the affinity of a HLA-PEPTIDE ABP for a non-target molecule is less than about 0.1% of the affinity for HLA-PEPTIDE.

The term “k_{d}” (sec⁻¹), as used herein, refers to the dissociation rate constant of a particular ABP—antigen interaction. This value is also referred to as the k_{off} value.

The term “kₐ” (M⁻¹×sec⁻¹), as used herein, refers to the association rate constant of a particular ABP-antigen interaction. This value is also referred to as the kₒₙ value.

The term “K_{D}” (M), as used herein, refers to the dissociation equilibrium constant of a particular ABP-antigen interaction. K_{D}=k_{d}/kₐ. In some embodiments, the affinity of an ABP is described in terms of the K_{D} for an interaction between such ABP and its antigen. For clarity, as known in the art, a smaller K_{D} value indicates a higher affinity interaction, while a larger K_{D} value indicates a lower affinity interaction.

The term “K_{A}” (M⁻¹), as used herein, refers to the association equilibrium constant of a particular ABP-antigen interaction. K_{A}=kₐ/k_{d}.

An “immunoconjugate” is an ABP conjugated to one or more heterologous molecule(s), such as a therapeutic (cytokine, for example) or diagnostic agent.

“Fc effector functions” refer to those biological activities mediated by the Fc region of an ABP having an Fc region, which activities may vary depending on isotype. Examples of ABP effector functions include C1q binding to activate complement dependent cytotoxicity (CDC), Fc receptor binding to activate ABP-dependent cellular cytotoxicity (ADCC), and ABP dependent cellular phagocytosis (ADCP).

When used herein in the context of two or more ABPs, the term “competes with” or “cross-competes with” indicates that the two or more ABPs compete for binding to an antigen (e.g., HLA-PEPTIDE). In one exemplary assay, HLA-PEPTIDE is coated on a surface and contacted with a first HLA-PEPTIDE ABP, after which a second HLA-PEPTIDE ABP is added. In another exemplary assay, a first HLA-PEPTIDE ABP is coated on a surface and contacted with HLA-PEPTIDE, and then a second HLA-PEPTIDE ABP is added. If the presence of the first HLA-PEPTIDE ABP reduces binding of the second HLA-PEPTIDE ABP, in either assay, then the ABPs compete with each other. The term “competes with” also includes combinations of ABPs where one ABP reduces binding of another ABP, but where no competition is observed when the ABPs are added in the reverse order. However, in some embodiments, the first and second ABPs inhibit binding of each other, regardless of the order in which they are added. In some embodiments, one ABP reduces binding of another ABP to its antigen by at least 25%, at least 50%, at least 60%, at least 70%, at least 80%, at least 85%, at least 90%, or at least 95%. A skilled artisan can select the concentrations of the ABPs used in the competition assays based on the affinities of the ABPs for HLA-PEPTIDE and the valency of the ABPs. The assays described in this definition are illustrative, and a skilled artisan can utilize any suitable assay to determine if ABPs compete with each other. Suitable assays are described, for example, in Cox et al., “Immunoassay Methods,” in Assay Guidance Manual [Internet], Updated Dec. 24, 2014 (www.ncbi.nlm.nih.gov/books/NBK92434/; accessed Sep. 29, 2015); Silman et al., Cytometry, 2001, 44:30-37; and Finco et al., J. Pharm. Biomed. Anal., 2011, 54:351-358; each of which is incorporated by reference in its entirety.

The term “epitope” means a portion of an antigen that specifically binds to an ABP. Epitopes frequently consist of surface-accessible amino acid residues and/or sugar side chains and may have specific three dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding, and other amino acid residues, which are not directly involved in the binding. The epitope to which an ABP binds can be determined using known techniques for epitope determination such as, for example, testing for ABP binding to HLA-PEPTIDE variants with different point-mutations, or to chimeric HLA-PEPTIDE variants.

Percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

A “conservative substitution” or a “conservative amino acid substitution,” refers to the substitution an amino acid with a chemically or functionally similar amino acid. Conservative substitution tables providing similar amino acids are well known in the art. By way of example, the groups of amino acids provided in Tables 15-17 are, in some embodiments, considered conservative substitutions for one another.

TABLE 2
Selected groups of amino acids that are considered conservative
substitutions for one another, in certain embodiments.

| | |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

TABLE 3
Additional selected groups of amino acids that
are considered conservative substitutions
for one another, in certain embodiments.

| | |
|---|---|
| Group 1 | A, S, and T |
| Group 2 | D and E |
| Group 3 | N and Q |
| Group 4 | R and K |
| Group 5 | I, L, and M |
| Group 6 | F, Y, and W |

TABLE 4
Further selected groups of amino acids that are considered conservative
substitutions for one another, in certain embodiments.

| | |
|---|---|
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions may be found, for example, in Creighton, Proteins: Structures and Molecular Properties 2nd ed. (1993) W. H. Freeman & Co., New York, N.Y. An ABP generated by making one or more conservative substitutions of amino acid residues in a parent ABP is referred to as a “conservatively modified variant.”

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which an exogenous nucleic acid has been introduced, and the progeny of such cells. Host cells include “transformants” (or “transformed cells”) and “transfectants” (or “transfected cells”), which each include the primary transformed or transfected cell and progeny derived therefrom. Such progeny may not be completely identical in nucleic acid content to a parent cell, and may contain mutations.

The term “treating” (and variations thereof such as “treat” or “treatment”) refers to clinical intervention in an attempt to alter the natural course of a disease or condition in a subject in need thereof. Treatment can be performed both for prophylaxis and during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “therapeutically effective amount” or “effective amount” refers to an amount of an ABP or pharmaceutical composition provided herein that, when administered to a subject, is effective to treat a disease or disorder.

As used herein, the term “subject” means a mammalian subject. Exemplary subjects include humans, monkeys, dogs, cats, mice, rats, cows, horses, camels, goats, rabbits, and sheep. In certain embodiments, the subject is a human. In some embodiments the subject has a disease or condition that can be treated with an ABP provided herein. In some aspects, the disease or condition is a cancer. In some aspects, the disease or condition is a viral infection.

The term “package insert” is used to refer to instructions customarily included in commercial packages of therapeutic or diagnostic products (e.g., kits) that contain information about the indications, usage, dosage, administration, combination therapy, contraindications and/or warnings concerning the use of such therapeutic or diagnostic products.

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein. The terms “cell proliferative disorder” and “proliferative disorder” refer to disorders that are associated with some degree of abnormal cell proliferation. In some embodiments, the cell proliferative disorder is a cancer. In some aspects, the tumor is a solid tumor. In some aspects, the tumor is a hematologic malignancy.

The term “pharmaceutical composition” refers to a preparation which is in such form as to permit the biological activity of an active ingredient contained therein to be effective in treating a subject, and which contains no additional components which are unacceptably toxic to the subject in the amounts provided in the pharmaceutical composition.

The terms “modulate” and “modulation” refer to reducing or inhibiting or, alternatively, activating or increasing, a recited variable.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

The term “agonize” refers to the activation of receptor signaling to induce a biological response associated with activation of the receptor. An “agonist” is an entity that binds to and agonizes a receptor.

The term “antagonize” refers to the inhibition of receptor signaling to inhibit a biological response associated with activation of the receptor. An “antagonist” is an entity that binds to and antagonizes a receptor.

The terms “nucleic acids” and “polynucleotides” may be used interchangeably herein to refer to polymeric form of nucleotides of any length, either deoxyribonucleotides or ribonucleotides, or analogs thereof. Polynucleotides can include, but are not limited to coding or non-coding regions of a gene or gene fragment, loci (locus) defined from linkage analysis, exons, introns, messenger RNA (mRNA), cDNA, recombinant polynucleotides, branched polynucleotides, plasmids, vectors, isolated DNA, isolated RNA, nucleic acid probes, and primers. A polynucleotide may comprise modified nucleotides, such as methylated nucleotides and nucleotide analogs. Exemplary modified nucleotides include, e.g., 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthioN6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine.

HLA-Peptide ABPs

Provided herein are ABPs, e.g., ABPs that specifically bind to an HLA-PEPTIDE target, wherein the HLA-PEPTIDE target comprises an HLA-restricted peptide complexed with an HLA Class I molecule, wherein the HLA-restricted peptide is located in the peptide binding groove of an α1/α2 heterodimer portion of the HLA Class I molecule, and wherein the HLA-PEPTIDE target corresponds to tumor-specific gene product KKLC-1.

In some embodiments of the HLA-PEPTIDE target, the HLA Class I molecule is HLA-A*01:01 and the HLA-restricted peptide comprises the sequence NTDNNLAVY (SEQ ID NO: 1). In some embodiments, the HLA Class I molecule is HLA-A*01:01 and the HLA-restricted peptide consists essentially of the sequence NTDNNLAVY (SEQ ID NO: 1). In some embodiments, the HLA Class I molecule is HLA-A*01:01 and the HLA-restricted peptide consists of the sequence NTDNNLAVY (SEQ ID NO: 1).

In some embodiments, the ABP is an ABP that selectively binds HLA-PEPTIDE target A*01:01_ NTDNNLAVY (SEQ ID NO: 1). HLA-PEPTIDE target A*01:01_NTDNNLAVY (SEQ ID NO: 1), also referred to herein as “G2”, refers to an HLA-PEPTIDE target comprising the HLA-restricted peptide NTDNNLAVY (SEQ ID NO: 1) complexed with the HLA Class I molecule A*01:01, wherein the HLA-restricted peptide is located in the peptide binding groove of an α1/α2 heterodimer portion of the HLA Class I molecule. In some embodiments, the restricted peptide is from tumor-specific gene product KKLC-1.

The HLA-PEPTIDE target may be expressed on the surface of any suitable target cell including a tumor cell.

In some embodiments, the ABP does not bind HLA class I in the absence of the HLA-restricted peptide. In some embodiments, the ABP does not bind the HLA-restricted peptide in the absence of human MHC class I. In some embodiments, the ABP binds tumor cells presenting human MHC class I being complexed with the HLA-restricted peptide. In some embodiments, the HLA restricted peptide is a tumor antigen characterizing the cancer.

An ABP can bind to each portion of an HLA-PEPTIDE complex (i.e., HLA and peptide representing each portion of the complex), which when bound together form a novel target and protein surface for interaction with and binding by the ABP, distinct from a surface presented by the peptide alone or HLA subtype alone. Generally the novel target and protein surface formed by binding of HLA to peptide does not exist in the absence of each portion of the HLA-PEPTIDE complex.

In some embodiments, an ABP specific for HLA-PEPTIDE target A*01:01_NTDNNLAVY (SEQ ID NO: 1) (G2) selectively binds G2 with greater affinity as compared to an off-target HLA-PEPTIDE complex. The off-target HLA-PEPTIDE complex may comprise an off-target restricted peptide complexed with an HLA Class I molecule, wherein the HLA-restricted peptide is located in the peptide binding groove of an α1/α2 heterodimer portion of the HLA Class I molecule.

In some embodiments, the HLA Class I molecule of the off-target HLA-PEPTIDE is HLA subtype A*01:01.

In some embodiments, the restricted peptide of the off-target HLA-PEPTIDE comprises a sequence that has no more than 5 amino acid mismatches from the G2 target restricted peptide NTDNNLAVY (SEQ ID NO: 1).

In some embodiments, the restricted peptide of the off-target HLA-PEPTIDE is 5-14 amino acids in length. In some embodiments, the restricted peptide of the off-target HLA-PEPTIDE is 7-12 amino acids in length. In some embodiments, the restricted peptide of the off-target HLA-PEPTIDE is 8-10 amino acids in length. In some embodiments, the restricted peptide of the off-target HLA-PEPTIDE is 9 amino acids in length.

In some embodiments, the restricted peptide of the off-target HLA-PEPTIDE is expressed in normal human tissue as indicated by the public GTEx database.

In some embodiments, the restricted peptide of the off-target HLA-PEPTIDE is derived from the gene product PTS, DSG3, DSG4, KDM7A, or ICE1. In particular embodiments, the restricted peptide of the off-target HLA-PEPTIDE is derived from the gene product PTS.

In some embodiments, the restricted peptide of the off-target HLA-PEPTIDE comprises the sequence ETDNNIVVY (SEQ ID NO: 2), YTDNWLAVY (SEQ ID NO: 3), GTDNWLAQY (SEQ ID NO: 4), PTDENLARY (SEQ ID NO: 5), or NTDNLLTEY (SEQ ID NO: 6). In some embodiments, the restricted peptide of the off-target HLA-PEPTIDE consists essentially of the sequence ETDNNIVVY (SEQ ID NO: 2), YTDNWLAVY (SEQ ID NO: 3), GTDNWLAQY (SEQ ID NO: 4), PTDENLARY (SEQ ID NO: 5), or NTDNLLTEY (SEQ ID NO: 6). In some embodiments, the restricted peptide of the off-target HLA-PEPTIDE consists of the sequence ETDNNIVVY (SEQ ID NO: 2), YTDNWLAVY (SEQ ID NO: 3), GTDNWLAQY (SEQ ID NO: 4), PTDENLARY (SEQ ID NO: 5), or NTDNLLTEY (SEQ ID NO: 6).

In some embodiments, the off-target HLA-PEPTIDE is HLA-A*01:01_ETDNNIVVY (SEQ ID NO: 2), HLA-A*01:01_YTDNWLAVY (SEQ ID NO: 3), HLA-A*01:01_GTDNWLAQY (SEQ ID NO: 4), HLA-A*01:01_PTDENLARY (SEQ ID NO: 5), or HLA-A*01:01_NTDNLLTEY (SEQ ID NO: 6).

In some embodiments, the ABP binds to the HLA-PEPTIDE target with more than 10-fold, 20-fold, 50-fold stronger affinity as compared to the off-target HLA-PEPTIDE. In some embodiments, the ABP binds to the HLA-PEPTIDE target with 100×-10,000× stronger affinity as compared to the off-target HLA-PEPTIDE.

In particular embodiments, the ABP binds to the HLA-PEPTIDE target with more than 10-fold, 20-fold, 50-fold stronger affinity as compared to the off-target HLA-PEPTIDE A*01:01_ETDNNIVVY (SEQ ID NO: 2). In particular embodiments, the ABP binds to the HLA-PEPTIDE target with 100×-10,000× stronger affinity as compared to the off-target HLA-PEPTIDE A*01:01_ETDNNIVVY (SEQ ID NO: 2).

In some embodiments, the ABP exhibits little or weak binding to the off-target HLA-PEPTIDE. For example, in some embodiments, the ABP binds to the off-target HLA-PEPTIDE with a Kd that is at least 1 μM or higher, 5 μM or higher, 10 μM or higher, 20 μM or higher, 50 μM or higher, 100 μM or higher, or 1000 μM or higher.

In particular embodiments, the ABP binds to the off-target HLA-PEPTIDE A*01:01_ETDNNIVVY (SEQ ID NO: 2) with a Kd that is at least 1 μM or higher, 5 μM or higher, 10 μM or higher, 20 μM or higher, 50 μM or higher, 100 μM or higher, or 1000 μM or higher.

In some embodiments, the ABP does not exhibit detectable binding to the off-target HLA-PEPTIDE. In some embodiments, the ABP does not bind to the off-target HLA-PEPTIDE.

In some embodiments, the ABP does not exhibit detectable binding to the off-target HLA-PEPTIDE A*01:01_ETDNNIVVY (SEQ ID NO: 2). In some embodiments, the ABP does not bind to the off-target HLA-PEPTIDE A*01:01_ETDNNIVVY (SEQ ID NO: 2).

The ABP can be capable of specifically binding a complex comprising the HLA-PEPTIDE target, e.g., derived from a tumor. In some embodiments, the ABP does not bind HLA in an absence of the HLA-restricted peptide derived from the tumor. In some embodiments, the ABP does not bind the HLA-restricted peptide derived from the tumor in an absence of HLA. In some embodiments, the ABP binds a complex comprising HLA and HLA-restricted peptide when naturally presented on a cell such as a tumor cell.

In some embodiments, an ABP provided herein modulates binding of the HLA-PEPTIDE to one or more ligands of the HLA-PEPTIDE.

Also provided herein is an ABP is an ABP that competes with an illustrative ABP disclosed herein. In some aspects, the ABP that competes with the illustrative ABP provided herein binds the same epitope as an illustrative ABP provided herein.

In some aspects, provided herein are ABPs referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining ABPs. In some embodiments, a variant is derived from any of the sequences provided herein, wherein one or more conservative amino acid substitutions are made. In some embodiments, a variant is derived from any of the sequences provided herein, wherein one or more nonconservative amino acid substitutions are made. Conservative amino acid substitutions are described herein. Exemplary nonconservative amino acid substitutions include those described in J Immunol. 2008 May 1; 180(9):6116-31, which is hereby incorporated by reference in its entirety. In preferred embodiments, the non-conservative amino acid substitution does not interfere with or inhibit the biological activity of the functional variant. In yet more preferred embodiments, the non-conservative amino acid substitution enhances the biological activity of the functional variant, such that the biological activity of the functional variant is increased as compared to the parent ABP.

Sequences of Exemplary ABPs

The ABP specific for A*01:01_ NTDNNLAVY (SEQ ID NO: 1) may comprise one or more sequences, as described in further detail below.

CDRs

The ABP specific for A*01:01_ NTDNNLAVY (SEQ ID NO: 1) may comprise one or more antibody complementarity determining region (CDR) sequences, e.g., may comprise three heavy chain CDRs (HCDR1, HCDR2, HCDR3) and three light chain CDRs (LCDR1, LCDR2, LCDR3). For example, the ABP specific for A*01:01_ NTDNNLAVY (SEQ ID NO: 1) may comprise one or more antibody complementarity determining region (CDR) sequences from the clone designated 1C08, 1A03, 1A07, 1B04, 1C11, 1H06, 1D11, 1G01, 1H04, 1G06, 1B05, 1A08, 1F11, 1A04, 1F08, 1G09, 1F02, 1F04, 1C10, 1E04, 1B07, 1E01, 1F07, 1A10, 1E10, 1D10, 1D07, 1E05, 1F01, 1E08, 1C06, 1H03, 1H08, 1C04, 1D02, 1D05, 1E11, 1F05, 1G11, 1A02, 1A06, 1C05, 1D04, 1E03, 1A11, 1E09, 1C01, 1D09, 1B03, 1H02, 1D03, 1B02, 1F03, 1C09, 1C07, 2G03, 2H05, 2C03, 2F05, 2F04, 2G04, 2C01, 2E01, 2A10, 2G06, 2H07, 2G07, 2B01, 2B10, 2D11, 2G02, 2H08, 2G01, 2E06, 2B02, 2D04, 2A07, 2H04, 2B11, 2F11, 2D08, 2F03, 2A05, 2D07, 2E09, 2G09, 2A04, 2H03, 2E04, 2A09, 2D09, 2B08, 2G11, 2C08, 2C05, 2C07, 2F01, 2H01, 2B07, 2C06, 2A11, 2E10, 2A06, 2C04, 2D02, 2E05, 2F02, 2F07, 2G10, 2E03, 2H06, 2F09, 2E08, 2F08, 2C09, 2B06, 2F10, 2E07, 3F02, 3H02, 3A09, 3G05, 3F07, 3A06, 3G01, 3D02, 3F01, 3B07, 3F06, 3A10, 3C02, 3E02, 3D11, 3A05, 3A08, 3F11, 3E11, 3A03, 3D03, 3E03, 3D06, 3G08, 3A01, 3B09, 3E04, 3B06, 3C04, 3D01, 3D09, 3E08, 3E09, 3B05, 3D04, 3D07, 3F04, 3B02, 3B04, 3C01, 3C06, 3F03, 3G04, G307, 3E01, 3F08, 3C09, 3A02, 3F05, 3E07, 3E05, 3F09, 3C07, 3C08, 3G10, 3H01, 3G03, 3D10, 3B10, 3D08, or 3B11.

CDR sequences of identified scFvs that specifically bind A*01:01_NTDNNLAVY (SEQ ID NO: 1) are found in Table 10. For clarity, each identified scFv hit is designated a clone name, and each row contains the CDR sequences for that particular clone name. For example, the scFv identified by clone name 1C08 comprises the heavy chain CDR1 sequence DYNIH (SEQ ID NO: 8), the heavy chain CDR2 sequence WINPNSGGTNYAQKFQG (SEQ ID NO: 9), the heavy chain CDR3 sequence DKVGLDY (SEQ ID NO: 10), the light chain CDR1 sequence RASQGINNWLA (SEQ ID NO: 11), the light chain CDR2 sequence AASSLQA (SEQ ID NO: 12), and the light chain CDR3 sequence QQSYLTPYT (SEQ ID NO: 13).

The ABP specific for A*01:01_ NTDNNLAVY (SEQ ID NO: 1) may comprise a particular HCDR3 sequence. In some embodiments, the ABP comprises the HCDR3 from the clone designated 1C08, 1A03, 1A07, 1B04, 1C11, 1H06, 1D11, 1G01, 1H04, 1G06, 1B05, 1A08, 1F11, 1A04, 1F08, 1G09, 1F02, 1F04, 1C10, 1E04, 1B07, 1E01, 1F07, 1A10, 1E10, 1D10, 1D07, 1E05, 1F01, 1E08, 1C06, 1H03, 1H08, 1C04, 1D02, 1D05, 1E11, 1F05, 1G11, 1A02, 1A06, 1C05, 1D04, 1E03, 1A11, 1E09, 1C01, 1D09, 1B03, 1H02, 1D03, 1B02, 1F03, 1C09, 1C07, 2G03, 2H05, 2C03, 2F05, 2F04, 2G04, 2C01, 2E01, 2A10, 2G06, 2H07, 2G07, 2B01, 2B10, 2D11, 2G02, 2H08, 2G01, 2E06, 2B02, 2D04, 2A07, 2H04, 2B11, 2F11, 2D08, 2F03, 2A05, 2D07, 2E09, 2G09, 2A04, 2H03, 2E04, 2A09, 2D09, 2B08, 2G11, 2C08, 2C05, 2C07, 2F01, 2H01, 2B07, 2C06, 2A11, 2E10, 2A06, 2C04, 2D02, 2E05, 2F02, 2F07, 2G10, 2E03, 2H06, 2F09, 2E08, 2F08, 2C09, 2B06, 2F10, 2E07, 3F02, 3H02, 3A09, 3G05, 3F07, 3A06, 3G01, 3D02, 3F01, 3B07, 3F06, 3A10, 3C02, 3E02, 3D11, 3A05, 3A08, 3F11, 3E11, 3A03, 3D03, 3E03, 3D06, 3G08, 3A01, 3B09, 3E04, 3B06, 3C04, 3D01, 3D09, 3E08, 3E09, 3B05, 3D04, 3D07, 3F04, 3B02, 3B04, 3C01, 3C06, 3F03, 3G04, G307, 3E01, 3F08, 3C09, 3A02, 3F05, 3E07, 3E05, 3F09, 3C07, 3C08, 3G10, 3H01, 3G03, 3D10, 3B10, 3D08, or 3B11.

The ABP specific for A*01:01_ NTDNNLAVY (SEQ ID NO: 1) may comprise a particular light chain CDR3 sequence. The LCDR3 sequence may be selected from the clone designated 1C08, 1A03, 1A07, 1B04, 1C11, 1H06, 1D11, 1G01, 1H04, 1G06, 1B05, 1A08, 1F11, 1A04, 1F08, 1G09, 1F02, 1F04, 1C10, 1E04, 1B07, 1E01, 1F07, 1A10, 1E10, 1D10, 1D07, 1E05, 1F01, 1E08, 1C06, 1H03, 1H08, 1C04, 1D02, 1D05, 1E11, 1F05, 1G11, 1A02, 1A06, 1C05, 1D04, 1E03, 1A11, 1E09, 1C01, 1D09, 1B03, 1H02, 1D03, 1B02, 1F03, 1C09, 1C07, 2G03, 2H05, 2C03, 2F05, 2F04, 2G04, 2C01, 2E01, 2A10, 2G06, 2H07, 2G07, 2B01, 2B10, 2D11, 2G02, 2H08, 2G01, 2E06, 2B02, 2D04, 2A07, 2H04, 2B11, 2F11, 2D08, 2F03, 2A05, 2D07, 2E09, 2G09, 2A04, 2H03, 2E04, 2A09, 2D09, 2B08, 2G11, 2C08, 2C05, 2C07, 2F01, 2H01, 2B07, 2C06, 2A11, 2E10, 2A06, 2C04, 2D02, 2E05, 2F02, 2F07, 2G10, 2E03, 2H06, 2F09, 2E08, 2F08, 2C09, 2B06, 2F10, 2E07, 3F02, 3H02, 3A09, 3G05, 3F07, 3A06, 3G01, 3D02, 3F01, 3B07, 3F06, 3A10, 3C02, 3E02, 3D11, 3A05, 3A08, 3F11, 3E11, 3A03, 3D03, 3E03, 3D06, 3G08, 3A01, 3B09, 3E04, 3B06, 3C04, 3D01, 3D09, 3E08, 3E09, 3B05, 3D04, 3D07, 3F04, 3B02, 3B04, 3C01, 3C06, 3F03, 3G04, G307, 3E01, 3F08, 3C09, 3A02, 3F05, 3E07, 3E05, 3F09, 3C07, 3C08, 3G10, 3H01, 3G03, 3D10, 3B10, 3D08, or 3B11.

The ABP specific for A*01:01_ NTDNNLAVY (SEQ ID NO: 1) may comprise a particular heavy chain CDR3 (HCDR3) sequence and a particular light chain CDR3 (LCDR3) sequence. In some embodiments, the ABP comprises the HCDR3 and the LCDR3 from the clone designated 1C08, 1A03, 1A07, 1B04, 1C11, 1H06, 1D11, 1G01, 1H04, 1G06, 1B05, 1A08, 1F11, 1A04, 1F08, 1G09, 1F02, 1F04, 1C10, 1E04, 1B07, 1E01, 1F07, 1A10, 1E10, 1D10, 1D07, 1E05, 1F01, 1E08, 1C06, 1H03, 1H08, 1C04, 1D02, 1D05, 1E11, 1F05, 1G11, 1A02, 1A06, 1C05, 1D04, 1E03, 1A11, 1E09, 1C01, 1D09, 1B03, 1H02, 1D03, 1B02, 1F03, 1C09, 1C07, 2G03, 2H05, 2C03, 2F05, 2F04, 2G04, 2C01, 2E01, 2A10, 2G06, 2H07, 2G07, 2B01, 2B10, 2D11, 2G02, 2H08, 2G01, 2E06, 2B02, 2D04, 2A07, 2H04, 2B11, 2F11, 2D08, 2F03, 2A05, 2D07, 2E09, 2G09, 2A04, 2H03, 2E04, 2A09, 2D09, 2B08, 2G11, 2C08, 2C05, 2C07, 2F01, 2H01, 2B07, 2C06, 2A11, 2E10, 2A06, 2C04, 2D02, 2E05, 2F02, 2F07, 2G10, 2E03, 2H06, 2F09, 2E08, 2F08, 2C09, 2B06, 2F10, 2E07, 3F02, 3H02, 3A09, 3G05, 3F07, 3A06, 3G01, 3D02, 3F01, 3B07, 3F06, 3A10, 3C02, 3E02, 3D11, 3A05, 3A08, 3F11, 3E11, 3A03, 3D03, 3E03, 3D06, 3G08, 3A01, 3B09, 3E04, 3B06, 3C04, 3D01, 3D09, 3E08, 3E09, 3B05, 3D04, 3D07, 3F04, 3B02, 3B04, 3C01, 3C06, 3F03, 3G04, G307, 3E01, 3F08, 3C09, 3A02, 3F05, 3E07, 3E05, 3F09, 3C07, 3C08, 3G10, 3H01, 3G03, 3D10, 3B10, 3D08, or 3B11.

The ABP specific for A*01:01_ NTDNNLAVY (SEQ ID NO: 1) may comprise all six CDRs from the clone designated 1C08, 1A03, 1A07, 1B04, 1C11, 1H06, 1D11, 1G01, 1H04, 1G06, 1B05, 1A08, 1F11, 1A04, 1F08, 1G09, 1F02, 1F04, 1C10, 1E04, 1B07, 1E01, 1F07, 1A10, 1E10, 1D10, 1D07, 1E05, 1F01, 1E08, 1C06, 1H03, 1H08, 1C04, 1D02, 1D05, 1E11, 1F05, 1G11, 1A02, 1A06, 1C05, 1D04, 1E03, 1A11, 1E09, 1C01, 1D09, 1B03, 1H02, 1D03, 1B02, 1F03, 1C09, 1C07, 2G03, 2H05, 2C03, 2F05, 2F04, 2G04, 2C01, 2E01, 2A10, 2G06, 2H07, 2G07, 2B01, 2B10, 2D11, 2G02, 2H08, 2G01, 2E06, 2B02, 2D04, 2A07, 2H04, 2B11, 2F11, 2D08, 2F03, 2A05, 2D07, 2E09, 2G09, 2A04, 2H03, 2E04, 2A09, 2D09, 2B08, 2G11, 2C08, 2C05, 2C07, 2F01, 2H01, 2B07, 2C06, 2A11, 2E10, 2A06, 2C04, 2D02, 2E05, 2F02, 2F07, 2G10, 2E03, 2H06, 2F09, 2E08, 2F08, 2C09, 2B06, 2F10, 2E07, 3F02, 3H02, 3A09, 3G05, 3F07, 3A06, 3G01, 3D02, 3F01, 3B07, 3F06, 3A10, 3C02, 3E02, 3D11, 3A05, 3A08, 3F11, 3E11, 3A03, 3D03, 3E03, 3D06, 3G08, 3A01, 3B09, 3E04, 3B06, 3C04, 3D01, 3D09, 3E08, 3E09, 3B05, 3D04, 3D07, 3F04, 3B02, 3B04, 3C01, 3C06, 3F03, 3G04, G307, 3E01, 3F08, 3C09, 3A02, 3F05, 3E07, 3E05, 3F09, 3C07, 3C08, 3G10, 3H01, 3G03, 3D10, 3B10, 3D08, or 3B11.

VL

The ABP specific for A*01:01_ NTDNNLAVY (SEQ ID NO: 1) may comprise a particular VL sequence. The VL sequence may be from the clone designated 1C08, 1A03, 1A07, 1B04, 1C11, 1H06, 1D11, 1G01, 1H04, 1G06, 1B05, 1A08, 1F11, 1A04, 1F08, 1G09, 1F02, 1F04, 1C10, 1E04, 1B07, 1E01, 1F07, 1A10, 1E10, 1D10, 1D07, 1E05, 1F01, 1E08, 1C06, 1H03, 1H08, 1C04, 1D02, 1D05, 1E11, 1F05, 1G11, 1A02, 1A06, 1C05, 1D04, 1E03, 1A11, 1E09, 1C01, 1D09, 1B03, 1H02, 1D03, 1B02, 1F03, 1C09, 1C07, 2G03, 2H05, 2C03, 2F05, 2F04, 2G04, 2C01, 2E01, 2A10, 2G06, 2H07, 2G07, 2B01, 2B10, 2D11, 2G02, 2H08, 2G01, 2E06, 2B02, 2D04, 2A07, 2H04, 2B11, 2F11, 2D08, 2F03, 2A05, 2D07, 2E09, 2G09, 2A04, 2H03, 2E04, 2A09, 2D09, 2B08, 2G11, 2C08, 2C05, 2C07, 2F01, 2H01, 2B07, 2C06, 2A11, 2E10, 2A06, 2C04, 2D02, 2E05, 2F02, 2F07, 2G10, 2E03, 2H06, 2F09, 2E08, 2F08, 2C09, 2B06, 2F10, 2E07, 3F02, 3H02, 3A09, 3G05, 3F07, 3A06, 3G01, 3D02, 3F01, 3B07, 3F06, 3A10, 3C02, 3E02, 3D11, 3A05, 3A08, 3F11, 3E11, 3A03, 3D03, 3E03, 3D06, 3G08, 3A01, 3B09, 3E04, 3B06, 3C04, 3D01, 3D09, 3E08, 3E09, 3B05, 3D04, 3D07, 3F04, 3B02, 3B04, 3C01, 3C06, 3F03, 3G04, G307, 3E01, 3F08, 3C09, 3A02, 3F05, 3E07, 3E05, 3F09, 3C07, 3C08, 3G10, 3H01, 3G03, 3D10, 3B10, 3D08, or 3B11.

VL and VH sequences of identified scFvs that specifically bind A*01:01_NTDNNLAVY (SEQ ID NO: 1) are found in Table 11. For clarity, each identified scFv hit is designated a clone name, and each row contains the VH and VL sequences for that particular clone name. For example, the scFv identified by clone name 1C08 comprises the VH sequence QVQLVQSGAEVKKPGASVKVSCKASGYTVTDYNIHWVRQAPGQGLEWMGWINPN SGGTNYAQKFQGRVTMTRDTSTSTVYMELSSLRSEDTAVYYCARDKVGLDYWGQG TLVTVSS (SEQ ID NO: 14) and the VL sequence DIQMTQSPSSLSASVGDRVTITCRASQGINNWLAWYQQKPGKAPKLLIYAASSLQAG VPSRFSGSGSGTDFTLTISSLQPEDFATYYCQQSYLTPYTFGQGTKLEIK (SEQ ID NO: 15).

VH

The ABP specific for A*01:01_ NTDNNLAVY (SEQ ID NO: 1) may comprise a VH sequence. The VH sequence may be from the clone designated 1C08, 1A03, 1A07, 1B04, 1C11, 1H06, 1D11, 1G01, 1H04, 1G06, 1B05, 1A08, 1F11, 1A04, 1F08, 1G09, 1F02, 1F04, 1C10, 1E04, 1B07, 1E01, 1F07, 1A10, 1E10, 1D10, 1D07, 1E05, 1F01, 1E08, 1C06, 1H03, 1H08, 1C04, 1D02, 1D05, 1E11, 1F05, 1G11, 1A02, 1A06, 1C05, 1D04, 1E03, 1A11, 1E09, 1C01, 1D09, 1B03, 1H02, 1D03, 1B02, 1F03, 1C09, 1C07, 2G03, 2H05, 2C03, 2F05, 2F04, 2G04, 2C01, 2E01, 2A10, 2G06, 2H07, 2G07, 2B01, 2B10, 2D11, 2G02, 2H08, 2G01, 2E06, 2B02, 2D04, 2A07, 2H04, 2B11, 2F11, 2D08, 2F03, 2A05, 2D07, 2E09, 2G09, 2A04, 2H03, 2E04, 2A09, 2D09, 2B08, 2G11, 2C08, 2C05, 2C07, 2F01, 2H01, 2B07, 2C06, 2A11, 2E10, 2A06, 2C04, 2D02, 2E05, 2F02, 2F07, 2G10, 2E03, 2H06, 2F09, 2E08, 2F08, 2C09, 2B06, 2F10, 2E07, 3F02, 3H02, 3A09, 3G05, 3F07, 3A06, 3G01, 3D02, 3F01, 3B07, 3F06, 3A10, 3C02, 3E02, 3D11, 3A05, 3A08, 3F11, 3E11, 3A03, 3D03, 3E03, 3D06, 3G08, 3A01, 3B09, 3E04, 3B06, 3C04, 3D01, 3D09, 3E08, 3E09, 3B05, 3D04, 3D07, 3F04, 3B02, 3B04, 3C01, 3C06, 3F03, 3G04, G307, 3E01, 3F08, 3C09, 3A02, 3F05, 3E07, 3E05, 3F09, 3C07, 3C08, 3G10, 3H01, 3G03, 3D10, 3B10, 3D08, or 3B11.

VH-VL Combinations

The ABP specific for A*01:01_NTDNNLAVY (SEQ ID NO: 1) may comprise a particular VH sequence and a particular VL sequence. In some embodiments, the ABP specific for A*01:01_NTDNNLAVY (SEQ ID NO: 1) comprises the VH sequence and the VL sequence from the clone designated 1C08, 1A03, 1A07, 1B04, 1C11, 1H06, 1D11, 1G01, 1H04, 1G06, 1B05, 1A08, 1F11, 1A04, 1F08, 1G09, 1F02, 1F04, 1C10, 1E04, 1B07, 1E01, 1F07, 1A10, 1E10, 1D10, 1D07, 1E05, 1F01, 1E08, 1C06, 1H03, 1H08, 1C04, 1D02, 1D05, 1E11, 1F05, 1G11, 1A02, 1A06, 1C05, 1D04, 1E03, 1A11, 1E09, 1C01, 1D09, 1B03, 1H02, 1D03, 1B02, 1F03, 1C09, 1C07, 2G03, 2H05, 2C03, 2F05, 2F04, 2G04, 2C01, 2E01, 2A10, 2G06, 2H07, 2G07, 2B01, 2B10, 2D11, 2G02, 2H08, 2G01, 2E06, 2B02, 2D04, 2A07, 2H04, 2B11, 2F11, 2D08, 2F03, 2A05, 2D07, 2E09, 2G09, 2A04, 2H03, 2E04, 2A09, 2D09, 2B08, 2G11, 2C08, 2C05, 2C07, 2F01, 2H01, 2B07, 2C06, 2A11, 2E10, 2A06, 2C04, 2D02, 2E05, 2F02, 2F07, 2G10, 2E03, 2H06, 2F09, 2E08, 2F08, 2C09, 2B06, 2F10, 2E07, 3F02, 3H02, 3A09, 3G05, 3F07, 3A06, 3G01, 3D02, 3F01, 3B07, 3F06, 3A10, 3C02, 3E02, 3D11, 3A05, 3A08, 3F11, 3E11, 3A03, 3D03, 3E03, 3D06, 3G08, 3A01, 3B09, 3E04, 3B06, 3C04, 3D01, 3D09, 3E08, 3E09, 3B05, 3D04, 3D07, 3F04, 3B02, 3B04, 3C01, 3C06, 3F03, 3G04, G307, 3E01, 3F08, 3C09, 3A02, 3F05, 3E07, 3E05, 3F09, 3C07, 3C08, 3G10, 3H01, 3G03, 3D10, 3B10, 3D08, or 3B11.

Isolated HLA-Peptide Targets

The major histocompatibility complex (MHC) is a complex of antigens encoded by a group of linked loci, which are collectively termed H-2 in the mouse and HLA in humans. The two principal classes of the MHC antigens, class I and class II, each comprise a set of cell surface glycoproteins which play a role in determining tissue type and transplant compatibility. In transplantation reactions, cytotoxic T-cells (CTLs) respond mainly against class I glycoproteins, while helper T-cells respond mainly against class II glycoproteins.

Human major histocompatibility complex (MHC) class I molecules, referred to interchangeably herein as HLA Class I molecules, are expressed on the surface of nearly all cells. These molecules function in presenting peptides which are mainly derived from endogenously synthesized proteins to, e.g., CD8+ T cells via an interaction with the alpha-beta T-cell receptor. The class I MHC molecule comprises a heterodimer composed of a 46-kDa a chain which is non-covalently associated with the 12-kDa light chain beta-2 microglobulin. The a chain generally comprises α1 and α2 domains which form a groove for presenting an HLA-restricted peptide, and an α3 plasma membrane-spanning domain which interacts with the CD8 co-receptor of T-cells. See, e.g., Kerry S E, Buslepp J, Cramer L A, et al. Interplay between TCR Affinity and Necessity of Coreceptor Ligation: High-Affinity Peptide-MHC/TCR Interaction Overcomes Lack of CD8 Engagement. Journal of immunology (Baltimore, Md.: 1950). 2003; 171(9):4493-4503.)

Class I MHC-restricted peptides (also referred to interchangeably herein as HLA-restricted antigens, HLA-restricted peptides, MHC-restricted antigens, restricted peptides, or peptides) generally bind to the heavy chain alpha1-alpha2 groove via about two or three anchor residues that interact with corresponding binding pockets in the MHC molecule. The beta-2 microglobulin chain plays an important role in MHC class I intracellular transport, peptide binding, and conformational stability. For most class I molecules, the formation of a heterotrimeric complex of the MHC class I heavy chain, peptide (self, non-self, and/or antigenic) and beta-2 microglobulin leads to protein maturation and export to the cell-surface.

Binding of a given HLA subtype to an HLA-restricted peptide forms a complex with a unique and novel surface that can be specifically recognized by an ABP such as, e.g., a TCR on a T cell or an antibody or antigen-binding fragment thereof. HLA complexed with an HLA-restricted peptide is referred to herein as an HLA-PEPTIDE or HLA-PEPTIDE target. In some cases, the restricted peptide is located in the α1/α2 groove of the HLA molecule. In some cases, the restricted peptide is bound to the α1/α2 groove of the HLA molecule via about two or three anchor residues that interact with corresponding binding pockets in the HLA molecule.

Accordingly, provided herein are antigens comprising an HLA-PEPTIDE target disclosed herein.

The HLA-PEPTIDE targets identified herein may be useful for cancer immunotherapy. In some embodiments, the HLA-PEPTIDE targets identified herein are presented on the surface of a tumor cell. The HLA-PEPTIDE targets identified herein may be expressed by tumor cells in a human subject. The HLA-PEPTIDE targets identified herein may be expressed by tumor cells in a population of human subjects. For example, the HLA-PEPTIDE targets identified herein may be shared antigens which are commonly expressed in a population of human subjects with cancer.

The HLA-PEPTIDE targets identified herein may have a prevalence with an individual tumor type The prevalence with an individual tumor type may be about 0.1%, 0.2%, 0.3%, 0.4%, 0.5%, 0.6%, 0.7%, 0.8%, 0.9%, 1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. The prevalence with an individual tumor type may be about 0.1%-100%, 0.2-50%, 0.5-25%, 2-20% or 1-10%.

Preferably, HLA-PEPTIDE targets are not generally expressed in most normal tissues. For example, the HLA-PEPTIDE targets may in some cases not be expressed in tissues in the Genotype-Tissue Expression (GTEx) Project, or may in some cases be expressed only in immune privileged or non-essential tissues. Exemplary immune privileged or non-essential tissues include testis, minor salivary glands, the endocervix, and the thyroid. In some cases, an HLA-PEPTIDE target may be deemed to not be expressed on essential tissues or non-immune privileged tissues if the median expression of a gene from which the restricted peptide is derived is less than 0.5 RPKM (Reads Per Kilobase of transcript per Million mapped reads) across GTEx samples, if the gene is not expressed with greater than 10 RPKM across GTEX samples, if the gene was expressed at >=5 RPKM in no more two samples across all essential tissue samples, or any combination thereof.

Also provided herein are off-target HLA-PEPTIDES. Such off-target HLA-PEPTIDES may be useful for identifying a cancer therapeutic, e.g., an ABP disclosed herein.

HLA-Restricted Peptides

The HLA-restricted peptides of an HLA-PEPTIDE target disclosed herein (referred to interchangeably herein) as “restricted peptides” can be peptide fragments of tumor-specific genes, e.g., cancer-specific genes. Preferably, the cancer-specific genes are expressed in cancer samples. Genes which are aberrantly expressed in cancer samples can be identified through a database. Exemplary databases include, by way of example only, The Cancer Genome Atlas (TCGA) Research Network: http://cancergenome.nih.gov/; the International Cancer Genome Consortium: https://dcc.icgc.org/. In some embodiments, the cancer-specific gene has an observed expression of at least 10 RPKM in at least 5 samples from the TCGA database. The cancer-specific gene may have an observable bimodal distribution.

The cancer-specific gene may have an observed expression of greater than 10, 20, 30, 40, 50, 60, 70, 80, 90, or 100 transcripts per million (TPM) in at least one TCGA tumor tissue. In preferred embodiments, the cancer-specific gene has an observed expression of greater than 100 TPM in at least one TCGA tumor tissue. In some cases, the cancer specific gene has an observed bimodal distribution of expression across TCGA samples. Without wishing to be bound by theory, such bimodal expression pattern is consistent with a biological model in which there is minimal expression at baseline in all tumor samples and higher expression in a subset of tumors experiencing epigenetic dysregulation.

Preferably, the cancer-specific gene is not generally expressed in most normal tissues. For example, the cancer-specific gene may in some cases not be expressed in tissues in the Genotype-Tissue Expression (GTEx) Project, or may in some cases be expressed in immune privileged or non-essential tissues. Exemplary immune privileged or non-essential tissues include testis, minor salivary glands, the endocervix, and thyroid. In some cases, an cancer-specific gene may be deemed to not be expressed an essential tissues or non-immune privileged tissue if the median expression of the cancer-specific gene is less than 0.5 RPKM (Reads Per Kilobase of transcript per Million mapped reads) across GTEx samples, if the gene is not expressed with greater than 10 RPKM across GTEX samples, if the gene was expressed at >=5 RPKM in no more two samples across all essential tissue samples, or any combination thereof.

In some embodiments, the cancer-specific gene meets the following criteria by assessment of the GTEx: (1) median GTEx expression in brain, heart, or lung is less than 0.1 transcripts per million (TPM), with no one sample exceeding 5 TPM, (2) median GTEx expression in other essential organs (excluding testis, thyroid, minor salivary gland) is less than 2 TPM with no one sample exceeding 10 TPM.

In some embodiments, the cancer-specific gene is not likely expressed in immune cells generally, e.g., is not an interferon family gene, is not an eye-related gene, not an olfactory or taste receptor gene, and is not a gene related to the circadian cycle (e.g., not a CLOCK, PERIOD, CRY gene).

The restricted peptide preferably may be presented on the surface of a tumor.

The restricted peptides may have a size of about 5, about 6, about 7, about 8, about 9, about 10, about 11, about 12, about 13, about 14, or about 15 amino molecule residues, and any range derivable therein. In particular embodiments, the restricted peptide has a size of about 8, about 9, about 10, about 11, or about 12 amino molecule residues. The restricted peptide may be about 5-15 amino acids in length, preferably may be about 7-12 amino acids in length, or more preferably may be about 8-11 amino acids in length.

A restricted peptide of an off-target HLA-PEPTIDE can be a fragment of a protein expressed in normal, e.g., non-tumor tissue. In some embodiments, a restricted peptide of an off-target HLA-PEPTIDE is indicated as being expressed in normal tissues according to the public GTEX database.

HLA Class I molecules which do not associate with a restricted peptide ligand are generally unstable. Accordingly, the association of the restricted peptide with the α1/α2 groove of the HLA molecule may stabilize the non-covalent association of the β2-microglobulin subunit of the HLA subtype with the α-subunit of the HLA subtype.

Stability of the non-covalent association of the β2-microglobulin subunit of the HLA subtype with the α-subunit of the HLA subtype can be determined using any suitable means. For example, such stability may be assessed by dissolving insoluble aggregates of HLA molecules in high concentrations of urea (e.g., about 8M urea), and determining the ability of the HLA molecule to refold in the presence of the restricted peptide during urea removal, e.g., urea removal by dialysis. Such refolding approaches are described in, e.g., Proc. Natl. Acad. Sci. USA Vol. 89, pp. 3429-3433, April 1992, hereby incorporated by reference in its entirety.

For other example, such stability may be assessed using conditional HLA Class I ligands. Conditional HLA Class I ligands are generally designed as short restricted peptides which stabilize the association of the β2 and a subunits of the HLA Class I molecule by binding to the α1/α2 groove of the HLA molecule, and which contain one or more amino acid modifications allowing cleavage of the restricted peptide upon exposure to a conditional stimulus. Upon cleavage of the conditional ligand, the β2 and α-subunits of the HLA molecule dissociate, unless such conditional ligand is exchanged for a restricted peptide which binds to the α1/α2 groove and stabilizes the HLA molecule. Conditional ligands can be designed by introducing amino acid modifications in either known HLA peptide ligands or in predicted high-affinity HLA peptide ligands. For HLA alleles for which structural information is available, water-accessibility of side chains may also be used to select positions for introduction of the amino acid modifications. Use of conditional HLA ligands may be advantageous by allowing the batch preparation of stable HLA-peptide complexes which may be used to interrogate test restricted peptides in a high throughput manner Conditional HLA Class I ligands, and methods of production, are described in, e.g., Proc Natl Acad Sci USA. 2008 Mar. 11; 105(10): 3831-3836; Proc Natl Acad Sci USA. 2008 Mar. 11; 105(10): 3825-3830; J Exp Med. 2018 May 7; 215(5): 1493-1504; Choo, J. A. L. et al. Bioorthogonal cleavage and exchange of major histocompatibility complex ligands by employing azobenzene-containing peptides. Angew Chem Int Ed Engl 53, 13390-13394 (2014); Amore, A. et al. Development of a Hypersensitive Periodate-Cleavable Amino Acid that is Methionine- and Disulfide-Compatible and its Application in MHC Exchange Reagents for T Cell Characterisation. ChemBioChem 14, 123-131 (2012); Rodenko, B. et al. Class I Major Histocompatibility Complexes Loaded by a Periodate Trigger. J Am Chem Soc 131, 12305-12313 (2009); and Chang, C. X. L. et al. Conditional ligands for Asian HLA variants facilitate the definition of CD8+ T-cell responses in acute and chronic viral diseases. Eur J Immunol 43, 1109-1120 (2013). These references are incorporated by reference in their entirety.

Accordingly, in some embodiments, the ability of an HLA-restricted peptide described herein to stabilize the association of the β2- and α-subunits of the HLA molecule, is assessed by performing a conditional ligand mediated-exchange reaction and assay for HLA stability. HLA stability can be assayed using any suitable method, including, e.g., mass spectrometry analysis, immunoassays (e.g., ELISA), size exclusion chromatography, and HLA multimer staining followed by flow cytometry assessment of T cells.

Other exemplary methods for assessing stability of the non-covalent association of the β2-microglobulin subunit of the HLA subtype with the α-subunit of the HLA subtype include peptide exchange using dipeptides. Peptide exchange using dipeptides has been described in, e.g., Proc Natl Acad Sci USA. 2013 Sep. 17, 110(38):15383-8; Proc Natl Acad Sci USA. 2015 Jan. 6, 112(1):202-7, which is hereby incorporated by reference in its entirety.

Provided herein are useful antigens comprising an HLA-PEPTIDE target. The HLA-PEPTIDE targets may comprise a specific HLA-restricted peptide having a defined amino acid sequence complexed with a specific HLA subtype allele.

The HLA-PEPTIDE target or off-target HLA-PEPTIDE may be isolated and/or in substantially pure form. For example, the HLA-PEPTIDE targets or off-target HLA-PEPTIDEs may be isolated from their natural environment, or may be produced by means of a technical process. In some cases, the HLA-PEPTIDE target or off-target HLA-PEPTIDE is provided in a form which is substantially free of other peptides or proteins.

THE HLA-PEPTIDE targets or off-target HLA-PEPTIDEs may be presented in soluble form, and optionally may be a recombinant HLA-PEPTIDE target complex. The skilled artisan may use any suitable method for producing and purifying recombinant HLA-PEPTIDE targets or off-target HLA-PEPTIDEs. Suitable methods include, e.g., use of E. coli expression systems, insect cells, and the like. Other methods include synthetic production, e.g., using cell free systems. An exemplary suitable cell free system is described in WO2017089756, which is hereby incorporated by reference in its entirety.

Also provided herein are compositions comprising an HLA-PEPTIDE target or off-target HLA-PEPTIDE.

In some cases, the composition comprises an HLA-PEPTIDE target or off-target HLA-PEPTIDE attached to a solid support. Exemplary solid supports include, but are not limited to, beads, wells, membranes, tubes, columns, plates, sepharose, magnetic beads, and chips. Exemplary solid supports are described in, e.g., Catalysts 2018, 8, 92; doi:10.3390/cata18020092, which is hereby incorporated by reference in its entirety.

The HLA-PEPTIDE target may be attached to the solid support by any suitable methods known in the art. In some cases, the HLA-PEPTIDE target is covalently attached to the solid support.

In some cases, the HLA-PEPTIDE target is attached to the solid support by way of an affinity binding pair. Affinity binding pairs generally involved specific interactions between two molecules. A ligand having an affinity for its binding partner molecule can be covalently attached to the solid support, and thus used as bait for immobilizing Common affinity binding pairs include, e.g., streptavidin and biotin, avidin and biotin; polyhistidine tags with metal ions such as copper, nickel, zinc, and cobalt; and the like.

The HLA-PEPTIDE target may comprise a detectable label.

### Pharmaceutical Compositions Comprising HLA-PEPTIDE Targets

The composition comprising an HLA-PEPTIDE target may be a pharmaceutical composition. Such a composition may comprise multiple HLA-PEPTIDE targets. Exemplary pharmaceutical compositions are described herein. The composition may be capable of eliciting an immune response. The composition may comprise an adjuvant. Suitable adjuvants include, but are not limited to 1018 ISS, alum, aluminium salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel vector system, PLG microparticles, resiquimod, SRL172, Virosomes and other Virus-like particles, YF-17D, VEGF trap, R848, beta-glucan, Pam3Cys, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Adjuvants such as incomplete Freund's or GM-CSF are useful. Several immunological adjuvants (e.g., MF59) specific for dendritic cells and their preparation have been described previously (Dupuis M, et al., Cell Immunol. 1998; 186(1):18-27; Allison A C; Dev Biol Stand. 1998; 92:3-11). Also cytokines can be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12) (Gabrilovich D I, et al., J Immunother Emphasis Tumor Immunol. 1996 (6):414-418). HLA surface expression and processing of intracellular proteins into peptides to present on HLA can also be enhanced by interferon-gamma (IFN-γ). See, e.g., York I A, Goldberg A L, Mo X Y, Rock K L. Proteolysis and class I major histocompatibility complex antigen presentation. Immunol Rev. 1999; 172:49-66; and Rock K L, Goldberg A L. Degradation of cell proteins and the generation of MHC class I-presented peptides. Ann Rev Immunol. 1999; 17: 12. 739-779, which are incorporated herein by reference in their entirety.

ABPs Comprising an Antibody or Antigen-Binding Fragment Thereof

In some embodiments, the ABP comprises an antibody or antigen-binding fragment thereof.

In some embodiments, the ABPs provided herein comprise a light chain. In some aspects, the light chain is a kappa light chain. In some aspects, the light chain is a lambda light chain.

In some embodiments, the ABPs provided herein comprise a heavy chain. In some aspects, the heavy chain is an IgA. In some aspects, the heavy chain is an IgD. In some aspects, the heavy chain is an IgE. In some aspects, the heavy chain is an IgG. In some aspects, the heavy chain is an IgM. In some aspects, the heavy chain is an IgG1. In some aspects, the heavy chain is an IgG2. In some aspects, the heavy chain is an IgG3. In some aspects, the heavy chain is an IgG4. In some aspects, the heavy chain is an IgA1. In some aspects, the heavy chain is an IgA2.

In some embodiments, the ABPs provided herein comprise an antibody fragment. In some embodiments, the ABPs provided herein consist of an antibody fragment. In some embodiments, the ABPs provided herein consist essentially of an antibody fragment. In some aspects, the ABP fragment is an Fv fragment. In some aspects, the ABP fragment is a Fab fragment. In some aspects, the ABP fragment is a F(ab′)₂ fragment. In some aspects, the ABP fragment is a Fab′ fragment. In some aspects, the ABP fragment is an scFv (sFv) fragment. In some aspects, the ABP fragment is an scFv-Fc fragment. In some aspects, the ABP fragment is a fragment of a single domain ABP.

In some embodiments, an ABP fragment provided herein is derived from an illustrative ABP provided herein. In some embodiments, an ABP fragments provided herein is not derived from an illustrative ABP provided herein and may, for example, be isolated de novo according to the methods provided herein for obtaining ABP fragments.

In some embodiments, an ABP fragment provided herein retains the ability to bind the HLA-PEPTIDE target, as measured by one or more assays or biological effects described herein. In some embodiments, an ABP fragment provided herein retains the ability to prevent HLA-PEPTIDE from interacting with one or more of its ligands, as described herein.

The ABP fragments provided herein may be made by any suitable method, including the illustrative methods described herein or those known in the art. Suitable methods include recombinant techniques and proteolytic digestion of whole ABPs.

In some embodiments, the ABPs provided herein are monoclonal ABPs. Monoclonal ABPs may be obtained, for example, using a hybridoma method or using phage or yeast-based libraries.

DNA encoding the monoclonal ABPs may be readily isolated and sequenced using conventional procedures.

In some embodiments, the ABPs provided herein are polyclonal ABPs.

In some embodiments, the ABPs provided herein comprise a chimeric ABP. In some embodiments, the ABPs provided herein consist of a chimeric ABP. In some embodiments, the ABPs provided herein consist essentially of a chimeric ABP. Chimeric ABPs can be made by any methods known in the art. In some embodiments, a chimeric ABP is made by using recombinant techniques to combine a non-human variable region (e.g., a variable region derived from a mouse, rat, hamster, rabbit, or non-human primate, such as a monkey) with a human constant region.

In some embodiments, the ABPs provided herein comprise a humanized ABP. In some embodiments, the ABPs provided herein consist of a humanized ABP. In some embodiments, the ABPs provided herein consist essentially of a humanized ABP. Humanized ABPs may be generated by replacing most, or all, of the structural portions of a non-human monoclonal ABP with corresponding human ABP sequences.

In some embodiments, the ABPs provided herein comprise a human ABP. In some embodiments, the ABPs provided herein consist of a human ABP. In some embodiments, the ABPs provided herein consist essentially of a human ABP. Human ABPs can be generated by a variety of techniques known in the art, for example by using transgenic animals (e.g., humanized mice), can be derived from phage-display libraries, can be generated by in vitro activated B cells, or can be derived from yeast-based libraries

In some embodiments, the ABPs provided herein comprise an alternative scaffold. In some embodiments, the ABPs provided herein consist of an alternative scaffold. In some embodiments, the ABPs provided herein consist essentially of an alternative scaffold. Any suitable alternative scaffold may be used. In some aspects, the alternative scaffold is selected from an Adnectin™, an iMab, an Anticalin®, an EETI-II/AGRP, a Kunitz domain, a thioredoxin peptide aptamer, an Affibody®, a DARPin, an Affilin, a Tetranectin, a Fynomer, and an Avimer. The alternative scaffolds provided herein may be made by any suitable method, including the illustrative methods described herein or those known in the art.

Also disclosed herein is an isolated humanized, human, or chimeric ABP that competes for binding to an HLA-PEPTIDE with an ABP disclosed herein.

Also disclosed herein is an isolated humanized, human, or chimeric ABP that binds an HLA-PEPTIDE epitope bound by an ABP disclosed herein.

In certain aspects, an ABP may comprise a human Fc region comprising at least one modification that reduces binding to a human Fc receptor.

It is known that when an ABP is expressed in cells, the ABP is modified after translation. Examples of the posttranslational modification include cleavage of lysine at the C terminus of the heavy chain by a carboxypeptidase; modification of glutamine or glutamic acid at the N terminus of the heavy chain and the light chain to pyroglutamic acid by pyroglutamylation; glycosylation; oxidation; deamidation; and glycation, and it is known that such posttranslational modifications occur in various ABPs (See Journal of Pharmaceutical Sciences, 2008, Vol. 97, p. 2426-2447, incorporated by reference in its entirety). In some embodiments, an ABP is an ABP or antigen-binding fragment thereof which has undergone posttranslational modification. Examples of an ABP or antigen-binding fragment thereof which have undergone posttranslational modification include an ABP or antigen-binding fragments thereof which have undergone pyroglutamylation at the N terminus of the heavy chain variable region and/or deletion of lysine at the C terminus of the heavy chain. It is known in the art that such posttranslational modification due to pyroglutamylation at the N terminus and deletion of lysine at the C terminus does not have any influence on the activity of the ABP or fragment thereof (Analytical Biochemistry, 2006, Vol. 348, p. 24-39, incorporated by reference in its entirety).

In some embodiments, the ABPs provided herein are multispecific ABPs.

In some embodiments, a multispecific ABP provided herein binds more than one antigen. In some embodiments, a multispecific ABP binds 2 antigens. In some embodiments, a multispecific ABP binds 3 antigens. In some embodiments, a multispecific ABP binds 4 antigens. In some embodiments, a multispecific ABP binds 5 antigens.

In some embodiments, a multispecific ABP provided herein binds more than one epitope on the HLA-PEPTIDE target. In some embodiments, a multispecific ABP binds 2 epitopes on the HLA-PEPTIDE target. In some embodiments, a multispecific ABP binds 3 epitopes on the HLA-PEPTIDE target.

In some embodiments, the multispecific ABP comprises an antigen-binding domain (ABD) that specifically binds to an HLA-PEPTIDE target disclosed herein and an additional ABD that binds to an additional antigen.

Many multispecific ABP constructs are known in the art, and the ABPs provided herein may be provided in the form of any suitable multispecific construct.

The multispecific ABPs provided herein may be made by any suitable method, including the illustrative methods described herein or those known in the art.

In certain embodiments, an ABP provided herein comprises an Fc region. An Fc region can be wild-type or a variant thereof. In certain embodiments, an ABP provided herein comprises an Fc region with one or more amino acid substitutions, insertions, or deletions in comparison to a naturally occurring Fc region. In some aspects, such substitutions, insertions, or deletions yield ABP with altered stability, glycosylation, or other characteristics. In some aspects, such substitutions, insertions, or deletions yield a glycosylated ABP.

In some embodiments, the Fc region is a variant Fc region. A “variant Fc region” or “engineered Fc region” comprises an amino acid sequence that differs from that of a native-sequence Fc region by virtue of at least one amino acid modification, preferably one or more amino acid substitution(s). Preferably, the variant Fc region has at least one amino acid substitution compared to a native-sequence Fc region or to the Fc region of a parent polypeptide, e.g., from about one to about ten amino acid substitutions, and preferably from about one to about five amino acid substitutions in a native-sequence Fc region or in the Fc region of the parent polypeptide. The variant Fc region herein will preferably possess at least about 80% homology with a native-sequence Fc region and/or with an Fc region of a parent polypeptide, and most preferably at least about 90% homology therewith, more preferably at least about 95% homology therewith.

The term “Fc-region-comprising ABP” refers to an ABP that comprises an Fc region. The C-terminal lysine (residue 447 according to the EU numbering system) of the Fc region may be removed, for example, during purification of the ABP or by recombinant engineering the nucleic acid encoding the ABP. Accordingly, an ABP having an Fc region can comprise an ABP with or without K447.

In some aspects, the Fc region of an ABP provided herein is modified to yield an ABP with altered affinity for an Fc receptor, or an ABP that is more immunologically inert. In some embodiments, the ABP variants provided herein possess some, but not all, effector functions. Such ABPs may be useful, for example, when the half-life of the ABP is important in vivo, but when certain effector functions (e.g., complement activation and ADCC) are unnecessary or deleterious.

In some embodiments, an ABP provided herein comprises one or more alterations that improves or diminishes C1q binding and/or CDC.

In some embodiments, an ABP provided herein comprises one or more alterations to increase half-life. In some embodiments, the ABP comprises one or more non-Fc modifications that extend half-life.

In some embodiments, the multispecific ABP comprises one or more Fc modifications that promote heteromultimerization. In some embodiments, the Fc modification comprises a set of mutations that renders homodimerization electrostatically unfavorable but heterodimerization favorable.

In some embodiments, the Fc modification comprises a modification in the C_{H3} sequence that affects the ability of the C_{H3} domain to bind an affinity agent, e.g., Protein A.

Among the provided ABPs, e.g., HLA-PEPTIDE ABPs, are receptors. The receptors can include antigen receptors and other chimeric receptors that specifically bind an HLA-PEPTIDE target disclosed herein. The receptor may be a chimeric antigen receptor (CAR).

Also provided are cells expressing the receptors and uses thereof in adoptive cell therapy, such as treatment of diseases and disorders associated with HLA-PEPTIDE expression, including cancer.

The chimeric receptors, such as CARs, generally include an extracellular antigen binding domain that includes, is, or is comprised within, one of the provided anti-HLA-PEPTIDE ABPs such as anti-HLA-PEPTIDE antibodies. Thus, the chimeric receptors, e.g., CARs, typically include in their extracellular portions one or more HLA-PEPTIDE-ABPs, such as one or more antigen-binding fragment, domain, or portion, or one or more antibody variable domains, and/or antibody molecules, such as those described herein. In some embodiments, the CAR includes a HLA-PEPTIDE-binding portion or portions of the ABP (e.g., antibody) molecule, such as a variable heavy (VH) chain region and/or variable light (VL) chain region of the antibody, e.g., an scFv antibody fragment.

In some embodiments, the CAR is a recombinant CAR.

The recombinant CAR may be a human CAR, comprising fully human sequences, e.g., natural human sequences.

Also provided are cells such as cells that contain an antigen receptor, e.g., that contains an extracellular domain including an anti-HLA-PEPTIDE ABP (e.g., a CAR), described herein. Also provided are populations of such cells, and compositions containing such cells. In some embodiments, compositions or populations are enriched for such cells, such as in which cells expressing the HLA-PEPTIDE ABP make up at least 1, 5, 10, 20, 30, 40, 50, 60, 70, 80, 90, 91, 92, 93, 94, 95, 96, 97, 98, 99, or more than 99 percent of the total cells in the composition or cells of a certain type such as T cells or CD8+ or CD4+ cells. In some embodiments, a composition comprises at least one cell containing an antigen receptor disclosed herein. Among the compositions are pharmaceutical compositions and formulations for administration, such as for adoptive cell therapy. Also provided are therapeutic methods for administering the cells and compositions to subjects, e.g., patients.

Thus also provided are genetically engineered cells expressing an ABP comprising a receptor, e.g., a CAR.

Nucleotides, Vectors, Host Cells, and Related Methods

Also provided are isolated nucleic acids encoding HLA-PEPTIDE ABPs, vectors comprising the nucleic acids, and host cells comprising the vectors and nucleic acids, as well as recombinant techniques for the production of the ABPs.

The nucleic acids may be recombinant. The recombinant nucleic acids may be constructed outside living cells by joining natural or synthetic nucleic acid segments to nucleic acid molecules that can replicate in a living cell, or replication products thereof. For purposes herein, the replication can be in vitro replication or in vivo replication.

For recombinant production of an ABP, the nucleic acid(s) encoding it may be isolated and inserted into a replicable vector for further cloning (i.e., amplification of the DNA) or expression. In some aspects, the nucleic acid may be produced by homologous recombination, for example as described in U.S. Pat. No. 5,204,244, incorporated by reference in its entirety.

Many different vectors are known in the art. The vector components generally include one or more of the following: a signal sequence, an origin of replication, one or more marker genes, an enhancer element, a promoter, and a transcription termination sequence, for example as described in U.S. Pat. No. 5,534,615, incorporated by reference in its entirety.

Exemplary vectors or constructs suitable for expressing an ABP, e.g., a CAR, antibody, or antigen binding fragment thereof, include, e.g., the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as AGTlO, AGTl 1, AZapII (Stratagene), AEMBL4, and ANMl 149, are also suitable for expressing an ABP disclosed herein.

Illustrative examples of suitable host cells are provided below. These host cells are not meant to be limiting, and any suitable host cell may be used to produce the ABPs provided herein.

Suitable host cells include any prokaryotic (e.g., bacterial), lower eukaryotic (e.g., yeast), or higher eukaryotic (e.g., mammalian) cells. Suitable prokaryotes include eubacteria, such as Gram-negative or Gram-positive organisms, for example, Enterobacteriaceae such as Escherichia (E. coli), Enterobacter, Erwinia, Klebsiella, Proteus, Salmonella (S. typhimurium), Serratia (S. marcescans), Shigella, Bacilli (B. subtilis and B. licheniformis), Pseudomonas (P. aeruginosa), and Streptomyces. One useful E. coli cloning host is E. coli 294, although other strains such as E. coli B, E. coli X1776, and E. coli W3110 are also suitable.

In addition to prokaryotes, eukaryotic microbes such as filamentous fungi or yeast are also suitable cloning or expression hosts for HLA-PEPTIDE ABP-encoding vectors. Saccharomyces cerevisiae, or common baker's yeast, is a commonly used lower eukaryotic host microorganism. However, a number of other genera, species, and strains are available and useful, such as Schizosaccharomyces pombe, Kluyveromyces (K. lactis, K. fragilis, K. bulgaricus K. wickeramii, K. waltii, K. drosophilarum, K. thermotolerans, and K. marxianus), Yarrowia, Pichia pastoris, Candida (C. albicans), Trichoderma reesia, Neurospora crassa, Schwanniomyces (S. occidentalis), and filamentous fungi such as, for example Penicillium, Tolypocladium, and Aspergillus (A. nidulans and A. niger).

Useful mammalian host cells include COS-7 cells, HEK293 cells; baby hamster kidney (BHK) cells; Chinese hamster ovary (CHO); mouse sertoli cells; African green monkey kidney cells (VERO-76), and the like.

The host cells used to produce the HLA-PEPTIDE ABP may be cultured in a variety of media. Commercially available media such as, for example, Ham's F10, Minimal Essential Medium (MEM), RPMI-1640, and Dulbecco's Modified Eagle's Medium (DMEM) are suitable for culturing the host cells. In addition, any of the media described in Ham et al., Meth. Enz., 1979, 58:44; Barnes et al., Anal. Biochem., 1980, 102:255; and U.S. Pat. Nos. 4,767,704, 4,657,866, 4,927,762, 4,560,655, and 5,122,469; or WO 90/03430 and WO 87/00195 may be used. Each of the foregoing references is incorporated by reference in its entirety.

Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics, trace elements (defined as inorganic compounds usually present at final concentrations in the micromolar range), and glucose or an equivalent energy source. Any other necessary supplements may also be included at appropriate concentrations that would be known to those skilled in the art.

The culture conditions, such as temperature, pH, and the like, are those previously used with the host cell selected for expression, and will be apparent to the ordinarily skilled artisan.

When using recombinant techniques, the ABP can be produced intracellularly, in the periplasmic space, or directly secreted into the medium. If the ABP is produced intracellularly, as a first step, the particulate debris, either host cells or lysed fragments, is removed, for example, by centrifugation or ultrafiltration. For example, Carter et al. (Bio/Technology, 1992, 10:163-167, incorporated by reference in its entirety) describes a procedure for isolating ABPs which are secreted to the periplasmic space of E. coli. Briefly, cell paste is thawed in the presence of sodium acetate (pH 3.5), EDTA, and phenylmethylsulfonylfluoride (PMSF) over about 30 min Cell debris can be removed by centrifugation.

In some embodiments, the ABP is produced in a cell-free system. In some aspects, the cell-free system is an in vitro transcription and translation system as described in Yin et al., mAbs, 2012, 4:217-225, incorporated by reference in its entirety. In some aspects, the cell-free system utilizes a cell-free extract from a eukaryotic cell or from a prokaryotic cell. In some aspects, the prokaryotic cell is E. coli. Cell-free expression of the ABP may be useful, for example, where the ABP accumulates in a cell as an insoluble aggregate, or where yields from periplasmic expression are low.

Where the ABP is secreted into the medium, supernatants from such expression systems are generally first concentrated using a commercially available protein concentration filter, for example, an Amicon® or Millipore® Pellcon® ultrafiltration unit. A protease inhibitor such as PMSF may be included in any of the foregoing steps to inhibit proteolysis and antibiotics may be included to prevent the growth of adventitious contaminants.

The ABP composition prepared from the cells can be purified using, for example, hydroxylapatite chromatography, gel electrophoresis, dialysis, and affinity chromatography, with affinity chromatography being a particularly useful purification technique. The suitability of protein A as an affinity ligand depends on the species and isotype of any immunoglobulin Fc domain that is present in the ABP. Protein A can be used to purify ABPs that comprise human γ1, γ2, or γ4 heavy chains (Lindmark et al., J. Immunol. Meth., 1983, 62:1-13, incorporated by reference in its entirety). Protein G is useful for all mouse isotypes and for human γ3 (Guss et al., EMBO J., 1986, 5:1567-1575, incorporated by reference in its entirety).

The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the ABP comprises a C_{H3} domain, the BakerBond ABX® resin is useful for purification.

Other techniques for protein purification, such as fractionation on an ion-exchange column, ethanol precipitation, Reverse Phase HPLC, chromatography on silica, chromatography on heparin Sepharose®, chromatofocusing, SDS-PAGE, and ammonium sulfate precipitation are also available, and can be applied by one of skill in the art.

Following any preliminary purification step(s), the mixture comprising the ABP of interest and contaminants may be subjected to low pH hydrophobic interaction chromatography using an elution buffer at a pH between about 2.5 to about 4.5, generally performed at low salt concentrations (e.g., from about 0 to about 0.25 M salt).

Methods of Identifying ABPs

Identification and/or preparation of an ABP described herein may comprise use of an HLA-PEPTIDE target or off-target HLA-PEPTIDE.

Such antigens may comprise intact HLA-PEPTIDE complexes or fragments thereof. Such antigen may be, for example, in the form of isolated protein or a protein expressed on the surface of a cell.

In some embodiments, the HLA-PEPTIDE antigen is a non-naturally occurring variant of HLA-PEPTIDE, such as a HLA-PEPTIDE protein having an amino acid sequence or post-translational modification that does not occur in nature.

In some embodiments, the HLA-PEPTIDE antigen is truncated by removal of, for example, intracellular or membrane-spanning sequences, or signal sequences. In some embodiments, the HLA-PEPTIDE antigen is fused at its C-terminus to a human IgG1 Fc domain or a polyhistidine tag.

ABPs that bind HLA-PEPTIDE can be identified using any method known in the art, e.g., phage display or immunization of a subject.

One method of identifying an antigen binding protein includes binding a target disclosed herein with an antigen binding protein, contacting the antigen binding protein with one or more off-target HLA-PEPTIDEs disclosed herein, and identifying the antigen binding protein if the antigen binding protein does not bind to the one or more off-target HLA-PEPTIDEs. The antigen binding protein can be present in a library comprising a plurality of distinct antigen binding proteins.

In some embodiments, the library is a phage display library. The phage display library can be developed so that it is substantially free of antigen binding proteins that non-specifically bind the HLA of the HLA-PEPTIDE target. The antigen binding protein can be present in a yeast display library comprising a plurality of distinct antigen binding proteins. The yeast display library can be developed so that it is substantially free of antigen binding proteins that non-specifically bind the HLA of the HLA-PEPTIDE target.

In some embodiments, the library is a yeast display library.

Another method of identifying an antigen binding protein can include obtaining at least one HLA-PEPTIDE target; administering the HLA-PEPTIDE target to a subject (e.g., a mouse, rabbit or a llama), optionally in combination with an adjuvant; and isolating the antigen binding protein from the subject.

In some aspects, isolating the antigen binding protein comprises isolating a B cell from the subject that expresses the antigen binding protein. The B cell can be used to create a hybridoma. The B cell can also be used for cloning one or more of its CDRs. The B cell can also be immortalized, for example, by using EBV transformation. Sequences encoding an antigen binding protein can be cloned from immortalized B cells or can be cloned directly from B cells isolated from an immunized subject. A library that comprises the antigen binding protein of the B cell can also be created, optionally wherein the library is phage display or yeast display.

Methods for Engineering Cells with ABPs

Also provided are methods, nucleic acids, compositions, and kits, for expressing the ABPs, including receptors comprising antibodies, and CARs, and for producing genetically engineered cells expressing such ABPs. The genetic engineering generally involves introduction of a nucleic acid encoding the recombinant or engineered component into the cell, such as by retroviral transduction, transfection, or transformation.

Preparation of Engineered Cells

In some embodiments, preparation of the engineered cells includes one or more culture and/or preparation steps. The cells for introduction of the HLA-PEPTIDE-ABP, e.g., CAR, can be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

In some embodiments, the cells are incubated and/or cultured prior to or in connection with genetic engineering.

Assays

A variety of assays known in the art may be used to identify and characterize an HLA-PEPTIDE ABP provided herein.

### Binding, Competition, and Epitope Mapping Assays

Specific antigen-binding activity of an ABP provided herein may be evaluated by any suitable method, including using SPR, BLI, RIA and MSD, as described elsewhere in this disclosure. Additionally, antigen-binding activity may be evaluated by ELISA assays, using flow cytometry, and/or Western blot assays.

Assays for measuring competition between two ABPs, or an ABP and another molecule (e.g., one or more ligands of HLA-PEPTIDE such as a TCR) are described elsewhere in this disclosure and, for example, in Harlow and Lane, ABPs: A Laboratory Manual ch. 14, 1988, Cold Spring Harbor Laboratory, Cold Spring Harbor, N.Y, incorporated by reference in its entirety.

Assays for mapping the epitopes to which an ABP provided herein bind are described, for example, in Morris “Epitope Mapping Protocols,” in Methods in Molecular Biology vol. 66, 1996, Humana Press, Totowa, N.J., incorporated by reference in its entirety. In some embodiments, the epitope is determined by peptide competition. In some embodiments, the epitope is determined by mass spectrometry. In some embodiments, the epitope is determined by mutagenesis. In some embodiments, the epitope is determined by crystallography.

### Assays for Effector Functions

Effector function following treatment with an ABP and/or cell provided herein may be evaluated using a variety of in vitro and in vivo assays known in the art, including those described in Ravetch and Kinet, Annu. Rev. Immunol., 1991, 9:457-492; U.S. Pat. Nos. 5,500,362, 5,821,337; Hellstrom et al., Proc. Nat'l Acad. Sci. USA, 1986, 83:7059-7063; Hellstrom et al., Proc. Nat'l Acad. Sci. USA, 1985, 82:1499-1502; Bruggemann et al., J. Exp. Med., 1987, 166:1351-1361; Clynes et al., Proc. Nat'l Acad. Sci. USA, 1998, 95:652-656; WO 2006/029879; WO 2005/100402; Gazzano-Santoro et al., J. Immunol. Methods, 1996, 202:163-171; Cragg et al., Blood, 2003, 101:1045-1052; Cragg et al. Blood, 2004, 103:2738-2743; and Petkova et al., Int'l. Immunol., 2006, 18:1759-1769; each of which is incorporated by reference in its entirety.

### Pharmaceutical Compositions

An ABP, cell, or HLA-PEPTIDE target provided herein can be formulated in any appropriate pharmaceutical composition and administered by any suitable route of administration. Suitable routes of administration include, but are not limited to, the intra-arterial, intradermal, intramuscular, intraperitoneal, intravenous, nasal, parenteral, pulmonary, and subcutaneous routes. These compositions can comprise, in addition to one or more of the antibodies disclosed herein, a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g., oral, intravenous, cutaneous or subcutaneous, nasal, intramuscular, intraperitoneal routes.

Therapeutic Applications

For therapeutic applications, ABPs and/or cells are administered to a mammal, generally a human, in a pharmaceutically acceptable dosage form such as those known in the art and those discussed above. For example, ABPs and/or cells may be administered to a human intravenously as a bolus or by continuous infusion over a period of time, by intramuscular, intraperitoneal, intra-cerebrospinal, subcutaneous, intra-articular, intrasynovial, intrathecal, or intratumoral routes. The ABPs also are suitably administered by peritumoral, intralesional, or perilesional routes, to exert local as well as systemic therapeutic effects. The intraperitoneal route may be particularly useful, for example, in the treatment of ovarian tumors.

The ABPs and/or cells provided herein can be useful for the treatment of any disease or condition involving HLA-PEPTIDE. In some embodiments, the disease or condition is a disease or condition that can benefit from treatment with an anti-HLA-PEPTIDE ABP and/or cell. In some embodiments, the disease or condition is a tumor. In some embodiments, the disease or condition is a cell proliferative disorder. In some embodiments, the disease or condition is a cancer.

In some embodiments, the ABPs and/or cells provided herein are provided for use as a medicament. In some embodiments, the ABPs and/or cells provided herein are provided for use in the manufacture or preparation of a medicament. In some embodiments, the medicament is for the treatment of a disease or condition that can benefit from an anti-HLA-PEPTIDE ABP and/or cell. In some embodiments, the disease or condition is a tumor. In some embodiments, the disease or condition is a cell proliferative disorder. In some embodiments, the disease or condition is a cancer.

In some embodiments, provided herein is a method of treating a disease or condition in a subject in need thereof by administering an effective amount of an ABP and/or cell provided herein to the subject. In some aspects, the disease or condition is a cancer.

In some embodiments, provided herein is a method of treating a disease or condition in a subject in need thereof by administering an effective amount of an ABP and/or cell provided herein to the subject, wherein the disease or condition is a cancer, and the cancer is selected from a solid tumor and a hematological tumor.

In some embodiments, provided herein is a method of modulating an immune response in a subject in need thereof, comprising administering to the subject an effective amount of an ABP and/or cell or a pharmaceutical composition disclosed herein. In some embodiments, the modulating of the immune response comprises increasing the immune response. Stimulating the immune response may comprise stimulating an immune response or enhancing an immune response.

In some embodiments of any one of the methods described herein, the presence of an HLA-PEPTIDE target described herein has been detected in the subject or a biological sample obtained from the subject. In some embodiments of any one of the methods described herein, the presence of a restricted peptide of an HLA-PEPTIDE target described herein has been detected in the subject or a biological sample obtained from the subject. In some embodiments of any one of the methods described herein, the presence of the HLA subtype of an HLA-PEPTIDE target described herein has been detected in the subject or a biological sample obtained from the subject. In some embodiments, the method comprises administering an ABP disclosed herein to the subject after having determined the presence of the HLA-PEPTIDE target, restricted peptide, or HLA in the biological sample obtained from the subject.

Diagnostic Methods

Also provided are methods for predicting and/or detecting the presence of a given HLA-PEPTIDE on a cell from a subject. Such methods may be used, for example, to predict and evaluate responsiveness to treatment with an ABP and/or cell provided herein.

In some embodiments, a blood or tumor sample is obtained from a subject and the fraction of cells expressing HLA-PEPTIDE is determined. In some aspects, the relative amount of HLA-PEPTIDE expressed by such cells is determined. The fraction of cells expressing HLA-PEPTIDE and the relative amount of HLA-PEPTIDE expressed by such cells can be determined by any suitable method. In some embodiments, flow cytometry is used to make such measurements. In some embodiments, fluorescence assisted cell sorting (FACS) is used to make such measurement. See Li et al., J. Autoimmunity, 2003, 21:83-92 for methods of evaluating expression of HLA-PEPTIDE in peripheral blood.

In some embodiments, detecting the presence of a given HLA-PEPTIDE on a cell from a subject is performed using immunoprecipitation and mass spectrometry. This can be performed by obtaining a tumor sample (e.g., a frozen tumor sample) such as a primary tumor specimen and applying immunoprecipitation to isolate one or more peptides. The HLA alleles of the tumor sample can be determined experimentally or obtained from a third party source. The one or more peptides can be subjected to mass spectrometry (MS) to determine their sequence(s). The spectra from the MS can then be searched against a database. An example is provided in the Examples section below.

In some embodiments, predicting the presence of a given HLA-PEPTIDE on a cell from a subject is performed using a computer-based model applied to the peptide sequence and/or RNA measurements of one or more genes comprising that peptide sequence (e.g., RNA seq or RT-PCR, or nanostring) from a tumor sample. The model used can be as described in international patent application no. PCT/US2016/067159, herein incorporated by reference, in its entirety, for all purposes.

Kits

Also provided are kits comprising an ABP and/or cell provided herein. The kits may be used for the treatment, prevention, and/or diagnosis of a disease or disorder, as described herein.

In some embodiments, the kit comprises a container and a label or package insert on or associated with the container. Suitable containers include, for example, bottles, vials, syringes, and IV solution bags. The containers may be formed from a variety of materials, such as glass or plastic. The container holds a composition that is by itself, or when combined with another composition, effective for treating, preventing and/or diagnosing a disease or disorder. The container may have a sterile access port. For example, if the container is an intravenous solution bag or a vial, it may have a port that can be pierced by a needle. At least one active agent in the composition is an ABP provided herein. The label or package insert indicates that the composition is used for treating the selected condition.

In some embodiments, the kit comprises (a) a first container with a first composition contained therein, wherein the first composition comprises an ABP and/or cell provided herein; and (b) a second container with a second composition contained therein, wherein the second composition comprises a further therapeutic agent. The kit in this embodiment can further comprise a package insert indicating that the compositions can be used to treat a particular condition, e.g., cancer.

Alternatively, or additionally, the kit may further comprise a second (or third) container comprising a pharmaceutically-acceptable excipient. In some aspects, the excipient is a buffer. The kit may further include other materials desirable from a commercial and user standpoint, including filters, needles, and syringes.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3rd Ed. (Plenum Press) Vols A and B(1992).

### Example 1: Identification of Predicted Off-Target Liability Peptides for HLA-PEPTIDE Target A*01:01_ NTDNNLAVY (SEQ ID NO: 1) (G2)

Peptides were prioritized for deselection and screening in the discovery campaign through our off-target liability analysis (OTLA) process. Briefly, given a target of interest, e.g., HLA-PEPTIDE target HLA-A*01:01_ NTDNNLAVY (SEQ ID NO: 1) (G2), off-target peptides from elsewhere in the human transcriptome were identified by sequence similarity searching. Off-target peptides with up to 5 amino acid mismatches were allowed from the target of interest. These peptides were then evaluated using the proprietary presentation prediction algorithm EDGE, as described in Bulik-Sullivan, et al. Nat Biotechnol. 2018 Dec. 17. doi: 10.1038/nbt.4313, which is hereby incorporated by reference in its entirety. The evaluation focused on predicted HLA-A*01:01 presentation and employed RNA expression levels found in normal tissues from the public GTEx database. An EDGE score cut-off was selected such that we would identify the vast majority of true positive peptides. The peptides prioritized by EDGE were further prioritized by similarity to the target, extent of peptide coverage, and expression in vital tissue types. A set of five prioritized peptides was generated as pHLA in the context of A*01:01 and used for the lead identification campaign as described in Example 3.

### Example 2: Validation of Predicted G2 Off-Target Liability Peptide-HLA Complexes

As an assessment to validate the predicted HLA-OTLA targets (Table 1) arising from the EDGE model analyses of HLA-PEPTIDE complex HLA-A*01:01_NTDNNLAVY (SEQ ID NO: 1) was completed using mass spectrometry (MS) on tumor samples known to be positive for each given HLA allele from the respective HLA-PEPTIDE complexes.

TABLE 5

| Protein Name | HLA-PEPTIDE (predicted G2 OTLA) |
|---|---|
| DSG3 | HLA-A*01:01_YTDNWLAVY (SEQ ID NO: 3) |
| DSG4 | HLA-A*01:01_GTDNWLAQY (SEQ ID NO: 4) |
| PTS | HLA-A*01:01_ETDNNIVVY (SEQ ID NO: 2) |
| KDM7A | HLA-A*01:01_PTDENLARY (SEQ ID NO: 5) |
| ICE1 | HLA-A*01:01_NTDNLLTEY (SEQ ID NO: 6) |

Isolation of HLA-peptide molecules was performed using classic immunoprecipitation (IP) methods after lysis and solubilization of the tissue sample (1-4). Fresh frozen tissue was first frozen in liquid nitrogen and pulverized (CryoPrep; Covaris, Woburn, Mass.). One tenth of the sample was aliquoted for genomic sequencing efforts and lysis buffer (1% CHAPS, 20 mM Tris-HCl, 150 mM NaCl, protease and phosphatase inhibitors, pH=8) was added to solubilize the remaining pulverized tissue. The sample lysate was spun at 4° C. for 2 hours to pellet debris. The clarified lysate was used for the HLA specific IP.

Immunoprecipitation was performed using antibodies coupled to beads where the antibody was specific for HLA molecules. For pan-Class I HLA immunoprecipitation, the antibody W6/32 (5) was used. Antibody was covalently attached to NHS-sepharose beads during overnight incubation. After covalent attachment, the beads were washed and aliquoted for IP. Additional methods for IP can be used including but not limited to Protein A/G capture of antibody, magnetic bead isolation, or other methods commonly used for immunoprecipitation.

The lysate was added to the antibody beads and rotated at 4° C. overnight for the immunoprecipitation. After immunoprecipitation, the beads were removed from the lysate and the lysate was stored for additional experiments, including additional IPs. The IP beads were washed to remove non-specific binding and the HLA/peptide complex was eluted from the beads with 2N acetic acid. The protein components were removed from the peptides using a molecular weight spin column or C18 cleanup step. The resultant peptides were taken to dryness by SpeedVac evaporation and stored at −20° C. prior to MS analysis.

Dried peptides were reconstituted in 0.1% formic acid in 3% acetonitrile containing 20-80 femtomoles synthesized heavy labeled peptides for the respective OTLA peptides (Table 5). The solution was then loaded onto a C-18 microcapillary HPLC column for gradient elution into the mass spectrometer. A gradient of 0-40% B (solvent A−0.1% formic acid, solvent B-0.1% formic acid in 80% acetonitrile) in 180 minutes was used to elute the peptides into the Fusion Lumos mass spectrometer (Thermo). MS1 spectra of peptide mass/charge (m/z) were collected in the Orbitrap detector with 60,000 resolution followed by targeted MS2 scans. Automatic gain control (AGC) for MS1 scans was set to 4×10⁵ and for MS2 scans was set to 1×10⁴. For sequencing HLA peptides, +1 and +2 charge states were selected for MS2 fragmentation. The MS2 spectra were acquired using methods where only masses targeting MS1 precursors listed in an inclusion list were selected for isolation and fragmentation. This is commonly referred to as Targeted Mass Spectrometry and was performed in a quantitative manner. Quantitation methods require each peptide to be quantitated to be synthesized using heavy labeled amino acids. (6)

Spectra from targeted MS2 experiments were analyzed using Skyline (7) or other method to analyze predicted fragment ions.

The presence of multiple peptides from the predicted HLA-OTLA complexes was determined using mass spectrometry (MS) on various patient tumor samples known to be positive for A*01:01 allele. Four of the five OTLA peptides in Table 5 were detected in tissue of 5 patients testing positive for HLA type A*01:01. An estimate of tissue density (copies/cell) for each of the observed OTLA peptides was determined by normalizing the observed endogenous peptide response to the known heavy peptide standard included in the MS experiment, then adjusting to the number of cell calculated to be in the amount of tissue processed (grams). The results are shown in Table 6.

TABLE 6

| OTLA | | | Tissue | HLA Class 1 Typing | | | | | | Tissue density |
|---|---|---|---|---|---|---|---|---|---|---|
| Peptide | Gene | Patient | type | A1 | A2 | B1 | B2 | C1 | C2 | Copies/cell |
| ETDNNIVVY | PTS | 1 | Lung | A*01:01 | A*23:01 | B*44:03 | B*37:01 | C*06:02 | C*04:01 | 9 |
| (SEQ ID NO: 2) | | | | | | | | | | |
| ETDNNIVVY | PTS | 2 | Lung | A*11:01 | A*01:01 | B*27:05 | B*57:01 | C*06:02 | C*02:02 | 462 |
| (SEQ ID NO: 2) | | | | | | | | | | |
| NTDNLLTEY | ICE1 | 2 | Lung | A*11:01 | A*01:01 | B*27:05 | B*57:01 | C*06:02 | C*02:02 | 95 |
| (SEQ ID NO: 6) | | | | | | | | | | |
| PTDENLARY | KDM7A | 2 | Lung | A*11:01 | A*01:01 | B*27:05 | B*57:01 | C*06:02 | C*02:02 | 5 |
| (SEQ ID NO: 5) | | | | | | | | | | |
| YTDNWLAVY | DSG3 | 2 | Lung | A*11:01 | A*01:01 | B*27:05 | B*57:01 | C*06:02 | C*02:02 | 23 |
| (SEQ ID NO: 3) | | | | | | | | | | |
| ETDNNIVVY | PTS | 3 | Lung | A*02:01 | A*01:01 | B*57:01 | B*52:01 | C*06:02 | C*12:02 | 240 |
| (SEQ ID NO: 2) | | | | | | | | | | |
| NTDNLLTEY | ICE1 | 3 | Lung | A*02:01 | A*01:01 | B*57:01 | B*52:01 | C*06:02 | C*12:02 | 74 |
| (SEQ ID NO: 6) | | | | | | | | | | |
| PTDENLARY | KDM7A | 3 | Lung | A*02:01 | A*01:01 | B*57:01 | B*52:01 | C*06:02 | C*12:02 | 2 |
| (SEQ ID NO: 5) | | | | | | | | | | |
| ETDNNIVVY | PTS | 4 | Lung | A*31:01 | A*01:01 | B*44:02 | B*52:01 | C*12:02 | C*05:01 | 89 |
| (SEQ ID NO: 2) | | | | | | | | | | |
| ETDNNIVVY | PTS | 5 | Ovary | A*02:01 | A*01:01 | B*08:01 | B*15:01 | C*07:01 | C*03:03 | 195 |
| (SEQ ID NO: 2) | | | | | | | | | | |
| NTDNLLTEY | ICE1 | 5 | Ovary | A*02:01 | A*01:01 | B*08:01 | B*15:01 | C*07:01 | C*03:03 | 63 |
| (SEQ ID NO: 6) | | | | | | | | | | |
| PTDENLARY | KDM7A | 5 | Ovary | A*02:01 | A*01:01 | B*08:01 | B*15:01 | C*07:01 | C*03:03 | 1 |
| (SEQ ID NO: 5) | | | | | | | | | | |

Of the tissues tested, OTLA peptide ETDNNIVVY (SEQ ID NO: 2) presented with highest density at median of 195 copies/cell.

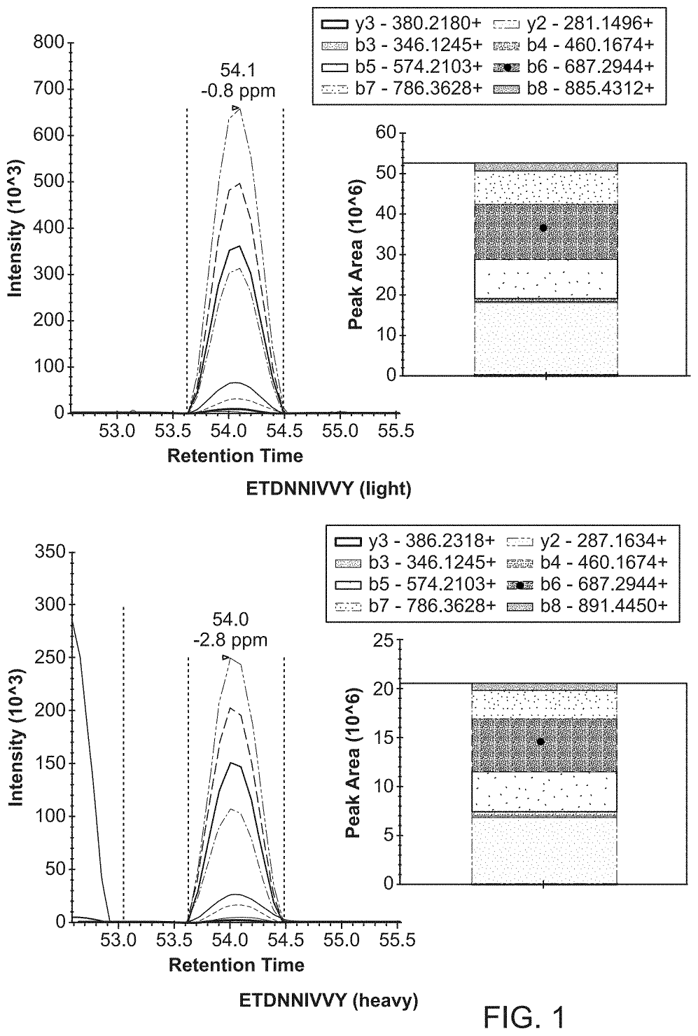

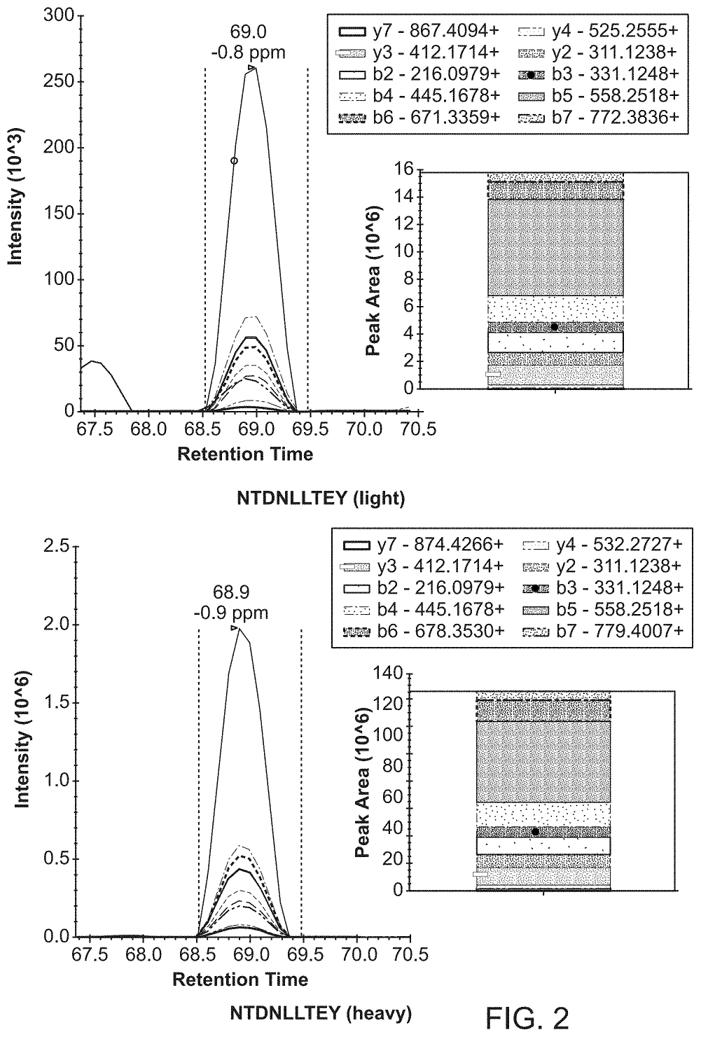

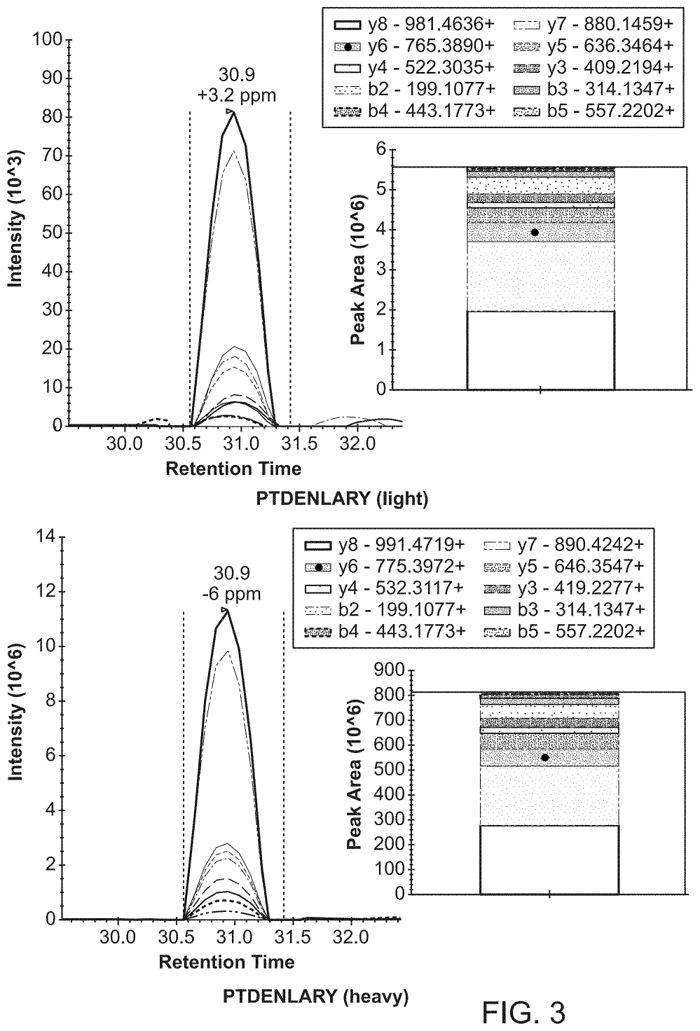

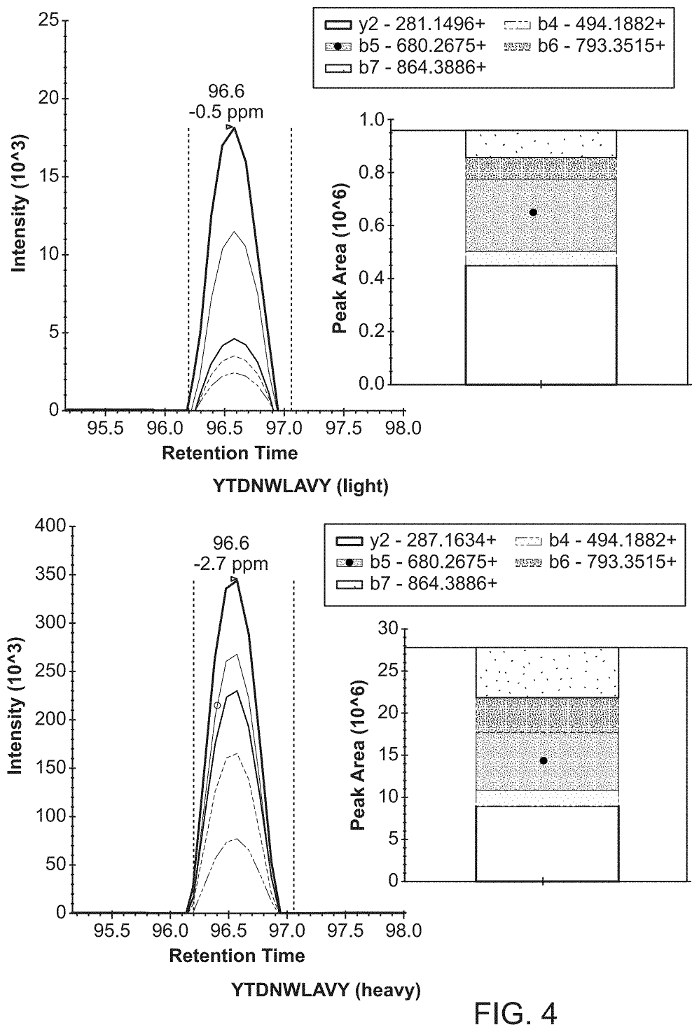

Representative spectra data for selected HLA-restricted peptides is shown in FIGS. 1-4. Each spectrum contains the peptide fragmentation information as well as relative peak area abundances of each transition for both heavy (standard) and light (sample) forms of the peptide.

The presentation of peptide ETDNNIVVY (SEQ ID NO: 2) in HLA peptidomes has also been reported in literature. In one example, one human B cell line GD149 which does carry the A*01:01 allele was found to present the ETDNNIVVY (SEQ ID NO: 2) peptide using mass spectrometry. (8)

### REFERENCES

(1) Hunt D F, Henderson R A, Shabanowitz J, Sakaguchi K, Michel H, Sevilir N, Cox A L, Appella E, Engelhard V H. Characterization of peptides bound to the class I MHC molecule HLA-A2.1 by mass spectrometry. Science 1992. 255: 1261-1263.
(2) Zarling A L, Polefrone J M, Evans A M, Mikesh L M, Shabanowitz J, Lewis S T, Engelhard V H, Hunt D R Identification of class I MHC-associated phosphopeptides as targets for cancer immunotherapy. Proc Natl Acad Sci USA. 2006 Oct. 3; 103(40):14889-94.
(3) Bassani-Sternberg M, Pletscher-Frankild S, Jensen U, Mann M. Mass spectrometry of human leukocyte antigen class I peptidomes reveals strong effects of protein abundance and turnover on antigen presentation. Mol Cell Proteomics. 2015 March; 14(3):658-73. doi: 10.1074/mcp.M114.042812.
(4) Abelin J G, Trantham P D, Penny S A, Patterson A M, Ward S T, Hildebrand W H, Cobbold M, Bai D L, Shabanowitz J, Hunt D R Complementary IMAC enrichment methods for HLA-associated phosphopeptide identification by mass spectrometry. Nat Protoc. 2015 September; 10(9):1308-18. doi: 10.1038/nprot.2015.086. Epub 2015 Aug. 6
(5) Barnstable C J, Bodmer W F, Brown G, Galfre G, Milstein C, Williams A F, Ziegler A. Production of monoclonal antibodies to group A erythrocytes, HLA and other human cell surface antigens-new tools for genetic analysis. Cell. 1978 May; 14(1):9-20.
(6) Doerr, A. (2013) Mass Spectrometry-based targeted proteomics. Nature Methods, 10, 23.
(7) Lindsay K. Pino, Brian C. Searle, James G. Bollinger, Brook Nunn, Brendan MacLean & M. J. MacCoss (2017) The Skyline ecosystem: Informatics for quantitative mass spectrometry proteomics. Mass Spectrometry Reviews.
(8) Michal Bassani-Sternberg, Chloe Chong, Philippe Guillaume, Marthe Solleder, HuiSong Pak, Phippe O. Gannon, Lana E. Kandalaft, George Coukos, David Gfeller. Deciphering HLA-1 motifs across HLA peptidomes improves neo-antigen predictions and identifies allostery regulating HLA specificity. PLOS Computational Biology. 2017 August; 13(8), doi: 10.1371/journal.pcbi.1005725.

### Example 3: Identification of Antigen-Binding Proteins (ABPs) that Selectively Bind HLA-PEPTIDE Target A*01:01_ NTDNNLAVY (SEQ ID NO: 1) Over Identified G2 Off-Target Liability Peptides

Phage Display Panning

The highly diverse SuperHuman 2.0 synthetic naïve scFv library from Distributed Bio Inc was used as input material for phage display, which has a 7.6×10¹⁰ total diversity on ultra-stable and diverse VH/VL scaffolds. Before the first round of panning, the library was depleted three times against Dynabead M-280 streptavidin beads (Life Technologies) followed by a depletion against Streptavidin beads pre-bound with 100 pmoles of pooled negative peptide-HLA complexes. For the first round of panning, 100 pmoles of peptide-HLA complex bound to streptavidin beads was incubated with depleted phage for 2 hours at room temperature with rotation. Three five-minute washes with 0.5% BSA in 1×PBST (PBS+0.05% Tween-20) followed by three five-minute washes with 0.5% BSA in 1×PBS were utilized to remove any unbound phage to the peptide-HLA complex bound beads. To elute the bound phage from the washed beads, 1 mL 0.1M TEA was added and incubated for 10 minutes at room temperature with rotation. The eluted phage was collected from the beads and neutralized with 0.5 mL 1M Tris-HCl pH 7.5. The neutralized phage was then used to infect log growth TG-1 cells (OD₆₀₀=0.5) and after an hour of infection at 37° C., cells were plated onto 2YT media with 100 μg/mL carbenicillin and 2% glucose (2YTCG) agar plates for output titer and bacterial growth for subsequent panning rounds.

The post-round-1 panning output was amplified by PCR. The amplified DNA as well as the pADL-23c phagemid vector were double digested with restriction enzymes EcoR1 and Spe1 (New England Biolabs) and subsequently ligated using T4 DNA ligase. After purification of DNA from the ligation reaction, the ligated product was electroporated into electrocompetent TG1 cells (Lucigen). The cells were plated on 2YTCG agar plates to generate the final post-round-1 panning library of 4.8E+07 clones.

For round two through four of panning, 3 depletion strategies were employed in parallel. Strategy 1 involved deselecting the library against 100 nM of A*0101 pHLA complex presenting ETDNNIVVY (SEQ ID NO: 2) peptide. For Strategy 2, the library was deselected against a pool of five negative control pHLAs (100 mM each) (Table: 7). For Strategy 3, no deselection step was included.

TABLE 7
List of G2 target and negative control peptides
presented on A*0101 pHLA complex.

| Peptide | Peptide Sequence | SEQ ID NO: |
|---|---|---|
| Target peptide | NTDNNLAVY | 1 |
| Control peptide-1 | YTDNWLAVY | 3 |
| Control peptide-2 | GTDNWLAQY | 4 |
| Control peptide-3 | ETDNNIVVY | 2 |
| Control peptide-4 | PTDENLARY | 5 |
| Control peptide-5 | NTDNLLTEY | 6 |

For the second round of panning, 50 nM of target pHLA complex was incubated with depleted (strategy one and two) or non-depleted (strategy three) library for 1 hour at room temperature with rotation. Dynabead M-280 streptavidin beads (Life Technologies), blocked in 3% milk/PBS for one hour, were added to the mixture and incubated for one additional hour with rotation. Beads were collected using magnetic separator and any non-specific phages were removed by washing the beads five times with PBST followed by five additional washes with PBS. To elute the bound phages from the washed beads, 1 mL 0.1M TEA was added and incubated for 10 minutes at room temperature. The eluted phage was collected from the beads and neutralized with 0.5 mL 1M Tris-HCl pH 7.5. The neutralized phages were then used to infect log growth TG-1 cells (OD₆₀₀=0.5) and after an hour of infection at 37° C., cells were plated onto 2YT media with 100 μg/mL carbenicillin and 2% glucose (2YTCG) agar plates for output titer and bacterial growth for subsequent panning rounds. For subsequent rounds of panning, selection antigen concentrations were lowered while washes increased by amount and length of wash times.

For each of panning Strategies 1-3, the four rounds of panning were carried out as shown in Table 8.

TABLE 8
Phage library screening strategy

| Round | Antigen concentration | Washes |
|---|---|---|
| R1 | 100 nM | 3× PBST + 3× PBS |
| R2 | 50 nM | 5× PBST + 5× PBS |
| R3 | 10 nM | 8× PBST + 8× PBS |
| R4 | 1 nM | 10× PBST + 10× PBS |

Input/Output Phage Titer

Each round of input titer was serially diluted in 2YT media to 1010. Log phase TG-1 cells are infected with diluted phage titers (10⁷-10¹⁰) and incubated at 37° C. for 30 minutes without shaking followed by another 30 minutes with gentle shaking. Infected cells are plated onto 2YTCG plates and incubated overnight at 30° C. Individual colonies were counted to determine input titer. Output titers were performed following 1 h infection of eluted phage into TG-1 cells. 1, 0.1, 0.01, and 0.001 μL of infected cells were plated onto 2YTCG platers and incubated overnight at 30° C. Individual colonies were counted to determine output titer.

Generation of Bacterial Supernatant

At the end of phage panning, individual colonies were picked from titer plates and grown in 96 well plates with 2YT media containing 2% glucose and 100 μg/mL carbenicillin. After an overnight growth at 37° C., plates were centrifuged at 4000×g for 30 minutes. The supernatant was collected to be used in a binding assay by Meso Scale Discovery (MSD) platform.

Meso Scale Discovery

G2 scFv screening was conducted using the Meso Scale Discovery U-PLEX Development Pack, 9-assay (cat. No. K15234N). The pack contains a 10-spot U-PLEX plate with 9 activated spots and 9 unique linkers as well as stop solution and read buffer. Biotinylated pHLA and biotinylated Protein A were each diluted to 33 nM using PBS+0.5% BSA. For each plate, 200 μL of the diluted pHLA or protein L was mixed with 300 μL of the corresponding Linker (See Tables 1) and incubated at room temperature for 30 minutes. For the initial scFv screening, only the target (NTDNNLAVY (SEQ ID NO: 1)), OTLA3 (ETDNNIVVY (SEQ ID NO: 2)) and Protein L were used. Follow-up testing of hits used all of the OTLAs shown in Table 9.

TABLE 9
A*0101 pHLA conjugation to unique
linker for G2 antibody screening

| Linker | Peptide sequence |
|---|---|
| 1 | NTDNNLAVY (SEQ ID NO: 1) |
| 2 | YTDNWLAVY (SEQ ID NO: 3) |
| 3 | GTDNWLAQY (SEQ ID NO: 4) |
| 4 | ETDNNIVVY (SEQ ID NO: 2) |
| 5 | PTDENLARY (SEQ ID NO: 5) |
| 7 | NTDNLLTEY (SEQ ID NO: 6) |
| 10 | Protein L |

Following the 30 minute incubation, 200 μL Stop solution was added to each linker-pHLA solution. They were again incubated for 30 minutes at room temperature. These volumes were scaled based on the number of plates. The linker-pHLA solutions are now a 10× solution. They were then pooled together and further diluted with stop solution to the final 1× concentration. By way of example only, for one plate with one linker, 600 μL pHLA would be diluted with 5.4 mL stop solution for the 1× concentration with a total volume of 6 mL. By way of other example only, for one plate using 8 linkers, 600 μL of each linker was pooled to give 4.8 mL volume and 1.2 mL additional stop solution added for the final 6 mL volume. All volumes were scaled for additional plates. The pooled linker-pHLA solution was then coated onto the 10-spot plate as 50 μL/well, the plate sealed and stored at 4° C. overnight.

Phage Supernatants were serially diluted 10-fold with PBS+1% BSA. The plate was washed 3 times with PBS+0.05% Tween and samples added as 50 μL/well. Plates were incubated at room temperature shaking for 2 hours. The plates were washed as before and 50 μL of 1 μg/mL SulfoTag anti-Myc tag, (Abcam, ab206486) was added to each well. The anti-Myc tag antibody was sulfo-tag labeled using the MSD Gold Sulfo-tag NHS-Ester Conjugation kit (Meso Scale Discovery, R31AA-2) at a challenge ratio of 10. The plates were incubated for 1 hour shaking at room temperature. The plate wash was repeated and 150 μL 2× Read Buffer T (Meso Scale Discovery, R92TC-2) was added to all wells and the plate read immediately on the Quickplex SQ 120.

Those scFvs that showed selectivity for target pHLA compared to the ETD negative control pHLA by MSD as phage supernatants, were selected and re-arrayed to repeat the MSD binding assay against five negative control pHLAs.

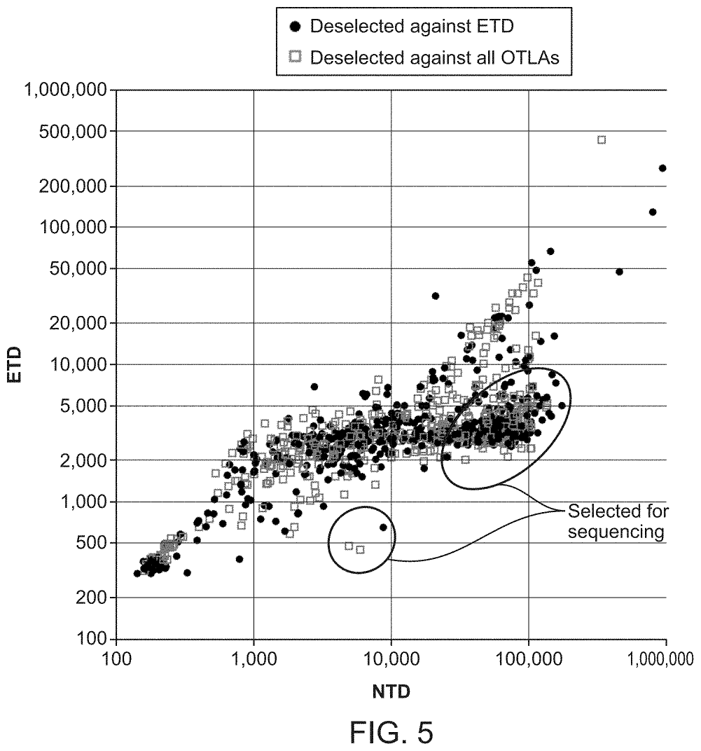

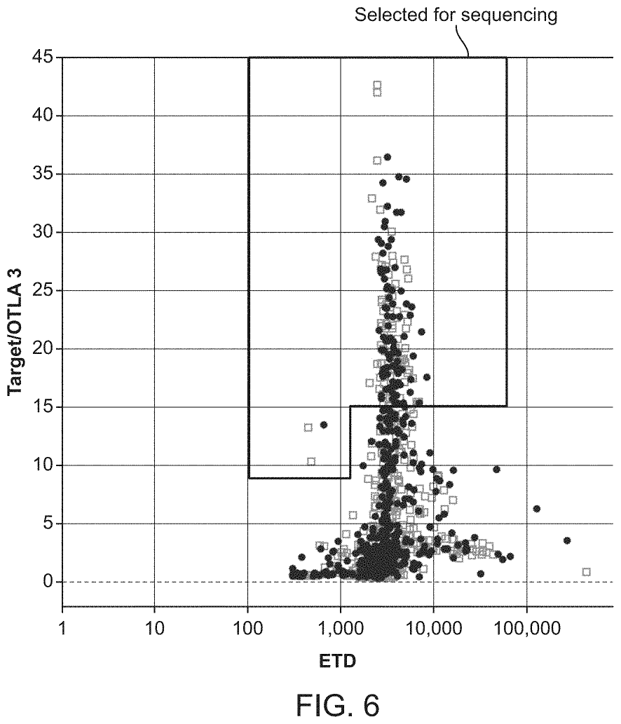

MSD results for each scFv supernatant selected via panning Strategy 1 (deselection against “ETD” peptide only) and panning Strategy 2 (deselection against all five OTLA peptides) are depicted in FIG. 5 and FIG. 6. FIG. 5 shows MSD results plotted as binding intensity for the “ETD” peptide (Y axis) vs. binding intensity for the “NTD” peptide (X axis). FIG. 6 shows MSD results plotted as Target/OTLA intensity ratio (Y axis) vs. ETD binding intensity (X axis). Clones that were selected for sequencing are outlined in FIGS. 5 and 6. These clones were sequenced by the Sanger sequencing and unique scFvs were identified for further downstream applications.

CDR sequences of the sequenced scFv clones are shown in Table 10.

VH and VL sequences of the sequenced scFv clones are shown in Table 11.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

### SEQUENCES

TABLE 10
CDR sequences of identified scFvs to G2, numbered according to the
Kabat numbering scheme

| scFv ID | HCDR1 | HCDR2 | HCDR3 | LCDR1 | LCDR2 | LCDR3 |
|---|---|---|---|---|---|---|
| 1C08 | DYNIH | WINPNS | DKVGLD | RASQGIN | AASSLQ | QQSYLT |
| | (SEQ ID | GGTNYA | Y (SEQ | NWLA | A (SEQ | PYT (SEQ |
| | NO: 8) | QKFQG | ID NO: | (SEQ ID | ID NO: | ID NO: |
| | | (SEQ ID | 10) | NO: 11) | 12) | 13) |
| | | NO: 9) | | | | |
| 1A03 | DYNIN | WINPNS | ANWLDY | RASQDIN | AASTLQ | QQSYGS |
| | (SEQ ID | GGTNYA | (SEQ ID | RWLA | S (SEQ ID | PYT (SEQ |
| | NO: 16) | QKLQG | NO: 18) | (SEQ ID | NO: 20) | ID NO: |
| | | (SEQ ID | | NO: 19) | | 21) |
| | | NO: 17) | | | | |
| 1A07 | DYYLH | WMNPNS | GEWFDP | RASQSIS | AASTLQ | QQSYSTP |
| | (SEQ ID | GGTNYA | (SEQ ID | RWLA | S (SEQ ID | YT (SEQ |
| | NO: 22) | QKFQG | NO: 24) | (SEQ ID | NO: 20) | ID NO: |
| | | (SEQ ID | | NO: 25) | | 26) |
| | | NO: 23) | | | | |
| 1B04 | DYYMH | WIFPNSG | EDWCXP | RASQDIS | AASXLQ | QQSYSSP |
| | (SEQ ID | GTNYAQ | (SEQ ID | RWLA | S (SEQ ID | YT (SEQ |
| | NO: 27) | KFQG | NO: 29) | (SEQ ID | NO: 31) | ID NO: |
| | | (SEQ ID | | NO: 30) | | 32) |
| | | NO: 28) | | | | |
| 1C11 | GKYMH | WMNPNS | GGYYGS | RASQSIS | DASNLE | QQTDSIP |
| | (SEQ ID | GNTGYA | GSWTYF | SYLN | T (SEQ ID | IT (SEQ |
| | NO: 33) | QKFQG | DL (SEQ | (SEQ ID | NO: 37) | ID NO: |
| | | (SEQ ID | ID NO: | NO: 36) | | 38) |
| | | NO: 34) | 35) | | | |
| 1H06 | GYLMH | WINPNS | ESSGWG | RASQGIG | GASSLQS | QQSYITP |
| | (SEQ ID | GGTNYA | N (SEQ | RWLA | (SEQ ID | YT (SEQ |
| | NO: 39) | PKFQG | ID NO: | (SEQ ID | NO: 43) | ID NO: |
| | | (SEQ ID | 41) | NO: 42) | | 44) |
| | | NO: 40) | | | | |
| 1D11 | GYNMH | WINPNT | ANWLDY | RAXQXI | AASTLQ | QQSYSIP |
| | (SEQ ID | GGTSYA | (SEQ ID | XRWLA | S (SEQ ID | IT (SEQ |
| | NO: 45) | QKFQG | NO: 18) | (SEQ ID | NO: 20) | ID NO: |
| | | (SEQ ID | | NO: 47) | | 48) |
| | | NO: 46) | | | | |
| 1G01 | GYNMH | WINPNS | ANWLDY | RASQSIG | AASRLQ | QQSYGV |
| | (SEQ ID | GGTNYA | (SEQ ID | RWLA | S (SEQ ID | PT (SEQ |
| | NO: 45) | QKFQG | NO: 18) | (SEQ ID | NO: 50) | ID NO: |
| | | (SEQ ID | | NO: 49) | | 51) |
| | | NO: 9) | | | | |
| 1H04 | GYNMH | WINPNS | ANWLDY | RASQSIN | AASTLQ | QQSYITP |
| | (SEQ ID | GGTNYA | (SEQ ID | RWLA | S (SEQ ID | YT (SEQ |
| | NO: 45) | QKFQG | NO: 18) | (SEQ ID | NO: 20) | ID NO: |
| | | (SEQ ID | | NO: 52) | | 44) |
| | | NO: 9) | | | | |
| 1G06 | GYQMH | WINPNS | DGGSYL | RASQSIN | AASTLQ | QQSYSIP |
| | (SEQ ID | GGTNYA | DY (SEQ | SWLA | S (SEQ ID | YN (SEQ |
| | NO: 53) | QKFQG | ID NO: | (SEQ ID | NO: 20) | ID NO: |
| | | (SEQ ID | 54) | NO: 55) | | 56) |
| | | NO: 9) | | | | |
| 1B05 | GYYMH | RIFPNSG | GDWFDP | RASQDIS | AASTLQ | QQSYSSP |
| | (SEQ ID | GTDYAQ | (SEQ ID | RWLA | S (SEQ ID | YT (SEQ |
| | NO: 57) | KFQG | NO: 59) | (SEQ ID | NO: 20) | ID NO: |
| | | (SEQ ID | | NO: 30) | | 32) |
| | | NO: 58) | | | | |
| 1A08 | HYGMS | WINPNS | DLGXYD | RASQSIS | AASSLQS | QQSYSY |
| | (SEQ ID | GGTNYA | Y (SEQ | SWLA | (SEQ ID | PT (SEQ |
| | NO: 60) | QKFQG | ID NO: | (SEQ ID | NO: 63) | ID NO: |
| | | (SEQ ID | 61) | NO: 62) | | 64) |
| | | NO: 9) | | | | |
| 1F11 | KYDIA | WIYPNS | ANWLDY | RASQSV | AASTLH | QQSYSTP |
| | (SEQ ID | GGTNYA | (SEQ ID | SSWLA | S (SEQ ID | YT (SEQ |
| | NO: 65) | QKFQG | NO: 18) | (SEQ ID | NO: 68) | ID NO: |
| | | (SEQ ID | | NO: 67) | | 26) |
| | | NO: 66) | | | | |
| 1A04 | NFGIS | WINPNS | ANWLDY | RASQSIS | AASSLQS | QQSYSTP |
| | (SEQ ID | GGTNYA | (SEQ ID | RWLA | (SEQ ID | YT (SEQ |
| | NO: 69) | QKFQG | NO: 18) | (SEQ ID | NO: 63) | ID NO: |
| | | (SEQ ID | | NO: 25) | | 26) |
| | | NO: 9) | | | | |
| 1F08 | NFPMH | WINPNS | GEWLGY | RASQSV | AASSLQS | QQSYSTP |
| | (SEQ ID | GGTNYA | (SEQ ID | SNWLA | (SEQ ID | YT (SEQ |
| | NO: 70) | QKFQG | NO: 71) | (SEQ ID | NO: 63) | ID NO: |
| | | (SEQ ID | | NO: 72) | | 26) |
| | | NO: 9) | | | | |
| 1G09 | NFPMX | WINPNS | GEWLGY | RASQSV | AASSLQS | QQSYSTP |
| | (SEQ ID | GGTNYA | (SEQ ID | SXWLA | (SEQ ID | YT (SEQ |
| | NO: 73) | QNFQG | NO: 71) | (SEQ ID | NO: 63) | ID NO: |
| | | (SEQ ID | | NO: 75) | | 26) |
| | | NO: 74) | | | | |
| 1F02 | NFVVS | WINPNS | SVGWFD | RASESIG | AASTLQ | QQSYISP |
| | (SEQ ID | GGTNYA | P (SEQ ID | RWLA | S (SEQ ID | YT (SEQ |
| | NO: 76) | QKFQG | NO: 77) | (SEQ ID | NO: 20) | ID NO: |
| | | (SEQ ID | | NO: 78) | | 79) |
| | | NO: 9) | | | | |
| 1F04 | NHNMH | WINPNS | ANWLDY | RASQSID | AASSLQS | QQSYSIP |
| | (SEQ ID | GGTNYA | (SEQ ID | RWLA | (SEQ ID | YT (SEQ |
| | NO: 80) | QKFQG | NO: 18) | (SEQ ID | NO: 63) | ID NO: |
| | | (SEQ ID | | NO: 81) | | 82) |
| | | NO: 9) | | | | |
| 1C10 | NSAIH | YISGSGD | DPYYYG | RASQSIS | YASQSIS | QQSYSTP |
| | (SEQ ID | TMFYAD | SGSYFD | SYLN | (SEQ ID | YT (SEQ |
| | NO: 83) | SVKG | Y (SEQ | (SEQ ID | NO: 86) | ID NO: |
| | | (SEQ ID | ID NO: | NO: 36) | | 26) |
| | | NO: 84) | 85) | | | |
| 1E04 | NSVIS | WISVYN | DRIDGM | RASQRIS | DASTLQ | QQSYSTP |
| | (SEQ ID | GETNYA | DV (SEQ | YWVA | S (SEQ ID | LT (SEQ |
| | NO: 87) | QKLQG | ID NO: | (SEQ ID | NO: 91) | ID NO: |
| | | (SEQ ID | 89) | NO: 90) | | 92) |
| | | NO: 88) | | | | |
| 1B07 | NYAFS | WINPNS | GDWLDY | RASQSIS | AASSLQS | QQSYNT |
| | (SEQ ID | GGTNYA | (SEQ ID | SWLA | (SEQ ID | PYT (SEQ |
| | NO: 93) | QKFQG | NO: 94) | (SEQ ID | NO: 63) | ID NO: |
| | | (SEQ ID | | NO: 62) | | 95) |
| | | NO: 9) | | | | |
| 1E01 | NYAIS | WINPNS | ANWLDY | RASQNIN | AASTLQ | QQSYSPP |
| | (SEQ ID | GGTNYA | (SEQ ID | RWLA | S (SEQ ID | YT (SEQ |
| | NO: 96) | QKFQG | NO: 18) | (SEQ ID | NO: 20) | ID NO: |
| | | (SEQ ID | | NO: 97) | | 98) |
| | | NO: 9) | | | | |
| 1F07 | NYDIN | WINPNS | GEWLSV | RASQEIR | AASSLQS | QQSYTT |
| | (SEQ ID | GGTNYA | (SEQ ID | RWLA | (SEQ ID | PYT (SEQ |
| | NO: 99) | QKFQG | NO: 100) | (SEQ ID | NO: 63) | ID NO: |
| | | (SEQ ID | | NO: 101) | | 102) |
| | | NO: 9) | | | | |
| 1A10 | NYGMS | WINPNS | GDFLDV | RASQGIS | GASRLQ | QQSYLPP |
| | (SEQ ID | GGTNYA | (SEQ ID | SWLA | S (SEQ ID | YT (SEQ |
| | NO: 103) | QKFQG | NO: 104) | (SEQ ID | NO: 106) | ID NO: |
| | | (SEQ ID | | NO: 105) | | 107) |
| | | NO: 9) | | | | |
| 1E10 | NYPIT | WINPNS | ANWLDY | RASQSIG | AASSLQS | QQSYSPP |
| | (SEQ ID | GGTNYA | (SEQ ID | TWLA | (SEQ ID | YA (SEQ |
| | NO: 108) | QKFQG | NO: 18) | (SEQ ID | NO: 63) | ID NO: |
| | | (SEQ ID | | NO: 109) | | 110) |
| | | NO: 9) | | | | |
| 1D10 | NYTIS | WINPNS | ANWLDY | RASQSIS | AASSLHS | QQSYSPP |
| | (SEQ ID | GGTNYA | (SEQ ID | SWLA | (SEQ ID | YT (SEQ |
| | NO: 111) | QKFQG | NO: 18) | (SEQ ID | NO: 112) | ID NO: |
| | | (SEQ ID | | NO: 62) | | 98) |
| | | NO: 9) | | | | |
| 1D07 | NYTVT | WINPNS | ANWLDY | RASQSIS | AASTLQ | QQSYITP |
| | (SEQ ID | GGTNYA | (SEQ ID | RWLA | S (SEQ ID | YT (SEQ |
| | NO: 113) | QKLQG | NO: 18) | (SEQ ID | NO: 20) | ID NO: |
| | | (SEQ ID | | NO: 25) | | 44) |
| | | NO: 17) | | | | |
| 1E05 | NYVIS | WINPNS | ANWLDY | RASQSIS | AASTVQ | QQSYSTP |
| | (SEQ ID | GGTISAP | (SEQ ID | RWLA | S (SEQ ID | YT (SEQ |
| | NO: 114) | NFQG | NO: 18) | (SEQ ID | NO: 116) | ID NO: |
| | | (SEQ ID | | NO: 25) | | 26) |
| | | NO: 115) | | | | |
| 1F01 | RHLLH | WINPNS | ANWLDY | RASQNIG | AASSLQS | QQSYSTP |
| | (SEQ ID | GGTNYA | (SEQ ID | SWLA | (SEQ ID | YT (SEQ |
| | NO: 117) | QKFQG | NO: 18) | (SEQ ID | NO: 63) | ID NO: |
| | | (SEQ ID | | NO: 118) | | 26) |
| | | NO: 9) | | | | |
| 1E08 | RYIMH | WINPNS | DTQSGM | RASQTIT | GASTLQ | QQSFSTP |
| | (SEQ ID | GGTNYA | DV (SEQ | RYLN | S (SEQ ID | LT (SEQ |
| | NO: 119) | QKFQG | ID NO: | (SEQ ID | NO: 122) | ID NO: |
| | | (SEQ ID | 120) | NO: 121) | | 123) |
| | | NO: 9) | | | | |
| 1C06 | RYNMH | WINPNS | DGYSGL | RASQSIS | GASSLQS | QQSYSV |
| | (SEQ ID | GGTNYA | DV (SEQ | SWLA | (SEQ ID | PYS (SEQ |
| | NO: 124) | QKFQG | ID NO: | (SEQ ID | NO: 43) | ID NO: |
| | | (SEQ ID | 125) | NO: 62) | | 126) |
| | | NO: 9) | | | | |
| 1H03 | RYVMN | WINPNS | ANWLDY | RASQSID | AASTLQ | QQSYSTP |
| | (SEQ ID | GGTNYA | (SEQ ID | SWLA | S (SEQ ID | YT (SEQ |
| | NO: 127) | QKFQG | NO: 18) | (SEQ ID | NO: 20) | ID NO: |
| | | (SEQ ID | | NO: 128) | | 26) |
| | | NO: 9) | | | | |
| 1H08 | SHLIH | WINPNS | DYGMD | RASQNV | AASSLQS | QQSYITP |
| | (SEQ ID | GGTNYA | V (SEQ | NRWLA | (SEQ ID | YT (SEQ |
| | NO: 129) | QKLQG | ID NO: | (SEQ ID | NO: 63) | ID NO: |
| | | (SEQ ID | 130) | NO: 131) | | 44) |
| | | NO: 17) | | | | |
| 1C04 | SYAIS | WINPNS | ARGMTT | RASQDIG | DASTLQ | QQSYRIP |
| | (SEQ ID | GGTRYA | GYMDV | TWLA | S (SEQ ID | YT (SEQ |
| | NO: 132) | QKFQG | (SEQ ID | (SEQ ID | NO: 91) | ID NO: |
| | | (SEQ ID | NO: 134) | NO: 135) | | 136) |
| | | NO: 133) | | | | |
| 1D02 | SYAIS | WIKPDS | DGNWFD | RASQAIS | AASTLQ | QESYIPP |
| | (SEQ ID | GGTIYA | P (SEQ ID | RWLA | S (SEQ ID | YT (SEQ |
| | NO: 132) | QKFQG | NO: 138) | (SEQ ID | NO: 20) | ID NO: |
| | | (SEQ ID | | NO: 139) | | 140) |
| | | NO: 137) | | | | |
| 1D05 | SYAIS | WINPNS | ANWLDY | RASQSIR | AASSLQS | QQSYSTP |
| | (SEQ ID | GGTNYA | (SEQ ID | RWLA | (SEQ ID | YT (SEQ |
| | NO: 132) | QKFQG | NO: 18) | (SEQ ID | NO: 63) | ID NO: |
| | | (SEQ ID | | NO: 141) | | 26) |
| | | NO: 9 | | | | |
| 1E11 | SYAIS | WINPNS | DEWLQH | RASQSIS | AASTLQ | QQSYSTP |
| | (SEQ ID | GGTNYA | (SEQ ID | RWLA | S (SEQ ID | YT (SEQ |
| | NO: 132) | QKFQG | NO: 142) | (SEQ ID | NO: 20) | ID NO: |
| | | (SEQ ID | | NO: 25) | | 26) |
| | | NO: 9) | | | | |
| 1F05 | SYAIS | WINPNS | ANWLDY | RASQSIS | AASRLH | QQSYSPP |
| | (SEQ ID | GGTNYA | (SEQ ID | RWLA | S (SEQ ID | YT (SEQ |
| | NO: 132) | QKFQG | NO: 18) | (SEQ ID | NO: 143) | ID NO: |
| | | (SEQ ID | | NO: 25) | | 98) |
| | | NO: 9) | | | | |
| 1G11 | SYAIS | WISPNSG | ANWLDY | RASQSIG | AASSLQS | QQSYTSP |
| | (SEQ ID | GTNYAQ | (SEQ ID | RWLA | (SEQ ID | YR (SEQ |
| | NO: 132) | KFQG | NO: 18) | (SEQ ID | NO: 63) | ID NO: |
| | | (SEQ ID | | NO: 49) | | 145) |
| | | NO: 144) | | | | |
| 1A02 | SYDIN | RINPYND | DTSGDG | RASQSV | AASSLSN | QHNYIRP |
| | (SEQ ID | ETHYAQ | RFHP | SRYLN | (SEQ ID | PT (SEQ |
| | NO: 146) | KFQG | (SEQ ID | (SEQ ID | NO: 150) | ID NO: |
| | | (SEQ ID | NO: 148) | NO: 149) | | 151) |
| | | NO: 147) | | | | |
| 1A06 | SYEIH | WINPNS | EGGTKL | RASQGIS | GASSLQS | QQSYIIP |
| | (SEQ ID | GGTNYA | DV (SEQ | NYLA | (SEQ ID | LT (SEQ |
| | NO: 152) | QKFQG | ID NO: | (SEQ ID | NO: 43) | ID NO: |
| | | (SEQ ID | 153) | NO: 154) | | 155) |
| | | NO: 9) | | | | |
| 1C05 | SYGIS | WINPNS | GSGWTD | RASQSV | AASNLQ | QQSYSTP |
| | (SEQ ID | GGTNYA | N (SEQ | DRWLA | S (SEQ ID | YT (SEQ |
| | NO: 156) | QKFQG | ID NO: | (SEQ ID | NO: 159) | ID NO: |
| | | (SEQ ID | 157) | NO: 158) | | 26) |
| | | NO: 9) | | | | |
| 1D04 | SYGIS | WINPNS | EMGELD | RASQSIS | AASSLQS | QQSYSTP |
| | (SEQ ID | GGTNYA | Y (SEQ | TWLA | (SEQ ID | YT (SEQ |
| | NO: 156) | RKFQG | ID NO: | (SEQ ID | NO: 63) | ID NO: |
| | | (SEQ ID | 161) | NO: 162) | | 26) |
| | | NO: 160) | | | | |
| 1E03 | SYGIT | WINPNS | ANWLDY | RASQSIN | AASTLQ | QQSYTT |
| | (SEQ ID | GGTNYA | (SEQ ID | RWLA | S (SEQ ID | PYT (SEQ |
| | NO: 163) | QKFQG | NO: 18) | (SEQ ID | NO: 20) | ID NO: |
| | | (SEQ ID | | NO: 52) | | 102) |
| | | NO: 9) | | | | |
| 1A11 | SYGMH | VISYDGS | TYSYGN | RASESIS | AASSLQS | QQSYSTP |
| | (SEQ ID | NQYYAD | YYYYG | TYLN | (SEQ ID | IT (SEQ |
| | NO: 164) | SVKG | MDV | (SEQ ID | NO: 63) | ID NO: |
| | | (SEQ ID | (SEQ ID | NO: 167) | | 168) |
| | | NO: 165) | NO: 166) | | | |
| 1E09 | SYNMH | WINPNS | EGAVAV | RASQSIS | AASTLQ | QQGYST |
| | (SEQ ID | GGTNYA | DY (SEQ | RWLA | S (SEQ ID | PYT (SEQ |
| | NO: 169) | QKFQG | ID NO: | (SEQ ID | NO: 20) | ID NO: |
| | | (SEQ ID | 170) | NO: 25) | | 171) |
| | | NO: 9) | | | | |
| 1C01 | SYTLS | WINPNS | DGGGGL | RASQSV | AASSLQS | QQSYSTP |
| | (SEQ ID | GGTNYA | DY (SEQ | GRWLA | (SEQ ID | YT (SEQ |
| | NO: 172) | QKFQG | ID NO: | (SEQ ID | NO: 63) | ID NO: |
| | | (SEQ ID | 173) | NO: 174) | | 26) |
| | | NO: 9) | | | | |
| 1D09 | SYVIN | WINPNS | ERGGAY | RASQSIG | AASSLQS | QQTYSPP |
| | (SEQ ID | GGTNYA | DY (SEQ | SWLA | (SEQ ID | YT (SEQ |
| | NO: 175) | QKLQG | ID NO: | (SEQ ID | NO: 63) | ID NO: |
| | | (SEQ ID | 176) | NO: 177) | | 178) |
| | | NO: 17) | | | | |
| 1B03 | SYVIS | WISPDSG | DSYGMD | RASQSIS | AASSLQS | QQSYSPP |
| | (SEQ ID | GTKYAQ | V (SEQ | SWLA | (SEQ ID | YT (SEQ |
| | NO: 179) | KFQG | ID NO: | (SEQ ID | NO: 63) | ID NO: |
| | | (SEQ ID | 181) | NO: 62) | | 98) |
| | | NO: 180) | | | | |
| 1H02 | SYYMH | WINPNS | GEWLDY | RASQTIS | AASSLQS | QQSYSTP |
| | (SEQ ID | GGTNYA | (SEQ ID | SWLA | (SEQ ID | YT (SEQ |
| | NO: 182) | QKFQG | NO: 183) | (SEQ ID | NO: 63) | ID NO: |
| | | (SEQ ID | | NO: 184) | | 26) |
| | | NO: 9) | | | | |
| 1D03 | THTIT | WINPDS | EGYSGL | RASQSIS | GASSLHS | QQSYSV |
| | (SEQ ID | GGTNYA | DY (SEQ | NWLA | (SEQ ID | PYT (SEQ |
| | NO: 185) | QKFQG | ID NO: | (SEQ ID | NO: 189) | ID NO: |
| | | (SEQ ID | 187) | NO: 188) | | 190) |
| | | NO: 186) | | | | |
| 1B02 | TYLIH | WINPNS | ERASGY | RASQSIS | GASVLQ | QQSYHV |
| | (SEQ ID | GGTNYA | DI (SEQ | SWLA | S (SEQ ID | PYT (SEQ |
| | NO: 191) | QKFQG | ID NO: | (SEQ ID | NO: 193) | ID NO: |
| | | (SEQ ID | 192) | NO: 62) | | 194) |
| | | NO: 9) | | | | |
| 1F03 | TYPLS | WINPNS | ANWLDY | RASQRIG | AASTLQ | QQSYSA |
| | (SEQ ID | GGTNYA | (SEQ ID | SWLA | S (SEQ ID | PYS (SEQ |
| | NO: 195) | QKFQG | NO: 18) | (SEQ ID | NO: 20) | ID NO: |
| | | (SEQ ID | | NO: 196) | | 197) |
| | | NO: 9) | | | | |
| 1C09 | TYPMX | WINPNS | ANWLDY | RASQGID | AASSLQS | QQSYSPP |
| | (SEQ ID | GGTNYA | (SEQ ID | RWLA | (SEQ ID | YT (SEQ |
| | NO: 198 | QKFQG | NO: 18) | (SEQ ID | NO: 63) | ID NO: |
| | | (SEQ ID | | NO: 199) | | 98) |
| | | NO: 9 | | | | |
| 1C07 | TYYMH | WINPNS | GEWLDY | RASQGIS | AASKLQ | QQSYTT |
| | (SEQ ID | GGTNYA | (SEQ ID | SWLA | G (SEQ | PYT (SEQ |
| | NO: 200) | QKFQG | NO: 183) | (SEQ ID | ID NO: | ID NO: |
| | | (SEQ ID | | NO: 105) | 201) | 102) |
| | | NO: 9) | | | | |
| 2G03 | AYHVH | WINPNS | ANWLDY | RASQNIN | AASTLQ | QQSYSPP |
| | (SEQ ID | GGTNYA | (SEQ ID | RWLA | S (SEQ ID | YT (SEQ |
| | NO: 202) | QKFQG | NO: 18) | (SEQ ID | NO: 20) | ID NO: |
| | | (SEQ ID | | NO: 97) | | 98) |
| | | NO: 9) | | | | |
| 2H05 | AYNMH | WINPNS | ANWLDY | RASQSIS | AASSLQS | QQSYSTP |
| | (SEQ ID | GGTNYA | (SEQ ID | RWLA | (SEQ ID | YT (SEQ |
| | NO: 203) | QKFQG | NO: 18) | (SEQ ID | NO: 63) | ID NO: |
| | | (SEQ ID | | NO: 25) | | 26) |
| | | NO: 9) | | | | |
| 2C03 | DYGLS | WINPNS | ANWLDY | RASQTIG | AASTLQ | QQSYSTP |
| | (SEQ ID | GGTNYA | (SEQ ID | PWLA | S (SEQ ID | YT (SEQ |
| | NO: 204) | QKFQG | NO: 18) | (SEQ ID | NO: 20) | ID NO: |
| | | (SEQ ID | | NO: 205) | | 26) |
| | | NO: 9) | | | | |
| 2F05 | DYGLS | WINPNS | ANWLDY | RASQTIG | AASTLQ | QQSYSTP |
| | (SEQ ID | GGTNYA | (SEQ ID | PWLA | S (SEQ ID | YT (SEQ |
| | NO: 204) | QKFQG | NO: 18) | (SEQ ID | NO: 20) | ID NO: |
| | | (SEQ ID | | NO: 205) | | 26) |
| | | NO: 9) | | | | |
| 2F04 | DYHLH | WINPNS | GEWLDY | RASQSIS | AASSLQS | QQSYSTP |
| | (SEQ ID | GGTNFA | (SEQ ID | SWLA | (SEQ ID | YT (SEQ |
| | NO: 206) | QKFQG | NO: 183) | (SEQ ID | NO: 63) | ID NO: |
| | | (SEQ ID | | NO: 62) | | 26) |
| | | NO: 207) | | | | |
| 2G04 | DYLMH | WINPNS | XNXXDY | RASQSIN | AASKLE | QQSYNT |
| | (SEQ ID | GGTNYA | | RWLA | T (SEQ ID | PYT (SEQ |
| | NO: 208) | QKXQG | | (SEQ ID | NO: 211) | ID NO: |
| | | (SEQ ID | | NO: 52) | | 95) |
| | | NO: 209) | | | | |
| 2C01 | DYNIH | WINPNS | ANWLDY | RASQSIS | AASTLQ | QQSYSTP |
| | (SEQ ID | GGTNYA | (SEQ ID | RWLA | T (SEQ ID | YT (SEQ |
| | NO: 8) | QKFQG | NO: 18) | (SEQ ID | NO: 212) | ID NO: |
| | | (SEQ ID | | NO: 25) | | 26) |
| | | NO: 9) | | | | |
| 2E01 | DYQIH | WISPYSG | DRVGLD | RASQSIS | AASSLQS | QQSYSIP |
| | (SEQ ID | GTNYAQ | Y (SEQ | TWLA | (SEQ ID | YT (SEQ |
| | NO: 213) | KFQG | ID NO: | (SEQ ID | NO: 63) | ID NO: |
| | | (SEQ ID | 215) | NO: 162) | | 82) |
| | | NO: 214) | | | | |
| 2A10 | DYTIH | WINPNS | ANWLDY | RASQNIR | AASSLRS | QQSYSTP |
| | (SEQ ID | GGTNYA | (SEQ ID | RWLA | (SEQ ID | YT (SEQ |
| | NO: 216) | QQFQG | NO: 18) | (SEQ ID | NO: 219) | ID NO: |
| | | (SEQ ID | | NO: 218) | | 26) |
| | | NO: 217) | | | | |
| 2G06 | ENEMH | WINPNS | ANWLDF | RASQEIR | AASSLQS | QQSYSPP |
| | (SEQ ID | GGTSYA | (SEQ ID | RWLA | (SEQ ID | YT (SEQ |
| | NO: 220) | PNFQG | NO: 222) | (SEQ ID | NO: 63) | ID NO: |
| | | (SEQ ID | | NO: 101) | | 98) |
| | | NO: 221) | | | | |
| 2H07 | FSTIH | WISPHSG | DSGSYL | QASQDI | AASSLQS | QQSYSTP |
| | (SEQ ID | GTNYAQ | DY (SEQ | NNYLN | (SEQ ID | YT (SEQ |
| | NO: 223) | EFQG | ID NO: | (SEQ ID | NO: 63) | ID NO: |
| | | (SEQ ID | 225) | NO: 226) | | 26) |
| | | NO: 224) | | | | |
| 2G07 | GYAIS | WINPNS | GDWFDP | RASQEIR | AASSLQS | QQSYSTP |
| | (SEQ ID | GGTNYA | (SEQ ID | RWLA | (SEQ ID | YT (SEQ |
| | NO: 227) | QKFQG | NO: 59) | (SEQ ID | NO: 63) | ID NO: |
| | | (SEQ ID | | NO: 101) | | 26) |
| | | NO: 9) | | | | |
| 2B01 | GYNMH | WINPNS | ANWLDY | RASQSIN | AASTLQ | QQSYITP |
| | (SEQ ID | GGTNYA | (SEQ ID | RWLA | S (SEQ ID | YT (SEQ |
| | NO: 45) | QKFQG | NO: 18) | (SEQ ID | NO: 20) | ID NO: |
| | | (SEQ ID | | NO: 52) | | 44) |
| | | NO: 9) | | | | |
| 2B10 | GYNMH | WINPNS | ANWLDY | RASQSIN | AASTLQ | QQSYITP |
| | (SEQ ID | GGTNYA | (SEQ ID | RWLA | S (SEQ ID | YT (SEQ |
| | NO: 45) | QKFQG | NO: 18) | (SEQ ID | NO: 20) | ID NO: |
| | | (SEQ ID | | NO: 52) | | 44) |
| | | NO: 9) | | | | |
| 2D11 | GYNMH | WINPNS | ANWLDY | RASQSIN | AASTLQ | QQSYITP |
| | (SEQ ID | GGTNYA | (SEQ ID | RWLA | S (SEQ ID | YT (SEQ |
| | NO: 45) | QKFQG | NO: 18) | (SEQ ID | NO: 20) | ID NO: |
| | | (SEQ ID | | NO: 52) | | 44) |
| | | NO: 9) | | | | |
| 2G02 | GYNMH | WINPNS | ANWLDY | RASQSIN | AASTLQ | QQSYITP |
| | (SEQ ID | GGTNYA | (SEQ ID | RWLA | S (SEQ ID | YT (SEQ |
| | NO: 45) | QKFQG | NO: 18) | (SEQ ID | NO: 20) | ID NO: |
| | | (SEQ ID | | NO: 52) | | 44) |
| | | NO: 9) | | | | |
| 2H08 | GYNMH | WINPNS | ANWLDY | RASQSIN | AASTLQ | QQSYITP |
| | (SEQ ID | GGTNYA | (SEQ ID | RWLA | S (SEQ ID | YT (SEQ |
| | NO: 45) | QKFQG | NO: 18) | (SEQ ID | NO: 20) | ID NO: |
| | | (SEQ ID | | NO: 52) | | 44) |
| | | NO: 9) | | | | |
| 2G01 | GYYLH | WINPKS | ERGATG | RASQRV | KASILES | QQTYST |
| | (SEQ ID | GGTNFA | YYYGM | DSWLA | (SEQ ID | PWT |
| | NO: 228) | QKFQG | DV (SEQ | (SEQ ID | NO: 232) | (SEQ ID |
| | | (SEQ ID | ID NO: | NO: 231) | | NO: 233) |
| | | NO: 229) | 230) | | | |
| 2E06 | GYYMH | WIYPNS | DDFDY | QASQDIS | VASTLQ | QQSYNT |
| | (SEQ ID | GGTNYA | (SEQ ID | NYLN | S (SEQ ID | PYT (SEQ |
| | NO: 57) | QNLQG | NO: 235) | (SEQ ID | NO: 237) | ID NO: |
| | | (SEQ ID | | NO: 236) | | 95) |
| | | NO: 234) | | | | |
| 2B02 | KYDIA | WIYPNS | ANWLDY | RASQSV | AASTLH | QQSYSTP |
| | (SEQ ID | GGTNYA | (SEQ ID | SSWLA | S (SEQ ID | YT (SEQ |
| | NO: 65) | QKFQG | NO: 18) | (SEQ ID | NO: 68) | ID NO: |
| | | (SEQ ID | | NO: 67) | | 26) |
| | | NO: 66) | | | | |
| 2D04 | KYGIS | WINPYS | GDGWY | RASQSIS | AASSLQS | QQSYSPP |
| | (SEQ ID | GGTNYA | DP (SEQ | NWLA | (SEQ ID | YT (SEQ |
| | NO: 238) | QNFQG | ID NO: | (SEQ ID | NO: 63) | ID NO: |
| | | (SEQ ID | 240) | NO: 188) | | 98) |
| | | NO: 239) | | | | |
| 2A07 | NFLIN | WINPNS | ERELPFD | RASQSIS | GASNLE | QQTYNP |
| | (SEQ ID | GGTNYA | I (SEQ ID | SWVA | S (SEQ ID | PYS (SEQ |
| | NO: 241) | QKFQG | NO: 242) | (SEQ ID | NO: 244) | ID NO: |
| | | (SEQ ID | | NO: 243) | | 245) |
| | | NO: 9) | | | | |
| 2H04 | NFPMH | WINPNS | GEWLGY | RASQSV | AASSLQS | QQSYSTP |
| | (SEQ ID | GGTNYA | (SEQ ID | SNWLA | (SEQ ID | YT (SEQ |
| | NO: 70) | QKFQG | NO: 71) | (SEQ ID | NO: 63) | ID NO: |
| | | (SEQ ID | | NO: 72) | | 26) |
| | | NO: 9) | | | | |
| 2B11 | NHIMH | WIYPDS | ATGFMD | RASQSIS | AASSLQS | QQSYNT |
| | (SEQ ID | GGTKYA | V (SEQ | NWLA | (SEQ ID | PFT (SEQ |
| | NO: 246) | QKFQG | ID NO: | (SEQ ID | NO: 63) | ID NO: |
| | | (SEQ ID | 248) | NO: 188) | | 249) |
| | | NO: 247) | | | | |
| 2F11 | NHNMH | WINPNS | ANWLDY | RASQSID | AASSLQS | QQSYSIP |
| | (SEQ ID | GGTNYA | (SEQ ID | RWLA | (SEQ ID | YT (SEQ |
| | NO: 80) | QKFQG | NO: 18) | (SEQ ID | NO: 63) | ID NO: |
| | | (SEQ ID | | NO: 81) | | 82) |
| | | NO: 9) | | | | |
| 2D08 | NYAFN | WIYPNT | ANWLDY | RASQSID | AASTLQ | QQSYITP |
| | (SEQ ID | GGTHYA | (SEQ ID | RWLA | S (SEQ ID | YT (SEQ |
| | NO: 250) | PKFQG | NO: 18) | (SEQ ID | NO: 20) | ID NO: |
| | | (SEQ ID | | NO: 81) | | 44) |
| | | NO: 251) | | | | |
| 2F03 | NYAFS | WIYPNS | GESWFD | RASQSIS | AASNLE | QQSYSTP |
| | (SEQ ID | GGTNYA | P (SEQ ID | RWLA | S (SEQ ID | YT (SEQ |
| | NO: 93) | QKFQG | NO: 252) | (SEQ ID | NO: 253) | ID NO: |
| | | (SEQ ID | | NO: 25) | | 26) |
| | | NO: 66) | | | | |
| 2A05 | NYAIS | WINPNT | SSGWYD | RASQEIR | AASSLQS | QQSYSPP |
| | (SEQ ID | GGTNYA | Y (SEQ | RWLA | (SEQ ID | YS (SEQ |
| | NO: 96) | QSLQG | ID NO: | (SEQ ID | NO: 63) | ID NO: |
| | | (SEQ ID | 255) | NO: 101) | | 256) |
| | | NO: 254) | | | | |
| 2D07 | NYGIS | WISPDSG | ANWLDY | RASQSIS | AASSLQS | QQSYSTP |
| | (SEQ ID | GTIYAQ | (SEQ ID | RWLA | (SEQ ID | YT (SEQ |
| | NO: 257) | RFQG | NO: 18) | (SEQ ID | NO: 63) | ID NO: |
| | | (SEQ ID | | NO: 25) | | 26) |
| | | NO: 258) | | | | |
| 2E09 | NYGIS | WINPYS | ANWLDY | RASQSV | AASSLQS | QQSYSIP |
| | (SEQ ID | GGTNYA | (SEQ ID | GRWLA | (SEQ ID | YS (SEQ |
| | NO: 257) | QKFQG | NO: 18) | (SEQ ID | NO: 63) | ID NO: |
| | | (SEQ ID | | NO: 174) | | 260) |
| | | NO: 259) | | | | |
| 2G09 | NYHMH | WIYPNS | GAWFDP | RASQSIN | GASNLQ | QQSYSTP |
| | (SEQ ID | GGTNYA | (SEQ ID | SWLA | S (SEQ ID | YT (SEQ |
| | NO: 261) | QKFQG | NO: 262) | (SEQ ID | NO: 263) | ID NO: |
| | | (SEQ ID | | NO: 55) | | 26) |
| | | NO: 66) | | | | |
| 2A04 | NYNIA | WINPNS | ANWLDY | RASQGIS | AASTLQ | QQSYSTP |
| | (SEQ ID | GGTNYA | (SEQ ID | SWLA | S (SEQ ID | YT (SEQ |
| | NO: 264) | QKFQG | NO: 18) | (SEQ ID | NO: 20) | ID NO: |
| | | (SEQ ID | | NO: 105) | | 26) |
| | | NO: 9) | | | | |
| 2H03 | NYPIH | WINPNS | DQGAG | RASQEIR | AASNLQ | QQSYSTP |
| | (SEQ ID | GGTNYA | WDY | RWLA | S (SEQ ID | YT (SEQ |
| | NO: 265) | QKLQG | (SEQ ID | (SEQ ID | NO: 159) | ID NO: |
| | | (SEQ ID | NO: 266) | NO: 101) | | 26) |
| | | NO: 17) | | | | |
| 2E04 | NYQIH | WINPNS | ANWLDY | RASQSIS | AASTVQ | QQSYSTP |
| | (SEQ ID | GGTISAP | (SEQ ID | RWLA | S (SEQ ID | YT (SEQ |
| | NO: 267) | NFQG | NO: 18) | (SEQ ID | NO: 116) | ID NO: |
| | | (SEQ ID | | NO: 25) | | 26) |
| | | NO: 115) | | | | |
| 2A09 | NYVIS | WINPNS | GEWFDP | RASQSIN | GASSLQS | QQSYSTP |
| | (SEQ ID | GGTIYA | (SEQ ID | SWLA | (SEQ ID | YT (SEQ |
| | NO: 114) | QKFQG | NO: 24) | (SEQ ID | NO: 43) | ID NO: |
| | | (SEQ ID | | NO: 55) | | 26) |
| | | NO: 268) | | | | |
| 2D09 | NYVIS | WIYPNT | DRGYGM | RASQDIS | AASSLQS | QQSYSS |
| | (SEQ ID | GGTNYA | DV (SEQ | TWLA | (SEQ ID | WT (SEQ |
| | NO: 114) | QKFQG | ID NO: | (SEQ ID | NO: 63) | ID NO: |
| | | (SEQ ID | 270) | NO: 271) | | 272) |
| | | NO: 269) | | | | |
| 2B08 | PYTFS | WINPSSG | GEWLDY | RASQSIR | AASSLQS | QQSYITP |
| | (SEQ ID | GAQYAQ | (SEQ ID | SWLA | (SEQ ID | YT (SEQ |
| | NO: 273) | KLQG | NO: 183) | (SEQ ID | NO: 63) | ID NO: |
| | | (SEQ ID | | NO: 275) | | 44) |
| | | NO: 274) | | | | |
| 2G11 | RHPMH | WIHPDS | ANWLDY | RASQSID | AASTLQ | QQSYSTP |
| | (SEQ ID | GGTNYS | (SEQ ID | RWLA | S (SEQ ID | YS (SEQ |
| | NO: 276) | QKFQG | NO: 18) | (SEQ ID | NO: 20) | ID NO: |
| | | (SEQ ID | | NO: 81) | | 278) |
| | | NO: 277) | | | | |
| 2C08 | SHGIT | WINPNS | GPWLDY | RASQSIS | AASSLQS | QQSYSTP |
| | (SEQ ID | GGTNYA | (SEQ ID | RWLA | (SEQ ID | YT (SEQ |
| | NO: 279) | QNVQG | NO: 281) | (SEQ ID | NO: 63) | ID NO: |
| | | (SEQ ID | | NO: 25) | | 26) |
| | | NO: 280) | | | | |
| 2C05 | SHLIH | WINPNS | GIFEDY | RASQGIG | ATSRLQS | QQSYGT |
| | (SEQ ID | GGTNYA | (SEQ ID | RWLA | (SEQ ID | PYT (SEQ |
| | NO: 129) | QKFQG | NO: 282) | (SEQ ID | NO: 283) | ID NO: |
| | | (SEQ ID | | NO: 42) | | 284) |
| | | NO: 9) | | | | |
| 2C07 | SHLIH | WINPNS | ANWLDY | RASQSID | AASRLQ | QQSYRT |
| | (SEQ ID | GGTNYA | (SEQ ID | RWLT | S (SEQ ID | PYT (SEQ |
| | NO: 129) | QKFQG | NO: 18) | (SEQ ID | NO: 50) | ID NO: |
| | | (SEQ ID | | NO: 285) | | 286) |
| | | NO: 9) | | | | |
| 2F01 | SHLIH | WINPNS | GDWYD | RASQSIG | AASTLQ | QQSYSSP |
| | (SEQ ID | GGTKYA | Y (SEQ | RWLA | S (SEQ ID | YT (SEQ |
| | NO: 129) | PKFQG | ID NO: | (SEQ ID | NO: 20) | ID NO: |
| | | (SEQ ID | 288) | NO: 49) | | 32) |
| | | NO: 287) | | | | |
| 2H01 | SHLIH | WINPNS | ANWLDY | RASQSIS | AASSLQS | QQSYSA |
| | (SEQ ID | GGTNYA | (SEQ ID | RWLA | (SEQ ID | PLT (SEQ |
| | NO: 129) | QSFQG | NO: 18) | (SEQ ID | NO: 63) | ID NO: |
| | | (SEQ ID | | NO: 25) | | 290) |
| | | NO: 289) | | | | |
| 2B07 | SYAIS | WINPNS | ANWLDY | RASQNIN | AASTLQ | QQSYSPP |
| | (SEQ ID | GGTNYA | (SEQ ID | RWLA | S (SEQ ID | YT (SEQ |
| | NO: 132) | QKFQG | NO: 18) | (SEQ ID | NO: 20) | ID NO: |
| | | (SEQ ID | | NO: 97) | | 98) |
| | | NO: 9) | | | | |
| 2C06 | SYAIS | WINPNS | ANWLDY | RASQSIS | AASRLH | QQSYSPP |
| | (SEQ ID | GGTNYA | (SEQ ID | RWLA | S (SEQ ID | YT (SEQ |
| | NO: 132) | QKFQG | NO: 18) | (SEQ ID | NO: 143) | ID NO: |
| | | (SEQ ID | | NO: 25) | | 98) |
| | | NO: 9) | | | | |
| 2A11 | SYAMH | GISGNG | DAYYYG | RASQSIS | AASTLQ | QQSYTT |
| | (SEQ ID | GSTNYA | SGSYFD | SYLN | T (SEQ ID | PYT (SEQ |
| | NO: 291) | DSVKG | Y (SEQ | (SEQ ID | NO: 212) | ID NO: |
| | | (SEQ ID | ID NO: | NO: 36) | | 102) |
| | | NO: 292) | 293) | | | |
| 2E10 | SYDIN | WINPNS | GEWLDY | RASQSIR | AASSLQS | QQSYSTP |
| | (SEQ ID | GGTNYA | (SEQ ID | SWLA | (SEQ ID | YT (SEQ |
| | NO: 146) | QKFQG | NO: 183) | (SEQ ID | NO: 63) | ID NO: |
| | | (SEQ ID | | NO: 275) | | 26) |
| | | NO: 9) | | | | |
| 2A06 | SYGIS | WINPNS | DYSWFD | RASQDIS | GASTLQ | QQSYSTP |
| | (SEQ ID | GGTNYA | P (SEQ ID | NWLA | S (SEQ ID | YT (SEQ |
| | NO: 156) | QKLQG | NO: 294) | (SEQ ID | NO: 122) | ID NO: |
| | | (SEQ ID | | NO: 295) | | 26) |
| | | NO: 17) | | | | |
| 2C04 | SYGIS | WINPNS | ANWLDY | RASQSIG | ATSSLQS | QQSYSTP |
| | (SEQ ID | GGTNYA | (SEQ ID | RWLA | (SEQ ID | YT (SEQ |
| | NO: 156) | QKFQG | NO: 18) | (SEQ ID | NO: 296) | ID NO: |
| | | (SEQ ID | | NO: 49) | | 26) |
| | | NO: 9) | | | | |
| 2D02 | SYGIS | WINPNS | DFSWFD | RASQSIS | GASNLQ | QQSYSTP |
| | (SEQ ID | GGTNYA | P (SEQ ID | NWLA | P (SEQ ID | YS (SEQ |
| | NO: 156) | QRLQG | NO: 298) | (SEQ ID | NO: 299) | ID NO: |
| | | (SEQ ID | | NO: 188) | | 278) |
| | | NO: 297) | | | | |
| 2E05 | SYGIS | WINPNS | GYNWFD | RASQSIS | AASNLQ | QQSYNT |
| | (SEQ ID | GGTNYA | P (SEQ ID | NWLA | S (SEQ ID | PFT (SEQ |
| | NO: 156) | QKFQG | NO: 300) | (SEQ ID | NO: 159) | ID NO: |
| | | (SEQ ID | | NO: 188) | | 249 |
| | | NO: 9) | | | | |
| 2F02 | SYGIS | WINPNS | ANWLDY | RASQDIS | AASSLQS | QQSYNT |
| | (SEQ ID | GGTNYA | (SEQ ID | RWLA | (SEQ ID | PYT (SEQ |
| | NO: 156) | QKFQG | NO: 18) | (SEQ ID | NO: 63) | ID NO: |
| | | (SEQ ID | | NO: 30) | | 95) |
| | | NO: 9) | | | | |
| 2F07 | SYGIS | WINPNS | SINWFDP | RASQSV | AASTLQ | QQSYSPP |
| | (SEQ ID | GGTNYA | (SEQ ID | GRWMA | T (SEQ ID | YT (SEQ |
| | NO: 156) | QKFQG | NO: 301) | (SEQ ID | NO: 212) | ID NO: |
| | | (SEQ ID | | NO: 302) | | 98) |
| | | NO: 9) | | | | |
| 2G10 | SYGIS | WINPHS | ANWLDY | RASQSIS | AASRLQ | QQSYSPP |
| | (SEQ ID | GGTFYA | (SEQ ID | SWLA | S (SEQ ID | YT (SEQ |
| | NO: 156) | QKFQG | NO: 18) | (SEQ ID | NO: 50) | ID NO: |
| | | (SEQ ID | | NO: 62) | | 98) |
| | | NO: 303) | | | | |
| 2E03 | SYLMH | WINPYS | DRGSGY | RASQTIG | GASSLQS | QQSYSPP |
| | (SEQ ID | GGTNYA | DY (SEQ | NWLA | (SEQ ID | LT (SEQ |
| | NO: 304) | QKFQG | ID NO: | (SEQ ID | NO: 43) | ID NO: |
| | | (SEQ ID | 305) | NO: 306) | | 307) |
| | | NO: 259) | | | | |
| 2H06 | SYNMH | WINPNS | ANWLDY | RASQSIS | AASTLQ | QQSYITP |
| | (SEQ ID | GGTNYA | (SEQ ID | RWLA | S (SEQ ID | YT (SEQ |
| | NO: 169) | QKLQG | NO: 18) | (SEQ ID | NO: 20) | ID NO: |
| | | (SEQ ID | | NO: 25) | | 44) |
| | | NO: 17) | | | | |
| 2F09 | SYPIH | WINPNS | ANWLDY | RASQSIS | AASNLQ | QQSYSPP |
| | (SEQ ID | GGTNYA | (SEQ ID | RWLA | S (SEQ ID | YT (SEQ |
| | NO: 308) | QKFQG | NO: 18) | (SEQ ID | NO: 159) | ID NO: |
| | | (SEQ ID | | NO: 25) | | 98) |
| | | NO: 9) | | | | |
| 2E08 | SYPIS | WINPSN | SNWLDY | RASQSIG | AASTLQ | QQSYSTP |
| | (SEQ ID | GGTNYA | (SEQ ID | RWLA | S (SEQ ID | YT (SEQ |
| | NO: 309) | QKFQG | NO: 311) | (SEQ ID | NO: 20) | ID NO: |
| | | (SEQ ID | | NO: 49) | | 26) |
| | | NO: 310) | | | | |
| 2F08 | SYTIS | WISPNSG | ANWLDY | RTSQGIG | AASSLQS | QQSYSTP |
| | (SEQ ID | GTNYAQ | (SEQ ID | TWLA | (SEQ ID | YT (SEQ |
| | NO: 312) | KFQG | NO: 18) | (SEQ ID | NO: 63) | ID NO: |
| | | (SEQ ID | | NO: 313) | | 26) |
| | | NO: 144) | | | | |
| 2C09 | TFPMH | WINPNS | GEWLGY | RASQSIS | AASTLQ | QQSYNP |
| | (SEQ ID | GGTNYA | (SEQ ID | SWLA | S (SEQ ID | PYT (SEQ |
| | NO: 314) | QKFQG | NO: 71) | (SEQ ID | NO: 20) | ID NO: |
| | | (SEQ ID | | NO: 62) | | 315) |
| | | NO: 9) | | | | |
| 2B06 | TYGIS | WINPNS | ANWLDY | RASQDIS | GASSLQS | QQSYSPP |
| | (SEQ ID | GGTNYA | (SEQ ID | RWSA | (SEQ ID | YT (SEQ |
| | NO: 316) | QKFQG | NO: 18) | (SEQ ID | NO: 43) | ID NO: |
| | | (SEQ ID | | NO: 317) | | 98) |
| | | NO: 9) | | | | |
| 2F10 | TYNIH | WINPNS | ANWLDY | RASQEIR | AASNLE | QQSYSIP |
| | (SEQ ID | GGTNYA | (SEQ ID | RWLA | T (SEQ ID | YT (SEQ |
| | NO: 318) | QKFQG | NO: 18) | (SEQ ID | NO: 319) | ID NO: |
| | | (SEQ ID | | NO: 101) | | 82) |
| | | NO: 9) | | | | |
| 2E07 | TYTVS | WINPNS | GDWFDP | RASQGIS | AASTLQ | QQSYET |
| | (SEQ ID | GGTNYA | (SEQ ID | RWLA | S (SEQ ID | PYT (SEQ |
| | NO: 320) | QKFQG | NO: 59) | (SEQ ID | NO: 20) | ID NO: |
| | | (SEQ ID | | NO: 321) | | 322) |
| | | NO: 9) | | | | |
| 3F02 | AYYMH | WISPNSG | GDWND | RASQDIS | AASTLQ | QQGYST |
| | (SEQ ID | GTNYAQ | W (SEQ | RWLA | S (SEQ ID | PYT (SEQ |
| | NO: 323) | KFQG | ID NO: | (SEQ ID | NO: 20) | ID NO: |
| | | (SEQ ID | 324) | NO: 30) | | 171) |
| | | NO: 144) | | | | |
| 3H02 | DYHLH | WINPNS | GEWFDP | RASQSIS | AASTLQ | QQSYSTP |
| | (SEQ ID | GGTNYA | (SEQ ID | SWLA | S (SEQ ID | YT (SEQ |
| | NO: 206) | QMFQG | NO: 24) | (SEQ ID | NO: 20) | ID NO: |
| | | (SEQ ID | | NO: 62) | | 26) |
| | | NO: 325) | | | | |
| 3A09 | DYIVH | WINPYS | VLQEGM | RASQSIS | GVSSLQS | QQSYSN |
| | (SEQ ID | GGTKYA | DV (SEQ | SYLN | (SEQ ID | PT (SEQ |
| | NO: 326) | QKFQG | ID NO: | (SEQ ID | NO: 329) | ID NO: |
| | | (SEQ ID | 328) | NO: 36) | | 330) |
| | | NO: 327) | | | | |
| 3G05 | DYIVH | WINPNS | ANWLDY | RASQSIS | GASSLQS | QQSYITP |
| | (SEQ ID | GGTNYA | (SEQ ID | RWLA | (SEQ ID | YT (SEQ |
| | NO: 326) | QNFQG | NO: 18) | (SEQ ID | NO: 43) | ID NO: |
| | | (SEQ ID | | NO: 25) | | 44) |
| | | NO: 74) | | | | |
| 3F07 | DYLIH | WINPAS | GYRWLH | RASQSIS | AASTLQ | QQSYSPP |
| | (SEQ ID | GGTNYA | DY (SEQ | RWLA | S (SEQ ID | YT (SEQ |
| | NO: 331) | QKFQG | ID NO: | (SEQ ID | NO: 20) | ID NO: |
| | | (SEQ ID | 333) | NO: 25) | | 98) |
| | | NO: 332) | | | | |
| 3A06 | DYNIH | WINPNS | ANWLDY | RASQDIS | AASTLQ | QQSYMT |
| | (SEQ ID | GGTNYA | (SEQ ID | LWLA | S (SEQ ID | PRT (SEQ |
| | NO: 8) | QRFQG | NO: 18) | (SEQ ID | NO: 20) | ID NO: |
| | | (SEQ ID | | NO: 335) | | 336) |
| | | NO: 334) | | | | |
| 3G01 | DYQMF | WINPNS | GGGGYG | RASQGIS | AASSLQS | QQSYSD |
| | (SEQ ID | GGTNYA | MDV | NYLA | (SEQ ID | QWT |
| | NO: 337) | QKFQG | (SEQ ID | (SEQ ID | NO: 63) | (SEQ ID |
| | | (SEQ ID | NO: 338) | NO: 154) | | NO: 339) |
| | | NO: 9) | | | | |
| 3D02 | FYAIS | WMNPNS | GVQDYY | RASQSIG | AASTLQ | QQGYSS |
| | (SEQ ID | GGTTYA | GSDY | RWLA | T (SEQ ID | PYT (SEQ |
| | NO: 340) | QKFQG | (SEQ ID | (SEQ ID | NO: 212) | ID NO: |
| | | (SEQ ID | NO: 342) | NO: 49) | | 343) |
| | | NO: 341) | | | | |
| 3F01 | GYNIH | WINPNS | DEAGYF | QASQDIS | AASTLES | QQSFTIP |
| | (SEQ ID | GGTNYA | DL (SEQ | NYLN | (SEQ ID | WT (SEQ |
| | NO: 344) | QKFQG | ID NO: | (SEQ ID | NO: 346) | ID NO: |
| | | (SEQ ID | 345) | NO: 236) | | 347 |
| | | NO: 9) | | | | |
| 3B07 | GYNMH | WINPNS | ANWLDY | RASQSIN | AASTLQ | QQSYITP |
| | (SEQ ID | GGTNYA | (SEQ ID | RWLA | S (SEQ ID | YT (SEQ |
| | NO: 45) | QKFQG | NO: 18) | (SEQ ID | NO: 20) | ID NO: |
| | | (SEQ ID | | NO: 52) | | 44) |
| | | NO: 9) | | | | |
| 3F06 | GYNMH | WINPNS | GDWFDP | RASQSIS | AASSLQS | QQSYSPP |
| | (SEQ ID | GGTNYA | (SEQ ID | SWLA | (SEQ ID | YT (SEQ |
| | NO: 45) | QKFQG | NO: 59) | (SEQ ID | NO: 63) | ID NO: |
| | | (SEQ ID | | NO: 62) | | 98) |
| | | NO: 9) | | | | |
| 3A10 | GYNVH | WINPNS | DHSSGY | RASQGIS | AASSLQS | QQSYSTP |
| | (SEQ ID | GGTNYA | DY (SEQ | SWLA | (SEQ ID | YT (SEQ |
| | NO: 348) | QKFQG | ID NO: | (SEQ ID | NO: 63) | ID NO: |
| | | (SEQ ID | 349) | NO: 105) | | 26) |
| | | NO: 9) | | | | |
| 3C02 | GYYIH | WINPNS | ENYFDX | RASQSIS | AASSLQS | QQSYSPP |
| | (SEQ ID | GGTNYA | XX (SEQ | RWLT | (SEQ ID | XT (SEQ |
| | NO: 350) | QKFQG | ID NO: | (SEQ ID | NO: 63) | ID NO: |
| | | (SEQ ID | 351) | NO: 352) | | 353) |
| | | NO: 9) | | | | |
| 3E02 | GYYIN | WINPNS | GEWFDP | RASQDIS | GASTLQ | QQSMIA |
| | (SEQ ID | GGTNYA | (SEQ ID | NWLA | S (SEQ ID | PYT (SEQ |
| | NO: 354) | QKFQG | NO: 24) | (SEQ ID | NO: 122) | ID NO: |
| | | (SEQ ID | | NO: 295) | | 355) |
| | | NO: 9) | | | | |
| 3D11 | GYYMH | WMNPNS | GSEIIDY | QASQSIY | AASSLQS | QQSYSV |
| | (SEQ ID | GNTGYA | DSSGLG | NYLN | (SEQ ID | PHT (SEQ |
| | NO: 57) | QKFQG | AFDI | (SEQ ID | NO: 63) | ID NO: |
| | | (SEQ ID | (SEQ ID | NO: 357) | | 358) |
| | | NO: 34) | NO: 356) | | | |
| 3A05 | NHNMH | WINPNS | ANWLDY | RASQSID | AASSLQS | QQSYSIP |
| | (SEQ ID | GGTNYA | (SEQ ID | RWLA | (SEQ ID | YT (SEQ |
| | NO: 80) | QKFQG | NO: 18) | (SEQ ID | NO: 63) | ID NO: |
| | | (SEQ ID | | NO: 81) | | 82) |
| | | NO: 9) | | | | |
| 3A08 | NHNMH | WINPNS | ANWLDY | RASQSID | AASSLQS | QQSYSIP |
| | (SEQ ID | GGTNYA | (SEQ ID | RWLA | (SEQ ID | YT (SEQ |
| | NO: 80) | QKFQG | NO: 18) | (SEQ ID | NO: 63) | ID NO: |
| | | (SEQ ID | | NO: 81) | | 82) |
| | | NO: 9) | | | | |
| 3F11 | NHNMH | WINPNS | ANWLDY | RASQSID | AASSLQS | QQSYSIP |
| | (SEQ ID | GGTNYA | (SEQ ID | RWLA | (SEQ ID | YT (SEQ |
| | NO: 80) | QKFQG | NO: 18) | (SEQ ID | NO: 63) | ID NO: |
| | | (SEQ ID | | NO: 81) | | 82) |
| | | NO: 9) | | | | |
| 3E11 | NSIIN | WIYPNS | RWMDA | RASQGIS | AASTLQ | QQTYSV |
| | (SEQ ID | GGTNYA | FDI (SEQ | NYLA | G (SEQ | PLT (SEQ |
| | NO: 359) | QKFQG | ID NO: | (SEQ ID | ID NO: | ID NO: |
| | | (SEQ ID | 360) | NO: 154) | 361) | 362) |
| | | NO: 66) | | | | |
| 3A03 | NYGIT | WINANS | ANWLDY | RASQSIG | AASSLQS | QQSYSA |
| | (SEQ ID | GGTNYA | (SEQ ID | SWLA | (SEQ ID | PYT (SEQ |
| | NO: 363) | QKFQG | NO: 18) | (SEQ ID | NO: 63) | ID NO: |
| | | (SEQ ID | | NO: 177) | | 365) |
| | | NO: 364) | | | | |
| 3D03 | NYILS | WINPDS | GSYGMD | RASQSIS | AASSLQS | QQSYSTP |
| | (SEQ ID | GGTNYA | V (SEQ | RWLA | (SEQ ID | YT (SEQ |
| | NO: 366) | QKFQG | ID NO: | (SEQ ID | NO: 63) | ID NO: |
| | | (SEQ ID | 367) | NO: 25) | | 26) |
| | | NO: 186) | | | | |
| 3E03 | NYNIH | WINPNS | EGLAAL | RASQSIS | AASTLQ | QQSYISP |
| | (SEQ ID | GGTNYA | DY (SEQ | NWLA | S (SEQ ID | YT (SEQ |
| | NO: 368) | QRFQG | ID NO: | (SEQ ID | NO: 20) | ID NO: |
| | | (SEQ ID | 369) | NO: 188) | | 79) |
| | | NO: 334) | | | | |
| 3D06 | NYPMT | WINPNS | DAAAGF | RASQDIS | AASKLQ | QQSYSPP |
| | (SEQ ID | GGTNYA | DP (SEQ | NWLA | S (SEQ ID | YS (SEQ |
| | NO: 370) | QKFQG | ID NO: | (SEQ ID | NO: 372) | ID NO: |
| | | (SEQ ID | 371) | NO: 295) | | 256) |
| | | NO: 9) | | | | |
| 3G08 | NYVIS | WINPNS | EGRSSY | RASQSIG | GASSVQ | QQSYSSP |
| | (SEQ ID | GGTNFA | DY (SEQ | RWLA | S (SEQ ID | YT (SEQ |
| | NO: 114) | QKFQG | ID NO: | (SEQ ID | NO: 374) | ID NO: |
| | | (SEQ ID | 373) | NO: 49) | | 32) |
| | | NO: 207) | | | | |
| 3A01 | NYYMH | WMNPNS | VGYSSG | QASQDIR | AASSLQS | QQSYTIP |
| | (SEQ ID | GHTSYA | WLGMD | KYLN | (SEQ ID | YT (SEQ |
| | NO: 375) | QKFQG | V (SEQ | (SEQ ID | NO: 63) | ID NO: |
| | | (SEQ ID | ID NO: | NO: 378) | | 379) |
| | | NO: 376) | 377) | | | |
| 3B09 | SHLIH | WINPNT | ANWLDY | RASQDIS | AASSLQS | QQSYTT |
| | (SEQ ID | GGTNYA | (SEQ ID | RWLA | (SEQ ID | PYT (SEQ |
| | NO: 129) | QKFQG | NO: 18) | (SEQ ID | NO: 63) | ID NO: |
| | | (SEQ ID | | NO: 30) | | 102) |
| | | NO: 380) | | | | |
| 3E04 | SSDIN | GIIPIFGT | ADTAMG | RSSQSLL | QGSNRA | MQALQT |
| | (SEQ ID | ANYAQK | HYYYYG | NKNGNN | S (SEQ ID | PPT (SEQ |
| | NO: 381) | FQG | MDV | YID (SEQ | NO: 385) | ID NO: |
| | | (SEQ ID | (SEQ ID | ID NO: | | 386) |
| | | NO: 382) | NO: 383) | 384) | | |
| 3B06 | SYAFS | VIYPSGG | GLHQLP | RASQSIS | AASSLQS | QQSYSSP |
| | (SEQ ID | TTGYAQ | NWFDP | RWLA | (SEQ ID | YT (SEQ |
| | NO: 387) | KFQG | (SEQ ID | (SEQ ID | NO: 63) | ID NO: |
| | | (SEQ ID | NO: 389) | NO: 25) | | 32) |
| | | NO: 388) | | | | |
| 3C04 | SYAIS | WINPNS | DLVGGV | RASQNIT | AASTLQ | QQSYSM |
| | (SEQ ID | GGTNYA | FAFDI | TYLN | S (SEQ ID | PYS (SEQ |
| | NO: 132) | QKFQG | (SEQ ID | (SEQ ID | NO: 20) | ID NO: |
| | | (SEQ ID | NO: 390) | NO: 391) | | 392) |
| | | NO: 9) | | | | |
| 3D01 | SYAIS | GIIPIFGA | DRGADA | KSSQSVL | WASTRA | QQYERT |
| | (SEQ ID | PVYAQD | FDI (SEQ | YSSNNK | S (SEQ ID | PLT (SEQ |
| | NO: 132) | FQG | ID NO: | NYLA | NO: 396) | ID NO: |
| | | (SEQ ID | 394) | (SEQ ID | | 397 |
| | | NO: 393) | | NO: 395) | | |
| 3D09 | SYAIS | WINPNS | ANWLDY | RASQNIN | AASTLQ | QQSYSPP |
| | (SEQ ID | GGTNYA | (SEQ ID | RWLA | S (SEQ ID | YT (SEQ |
| | NO: 132) | QKFQG | NO: 18) | (SEQ ID | NO: 20) | ID NO: |
| | | (SEQ ID | | NO: 97) | | 98) |
| | | NO: 9) | | | | |
| 3E08 | SYAIS | WINPNS | ANWLDY | RASQSIS | AASSLQS | QQSYSPP |
| | (SEQ ID | GGTNYA | (SEQ ID | RWLA | (SEQ ID | YT (SEQ |
| | NO: 132) | QKFQG | NO: 18) | (SEQ ID | NO: 63) | ID NO: |
| | | (SEQ ID | | NO: 25) | | 98) |
| | | NO: 9) | | | | |
| 3E09 | SYAIS | WINPNS | EISFLDY | RASQSIS | GASSLQS | QQSYTT |
| | (SEQ ID | GGTKYA | (SEQ ID | NWLA | (SEQ ID | PYT (SEQ |
| | NO: 132) | QKFQG | NO: 399) | (SEQ ID | NO: 43) | ID NO: |
| | | (SEQ ID | | NO: 188) | | 102) |
| | | NO: 398) | | | | |
| 3B05 | SYAVS | WIIPDSG | GSGWYD | RASQSIS | GASTLQ | QQSYSK |
| | (SEQ ID | GTNYAQ | Y (SEQ | NWLA | S (SEQ ID | PT (SEQ |
| | NO: 400) | KLQG | ID NO: | (SEQ ID | NO: 122) | ID NO: |
| | | (SEQ ID | 402) | NO: 188) | | 403) |
| | | NO: 401) | | | | |
| 3D04 | SYDIN | WINPNS | ANWLDY | RASQNIN | AASSLQS | QQSYSPP |
| | (SEQ ID | GGTNYA | (SEQ ID | RWLA | (SEQ ID | YT (SEQ |
| | NO: 146) | QKFQG | NO: 18) | (SEQ ID | NO: 63) | ID NO: |
| | | (SEQ ID | | NO: 97) | | 98) |
| | | NO: 9) | | | | |
| 3D07 | SYDIN | WINPNS | GEWLDY | RASQSIR | AASSLQS | QQSYSTP |
| | (SEQ ID | GGTNYA | (SEQ ID | SWLA | (SEQ ID | YT (SEQ |
| | NO: 146) | QKFQG | NO: 183) | (SEQ ID | NO: 63) | ID NO: |
| | | (SEQ ID | | NO: 275) | | 26) |
| | | NO: 9) | | | | |
| 3F04 | SYGIF | WINPNS | ANWLDY | RASQDIG | AASSLQS | QQSYSM |
| | (SEQ ID | GGTNYA | (SEQ ID | SWLA | (SEQ ID | IT (SEQ |
| | NO: 404) | QKFQG | NO: 18) | (SEQ ID | NO: 63) | ID NO: |
| | | (SEQ ID | | NO: 405) | | 406) |
| | | NO: 9) | | | | |
| 3B02 | SYGIS | WINPNS | DFDAFDI | RASEAIG | AASSLQS | QQSYSPP |
| | (SEQ ID | GGTNYA | (SEQ ID | TWLA | (SEQ ID | YT (SEQ |
| | NO: 156) | QKFQG | NO: 407) | (SEQ ID | NO: 63) | ID NO: |
| | | (SEQ ID | | NO: 408) | | 98) |
| | | NO: 9) | | | | |
| 3B04 | SYGIS | WINPNS | STDYYY | RASQNIR | AASSLQS | QQSYSN |
| | (SEQ ID | GGTNYA | MDV | GYLN | (SEQ ID | PWT |
| | NO: 156) | QKFQG | (SEQ ID | (SEQ ID | NO: 63) | (SEQ ID |
| | | (SEQ ID | NO: 409) | NO: 410) | | NO: 411) |
| | | NO: 9) | | | | |
| 3C01 | SYGIS | WINPNS | NYDSYD | RASQSV | GASTLQ | QQSYNT |
| | (SEQ ID | GGTNYA | Y (SEQ | GSWLA | D (SEQ | PYT (SEQ |
| | NO: 156) | QNFQG | ID NO: | (SEQ ID | ID NO: | ID NO: |
| | | (SEQ ID | 412) | NO: 413) | 414) | 95) |
| | | NO: 74) | | | | |
| 3C06 | SYGIS | WLNPNS | ANWLDY | RASQRIG | AASSLQS | LQDYNY |
| | (SEQ ID | GGTNYA | (SEQ ID | NWLA | (SEQ ID | PYT (SEQ |
| | NO: 156) | QQFQG | NO: 18) | (SEQ ID | NO: 63) | ID NO: |
| | | (SEQ ID | | NO: 416) | | 417) |
| | | NO: 415) | | | | |
| 3F03 | SYGIS | WINPNS | DYGALD | RASQAIG | AASSLQS | QQSYSA |
| | (SEQ ID | GGTNYA | Y (SEQ | SWLA | (SEQ ID | PYT (SEQ |
| | NO: 156) | QKLQG | ID NO: | (SEQ ID | NO: 63) | ID NO: |
| | | (SEQ ID | 418) | NO: 419) | | 365) |
| | | NO: 17) | | | | |
| 3G04 | SYGIS | WIIPNSG | GSGWSD | RASQSIS | AASSLQS | QQGYST |
| | (SEQ ID | GTNYAQ | Y (SEQ | SWLA | (SEQ ID | PYT (SEQ |
| | NO: 156) | KFQG | ID NO: | (SEQ ID | NO: 63) | ID NO: |
| | | (SEQ ID | 421) | NO: 62) | | 171) |
| | | NO: 420) | | | | |
| G307 | SYGIS | WINPNS | ANWLDY | RASQNIG | AASSLQS | RQSYSPP |
| | (SEQ ID | GGTNYA | (SEQ ID | RWLA | (SEQ ID | YT (SEQ |
| | NO: 156) | QNFQG | NO: 18) | (SEQ ID | NO: 63) | ID NO: |
| | | (SEQ ID | | NO: 422) | | 423) |
| | | NO: 74) | | | | |
| 3E01 | SYNMH | WINPNS | ANWLDY | RASQSIS | GASNLQ | QQSYSPP |
| | (SEQ ID | GGTNYA | (SEQ ID | SWLA | T (SEQ ID | YT (SEQ |
| | NO: 169) | QKFQG | NO: 18) | (SEQ ID | NO: 424) | ID NO: |
| | | (SEQ ID | | NO: 62) | | 98) |
| | | NO: 9) | | | | |
| 3F08 | SYPIS | WINPNS | ANWLDY | RASQSIS | ATSTLQS | QQSYSIP |
| | (SEQ ID | GGTNYA | (SEQ ID | RWLA | (SEQ ID | YA (SEQ |
| | NO: 309) | QNFQG | NO: 18) | (SEQ ID | NO: 425) | ID NO: |
| | | (SEQ ID | | NO: 25) | | 426) |
| | | NO: 74) | | | | |
| 3C09 | SYTIS | WINPNS | ANWLDY | RASQTIS | AASTLQ | QQSYSTP |
| | (SEQ ID | GGTNYA | (SEQ ID | RWLA | S (SEQ ID | YT (SEQ |
| | NO: 312) | QKFQG | NO: 18) | (SEQ ID | NO: 20) | ID NO: |
| | | (SEQ ID | | NO: 427) | | 26) |
| | | NO: 9) | | | | |
| 3A02 | SYVIS | WINPNN | DSGVGM | RASQSIS | AASSLQS | QQSYSA |
| | (SEQ ID | GGTNYA | DV (SEQ | KWLA | (SEQ ID | PYT (SEQ |
| | NO: 179) | QKFQG | ID NO: | (SEQ ID | NO: 63) | ID NO: |
| | | (SEQ ID | 429) | NO: 430) | | 365) |
| | | NO: 428) | | | | |
| 3F05 | SYVIS | WINPNS | DSAGMD | RASQSIS | AASSLQS | QQSYSSP |
| | (SEQ ID | GGTNYA | V (SEQ | SWLA | (SEQ ID | YT (SEQ |
| | NO: 179) | QKFQG | ID NO: | (SEQ ID | NO: 63) | ID NO: |
| | | (SEQ ID | 431) | NO: 62) | | 32) |
| | | NO: 9) | | | | |
| 3E07 | SYVIT | WINPNS | GHMALD | RASQNIN | AASTLQ | QQSYIAP |
| | (SEQ ID | GGTNYA | Y (SEQ | TWLA | S (SEQ ID | YT (SEQ |
| | NO: 432) | QEFQG | ID NO: | (SEQ ID | NO: 20) | ID NO: |
| | | (SEQ ID | 434) | NO: 435) | | 436) |
| | | NO: 433) | | | | |
| 3E05 | SYVMN | WINPNS | ANWLDY | RASQGIS | AASSLQS | QQSYSPP |
| | (SEQ ID | GGTSYA | (SEQ ID | SWLA | (SEQ ID | YT (SEQ |
| | NO: 437) | QKFQG | NO: 18) | (SEQ ID | NO: 63) | ID NO: |
| | | (SEQ ID | | NO: 105) | | 98) |
| | | NO: 438) | | | | |
| 3F09 | SYYLH | IINPSGG | DTEGYG | RASQGIS | AASSLQS | QQAYSF |
| | (SEQ ID | STSYAQ | SGSRPY | SWLA | (SEQ ID | PYT (SEQ |
| | NO: 439) | KFQG | GMDV | (SEQ ID | NO: 63) | ID NO: |
| | | (SEQ ID | (SEQ ID | NO: 105) | | 442) |
| | | NO: 440) | NO: 441) | | | |
| 3C07 | SYYMH | WINPNS | GEWFDP | RASQSIG | AASTLQ | QQSYSTP |
| | (SEQ ID | GGTNYA | (SEQ ID | TWLA | S (SEQ ID | YT (SEQ |
| | NO: 182) | QKFQG | NO: 24) | (SEQ ID | NO: 20) | ID NO: |
| | | (SEQ ID | | NO: 109) | | 26) |
| | | NO: 9) | | | | |
| 3C08 | SYYMH | WINPNS | GEWLDY | RASQSIS | AASSLQS | QQSYRT |
| | (SEQ ID | GGTNYA | (SEQ ID | SWLA | (SEQ ID | PYT (SEQ |
| | NO: 182) | QKFQG | NO: 183) | (SEQ ID | NO: 63) | ID NO: |
| | | (SEQ ID | | NO: 62) | | 286) |
| | | NO: 9) | | | | |
| 3G10 | SYYMH | WINPNS | GEWFDP | RASQSIG | AASTLQ | QQSYSTP |
| | (SEQ ID | GGTNYA | (SEQ ID | TWLA | S (SEQ ID | YT (SEQ |
| | NO: 182) | QKFQG | NO: 24) | (SEQ ID | NO: 20) | ID NO: |
| | | (SEQ ID | | NO: 109) | | 26) |
| | | NO: 9) | | | | |
| 3H01 | TYDIS | WMSPNS | SLHPYSN | QASQDIS | AASSLR | QQSYNT |
| | (SEQ ID | GGTNYA | YWVY | NYLN | G (SEQ | PYS (SEQ |
| | NO: 443) | QNFQG | (SEQ ID | (SEQ ID | ID NO: | ID NO: |
| | | (SEQ ID | NO: 445) | NO: 236) | 446) | 447) |
| | | NO: 444) | | | | |
| 3G03 | TYLIH | WINPNS | ANWLDY | RASQSIS | AASSLQS | QQSYSA |
| | (SEQ ID | GGTNYA | (SEQ ID | TWLA | (SEQ ID | PYT (SEQ |
| | NO: 191) | QKFQG | NO: 18) | (SEQ ID | NO: 63) | ID NO: |
| | | (SEQ ID | | NO: 162) | | 365) |
| | | NO: 9) | | | | |
| 3D10 | TYTIS | WMNPNS | GDWQDI | RASQSIS | AASTLQ | QQSYSTP |
| | (SEQ ID | GGTNYA | (SEQ ID | RWLA | S (SEQ ID | YT (SEQ |
| | NO: 448) | QKFQG | NO: 449) | (SEQ ID | NO: 20) | ID NO: |
| | | (SEQ ID | | NO: 25) | | 26) |
| | | NO: 23) | | | | |
| 3B10 | TYTIT | WINPKS | GEWLDY | RASQSIG | AASTLQ | QQSYGT |
| | (SEQ ID | GGTNYA | (SEQ ID | TWLA | S (SEQ ID | PWT |
| | NO: 450) | QKFQG | NO: 183) | (SEQ ID | NO: 20) | (SEQ ID |
| | | (SEQ ID | | NO: 109) | | NO: 452) |
| | | NO: 451) | | | | |
| 3D08 | TYYVH | RISPNSG | DWSMD | QASQDIS | AASTLQ | QQSYST |
| | (SEQ ID | GTNYAQ | V (SEQ | NYLN | S (SEQ ID | WT (SEQ |
| | NO: 453) | KFQG | ID NO: | (SEQ ID | NO: 20) | ID NO: |
| | | (SEQ ID | 455) | NO: 236) | | 456) |
| | | NO: 454) | | | | |
| 3B11 | XHLIH | WINPNS | DYGMD | RASQXV | AASSLQS | QQSYITP |
| | (SEQ ID | GGTNYA | V (SEQ | NRWLA | (SEQ ID | YT (SEQ |
| | NO: 457) | QKLQG | ID NO: | (SEQ ID | NO: 63) | ID NO: |
| | | (SEQ ID | 130) | NO: 458) | | 44) |
| | | NO: 17) | | | | |

*X refers to an unidentified amino acid residue

TABLE 11
VH and VL sequences of identified scFv hits that bind target G2

| scFv ID | VH | VL |
|---|---|---|
| 1C08 | QVQLVQSGAEVKKPGASVKVS | DIQMTQSPSSLSASVGDRVTITCRAS |
| | CKASGYTVTDYNIHWVRQAPG | QGINNWLAWYQQKPGKAPKLLIYA |
| | QGLEWMGWINPNSGGTNYAQ | ASSLQAGVPSRFSGSGSGTDFTLTISS |
| | KFQGRVTMTRDTSTSTVYMEL | LQPEDFATYYCQQSYLTPYTFGQGT |
| | SSLRSEDTAVYYCARDKVGLD | KLEIK (SEQ ID NO: 15) |
| | YWGQGTLVTVSS (SEQ ID NO: | |
| | 14) | |
| 1A03 | QVQLVQSGAEVKKPGASVKVS | DIQMTQSPSSLSASVGDRVTITCRAS |
| | CKASGYTFIDYNINWVRQAPG | QDINRWLAWYQQKPGKAPKLLIYA |
| | QGLEWMGWINPNSGGTNYAQ | ASTLQSGVPSRFSGSGSGTDFTLTISS |
| | KLQGRVTMTRDTSTSTVYMEL | LQPEDFATYYCQQSYGSPYTFGQGT |
| | SSLRSEDTAVYYCARANWLDY | KVEIK (SEQ ID NO: 460) |
| | WGQGTLVTVSS (SEQ ID NO: | |
| | 459) | |
| 1A07 | QVQLVQSGAEVKKPGASVKVS | DIQMTQSPSSLSASVGDRVTITCRAS |
| | CKASGYTFTDYYLHWVRQAPG | QSISRWLAWYQQKPGKAPKLLIYAA |
| | QGLEWMGWMNPNSGGTNYA | STLQSGVPSRFSGSGSGTDFTLTISSL |
| | QKFQGRVTMTLDTSTSTVYME | QPEDFATYYCQQSYSTPYTFGQGTR |
| | LSSLRSEDTAVYYCARGEWFD | LEIK (SEQ ID NO: 462) |
| | PWGQGTLVTVSS (SEQ ID NO: | |
| | 461) | |
| 1B04 | QVQLVQSGAEVKKPGASVKVS | DIQMTQXPSSLXASVGDRVTITCRAS |
| | CKASGYTFTDYYMHWVRQAP | QDISRWLAWYQQKPGKAXKLLXYA |
| | GQGLEWMGWIFPNSGGTNYA | ASXLQSGVPSRFSGSGXGTDFTLTIS |
| | QKFQGRVTMTRDTSTNTVYME | SLQPEDFATYYCQQSYSSPYTFGQG |
| | LSSLRSEDTAVYYCAREDWCX | TKVEIK (SEQ ID NO: 464) |
| | PWGRGTLVTVSS (SEQ ID NO: | |
| | 463) | |
| 1C11 | QVQLVQSGAEVKKPGASVKVS | DIQMTQSPSSLSASVGDRVTITCRAS |
| | CKASGYSFTGKYMHWVRQAP | QSISSYLNWYQQKPGKAPKLLIYDA |
| | GQGLEWMGWMNPNSGNTGY | SNLETGVPSRFSGSGSGTDFTLTISSL |
| | AQKFQGRVTMTRDTSTSTVYM | QPEDFATYYCQQTDSIPITFGQGTKV |
| | ELSSLRSEDTAVYYCARGGYY | EIK (SEQ ID NO: 466) |
| | GSGSWTYFDLWGRGTLVTVSS | |
| | (SEQ ID NO: 465) | |
| 1H06 | QVQLVQSGAEVKKPGSSVKVS | DIQMTQSPSSLSASVGDRVTITCRAS |
| | CKASGYTFTGYLMHWVRQAP | QGIGRWLAWYQQKPGKAPKLLIYG |
| | GQGLEWMGWINPNSGGTNYA | ASSLQSGVPSRFSGSGSGTDFTLTISS |
| | PKFQGRVTMTRDTSTSTVYME | LQPEDFATYYCQQSYITPYTFGQGT |
| | LSSLRSEDTAVYYCALESSGW | KLEIK (SEQ ID NO: 468) |
| | GNWGQGTLVTVSS (SEQ ID | |
| | NO: 467) | |
| 1D11 | QVQLVQSGAEVKKPGASVKVS | DIQMTQSPSSLSASVGDRVTITCRAX |
| | CKASGYTFIGYNMHWVRQAPG | QXIXRWLAWYQQKPGKAPKLLIYA |
| | QGLEWMGWINPNTGGTSYAQ | ASTLQSGVPSRFSGSGSGTDFTLTISS |
| | KFQGRVTMTRDTSTSTVYMEL | LQPEDFATYYCQQSYSIPITFGQGTK |
| | SSLRSEDTAVYYCARANWLDY | VEIK (SEQ ID NO: 470) |
| | WGQGTLVTVSS (SEQ ID NO: | |
| | 469) | |
| 1G01 | QVQLVQSGAEVKKPGASVKVS | DIQMTQSPSSLSASVGDRVTITCRAS |
| | CKASGYTFTGYNMHWVRQAP | QSIGRWLAWYQQKPGKAPKLLIYA |
| | GQGLEWMGWINPNSGGTNYA | ASRLQSGVPSRFSGSGSGTDFTLTISS |
| | QKFQGRVTMTRDTSTSTVYME | LQPEDFATYYCQQSYGVPTFGQGTK |
| | LSSLRSEDTAVYYCARANWLD | VEIK (SEQ ID NO: 472) |
| | YWGQGTLVTVSS (SEQ ID NO: | |
| | 471) | |
| 1H04 | QVQLVQSGAEVKKPGASVKVS | DIQMTQSPSSLSASVGDRVTITCRAS |
| | CKASGYTFTGYNMHWVRQAP | QSINRWLAWYQQKPGKAPKLLIYA |
| | GQGLEWMGWINPNSGGTNYA | ASTLQSGVPSRFSGSGSGTDFTLTISS |
| | QKFQGRVTMTRDTSTSTVYME | LQPEDFATYYCQQSYITPYTFGQGT |
| | LSSLRSEDTAVYYCARANWLD | KLEIK (SEQ ID NO: 473) |
| | YWGQGTLVTVSS (SEQ ID NO: | |
| | 471) | |
| 1G06 | EVQLVQSGAEVKKPGSSVKVS | DIQMTQSPSSLSASVGDRVTITCRAS |
| | CKASGYTFTGYQMHWVRQAP | QSINSWLAWYQQKPGKAPKLLIYAA |
| | GQGLEWMGWINPNSGGTNYA | STLQSGVPSRFSGSGSGTDFTLTISSL |
| | QKFQGRVTMTRDTSTSTVYME | QPEDFATYYCQQSYSIPYNFGQGTK |
| | VSSLRSEDTAVYYCARDGGSY | LEIK (SEQ ID NO: 475) |
| | LDYWGQGTLVTVSS (SEQ ID | |
| | NO: 474) | |
| 1B05 | QVQLVQSGAEVKKPGASVKVS | DIQMTQSPSSLSASVGDRVTITCRAS |
| | CKASGYTFTGYYMHWVRQAP | QDISRWLAWYQQKPGKAPKLLIYA |
| | GQGLEWMGRIFPNSGGTDYAQ | ASTLQSGVPSRFSGSGSGTDFTLTISS |
| | KFQGRVTMTRDTSTNTVYMEL | LQPEDFATYYCQQSYSSPYTFGQGT |
| | SSLRSEDTAVYYCARGDWFDP | KVEIK (SEQ ID NO: 477) |
| | WGQGTLVTVSS (SEQ ID NO: | |
| | 476) | |
| 1A08 | QVQLVQSGAEVKKPGASVKVS | DIQMTQSPSSLSASVGDRVTITCRAS |
| | CKASGYTLTHYGMSWVRQAP | QSISSWLAWYQQKPGKAPKLLIYAA |
| | GQGLEWMGWINPNSGGTNYA | SSLQSGVPSRFSGSGSGTDFTLTISSL |
| | QKFQGRVTMTRDTSTSTVYME | QPEDFATYYCQQSYSYPTFGQGTKV |
| | LSSLRSEDTAVYYCARDLGXY | DIK (SEQ ID NO: 479) |
| | DYWGQGTLVTVSS (SEQ ID | |
| | NO: 478) | |
| 1F11 | QVQLVQSGAEVKKPGASVKVS | DIQMTQSPSSLSASVGDRVTITCRAS |
| | CKASGYNFNKYDIAWVRQAPG | QSVSSWLAWYQQKPGKAPKLLIYA |
| | QGLEWMGWIYPNSGGTNYAQ | ASTLHSGVPSRFSGSGSGTDFTLTISS |
| | KFQGRVTMTRDTSTSTVYMEL | LQPEDFATYYCQQSYSTPYTFGQGT |
| | SSLRSEDTAVYYCARANWLDY | KLEIK (SEQ ID NO: 481) |
| | WGQGTLVTVSS (SEQ ID NO: | |
| | 480) | |
| 1A04 | EVQLLESGAEVKKPGSSVKVSC | DIQMTQSPSSLSASVGDRVTITCRAS |
| | KASGYTFKNFGISWVRQAPGQ | QSISRWLAWYQQKPGKAXKLXIYA |
| | GLEWMGWINPNSGGTNYAQK | ASSLQSGVPSRFSGSGSGTDFTLTISS |
| | FQGRVTMTRDTSTSTVYMELS | LQPEDFATYYCQQSYSTPYTFGQGT |
| | SLRSEDTAVYYCARANWLDY | KLEIK (SEQ ID NO: 483) |
| | WGQGTLVTVSS (SEQ ID NO: | |
| | 482) | |
| 1F08 | QVQLVQSGAEVKKPGASVKVS | DIQMTQSPSSLSASVGDRVTITCRAS |
| | CKASGYTFTNFPMHWVRQAPG | QSVSNWLAWYQQKPGKAPKLLIYA |
| | QGLEWMGWINPNSGGTNYAQ | ASSLQSGVPSRFSGSGSGTDFTLTISS |
| | KFQGRVTMTRDTSTSTVYMEL | LQPEDFATYYCQQSYSTPYTFGQGT |
| | SSLRSEDTAVYYCARGEWLGY | KVEIK (SEQ ID NO: 485) |
| | WGQGTLVTVSS (SEQ ID NO: | |
| | 484) | |
| 1G09 | QVQLVQSGAEVKKPGASVKVS | DIQMTQSPSSLSASVGDRVTITCRAS |
| | CKASGYTFTNFPMXWVRQAPG | QSVSXWLAWYQQKPGKAPKLLIYA |
| | QGLEWMGWINPNSGGTNYAQ | ASSLQSGVPSRFSGSGSGTDFTLTISS |
| | NFQGRVTMTRDTSTSTVYMEL | LQPEDFATYYCQQSYSTPYTFGQGT |
| | SSLRSEDTAVYYCARGEWLGY | KVEIK (SEQ ID NO: 487) |
| | WGRGTLXTVSS (SEQ ID NO: | |
| | 486) | |
| 1F02 | QVQLVQSGAEVKKPGASVKVS | DIQMTQSPSSLSASVGDRVTITCRAS |
| | CKASGGTFGNFVVSWVRQAPG | ESIGRWLAWYQQKPGKAPKLLIYAA |
| | QGLEWMGWINPNSGGTNYAQ | STLQSGVPSRFSGSGSGTDFTLTISSL |
| | KFQGRVTMTRDTSTSTVYMEL | QPEDFATYYCQQSYISPYTFGQGTK |
| | SSLRSEDTAVYYCANSVGWFD | LEIK (SEQ ID NO: 489) |
| | PWGQGTLVTVSS (SEQ ID NO: | |
| | 488) | |
| 1F04 | EVQLLESGAEVKKPGASVKVS | DIQMTQSPSSLSASVGDRVTITCRAS |
| | CKASGFTFTNHNMHWVRQAP | QSIDRWLAWYQQKPGKAPKLLIYA |
| | GQGLEWMGWINPNSGGTNYA | ASSLQSGVPSRFSGSGSGTDFTLTISS |
| | QKFQGRVTMTRDTSTSTVYME | LQPEDFATYYCQQSYSIPYTFGQGT |
| | LSSLRSEDTAVYYCARANWLD | KLEIK (SEQ ID NO: 491) |
| | YWGQGTLVTVSS (SEQ ID NO: | |
| | 490) | |
| 1C10 | EVQLLESGGGLVQPGGSLRLSC | DIQMTQSPSSLSASVGDRVTITCRAS |
| | AASGFTFSNSAIHWVRQAPGK | QSISSYLNWYQQKPGKAPKLLIKYA |
| | GLEWVSYISGSGDTMFYADSV | SQSISGVPSRFSGSGSGTDFTLTISSL |
| | KGRFTISRDNSKNTLYLQMNSL | QPEDFATYYCQQSYSTPYTFGQGTK |
| | RAEDTAVYYCARDPYYYGSGS | VEIK (SEQ ID NO: 493) |
| | YFDYWGQGTLVTVSS (SEQ ID | |
| | NO: 492) | |
| 1E04 | QVQLVQSGAEVKKPGASVKVS | DIQMTQSPSSLSASVGDRVTITCRAS |
| | CKASGGTFSNSVISWVRQAPG | QRISYWVAWYQQKPGKAPKLLIYD |
| | QGLEWMGWISVYNGETNYAQ | ASTLQSGVPSRFSGSGSGTDFTLTISS |
| | KLQGRVTMTRDTSTSTVYMEL | LQPEDFATYYCQQSYSTPLTFGGGT |
| | SSLRSEDTAVYYCARDRIDGM | KVEIK (SEQ ID NO: 495) |
| | DVWGQGTTVTVSS (SEQ ID | |
| | NO: 494) | |
| 1B07 | QVQLVQSGAEVKKPGASVKVS | DIQMTQSPSSLSASVGDRVTITCRAS |
| | CKASGGTFSNYAFSWVRQAPG | QSISSWLAWYQQKPGKAPKLLIYAA |
| | QGLEWMGWINPNSGGTNYAQ | SSLQSGVPSRFSGSGSGTDFTLTISSL |
| | KFQGRVTMTRDTSTSTVYMEL | QPEDFATYYCQQSYNTPYTFGQGTK |
| | SSLRSEDTAVYYCARGDWLDY | LEIK (SEQ ID NO: 497) |
| | WGQGTLVTVSS (SEQ ID NO: | |
| | 496) | |
| 1E01 | QVQLVQSGAEVKKPGASVKVS | DIQMTQSPSSLSASVGDRVTITCRAS |
| | CKASGGTFSNYAISWVRQAPG | QNINRWLAWYQQKPGKAPKLLIYA |
| | QGLEWMGWINPNSGGTNYAQ | ASTLQSGVPSRFSGSGSGTDFTLTISS |
| | KFQGRVTMTRDTSTSTVYMEL | LQPEDFATYYCQQSYSPPYTFGQGT |
| | SSLRSEDTAVYYCARANWLDY | KLEIK (SEQ ID NO: 499) |
| | WGQGTLVTVSS (SEQ ID NO: | |
| | 498) | |
| 1F07 | QVQLVQSGAEVKKPGASVKVS | DIQMTQSPSSLSASVGDRVTITCRAS |
| | CKASGYTFTNYDINWVRQAPG | QEIRRWLAWYQQKPGKAPKLLIYA |
| | QGLEWMGWINPNSGGTNYAQ | ASSLQSGVPSRFSGSGSGTDFTLTISS |
| | KFQGRVTMTRDTSTSTVYMEL | LQPEDFATYYCQQSYTTPYTFGQGT |
| | SSLRSEDTAVYYCARGEWLSV | KLEIK (SEQ ID NO: 501) |
| | WGQGTLVTVSS (SEQ ID NO: | |
| | 500) | |
| 1A10 | QVQLVQSGAEVKKPGASVKVS | DIQMTQSPSSLSASVGDRVTITCRAS |
| | CKASGGTFSNYGMSWVRQAP | QGISSWLAWYQQKPGKAPKLLIYGA |
| | GQGLEWMGWINPNSGGTNYA | SRLQSGVPSRFSGSGSGTDFTLTISSL |
| | QKFQGRVTMTRDTSTSTVYME | QPEDFATYYCQQSYLPPYTFGQGTK |
| | LSSLRSEDTAVYYCALGDFLDV | LEIK (SEQ ID NO: 503) |
| | WGQGTTVTVSS (SEQ ID NO: | |
| | 502) | |
| 1E10 | QVQLVQSGAEVKKPGASVKVS | DIQMTQSPSSLSASVGDRVTITCRAS |
| | CKASGGTFSNYPITWVRQAPG | QSIGTWLAWYQQKPGKAPKLLIYA |
| | QGLEWMGWINPNSGGTNYAQ | ASSLQSGVPSRFSGSGSGTDFTLTISS |
| | KFQGRVTMTRDTSTSTVYMEL | LQPEDFATYYCQQSYSPPYAFGQGT |
| | SSLRSEDTAVYYCARANWLDY | KLEIK (SEQ ID NO: 505) |
| | WGQGTLVTVSS (SEQ ID NO: | |
| | 504) | |
| 1D10 | QVQLVQSGAEVKKPGSSVKVS | DIQMTQSPSSLSASVGDRVTITCRAS |
| | CKASGGTFSNYTISWVRQAPG | QSISSWLAWYQQKPGKAPKLLIYAA |
| | QGLEWMGWINPNSGGTNYAQ | SSLHSGVPSRFSGSGSGTDFTLTISSL |
| | KFQGRVTMTRDTSTSTVYMEL | QPEDFATYYCQQSYSPPYTFGQGTK |
| | SSLRSEDTAVYYCARANWLDY | LEIK (SEQ ID NO: 507) |
| | WGQGTLVTVSS (SEQ ID NO: | |
| | 506) | |
| 1D07 | QVQLVQSGAEVKKPGASVKVS | DIQMTQSPSSLSASVGDRVTITCRAS |
| | CKASGGTFNNYTVTWVRQAPG | QSISRWLAWYQQKPGKAPKLLIYAA |
| | QGLEWMGWINPNSGGTNYAQ | STLQSGVPSRFSGSGSGTDFTLTISSL |
| | KLQGRVTMTRDTSTSTVYMEL | QPEDFATYYCQQSYITPYTFGQGTK |
| | SSLRSEDTAVYYCARANWLDY | LEIK (SEQ ID NO: 509) |
| | WGQGTLVTVSS (SEQ ID NO: | |
| | 508) | |
| 1E05 | QVQLVQSGAEVKKPGASVKVS | DIQMTQSPSSLSASVGDRVTITCRAS |
| | CKASGGTFSNYVISWVRQAPG | QSISRWLAWYQQKPGKAPKLLIYAA |
| | QGLEWMGWINPNSGGTISAPN | STVQSGVPSRFSGSGSGTDFTLTISSL |
| | FQGRVTMTRDTSTSTVYMELS | QPEDFATYYCQQSYSTPYTFGQGTK |
| | SLRSEDTAVYYCARANWLDY | LEIK (SEQ ID NO: 511) |
| | WGQGTLVTVSS (SEQ ID NO: | |
| | 510) | |
| 1F01 | EVQLLESGAEVKKPGASVKVS | DIQMTQSPSSLSASVGDRVTITCRAS |
| | CKASGYTFTRHLLHWVRQAPG | QNIGSWLAWYQQKPGKAPKLLXYA |
| | QGLEWMGWINPNSGGTNYAQ | ASSLQSGVPSRFSGSGSGTDFTLTISS |
| | KFQGRVTMTRDTSTSTVYMEL | LQPEDFATYYCQQSYSTPYTFGQGT |
| | SSLRSEDTAVYYCARANWLDY | KVEIK (SEQ ID NO: 513) |
| | WGQGTLVTVSS (SEQ ID NO: | |
| | 512) | |
| 1E08 | QVQLVQSGAEVKKPGASVKVS | DIQMTQSPSSLSASVGDRVTITCRAS |
| | CKASGYTLTRYIMHWVRQAPG | QTITRYLNWYQQKPGKAPKLLIYGA |
| | QGLEWMGWINPNSGGTNYAQ | STLQSGVPSRFSGSGSGTDFTLTISSL |
| | KFQGRVTMTRDTSTSTVYMEL | QPEDFATYYCQQSFSTPLTFGQGTK |
| | SSLRSEDTAVYYCAKDTQSGM | VEIK (SEQ ID NO: 515) |
| | DVWGQGTTVTVSS (SEQ ID | |
| | NO: 514) | |
| 1C06 | QVQLVQSGAEVKKPGASVKVS | DIQMTQSPSSLSASVGDRVTITCRAS |
| | CKASGYSFTRYNMHWVRQAP | QSISSWLAWYQQKPGKAPKLLIYGA |
| | GQGLEWMGWINPNSGGTNYA | SSLQSGVPSRFSGSGSGTDFTLTISSL |
| | QKFQGRVTMTRDTSTSTVYME | QPEDFATYYCQQSYSVPYSFGQGTK |
| | LSSLRSEDTAVYYCARDGYSG | LEIK (SEQ ID NO: 517) |
| | LDVWGKGTTVTVSS (SEQ ID | |
| | NO: 516) | |
| 1H03 | QVQLVQSGAEVKKPGASVKVS | DIQMTQSPSSLSASVGDRVTITCRAS |
| | CKASGDTFNRYVMNWVRQAP | QSIDSWLAWYQQKPGKAPKLLIYAA |
| | GQGLEWMGWINPNSGGTNYA | STLQSGVPSRFSGSGSGTDFTLTISSL |
| | QKFQGRVTMTRDTSTSTVYME | QPEDFATYYCQQSYSTPYTFGQGTK |
| | LSSLRSEDTAVYYCARANWLD | LEIK (SEQ ID NO: 519) |
| | YWGQGTLVTVSS (SEQ ID NO: | |
| | 518) | |
| 1H08 | QVQLVQSGAEVKKPGASVKVS | DIQMTQSPSSLSASVGDRVTITCRAS |
| | CKASGYTLTSHLIHWVRQAPG | QNVNRWLAWYQQKPGKAPKLLIYA |
| | QGLEWMGWINPNSGGTNYAQ | ASSLQSGVPSRFSGSGSGTDFTLTISS |
| | KLQGRVTMTRDTSTSTVYMEL | LQPEDFATYYCQQSYITPYTFGQGT |
| | SSLRSEDTAVYYCARDYGMDV | KLEIK (SEQ ID NO: 521) |
| | WGQGTTVTVSS (SEQ ID NO: | |
| | 520) | |
| 1C04 | QVQLVQSGAEVKKPGASVKVS | DIQMTQSPSSLSASVGDRVTITCRAS |
| | CKASGGTFSSYAISWVRQAPG | QDIGTWLAWYQQKPGKAPKLLIYD |
| | QGLEWMGWINPNSGGTRYAQ | ASTLQSGVPSRFSGSGSGTDFTLTISS |
| | KFQGRVTMTRDTSTSTVYMEL | LQPEDFATYYCQQSYRIPYTFGQGT |
| | SSLRSEDTAVYYCARARGMTT | KLEIK (SEQ ID NO: 523) |
| | GYMDVWGKGTTVTVSS (SEQ | |
| | ID NO: 522) | |
| 1D02 | QVQLVQSGAEVKKPGASVKVS | DIQMTQSPSSLSASVGDRVTITCRAS |
| | CKASGGTFSSYAISWVRQAPG | QAISRWLAWYQQKPGKAPKLLIYA |
| | QGLEWMGWIKPDSGGTIYAQK | ASTLQSGVPSRFSGSGSGTDFTLTISS |
| | FQGRVTMTRDTSTSTVYMELS | LQPEDFATYYCQESYIPPYTFGQGTK |
| | SLRSEDTAVYYCARDGNWFDP | VEIK (SEQ ID NO: 525) |
| | WGQGTLVTVSS (SEQ ID NO: | |
| | 524 | |
| 1D05 | QVQLVQSGAEVKKPGASVKVS | DIQMTQSPSSLSASVGDRVTITCRAS |
| | CKASGYTFSSYAISWVRQAPG | QSIRRWLAWYQQKPGKAPKLLIYA |
| | QGLEWMGWINPNSGGTNYAQ | ASSLQSGVPSRFSGSGSGTDFTLTISS |
| | KFQGRVTMTRDTSTSTVYMEL | LQPEDFATYYCQQSYSTPYTFGQGT |
| | SSLRSEDTAVYYCARANWLDY | KLEIK (SEQ ID NO: 527) |
| | WGQGTLVTVSS (SEQ ID NO: | |
| | 526) | |
| 1E11 | QVQLVQSGAEVKKPGASVKVS | DIQMTQSPSSLSASVGDRVTITCRAS |
| | CKASGGTFSSYAISWVRQAPG | QSISRWLAWYQQKPGKAPKLLIYAA |
| | QGLEWMGWINPNSGGTNYAQ | STLQSGVPSRFSGSGSGTDFTLTISSL |
| | KFQGRVTMTRDTSTSTVYMEL | QPEDFATYYCQQSYSTPYTFGQGTK |
| | SSLRSEDTAVYYCARDEWLQH | LEIK (SEQ ID NO: 529) |
| | WGQGTLVTVSS (SEQ ID NO: | |
| | 528) | |
| 1F05 | QVQLVQSGAEVKKPGASVKVS | DIQMTQSPSSLSASVGDRVTITCRAS |
| | CKASGGTFSSYAISWVRQAPG | QSISRWLAWYQQKPGKAPKLLIYAA |
| | QGLEWMGWINPNSGGTNYAQ | SRLHSGVPSRFSGSGSGTDFTLTISSL |
| | KFQGRVTMTRDTSTSTVYMEL | QPEDFATYYCQQSYSPPYTFGQGTK |
| | SSLRSEDTAVYYCARANWLDY | VEIK (SEQ ID NO: 531) |
| | WGQGTLVTVSS (SEQ ID NO: | |
| | 530) | |
| 1G11 | QVQLVQSGAEVKKPGASVKVS | DIQMTQSPSSLSASVGDRVTITCRAS |
| | CKASGGTFSSYAISWVRQAPG | QSIGRWLAWYQQKPGKAPKLLIYA |
| | QGLEWMGWISPNSGGTNYAQ | ASSLQSGVPSRFSGSGSGTDFTLTISS |
| | KFQGRVTMTRDTSTSTVYMEL | LQPEDFATYYCQQSYTSPYRFGQGT |
| | SSLRSEDTAVYYCARANWLDY | KLEIK (SEQ ID NO: 533) |
| | WGQGTLVTVSS (SEQ ID NO: | |
| | 532) | |
| 1A02 | QVQLVQSGAEVKKPGASVKVS | DIQMTQSPSSLSASVGDRVTITCRAS |
| | CKASGYTFISYDINWVRQAPGQ | QSVSRYLNWYQQKPGKAPKLLIYA |
| | GLEWMGRINPYNDETHYAQKF | ASSLSNGVPSRFSGSGSGTDFTLTISS |
| | QGRVTMTRDTSTSTVYMELSS | LQPEDFATYYCQHNYIRPPTFGRGT |
| | LRSEDTAVYYCARDTSGDGRF | KVEIK (SEQ ID NO: 535) |
| | HPWGQGTLVTVSS (SEQ ID | |
| | NO: 534) | |
| 1A06 | QVQLVQSGAEVKKPGASVKVS | DIQMTQSPSSLSASVGDRVTITCRAS |
| | CKASGYSFTSYEIHWVRQAPG | QGISNYLAWYQQKPGKAPKLLIYGA |
| | QGLEWMGWINPNSGGTNYAQ | SSLQSGVPSRFSGSGSGTDFTLTISSL |
| | KFQGRVTMTRDTSTSTVYMEL | QPEDFATYYCQQSYIIPLTFGQGTKV |
| | SSLRSEDTAVYYCAREGGTKL | EIK (SEQ ID NO: 537) |
| | DVWGQGTTVTVSS (SEQ ID | |
| | NO: 536) | |
| 1C05 | QVQLVQSGAEVKKPGASVKVS | DIQMTQSPSSLSASVGDRVTITCRAS |
| | CKASGYTFTSYGISWVRQAPG | QSVDRWLAWYQQKPGKAPKLLIYA |
| | QGLEWMGWINPNSGGTNYAQ | ASNLQSGVPSRFSGSGSGTDFTLTISS |
| | KFQGRVTMTRDTSTSTVYMEL | LQPEDFATYYCQQSYSTPYTFGQGT |
| | SSLRSEDTAVYYCTRGSGWTD | KLEIK (SEQ ID NO: 539) |
| | NWGQGTLVTVSS (SEQ ID NO: | |
| | 538) | |
| 1D04 | QVQLVQSGAEVKKPGASVKVS | DIQMTQSPSSLSASVGDRVTITCRAS |
| | CKASGYTFTSYGISWVRQAPG | QSISTWLAWYQQKPGKAPKLLIYAA |
| | QGLEWMGWINPNSGGTNYAR | SSLQSGVPSRFSGSGSGTDFTLTISSL |
| | KFQGRVTMTRDTSTSTVYMEL | QPEDFATYYCQQSYSTPYTFGQGTK |
| | SSLRSEDTAVYYCAKEMGFLD | LEIK (SEQ ID NO: 541) |
| | YWGQGTTVTVSS (SEQ ID NO: | |
| | 540) | |
| 1E03 | QVQLVQSGAEVKKPGASVKVS | DIQMTQSPSSLSASVGDRVTITCRAS |
| | CKASGYTFSSYGITWVRQAPG | QSINRWLAWYQQKPGKAPKLLIYA |
| | QGLEWMGWINPNSGGTNYAQ | ASTLQSGVPSRFSGSGSGTDFTLTISS |
| | KFQGRVTMTRDTSTSTVYMEL | LQPEDFATYYCQQSYTTPYTFGQGT |
| | SSLRSEDTAVYYCARANWLDY | KLEIK (SEQ ID NO: 543) |
| | WGQGTLVTVSS (SEQ ID NO: | |
| | 542) | |
| 1A11 | EVQLLESGGGLVQPGGSLRLSC | DIQMTQSPSSLSASVGDRVTITCRAS |
| | AASGFTFSSYGMHWVRQAPGK | ESISTYLNWYQQKPGKAPKLLIYAA |
| | GLEWVAVISYDGSNQYYADSV | SSLQSGVPSRFSGSGSGTDFTLTISSL |
| | KGRFTISRDNSKNTLYLQMNSL | QPEDFATYYCQQSYSTPITFGQGTRL |
| | RAEDTAVYYCARTYSYGNYY | EIK (SEQ ID NO: 545) |
| | YYGMDVWGQGTTVTVSS (SEQ | |
| | ID NO: 544) | |
| 1E09 | QVQLVQSGAEVKKPGASVKVS | DIQMTQSPSSLSASVGDRVTITCRAS |
| | CKASGYTFTSYNMHWVRQAP | QSISRWLAWYQQKPGKAPKLLIYAA |
| | GQGLEWMGWINPNSGGTNYA | STLQSGVPSRFSGSGSGTDFTLTISSL |
| | QKFQGRVTMTRDTSTSTVYME | QPEDFATYYCQQGYSTPYTFGQGTK |
| | LSSLRSEDTAVYYCAREGAVA | VEIK (SEQ ID NO: 547) |
| | VDYWGRGTLVTVSS (SEQ ID | |
| | NO: 546) | |
| 1C01 | QVQLVQSGAEVKKPGASVKVS | DIQMTQSPSSLSASVGDRVTITCRAS |
| | CKASGGTLSSYTLSWVRQAPG | QSVGRWLAWYQQKPGKAPKLLIYA |
| | QGLEWMGWINPNSGGTNYAQ | ASSLQSGVPSRFSGSGSGTDFTLTISS |
| | KFQGRVTMTRDTSTSTVYMEL | LQPEDFATYYCQQSYSTPYTFGQGT |
| | SSLRSEDTAVYYCARDGGGGL | KLEIK (SEQ ID NO: 549) |
| | DYWGQGTLVTVSS (SEQ ID | |
| | NO: 548) | |
| 1D09 | QVQLVQSGAEVKKPGASVKVS | DIQMTQSPSSLSASVGDRVTITCRAS |
| | CKASGYTFTSYVINWVRQAPG | QSIGSWLAWYQQKPGKAPKLLIYAA |
| | QGLEWMGWINPNSGGTNYAQ | SSLQSGVPSRFSGSGSGTDFTLTISSL |
| | KLQGRVTMTRDTSTSTVYMEL | QPEDFATYYCQQTYSPPYTFGQGTK |
| | SSLRSEDTAVYYCARERGGAY | LEIK (SEQ ID NO: 551) |
| | DYWGQGTLVTVSS (SEQ ID | |
| | NO: 550) | |
| 1B03 | QVQLVQSGAEVKKPGASVKVS | EIVMTQSPSSLSASVGDRVTITCRAS |
| | CKASGGTFRSYVISWVRQAPG | QSISSWLAWYQQKPGKAPKLLIYAA |
| | QGLEWMGWISPDSGGTKYAQ | SSLQSGVPSRFSGSGSGTDFTLTISSL |
| | KFQGRVTMTRDTSTSTVYMEL | QPEDFATYYCQQSYSPPYTFGQGTK |
| | SSLRSEDTAVYYCARDSYGMD | LEIK (SEQ ID NO: 553) |
| | VWGQGTTVTVSS (SEQ ID NO: | |
| | 552) | |
| 1H02 | QVQLVQSGAEVKKPGASVKVS | DIQMTQSPSSLSASVGDRVTITCRAS |
| | CKASGYTFTSYYMHWVRQAP | QTISSWLAWYQQKPGKAPKLLIYAA |
| | GQGLEWMGWINPNSGGTNYA | SSLQSGVPSRFSGSGSGTDFTLTISSL |
| | QKFQGRVTMTRDTSTSTVYME | QPEDFATYYCQQSYSTPYTFGQGTK |
| | LSSLRSEDTAVYYCARGEWLD | LEIK (SEQ ID NO: 555) |
| | YWGQGTLVTVSS (SEQ ID NO: | |
| | 554) | |
| 1D03 | QVQLVQSGAEVKKPGASVKVS | DIQMTQSPSSLSASVGDRVTITCRAS |
| | CKASGGTFSTHTITWVRQAPG | QSISNWLAWYQQKPGKAPKLLIYGA |
| | QGLEWMGWINPDSGGTNYAQ | SSLHSGVPSRFSGSGSGTDFTLTISSL |
| | KFQGRVTMTRDTSTSTVYMEL | QPEDFATYYCQQSYSVPYTFGQGTK |
| | SSLRSEDTAVYYCAREGYSGL | VEIK (SEQ ID NO: 557) |
| | DYWGQGTLVTVSS (SEQ ID | |
| | NO: 556) | |
| 1B02 | QVQLVQSGAEVKKPGASVKVS | DIQMTQSPSSLSASVGDRVTITCRAS |
| | CKASGYTFTTYLIHWVRQAPG | QSISSWLAWYQQKPGKAPKLLIYGA |
| | QGLEWMGWINPNSGGTNYAQ | SVLQSGVPSRFSGSGSGTDFTLTISSL |
| | KFQGRVTMTRDTSTSTVYMEL | QPEDFATYYCQQSYHVPYTFGQGTK |
| | SSLRSEDTAVYYCARERASGY | LEIK (SEQ ID NO: 559) |
| | DIWGRGTLVTVSS (SEQ ID NO: | |
| | 558) | |
| 1F03 | QVQLVQSGAEVKKPGASVKVS | DIQMTQSPSSLSASVGDRVTITCRAS |
| | CKASGYTFTTYPLSWVRQAPG | QRIGSWLAWYQQKPGKAPKLLIYA |
| | QGLEWMGWINPNSGGTNYAQ | ASTLQSGVPSRFSGSGSGTDFTLTISS |
| | KFQGRVTMTRDTSTSTVYMEL | LQPEDFATYYCQQSYSAPYSFGQGT |
| | SSLRSEDTAVYYCARANWLDY | KLEIK (SEQ ID NO: 561) |
| | WGQGTLVTVSS (SEQ ID NO: | |
| | 560) | |
| 1C09 | QVQLVQSGAEVKKPGSSVKVS | DIQMTQSPSSLSASVGDRVTITCRAS |
| | CKASGYTFTTYPMXWVRQAPG | QGIDRWLAWYQQKPGKAPKLLIYA |
| | QGLEWMGWINPNSGGTNYAQ | ASSLQSGVPSRFSGSGSGTDFTLTISS |
| | KFQGRVTMTRDTSTSTVYMEL | LQPEDFATYYCQQSYSPPYTFGQGT |
| | SSLRSEDTAVYYCAXANWLDY | KVEIK (SEQ ID NO: 563) |
| | WGQGTLVTVSS (SEQ ID NO: | |
| | 562) | |
| 1C07 | QVQLVQSGAEVKKPGASVKVS | DIQMTQSPSSLSASVGDRVTITCRAS |
| | CKASGYPFSTYYMHWVRQAP | QGISSWLAWYQQKPGKAPKLLIYAA |
| | GQGLEWMGWINPNSGGTNYA | SKLQGGVPSRFSGSGSGTDFTLTISS |
| | QKFQGRVTMTRDTSTSTVYME | LQPEDFATYYCQQSYTTPYTFGQGT |
| | LSSLRSEDTAVYYCARGEWLD | KLEIK (SEQ ID NO: 565) |
| | YWGQGTLVTVSS (SEQ ID NO: | |
| | 564) | |
| 2G03 | QVQLVQSGAEVKKPGASVKVS | DIQMTQSPSSLSASVGDRVTITCRAS |
| | CKASGYSFTAYHVHWVRQAP | QNINRWLAWYQQKPGKAPKLLIYA |
| | GQGLEWMGWINPNSGGTNYA | ASTLQSGVPSRFSGSGSGTDFTLTISS |
| | QKFQGRVTMTRDTSTSTVYME | LQPEDFATYYCQQSYSPPYTFGQGT |
| | LSSLRSEDTAVYYCARANWLD | KLEIK (SEQ ID NO: 499) |
| | YWGQGTLVTVSS (SEQ ID NO: | |
| | 566) | |
| 2H05 | QVQLVQSGAEVKKPGASVKVS | DIQMTQSPSSLSASVGDRVTITCRAS |
| | CKASGYTFTAYNMHWVRQAP | QSISRWLAWYQQKPGKAPKLLIYAA |
| | GQGLEYMGWINPNSGGTNYA | SSLQSGVPSRFSGSGSGTDFTLTISSL |
| | QKFQGRVTMTRDTSTSTVYME | QPEDFATYYCQQSYSTPYTFGQGTK |
| | LSSLRSEDTAVYYCARANWLD | LEIK (SEQ ID NO: 568) |
| | YWGQGTLVTVSS (SEQ ID NO: | |
| | 567) | |
| 2C03 | EVQLLESGAEVKKPGASVKVS | DIQMTQSPSSLSASVGDRVTITCRAS |
| | CKASGYTFTDYGLSWVRQAPG | QTIGPWLAWYQQKPGKAPKLLIYA |
| | QGLEWMGWINPNSGGTNYAQ | ASTLQSGVPSRFSGSGSGTDFTLTISS |
| | KFQGRVTMTRDTSTSTVYMEL | LQPEDFATYYCQQSYSTPYTFGQGT |
| | SSLRSEDTAVYYCARANWLDY | KLEIK (SEQ ID NO: 570) |
| | WGQGTLVTVSS (SEQ ID NO: | |
| | 569) | |
| 2F05 | EVQLLESGAEVKKPGASVKVS | DIQMTQSPSSLSASVGDRVTITCRAS |
| | CKASGYTFTDYGLSWVRQAPG | QTIGPWLAWYQQKPGKAPKLLIYA |
| | QGLEWMGWINPNSGGTNYAQ | ASTLQSGVPSRFSGSGSGTDFTLTISS |
| | KFQGRVTMTRDTSTSTVYMEL | LQPEDFATYYCQQSYSTPYTFGQGT |
| | SSLRSEDTAVYYCARANWLDY | KLEIK (SEQ ID NO: 570) |
| | WGQGTLVTVSS (SEQ ID NO: | |
| | 569) | |
| 2F04 | QVQLVQSGAEVKKPGSSVKVS | DIQMTQSPSSLSASVGDRVTITCRAS |
| | CKASGYTFTDYHLHWVRQAPG | QSISSWLAWYQQKPGKAPKLLIYAA |
| | QGLEWVGWINPNSGGTNFAQK | SSLQSGVPSRFSGSGSGTDFTLTISSL |
| | FQGRVTMTRDTSTSTVYMELS | QPEDFATYYCQQSYSTPYTFGQGTK |
| | SLRSEDTAVYYCARGEWLDY | LEIK (SEQ ID NO: 572) |
| | WGQGTLVTVSS (SEQ ID NO: | |
| | 571) | |
| 2G04 | QVQLVQSGAEVKKPGASVKVS | DIQMTQSPSSLSASVGDRVTITCRAS |
| | CKASGYTFTDYLMHWVRQAP | QSINRWLAWYQQKPGKAPKLLIYA |
| | GQGLEWMGWINPNSGGTNYA | ASKLETGVPSRFSGSGSGTDFTLTISS |
| | QKXQGRVTMTRDTSTSTVYME | LQPEDFATYYCQQSYNTPYTFGQGT |
| | LXSLRSEDTAVYYCARXNXXD | KLEIK (SEQ ID NO: 574) |
| | YWGXGXLXXVSS (SEQ ID NO: | |
| | 573) | |
| 2C01 | QVQLVQSGAEVKKPGASVKVS | DIQMTQSPSSLSASVGDRVTITCRAS |
| | CKASGFTFTDYNIHWVRQAPG | QSISRWLAWYQQKPGKAPKLLIYAA |
| | QGLEWMGWINPNSGGTNYAQ | STLQTGVPSRFSGSGSGTDFTLTISSL |
| | KFQGRVTMTRDTSTSTVYMEL | QPEDFATYYCQQSYSTPYTFGQGTK |
| | SSLRSEDTAVYYCARANWLDY | LEIK (SEQ ID NO: 576) |
| | WGRGTLVTVSS (SEQ ID NO: | |
| | 575) | |
| 2E01 | QVQLVQSGAEVKKPGASVKVS | DIQMTQSPSSLSASVGDRVTITCRAS |
| | CKASGYTFTDYQIHWVRQAPG | QSISTWLAWYQQKPGKAPKLLIYAA |
| | QGLEWMGWISPYSGGTNYAQ | SSLQSGVPSRFSGSGSGTDFTLTISSL |
| | KFQGRVTMTRDTSTSTVYMEL | QPEDFATYYCQQSYSIPYTFGQGTK |
| | SSLRSEDTAVYYCARDRVGLD | VEIK (SEQ ID NO: 578) |
| | YWGQGTLVTVSS (SEQ ID NO: | |
| | 577) | |
| 2A10 | EVQLLESGAEVKKPGASVKVS | DIQMTQSPSSLSASVGDRVTITCRAS |
| | CKASGYTFTDYTIHWVRQAPG | QNIRRWLAWYQQKPGKAPKLLIYA |
| | QGLEWMGWINPNSGGTNYAQ | ASSLRSGVPSRFSGSGSGTDFTLTISS |
| | QFQGRVTMTRDTSTSTVYMEL | LQPEDFATYYCQQSYSTPYTFGQGT |
| | SSLRSEDTAVYYCARANWLDY | KVEIK (SEQ ID NO: 580) |
| | WGQGTLVTVSS (SEQ ID NO: | |
| | 579) | |
| 2G06 | QVQLVQSGAEVKKPGASVKVS | DIQMTQSPSSLSASVGDRVTITCRAS |
| | CKASGYTFTENEMHWVRQAP | QEIRRWLAWYQQKPGKAPKLLIYA |
| | GQGLEWMGWINPNSGGTSYAP | ASSLQSGVPSRFSGSGSGTDFTLTISS |
| | NFQGRVTMTRDTSTSTVYMEL | LQPEDFATYYCQQSYSPPYTFGQGT |
| | SSLRSEDTAVYYCARANWLDF | KLEIK (SEQ ID NO: 582) |
| | WGQGTLVTVSS (SEQ ID NO: | |
| | 581) | |
| 2H07 | QVQLVQSGAEVKKPGASVKVS | DIQMTQSPSSLSASVGDRVTITCQAS |
| | CKASGYAFTFSTIHWVRQAPG | QDINNYLNWYQQKPGKAPKLLIYA |
| | QGLEWMGWISPHSGGTNYAQE | ASSLQSGVPSRFSGSGSGTDFTLTISS |
| | FQGRVTMTRDTSTSTVYMELS | LQPEDFATYYCQQSYSTPYTFGQGT |
| | SLRSEDTAVYYCARDSGSYLD | KLEIK (SEQ ID NO: 584) |
| | YWGQGTLVTVSS (SEQ ID NO: | |
| | 583) | |
| 2G07 | QVQLVQSGAEVKKPGASVKVS | DIQMTQSPSSLSASVGDRVTITCRAS |
| | CKASGGTFSGYAISWVRQAPG | QEIRRWLAWYQQKPGKAPKLLIYA |
| | QGLEWMGWINPNSGGTNYAQ | ASSLQSGVPSRFSGSGSGTDFTLTISS |
| | KFQGRVTMTHDTSTSTVYMDL | LQPEDFATYYCQQSYSTPYTFGQGT |
| | SSLRSEDTAVYYCARGDWFDP | KLEIK (SEQ ID NO: 586) |
| | WGQGTLVTVSS (SEQ ID NO: | |
| | 585) | |
| 2B01 | QVQLVQSGAEVKKPGASVKVS | DIQMTQSPSSLSASVGDRVTITCRAS |
| | CKASGYTFTGYNMHWVRQAP | QSINRWLAWYQQKPGKAPKLLIYA |
| | GQGLEWMGWINPNSGGTNYA | ASTLQSGVPSRFSGSGSGTDFTLTISS |
| | QKFQGRVTMTRDTSTSTVYME | LQPEDFATYYCQQSYITPYTFGQGT |
| | LSSLRSEDTAVYYCARANWLD | KLEIK (SEQ ID NO: 473) |
| | YWGQGTLVTVSS (SEQ ID NO: | |
| | 471) | |
| 2B10 | EVQLLESGAEVKKPGASVKVS | DIQMTQSPSSLSASVGNRVTITCRAS |
| | CKASGYTFTGYNMHWVRQAP | QSINRWLAWYQQKPGKAPKLLIYA |
| | GQGLEWMGWINPNSGGTNYA | ASTLQSGVPSRFSGSGSGTDFTLTISS |
| | QKFQGRVTMTRDTSTSTVYME | LQPEDFATYYCQQSYITPYTFGQGT |
| | LSSLRSEDTAVYYCARANWLD | KLEIK (SEQ ID NO: 588) |
| | YWGQGTLVTVSS (SEQ ID NO: | |
| | 587) | |
| 2D11 | EVQLLESGAEVKKPGASVKVS | DIQMTQSPSSLSASVGDRVTITCRAS |
| | CKASGYTFTGYNMHWVRQAP | QSINRWLAWYQQKPGKAPKLLIYA |
| | GQGLEWMGWINPNSGGTNYA | ASTLQSGVPSRFSGSGSGTDFTLTISS |
| | QKFQGRVTMTRDTSTSTVYME | LQPEDFATYYCQQSYITPYTFGQGT |
| | LSSLRSEDTAVYYCARANWLD | KLEIK (SEQ ID NO: 473) |
| | YWGQGTLVTVSS (SEQ ID NO: | |
| | 587) | |
| 2G02 | QVQLVQSGAEVKKPGASVKVS | DIQMTQSPSSLSASVGDRVTITCRAS |
| | CKASGYTFTGYNMHWVRQAP | QSINRWLAWYQQKPGKAPKLLIYA |
| | GQGLEWMGWINPNSGGTNYA | ASTLQSGVPSRFSGSGSGTDFTLTISS |
| | QKFQGRVTMTRDTSTSTVYME | LQPEDFATYYCQQSYITPYTFGQGT |
| | LSSLRSEDTAVYYCARANWLD | KLEIK (SEQ ID NO: 473) |
| | YWGQGTLVTVSS (SEQ ID NO: | |
| | 471) | |
| 2H08 | QVQLVQSGAEVKKPGASVKVS | DIQMTQSPSSLSASVGDRVTITCRAS |
| | CKASGYTFTGYNMHWVRQAP | QSINRWLAWYQQKPGKAPKLLIYA |
| | GQGLEWMGWINPNSGGTNYA | ASTLQSGVPSRFSGSGSGTDFTLTISS |
| | QKFQGRVTMTRDTSTSTVYME | LQPEDFATYYCQQSYITPYTFGQGT |
| | LSSLRSEDTAVYYCARANWLD | KLEIK (SEQ ID NO: 473) |
| | YWGQGTLVTVSS (SEQ ID NO: | |
| | 471) | |
| 2G01 | QVQLVQSGAEVKKPGASVKVS | DIQMTQSPSSLSASVGDRVTITCRAS |
| | CKASGYTFTGYYLHWVRQAPG | QRVDSWLAWYQQKPGKAPKLLIYK |
| | QGLEWMGWINPKSGGTNFAQ | ASILESGVPSRFSGSGSGTDFTLTISS |
| | KFQGRVTMTRDTSTSTVYMEL | LQPEDFATYYCQQTYSTPWTFGQGT |
| | SSLRSEDTAVYYCARERGATG | KVEIK (SEQ ID NO: 590) |
| | YYYGMDVWGQGTTVTVSS | |
| | (SEQ ID NO: 589) | |
| 2E06 | QVQLVQSGAEVKKPGASVKVS | DIQMTQSPSSLSASVGDRVTITCQAS |
| | CKASGYTFTGYYMHWVRQAP | QDISNYLNWYQQKPGKAPKLLISVA |
| | GQGLEWMGWIYPNSGGTNYA | STLQSGVPSRFSGSGSGTDFTLTISSL |
| | QNLQGRVTMTRDTSTSTVYME | QPEDFATYYCQQSYNTPYTFGQGTK |
| | LSSLRSEDTAVYYCARDDFDY | LEIK (SEQ ID NO: 592) |
| | WGQGTLVTVSS (SEQ ID NO: | |
| | 591) | |
| 2B02 | QVQLVQSGAEVKKPGASVKVS | DIQMTQSPSSLSASVGDRVTITCRAS |
| | CKASGYNFNKYDIAWVRQAPG | QSVSSWLAWYQQKPGKAPKLLIYA |
| | QGLEWMGWIYPNSGGTNYAQ | ASTLHSGVPSRFSGSGSGTDFTLTISS |
| | KFQGRVTMTRDTSTSTVYMEL | LQPEDFATYYCQQSYSTPYTFGQGT |
| | SSLRSEDTAVYYCARANWLDY | KLEIK (SEQ ID NO: 481) |
| | WGQGTLVTVSS (SEQ ID NO: | |
| | 480) | |
| 2D04 | QVQLVQSGAEVKKPGASVKVS | DIQMTQSPSSLSASVGDRVTITCRAS |
| | CKASGYTFTKYGISWVRQAPG | QSISNWLAWYQQKPGKAPKLLIYAA |
| | QGLEWMGWINPYSGGTNYAQ | SSLQSGVPSRFSGSGSGTDFTLTISSL |
| | NFQGRVTMTRDTSTSTVYMEL | QPEDFATYYCQQSYSPPYTFGQGTK |
| | SSLRSEDTAVYYCARGDGWYD | LEIK (SEQ ID NO: 594) |
| | PWGQGTLVTVSS (SEQ ID NO: | |
| | 593) | |
| 2A07 | QVQLVQSGAEVKKPGASVKVS | DIQMTQSPSSLSASVGDRVTITCRAS |
| | CKASGYTFSNFLINWVRQAPG | QSISSWVAWYQQKPGKAPKLLIYGA |
| | QGLEWMGWINPNSGGTNYAQ | SNLESGVPSRFSGSGSGTDFTLTISSL |
| | KFQGRVTMTRDTSTSTVYMEL | QPEDFATYYCQQTYNPPYSFGQGTK |
| | SSLRSEDTAVYYCASERELPFDI | LEIK (SEQ ID NO: 596) |
| | WGQGTMVTVSS (SEQ ID NO: | |
| | 595) | |
| 2H04 | QVQLVQSGAEVKKPGASVKVS | DIQMTQSPSSLSASVGDRVTITCRAS |
| | CKASGYTFTNFPMHWVRQAPG | QSVSNWLAWYQQKPGKAPKLLIYA |
| | QGLEWMGWINPNSGGTNYAQ | ASSLQSGVPSRFSGSGSGTDFTLTISS |
| | KFQGRVTMTRDTSTSTVYMEL | LQPEDFATYYCQQSYSTPYTFGQGT |
| | SSLRSEDTAVYYCARGEWLGY | KVEIK (SEQ ID NO: 485) |
| | WGQGTLVTVSS (SEQ ID NO: | |
| | 484) | |
| 2B11 | QVQLVQSGAEVKKPGASVKVS | DIQMTQSPSSLSASVGDRVTITCRAS |
| | CKASGYTFSNHIMHWVRQAPG | QSISNWLAWYQQKPGKAPKLLIYAA |
| | QGLEWMGWIYPDSGGTKYAQ | SSLQSGVPSRFSGSGSGTDFTLTISSL |
| | KFQGRVTMTRDTSTSTVYMEL | QPEDFATYYCQQSYNTPFTFGPGTK |
| | SSLRSEDTAVYYCARATGFMD | VDIK (SEQ ID NO: 598) |
| | VWGQGTTVTVSS (SEQ ID NO: | |
| | 597) | |
| 2F11 | QVQLVQSGAEVKKPGASVKVS | DIQMTQSPSSLSASVGDRVTITCRAS |
| | CKASGFTFTNHNMHWVRQAP | QSIDRWLAWYQQKPGKAPKLLIYA |
| | GQGLEWMGWINPNSGGTNYA | ASSLQSGVPSRFSGSGSGTDFTLTISS |
| | QKFQGRVTMTRDTSTSTVYME | LQPEDFATYYCQQSYSIPYTFGQGT |
| | LSSLRSEDTAVYYCARANWLD | KLEIK (SEQ ID NO: 491) |
| | YWGQGTLVTVSS (SEQ ID NO: | |
| | 599) | |
| 2D08 | QVQLVQSGAEVKKPGSSVKVS | DIQMTQSPSSLSASVGDRVTITCRAS |
| | CKASGGTFSNYAFNWVRQAPG | QSIDRWLAWYQQKPGKAPKLLIYA |
| | QGLEWMGWIYPNTGGTHYAP | ASTLQSGVPSRFSGSGSGTDFTLTISS |
| | KFQGRVTMTRDTSTSTVYMEL | LQPEDFATYYCQQSYITPYTFGQGT |
| | SSLRSEDTAVYYCARANWLDY | KLEIK (SEQ ID NO: 601) |
| | WGQGTLVTVSS (SEQ ID NO: | |
| | 600) | |
| 2F03 | QVQLVQSGAEVKKPGASVKVS | DIQMTQSPSSLSASVGDRVTITCRAS |
| | CKASGGTFSNYAFSWVRQAPG | QSISRWLAWYQQKPGKAPKLLIYAA |
| | QGLEWMGWIYPNSGGTNYAQ | SNLESGVPSRFSGSGSGTDFTLTISSL |
| | KFQGRVTMTRDTSTSTVYMEL | QPEDFATYYCQQSYSTPYTFGQGTK |
| | SSLRSEDTAVYYCARGESWFD | LEIK (SEQ ID NO: 603) |
| | PWGQGTLVTVSS (SEQ ID NO: | |
| | 602) | |
| 2A05 | QVQLVQSGAEVKKPGASVKVS | DIQMTQSPSSLSASVGDRVTITCRAS |
| | CKASGGTFSNYAISWVRQAPG | QEIRRWLAWYQQKPGKAPKLLIYA |
| | QGLEWMGWINPNTGGTNYAQ | ASSLQSGVPSRFSGSGSGTDFTLTISS |
| | SLQGRVTMTRDTSTSTVYMEL | LQPEDFATYYCQQSYSPPYSFGQGT |
| | SSLRSEDTAVYYCARSSGWYD | KLEIK (SEQ ID NO: 605) |
| | YWGQGTLVTVSS (SEQ ID NO: | |
| | 604) | |
| 2D07 | QVQLVQSGAEVKKPGASVKVS | DIQMTQSPSSLSASVGDRVTITCRAS |
| | CKASGGTFSNYGISWVRQAPG | QSISRWLAWYQQKPGKAPKLLIYAA |
| | QGLEWMGWISPDSGGTIYAQR | SSLQSGVPSRFSGSGSGTDFTLTISSL |
| | FQGRVTMTRDTSTSTVYMELS | QPEDFATYYCQQSYSTPYTFGQGTK |
| | SLRSEDTAVYYCARANWLDY | VEIK (SEQ ID NO: 607) |
| | WGQGTLVTVSS (SEQ ID NO: | |
| | 606) | |
| 2E09 | EVQLLESGAEVKKPGASVKVS | DIQMTQSPSSLSASVGDRVTITCRAS |
| | CKASGYTFTNYGISWVRQAPG | QSVGRWLAWYQQKPGKAPKLLIYA |
| | QGLEWMGWINPYSGGTNYAQ | ASSLQSGVPSRFSGSGSGTDFTLTISS |
| | KFQGRVTMTRDTSTSTVYMEL | LQPEDFATYYCQQSYSIPYSFGQGTK |
| | SSLRSEDTAVYYCARANWLDY | LEIK (SEQ ID NO: 609) |
| | WGQGTLVTVSS (SEQ ID NO: | |
| | 608) | |
| 2G09 | QVQLVQSGAEVKKPGASVKVS | DIQMTQSPSSLSASVGDRVTITCRAS |
| | CKASGYSFTNYHMHWVRQAP | QSINSWLAWYQQKPGKAPKLLIYGA |
| | GQGLEWMGWIYPNSGGTNYA | SNLQSGVPSRFSGSGSGTDFTLTISSL |
| | QKFQGRVTMTRDTSTSTVYME | QPEDFATYYCQQSYSTPYTFGQGTK |
| | LSSLRSEDTAVYYCARGAWFD | LEIK (SEQ ID NO: 611) |
| | PWGQGTLVTVSS (SEQ ID NO: | |
| | 610) | |
| 2A04 | EVQLLESGAEVKKPGASVKVS | DIQMTQSPSSLSASVGDRVTITCRAS |
| | CKASGYNFINYNIAWVRQAPG | QGISSWLAWYQQKPGKAPKLLIYAA |
| | QGLEWMGWINPNSGGTNYAQ | STLQSGVPSRFSGSGSGTDFTLTISSL |
| | KFQGRVTMTRDTSTSTVYMEL | QPEDFATYYCQQSYSTPYTFGQGTK |
| | SSLRSEDTAVYYCARANWLDY | LEIK (SEQ ID NO: 613) |
| | WGQGTLVTVSS (SEQ ID NO: | |
| | 612) | |
| 2H03 | QVQLVQSGAEVKKPGASVKVS | DIQMTQSPSSLSASVGDRVTITCRAS |
| | CKASGYTFTNYPIHWVRQAPG | QEIRRWLAWYQQKPGKAPKLLIYA |
| | QGLEWMGWINPNSGGTNYAQ | ASNLQSGVPSRFSGSGSGTDFTLTISS |
| | KLQGRVTMTRDTSTSTVYMEL | LQPEDFATYYCQQSYSTPYTFGQGT |
| | SSLRSEDTAVYYCARDQGAGW | KLEIK (SEQ ID NO: 615) |
| | DYWGQGTLVTVSS (SEQ ID | |
| | NO: 614) | |
| 2E04 | QVQLVQSGAEVKKPGASVKVS | DIQMTQSPSSLSASVGDRVTITCRAS |
| | CKASGYTFTNYQIHWVRQAPG | QSISRWLAWYQQKPGKAPKLLIYAA |
| | QGLEWMGWINPNSGGTISAPN | STVQSGVPSRFSGSGSGTDFTLTISSL |
| | FQGRVTMTRDTSTSTVYMELS | QPEDFATYYCQQSYSTPYTFGQGTK |
| | SLRSEDTAVYYCARANWLDY | LEIK (SEQ ID NO: 511) |
| | WGQGTLVTVSS (SEQ ID NO: | |
| | 616) | |
| 2A09 | QVQLVQSGAEVKKPGASVKVS | DIQMTQSPSSLSASVGDRVTITCRAS |
| | CKASGGTFSNYVISWVRQAPG | QSINSWLAWYQQKPGKAPKLLIYGA |
| | QGLEWMGWINPNSGGTIYAQK | SSLQSGVPSRFSGSGSGTDFTLTISSL |
| | FQGRVTMTRDTSTSTVYMELS | QPEDFATYYCQQSYSTPYTFGQGTK |
| | SLRSEDTAVYYCARGEWFDPW | LEIK (SEQ ID NO: 618) |
| | GQGTLVTVSS (SEQ ID NO: 617) | |
| 2D09 | QVQLVQSGAEVKKPGASVKVS | DIQMTQSPSSLSASVGDRVTITCRAS |
| | CKASGGTFSNYVISWVRQAPG | QDISTWLAWYQQKPGKAPKLLIYA |
| | QGLEWMGWIYPNTGGTNYAQ | ASSLQSGVPSRFSGSGSGTDFTLTISS |
| | KFQGRVTMTRDTSTSTVYMEL | LQPEDFATYYCQQSYSSWTFGQGTK |
| | SSLRSEDTAVYYCARDRGYGM | VEIK (SEQ ID NO: 620) |
| | DVWGQGTKVTVSS (SEQ ID | |
| | NO: 619) | |
| 2B08 | QVQLVQSGAEVKKPGASVKVS | DIQMTQSPSSLSASVGDRVTITCRAS |
| | CKASGGNFTPYTFSWVRQAPG | QSIRSWLAWYQQKPGKAPKLLIYAA |
| | QGLEWMGWINPSSGGAQYAQ | SSLQSGVPSRFSGSGSGTDFTLTISSL |
| | KLQGRVTMTRDTSTSTVYMEL | QPEDFATYYCQQSYITPYTFGQGTK |
| | SSLRSEDTAVYYCARGEWLDY | LEIK (SEQ ID NO: 622) |
| | WGQGTLVTVSS (SEQ ID NO: | |
| | 621) | |
| 2G11 | QVQLVQSGAEVKKPGASVKFS | DIQMTQSPSSLSASVGDRVTITCRAS |
| | CKASGYTFTRHPMHWVRQAP | QSIDRWLAWYQQKPGKAPKLLIYA |
| | GQGLEWMGWIHPDSGGTNYS | ASTLQSGVPSRFSGSGSGTDFTLTISS |
| | QKFQGRVTMTRDTSTSTVYME | LQPEDFATYYCQQSYSTPYSFGQGT |
| | LSSLRSEDTAVYYCARANWLD | KVEIK (SEQ ID NO: 624) |
| | YWGQGTLVTVSS (SEQ ID NO: | |
| | 623) | |
| 2C08 | QVQLVQSGAEVKKHGASVKVS | DIQMTQSPSSLSASVGDRVTITCRAS |
| | CKASGYTFPSHGITWVRQAPG | QSISRWLAWYQQKPGKAPKLLIYAA |
| | QGLEWMGWINPNSGGTNYAQ | SSLQSGVPSRFSGSGSGTDFTLTISSL |
| | NVQGRVTMTRDTSTSTVYMEL | QPEDFATYYCQQSYSTPYTFGQGTK |
| | SSLRSEDTAVYYCARGPWLDY | LEIK (SEQ ID NO: 568) |
| | WGQGTLVTVSS (SEQ ID NO: | |
| | 625) | |
| 2C05 | QVQLVQSGAXVXKPGASLKVS | DIQMTQSPSSLSASVGDRVTITCRAS |
| | CRASGYTLTSHLIHWVRQAPG | QGIGRWLAWYQQKPGKAPKLLIYA |
| | QGLEWMGWINPNSGGTNYAQ | TSRLQSGVPSRFSGSGSGTDFTLTISS |
| | KFQGRVTMTRDTSTSTXYMEL | LQPEDFATYYCQQSYGTPYTFGQGT |
| | SSLRSEDTAVYYCARGIFEDYW | KVEIK (SEQ ID NO: 627) |
| | GQGTLVTGSS (SEQ ID NO: 626) | |
| 2C07 | EVQLLESGAEVKKPGASVKVS | DIQMTQSPSSLSASVGDRVTITCRAS |
| | CKASGYTFTSHLIHWVRQAPG | QSIDRWLTWYQQKPGKAPKLLIYAA |
| | QGLEWMGWINPNSGGTNYAQ | SRLQSGVPSRFSGSGSGTDFTLTISSL |
| | KFQGRVTMTRDTSTSTVYMEL | QPEDFATYYCQQSYRTPYTFGQGTK |
| | SSLRSEDTAVYYCARANWLDY | VEIK (SEQ ID NO: 629) |
| | WGQGTLVTVSS (SEQ ID NO: | |
| | 628) | |
| 2F01 | QVQLVQSGVEVKKPGASVKVS | DIQMTQSPSSLSASVGDRVTITCRAS |
| | CKASGYTLTSHLIHWVRQAPG | QSIGRWLAWYQQKPGKAPKLLIYA |
| | QGLEWMGWINPNSGGTKYAP | ASTLQSGVPSRFSGSGSGTDFTLTISS |
| | KFQGRVTMTRDTSTSTVYMEV | LQPEDFATYYCQQSYSSPYTFGQGT |
| | SSLRSEDTAVYYCARGDWYDY | KVEIK (SEQ ID NO: 631) |
| | WGQGTLVTVSS (SEQ ID NO: | |
| | 630) | |
| 2H01 | QVQLVQSGAEVKKPGASVKVS | DIQMTQSPSSLSASVGDRVTITCRAS |
| | CKASGYTLTSHLIHWVRQAPG | QSISRWLAWYQQKPGKAPKLLIYAA |
| | QGLEWMGWINPNSGGTNYAQ | SSLQSGVPSRFSGSGSGTDFTLTISSL |
| | SFQGRVTMTRDTSTSTVYMEL | QPEDFATYYCQQSYSAPLTFGGGTK |
| | SSLRSEDTAVYYCARANWLDY | VEIK (SEQ ID NO: 633) |
| | WGQGTLVTVSS (SEQ ID NO: | |
| | 632) | |
| 2B07 | EVQLVQSGAEVKKPGASVKVS | DIQMTQSPSSLSASVGDRVTITCRAS |
| | CKASGGTFSSYAISWVRQAPG | QNINRWLAWYQQKPGKAPKLLIYA |
| | QGLEWMGWINPNSGGTNYAQ | ASTLQSGVPSRFSGSGSGTDFTLTISS |
| | KFQGRVTMTRDTSTSTVYMEL | LQPEDFATYYCQQSYSPPYTFGQGT |
| | SSLRSEDTAVYYCARANWLDY | KLEIK (SEQ ID NO: 499) |
| | WGQGTLVTVSS (SEQ ID NO: | |
| | 634) | |
| 2C06 | QVQLVQSGAEVKKPGASVKVS | DIQMTQSPSSLSASVGDRVTITCRAS |
| | CKASGYTFSSYAISWVRQAPG | QSISRWLAWYQQKPGKAPKLLIYAA |
| | QGLEWMGWINPNSGGTNYAQ | SRLHSGVPSRFSGSGSGTDFTLTISSL |
| | KFQGRVTMTRDTSTSTVYMEL | QPEDFATYYCQQSYSPPYTFGQGTK |
| | SSLRSEDTAVYYCARANWLDY | VEIK (SEQ ID NO: 531) |
| | WGQGTLVTVSS (SEQ ID NO: | |
| | 526) | |
| 2A11 | EVQLLESGGGLVQPGGSLRLSC | DIQMTQSPSSLSASVGDRVTITCRAS |
| | AASGFTFSSYAMHWVRQAPGK | QSISSYLNWYQQKPGKAPKLLIYAA |
| | GLEWVSGISGNGGSTNYADSV | STLQTGVPSRFSGSGSGTDFTLTISSL |
| | KGRFTISRDNSKNTLYLQMNSL | QPEDFATYYCQQSYTTPYTFGQGTK |
| | RAEDTAVYYCAKDAYYYGSG | LEIK (SEQ ID NO: 636) |
| | SYFDYWGQGTLVTVSS (SEQ | |
| | ID NO: 635) | |
| 2E10 | QVQLVQSGAEVKKPGASVKVS | DIQMTQSPSSLSASVGDRVTITCRAS |
| | CKASGYTFTSYDINWVRQAPG | QSIRSWLAWYQQKPGKAPKLLIYAA |
| | QGLEWMGWINPNSGGTNYAQ | SSLQSGVPSRFSGSGSGTDFTLTISSL |
| | KFQGRVTMTRDTSTSTVYMEL | QPEDFATYYCQQSYSTPYTFGQGTK |
| | SSLRSEDTAVYYCARGEWLDY | LEIK (SEQ ID NO: 638) |
| | WGQGTLVTVSS (SEQ ID NO: | |
| | 637) | |
| 2A06 | QVQLVQSGAEVKKPGASVKVS | DIQMTQSPSSLSASVGDRVTITCRAS |
| | CKASGYTFSSYGISWVRQAPG | QDISNWLAWYQQKPGKAPKLLIYG |
| | QGLEWMGWINPNSGGTNYAQ | ASTLQSGVPSRFSGSGSGTDFTLTISS |
| | KLQGRVTMTRDTSTSTVYMEL | LQPEDFATYYCQQSYSTPYTFGQGT |
| | SSLRSEDTAVYYCTGDYSWFD | KLEIK (SEQ ID NO: 640) |
| | PWGQGTLVTVSS (SEQ ID NO: | |
| | 639) | |
| 2C04 | EVQLLESGAEVKKPGASVKVS | DIQMTQSPSSLSASVGDRVTITCRAS |
| | CKASGYTFTSYGISWVRQAPG | QSIGRWLAWYQQKPGKAPKLLIYAT |
| | QGLEWMGWINPNSGGTNYAQ | SSLQSGVPSRFSGSGSGTDFTLTISSL |
| | KFQGRVTMTRDTSTSTVYMEL | QPEDFATYYCQQSYSTPYTFGQGTK |
| | SSLRSEDTAVYYCARANWLDY | LEIK (SEQ ID NO: 642) |
| | WGQGTLVTVSS (SEQ ID NO: | |
| | 641) | |
| 2D02 | QVQLVQSGAEVKKPGASVKVS | DIQMTQSPSSLSASVGDRVTITCRAS |
| | CKASGYTFTSYGISWVRQAPG | QSISNWLAWYQQKPGKAPKLLIYGA |
| | QGLEWMGWINPNSGGTNYAQ | SNLQPGVPSRFSGSGSGTDFTLTISSL |
| | RLQGRVTMTRDTSTSTVYMEL | QPEDFATYYCQQSYSTPYSFGQGTK |
| | SSLRSEDTAVYYCARDFSWFDP | LEIK (SEQ ID NO: 644) |
| | WGQGTLVTVSS (SEQ ID NO: | |
| | 643) | |
| 2E05 | QVQLVQSGAEVKKPGASVKVS | DIQMTQSPSSLSASVGDRVTITCRAS |
| | CKASGYTFTSYGISWVRQAPG | QSISNWLAWYQQKPGKAPKLLIYAA |
| | QGLEWMGWINPNSGGTNYAQ | SNLQSGVPSRFSGSGSGTDFTLTISSL |
| | KFQGRVTMTRDTSTSTVYMEL | QPEDFATYYCQQSYNTPFTFGPGTK |
| | SSLRSEDTAVYYCARGYNWFD | VDIK (SEQ ID NO: 646) |
| | PWGQGTLVTVSS (SEQ ID NO: | |
| | 645) | |
| 2F02 | EVQLLESGAEVKKPGASVKVS | DIQMTQSPSSLSASVGDRVTITCRAS |
| | CKASGYTFTSYGISWVRQAPG | QDISRWLAWYQQKPGKAPKLLIYA |
| | QGLEWMGWINPNSGGTNYAQ | ASSLQSGVPSRFSGSGSGTDFTLTISS |
| | KFQGRVTMTRDTSTSTVYMEL | LQPEDFATYYCQQSYNTPYTFGQGT |
| | SSLRSEDTAVYYCARANWLDY | KLEIK (SEQ ID NO: 647) |
| | WGQGTLVTVSS (SEQ ID NO: | |
| | 641) | |
| 2F07 | QVQLVQSGAEVKKPGASVKVS | DIQMTQSPSSLSASVGDRVTITCRAS |
| | CKASGYTFTSYGISWVRQAPG | QSVGRWMAWYQQKPGKAPKLLIY |
| | QGLEWMGWINPNSGGTNYAQ | AASTLQTGVPSRFSGSGSGTDFTLTI |
| | KFQGRVTMTRDTSTSTVYMEL | SSLQPEDFATYYCQQSYSPPYTFSQG |
| | SSLRSEDTAVYYCARSINWFDP | TKLEIK (SEQ ID NO: 649) |
| | WGQGTLVTVSS (SEQ ID NO: | |
| | 648) | |
| 2G10 | EVQLLESGAEVKKPGASVKVS | DIQMTQSPSSLSASVGDRVTITCRAS |
| | CKASGYTFTSYGISWVRQAPG | QSISSWLAWYQQKPGKAPKLLIYAA |
| | QGLEWMGWINPHSGGTFYAQ | SRLQSGVPSRFSGSGSGTDFTLTISSL |
| | KFQGRVTMTRDTSTSTVYMEL | QPEDFATYYCQQSYSPPYTFGQGTK |
| | SSLRSEDTAVYYCARANWLDY | LEIK (SEQ ID NO: 651) |
| | WGQGTLVTVSS (SEQ ID NO: | |
| | 650) | |
| 2E03 | QVQLVQSGAEVKKPGASVKVS | DIQMTQSPSSLSASVGDRVTITCRAS |
| | CKASGYTLTSYLMHWVRQAP | QTIGNWLAWYQQKPGKAPKLLIYG |
| | GQGLEWMGWINPYSGGTNYA | ASSLQSGVPSRFSGSGSGTDFTLTISS |
| | QKFQGRVTMTRDTSTSTVYME | LQPEDFATYYCQQSYSPPLTFGPGT |
| | LSSRRSEDTAVYYCARDRGSG | KVDIK (SEQ ID NO: 653) |
| | YDYWGQGTLVTVSS (SEQ ID | |
| | NO: 652) | |
| 2H06 | QVQLVQSGAEVKKPGASVKVS | DIQMTQSPSSLSASVGDRVTITCRAS |
| | CKASGYTFTSYNMHWVRQAP | QSISRWLAWYQQKPGKAPKLLIYAA |
| | GQGLEWMGWINPNSGGTNYA | STLQSGVPSRFSGSGSGTDFTLTISSL |
| | QKLQGRVTMTRDTSTSTVYME | QPEDFATYYCQQSYITPYTFGQGTK |
| | LSSLRSEDTAVYYCARANWLD | LEIK (SEQ ID NO: 509) |
| | YWGQGTLVTVSS (SEQ ID NO: | |
| | 654) | |
| 2F09 | EVQLLESGAEVKKPGASVKVS | DIQMTQSPSSLSASVGDRVTITCRAS |
| | CKASGYIFTSYPIHWVRQAPGQ | QSISRWLAWYQQKPGKAPKLLIYAA |
| | GLEWMGWINPNSGGTNYAQK | SNLQSGVPSRFSGSGSGTDFTLTISSL |
| | FQGRVTMTRDTSTSTVYMELS | QPEDFATYYCQQSYSPPYTFGQGTK |
| | SLRSEDTAVYYCARANWLDY | LEIK (SEQ ID NO: 656) |
| | WGQGTLVTVSS (SEQ ID NO: | |
| | 655) | |
| 2E08 | QVQLVQSGAEVKKPGASVKVS | DIQMTQSPSSLSASVGDRVTITCRAS |
| | CKASGGTFTSYPISWVRQAPGQ | QSIGRWLAWYQQKPGKAPKLLIYA |
| | GLEWMGWINPSNGGTNYAQK | ASTLQSGVPSRFSGSGSGTDFTLTISS |
| | FQGRVTMTRETSTSTAYMELSS | LQPEDFATYYCQQSYSTPYTFGQGT |
| | LRSEDTAVYYCARSNWLDYW | KLEIK (SEQ ID NO: 658) |
| | GQGTLVTVSS (SEQ ID NO: 657) | |
| 2F08 | EVQLVQSGAEVKKPGASVKVS | DIQMTQSPSSLSASVGDRVTITCRTS |
| | CKASGGTFSSYTISWVRQAPGQ | QGIGTWLAWYQQKPGKAPKLLIYA |
| | GLEWMGWISPNSGGTNYAQKF | ASSLQSGVPSRFSGSGSGTDFTLTISS |
| | QGRVTMTRDTSTSTVYMELSS | LQPEDFATYYCQQSYSTPYTFGQGT |
| | LRSEDTAVYYCARANWLDYW | KLEIK (SEQ ID NO: 660) |
| | GQGTLVTVSS (SEQ ID NO: 659) | |
| 2C09 | QVQLVQSGAEVKKPGASVKVS | DIQMTQSPSSLSASVGDRVTITCRAS |
| | CKASGGTFSTFPMHWVRQAPG | QSISSWLAWYQQKPGKAPKLLIYAA |
| | QGLEWMGWINPNSGGTNYAQ | STLQSGVPSRFSGSGSGTDFTLTISSL |
| | KFQGRVTMTRDTSTSTVYMEL | QPEDFATYYCQQSYNPPYTFGQGTK |
| | SSLRSEDTAVYYCARGEWLGY | LEIK (SEQ ID NO: 662) |
| | WGQGTLVTVSS (SEQ ID NO: | |
| | 661) | |
| 2B06 | EVQLLESGAEVKKPGASVKVS | DIQMTQSPSSLSASVGDRVTITCRAS |
| | CKASGYSFTTYGISWVRQAPG | QDISRWSAWYQQKPGKAPKLLIYG |
| | QGLEWMGWINPNSGGTNYAQ | ASSLQSGVPSRFSGSGSGTDFTLTISS |
| | KFQGRVTMTRDTSTSTVYMEL | LQPEDFATYYCQQSYSPPYTFGQGT |
| | SSLRSEDTAVYYCARANWLDY | KLEIK (SEQ ID NO: 664) |
| | WGQGTLVTVSS (SEQ ID NO: | |
| | 663) | |
| 2F10 | QVQLVQSGAEVKKPGASVKVS | DIQMTQSPSSLSASVGDRVTITCRAS |
| | CKASGYTFTTYNIHWVRQAPG | QEIRRWLAWYQQKPGKAPKLLIYA |
| | QGLEWMGWINPNSGGTNYAQ | ASNLETGVPSRFSGSGSGTDFTLTISS |
| | KFQGRVTMTRDTSTSTVYMEL | LQPEDFATYYCQQSYSIPYTFGQGT |
| | SSLRSEDTAVYYCARANWLDY | KLEIK (SEQ ID NO: 666) |
| | WGQGTLVTVSS (SEQ ID NO: | |
| | 665) | |
| 2E07 | QVQLVQSGAEVKKPGASVKVS | DIQMTQSPSSLSASVGDRVTITCRAS |
| | CKASGGTFSTYTVSWVRQAPG | QGISRWLAWYQQKPGKAPKLLIYA |
| | QGLEWMGWINPNSGGTNYAQ | ASTLQSGVPSRFSGSGSGTDFTLTISS |
| | KFQGRVTMTRDTSTSTVYMEL | LQPEDFATYYCQQSYETPYTFGQGT |
| | SSLRSEDTAVYYCARGDWFDP | KVEIK (SEQ ID NO: 668) |
| | WGQGTLVTVSS (SEQ ID NO: | |
| | 667) | |
| 3F02 | QVQLVQSGAEVKKPGASVKVS | DIQMTQSPSSLSASVGDRVTITCRAS |
| | CKASGYTFTAYYMHWVRQAP | QDISRWLAWYQQKPGKAPKLLIYA |
| | GQGLEWMGWISPNSGGTNYA | ASTLQSGVPSRFSGSGSGTDFTLTISS |
| | QKFQGRVTMTRDTSTSTVYME | LQPEDFATYYCQQGYSTPYTFGQGT |
| | LSSLRSEDTAVYYCARGDWND | KLEIK (SEQ ID NO: 670) |
| | WWGQGTLVTVSS (SEQ ID NO: | |
| | 669) | |
| 3H02 | QVQLVQSGAEVKKPGASVKVS | DIQMTQSPSSLSASVGDRVTITCRAS |
| | CKASGYTFTDYHLHWVRQAPG | QSISSWLAWYQQKPGKAPKLLIYAA |
| | QGLEWMGWINPNSGGTNYAQ | STLQSGVPSRFSGSGSGTDFTLTISSL |
| | MFQGRVTMTRDTSTSTVYMEL | QPEDFATYYCQQSYSTPYTFGQGTK |
| | SSLRSEDTAVYYCARGEWFDP | LEIK (SEQ ID NO: 672) |
| | WGQGTLVTVSS (SEQ ID NO: | |
| | 671) | |
| 3A09 | QVQLVQSGAEVKKPGASVKVS | DIQMTQSPSSLSASVGDRVTITCRAS |
| | CKASGYTFTDYIVHWVRQAPG | QSISSYLNWYQQKPGKAPKLLIYGV |
| | QGLEWMGWINPYSGGTKYAQ | SSLQSGVPSRFSGSGSGTDFTLTISSL |
| | KFQGRVTMTRDTSTSTVYMEL | QPEDFATYYCQQSYSNPTFGQGTKV |
| | SSLRSEDTAVYYCARVLQEGM | EIK (SEQ ID NO: 674) |
| | DVWGQGTLVTVSS (SEQ ID | |
| | NO: 673) | |
| 3G05 | EVQLLESGAEVKKPGASVKVS | DIQMTQSPSSLSASVGDRVTITCRAS |
| | CKASGYTFTDYIVHWLRQAPG | QSISRWLAWYQQKPGKAPKLLIYGA |
| | QGLEWMGWINPNSGGTNYAQ | SSLQSGVPSRFSGSGSGTDFTLTISSL |
| | NFQGRVTMTRDTSTSTVYMEL | QPEDFATYYCQQSYITPYTFGQGTK |
| | SSLRSEDTAVYYCARANWLDY | VEIK (SEQ ID NO: 676) |
| | WGQGTLVTVSS (SEQ ID NO: | |
| | 675) | |
| 3F07 | QVQLVQSGAEVKKPGASVKVS | DIQMTQSPSSLSASVGDRVTITCRAS |
| | CKASGYTFTDYLIHWVRQAPG | QSISRWLAWYQQKPGKAPKLLIYAA |
| | QGLEWMGWINPASGGTNYAQ | STLQSGVPSRFSGSGSGTDFTLTISSL |
| | KFQGRVTMTRDTSTSTVYMEL | QPEDFATYYCQQSYSPPYTFGQGTK |
| | SSLRSEDTAVYYCAGGYRWLH | LEIK (SEQ ID NO: 678) |
| | DYWGQGTLVTVSS (SEQ ID | |
| | NO: 677) | |
| 3A06 | EVQLLESGAEVKKPGSSVKVSC | DIQMTQSPSSLSASVGDRVTITCRAS |
| | KASGYTFTDYNIHWVRQAPGQ | QDISLWLAWYQQKPGKAPKLLIYA |
| | GLEWMGWINPNSGGTNYAQR | ASTLQSGVPSRFSGSGSGTDFTLTISS |
| | FQGRVTMTRDTSTSTVYMELS | LQPEDFATYYCQQSYMTPRTFGPGT |
| | SLRSEDTAVYYCARANWLDY | KVDIK (SEQ ID NO: 680) |
| | WGQGTLVTVSS (SEQ ID NO: | |
| | 679) | |
| 3G01 | QVQLVQSGAEVKKPGASVKVS | DIQMTQSPSSLSASVGDRVTITCRAS |
| | CKASGYTFTDYQMFWVRQAP | QGISNYLAWYQQKPGKAPKLLIYAA |
| | GQGLEWMGWINPNSGGTNYA | SSLQSGVPSRFSGSGSGTDFTLTISSL |
| | QKFQGRVTMTRDTSTSTVYME | QPEDFATYYCQQSYSDQWTFGQGT |
| | LSSLRSEDTAVYYCAKGGGGY | KVEIK (SEQ ID NO: 682) |
| | GMDVWGQGTTVTVSS (SEQ ID | |
| | NO: 681) | |
| 3D02 | QVQLVQSGAEVKKPGASVKVS | DIQMTQSPSSLSASVGDRVTITCRAS |
| | CKASGDTFSFYAISWVRQAPG | QSIGRWLAWYQQKPGKAPKLLIYA |
| | QGLEWMGWMNPNSGGTTYAQ | ASTLQTGVPSRFSGSGSGTDFTLTISS |
| | KFQGRVTMTRDTSTSTVYMEL | LQPEDFATYYCQQGYSSPYTFGQGT |
| | SSLRSEDTAVYYCARGVQDYY | KVEIK (SEQ ID NO: 684) |
| | GSDYWGQGTLXTVSS (SEQ ID | |
| | NO: 683) | |
| 3F01 | QVQLVQSGAEVKKPGASVQVS | DIQMTQSPSSLSASVGDRVTITCQAS |
| | CKASGYTFTGYNIHWVRQAPG | QDISNYLNWYQQKPGKAPKLLIYAA |
| | QGLEWMGWINPNSGGTNYAQ | STLESGVPSRFSGSGSGTDFTLTISSL |
| | KFQGRVTMTRDTSTSTVYMEL | QPEDFATYYCQQSFTIPWTFGQGTK |
| | SSLRSEDTAVYYCARDEAGYF | LEIK (SEQ ID NO: 686) |
| | DLWGRGTLVTVSS (SEQ ID | |
| | NO: 685) | |
| 3B07 | EVQLLESGAEVKKPGASVKVS | DIQMXQSPSSLSASVGDRVTITCRAS |
| | CKASGYTFTGYNMHWVRQAP | QSINRWLAWYQQKPGKAPKLLIYA |
| | GQGLEWMGWINPNSGGTNYA | ASTLQSGVPSRFSGSGSGTDFTLTISS |
| | QKFQGRVTMTRDTSTSTVYME | LQPEDFATYYCQQSYITPYTFGQGT |
| | LSSLRSEDTAVYYCARANWLD | KLEIK (SEQ ID NO: 687) |
| | YWGQGTLVTVSS (SEQ ID NO: | |
| | 587) | |
| 3F06 | QVQLVQSGAEVKKPGASVKVS | DIQMTQSPSSLSASVGDRVTITCRAS |
| | CKASGYTFTGYNMHWVRQAP | QSISSWLAWYQQKPGKAPKLLIYAA |
| | GQGLEWMGWINPNSGGTNYA | SSLQSGVPSRFSGSGSGTDFTLTISSL |
| | QKFQGRVTMTRDTSTSTVYME | QPEDFATYYCQQSYSPPYTFGQGTK |
| | LSSLRSEDTAVYYCARGDWFD | VDIK (SEQ ID NO: 689) |
| | PWGQGTLVTVSS (SEQ ID NO: | |
| | 688) | |
| 3A10 | QVQLVQSGAEVKKPGASVKVS | DIQMTQSPSSLSASVGDRVTITCRAS |
| | CKASGYMFTGYNVHWVRQAP | QGISSWLAWYQQKPGKAPKLLIYAA |
| | GQGLEWMGWINPNSGGTNYA | SSLQSGVPSRFSGSGSGTDFTLTISSL |
| | QKFQGRVTMTRDTSTSTVYME | QPEDFATYYCQQSYSTPYTFGQGTK |
| | LSSLRSEDTAVYYCARDHSSGY | LEIK (SEQ ID NO: 691) |
| | DYWGQGTLVTVSS (SEQ ID | |
| | NO: 690) | |
| 3C02 | QVQLVQSGAEVKKPGASVKVS | DIQMTQSPSSLSASVGDRVTITCRAS |
| | CKASGYTLTGYYIHWVRQAPG | QSISRWLTWYQQKPGKAPKLLIYAA |
| | QGLEWMGWINPNSGGTNYAQ | SSLQSGVPSRFSGSGSGTDFTLTISSL |
| | KFQGRVTMTRDTSTSTVYMEL | QPEDFATYYCQQSYSPPXTFGRGTK |
| | SSLRSEDTAVYYCARENYFDX | VEIK (SEQ ID NO: 693) |
| | XXWGXXTTVTASS (SEQ ID | |
| | NO: 692) | |
| 3E02 | QVQLVQSGAEVKKPGASVKVS | DIQMTQSPSSLSASVGDRVTITCRAS |
| | CKASGYSFTGYYINWVRQAPG | QDISNWLAWYQQKPGKAPKLLIYG |
| | QGLEWMGWINPNSGGTNYAQ | ASTLQSGVPSRFSGSGSGTDFTLTISS |
| | KFQGRVTMTRDTSTSTVYMEL | LQPEDFATYYCQQSMIAPYTFGQGT |
| | SSLRSEDTAVYYCAAGEWFDP | KVEIK (SEQ ID NO: 695) |
| | WGQGTLVTVSS (SEQ ID NO: | |
| | 694) | |
| 3D11 | QVQLVQSGAEVKKPGASVKVS | DIQMTQSPSSLSASVGDRVTITCQAS |
| | CKASGYTFTGYYMHWVRQAP | QSIYNYLNWYQQKPGKAPKLLIYAA |
| | GQGLEWMGWMNPNSGNTGY | SSLQSGVPSRFSGSGSGTDFTLTISSL |
| | AQKFQGRVTMTRDTSTSTVYM | QPEDFATYYCQQSYSVPHTFGQGTK |
| | ELSSLRSEDTAVYYCARGSEIID | LEIK (SEQ ID NO: 697) |
| | YDSSGLGAFDIWGQGTMVTVS | |
| | S (SEQ ID NO: 696) | |
| 3A05 | EVQLLESGAEVKKPGASVKVS | DIQMTQSPSSLSASVGDRVTITCRAS |
| | CKASGFTFTNHNMHWVRQAP | QSIDRWLAWYQQKPGKAPKLLIYA |
| | GQGLEWMGWINPNSGGTNYA | ASSLQSGVPSRFSGSGSGTDFTLTISS |
| | QKFQGRVTMTRDTSTSTVYME | LQPEDFATYYCQQSYSIPYTFGQGT |
| | LSSLRSEDTAVYYCARANWLD | KLEIK (SEQ ID NO: 491) |
| | YWGQGTLVTVSS (SEQ ID NO: | |
| | 490) | |
| 3A08 | EVQLLESGAEVKKPGASVKVS | DIQMTQSPSSLSASVGDRVTITCRAS |
| | CKASGFTFTNHNMHWVRQAP | QSIDRWLAWYQQKPGKAPKLLIYA |
| | GQGLEWMGWINPNSGGTNYA | ASSLQSGVPSRFSGSGSGTDFTLTISS |
| | QKFQGRVTMTRDTSTSTVYME | LQPEDFATYYCQQSYSIPYTFGQGT |
| | LSSLRSEDTAVYYCARANWLD | KLEIK (SEQ ID NO: 491) |
| | YWGQGTLVTVSS (SEQ ID NO: | |
| | 490) | |
| 3F11 | EVQLLESGAEVKKPGASVKVS | DIQMTQSPSSLSASVGDRVTITCRAS |
| | CKASGFTFTNHNMHWVRQAP | QSIDRWLAWYQQKPGKAPKLLIYA |
| | GQGLEWMGWINPNSGGTNYA | ASSLQSGVPSRFSGSGSGTDFTLTISS |
| | QKFQGRVTMTRDTSTSTVYME | LQPEDFATYYCQQSYSIPYTFGQGT |
| | LSSLRSEDTAVYYCARANWLD | KLEIK (SEQ ID NO: 491) |
| | YWGQGTLVTVSS (SEQ ID NO: | |
| | 490) | |
| 3E11 | QVQLVQSGAEVKKPGASVKVS | DIQMTQSPSSLSASVGDRVTITCRAS |
| | CKASGASFSNSIINWVRQAPGQ | QGISNYLAWYQQKPGKAPKLLIYAA |
| | GLEWMGWIYPNSGGTNYAQK | STLQGGVPSRFSGSGSGTDFTLTISSL |
| | FQGRVTMTRDTSTSTVYMELS | QPEDFATYYCQQTYSVPLTFGGGTK |
| | SLRSEDTAVYYCARRWMDAF | VEIK (SEQ ID NO: 699) |
| | DIWGQGTTVTVSS (SEQ ID NO: | |
| | 698) | |
| 3A03 | EVQLLESGAEVKKPGASVKVS | DIQMTQSPSSLSASVGDRVTITCRAS |
| | CKASGYTFINYGITWVRQAPG | QSIGSWLAWYQQKPGKAPKLLIYAA |
| | QGLEWMGWINANSGGTNYAQ | SSLQSGVPSRFSGSGSGTDFTLTISSL |
| | KFQGRVTMTRDTSTSTVYMEL | QPEDFATYYCQQSYSAPYTFGQGTK |
| | SSLRSEDTAVYYCARANWLDY | LEIK (SEQ ID NO: 701) |
| | WGQGTLVTVSS (SEQ ID NO: | |
| | 700) | |
| 3D03 | QVQLVQSGAEVKKPGASVKVS | DIQMTQSPSSLSASVGDRVTITCRAS |
| | CKASGGTFSNYILSWVRQAPG | QSISRWLAWYQQKPGKAPKLLIYAA |
| | QGLEWMGWINPDSGGTNYAQ | SSLQSGVPSRFSGSGSGTDFTLTISSL |
| | KFQGRVTMTRDTSTSTVYMEL | QPEDFATYYCQQSYSTPYTFGQGTK |
| | SSLRSEDTAVYYCARGSYGMD | LEIK (SEQ ID NO: 568) |
| | VWGQGTTVTVSS (SEQ ID NO: | |
| | 702) | |
| 3E03 | QVQLVQSGAEVKKPGASVKVS | DIQMTQSPSSLSASVGDRVTITCRAS |
| | CKASGYTFTNYNIHWVRQAPG | QSISNWLAWYQQKPGKAPKLLIYAA |
| | QGLEWMGWINPNSGGTNYAQ | STLQSGVPSRFSGSGSGTDFTLTISSL |
| | RFQGRVTMTRDTSTSTVYMEL | QPEDFATYYCQQSYISPYTFGPGTKV |
| | SSLRSEDTAVYYCAREGLAAL | DIK (SEQ ID NO: 704) |
| | DYWGQGTLVTVSS (SEQ ID | |
| | NO: 703) | |
| 3D06 | QVQLVQSGAEVKKPGASVKVS | DIQMTQSPSSLSASVGDRVTITCRAS |
| | CKASGYMFTNYPMTWVRQAP | QDISNWLAWYQQKPGKAPKLLIYA |
| | GQGLEWMGWINPNSGGTNYA | ASKLQSGVPSRFSGSGSGTDFTLTISS |
| | QKFQGRVTMTRDTSTSTVYME | LQPEDFATYYCQQSYSPPYSFGQGT |
| | LSSLRSEDTAVYYCARDAAAG | KLEIK (SEQ ID NO: 706) |
| | FDPWGQGTLVTVSS (SEQ ID | |
| | NO: 705) | |
| 3G08 | QVQLVQSGAEVKKPGASVKVS | DIQMTQSPSSLSASVGDRVTITCRAS |
| | CKASGGTFSNYVISWVRQAPG | QSIGRWLAWYQQKPGKAPKLLIYG |
| | QGLEWMGWINPNSGGTNFAQ | ASSVQSGVPSRFSGSGSGTDFTLTISS |
| | KFQGRVTMTRDTSTSTVYMEL | LQPEDFATYYCQQSYSSPYTFGQGT |
| | SSLRSEDTAVYYCAREGRSSYD | KLEIK (SEQ ID NO: 708) |
| | YWGQGTLVTVSS (SEQ ID NO: | |
| | 707) | |
| 3A01 | QVQLVQSGAEVKKPGASVKVS | DIQMTQSPSSLSASVGDRVTITCQAS |
| | CKASGYTFTNYYMHWVRQAP | QDIRKYLNWYQQKPGKAPKLLIYA |
| | GQGLEWMGWMNPNSGHTSYA | ASSLQSGVPSRFSGSGSGTDFTLTISS |
| | QKFQGRVTMTRDTSTSTVYME | LQPEDFATYYCQQSYTIPYTFGQGT |
| | LSSLRSEDTAVYYCARVGYSSG | KLEIK (SEQ ID NO: 710) |
| | WLGMDVWGQGTTVTVSS | |
| | (SEQ ID NO: 709) | |
| 3B09 | EVQLLESGAEVKKPGASVKVS | DIQMTQSPSSLSASVGDRVTITCRAS |
| | CKASGYTLTSHLIHWVRQAPG | QDISRWLAWYQQKPGKAPKLLIYA |
| | QGLEWVGWINPNTGGTNYAQ | ASSLQSGVPSRFSGSGSGTDFTLTISS |
| | KFQGRVTMTRDTSTSTVYMEL | LQPEDFATYYCQQSYTTPYTFGQGT |
| | SSLRSEDTAVYYCARANWLDY | KLEIK (SEQ ID NO: 712) |
| | WGQGTLVTVSS (SEQ ID NO: | |
| | 711) | |
| 3E04 | QVQLVQSGAEVKKPGSSVKVS | DIVMTQSPLSLPVTPGEPASISCRSSQ |
| | CKASGYTFSSSDINWVRQAPG | SLLNKNGNNYIDWYLQKPGQSPQLL |
| | QGLEWMGGIIPIFGTANYAQKF | IYQGSNRASGVPDRFSGSGSGTDFTL |
| | QGRVTITADESTSTAYMELSSL | KISRVEAEDVGVYYCMQALQTPPTF |
| | RSEDTAVYYCARADTAMGHY | GGGTKVEIK (SEQ ID NO: 714) |
| | YYYGMDVWGQGTTVTVSS | |
| | (SEQ ID NO: 713) | |
| 3B06 | QVQLVQSGAEVKKPGASVKVS | DIQMTQSPSSLSASVGDRVTITCRAS |
| | CKASGGTFSSYAFSWVRQAPG | QSISRWLAWYQQKPGKAPKLLIYAA |
| | QGLEWMGVIYPSGGTTGYAQK | SSLQSGVPSRFSGSGSGTDFTLTISSL |
| | FQGRVTMTRDTSTSTVYMELS | QPEDFATYYCQQSYSSPYTFGQGTK |
| | SLRSEDTAVYYCARGLHQLPN | VEIK (SEQ ID NO: 716) |
| | WEDPWGQGTLVTVSS (SEQ ID | |
| | NO: 715) | |
| 3C04 | QVQLVQSGAEVKKPGASVKVS | DIQMTQSPSSLSASVGDRVTITCRAS |
| | CKASGGTFSSYAISWVRQAPG | QNITTYLNWYQQKPGKAPKLLIYAA |
| | QGLEWMGWINPNSGGTNYAQ | STLQSGVPSRFSGSGSGTDFTLTISSL |
| | KFQGRVTMTRDTSTSTVYMEL | QPEDFATYYCQQSYSMPYSFGQGTK |
| | SSLRSEDTAVYYCARDLVGGV | LEIK (SEQ ID NO: 718) |
| | FAFDIWGQGTTVTVSS (SEQ ID | |
| | NO: 717) | |
| 3D01 | QVQLVQSGAEVKKPGASVKVS | DIVMTQSPDSLAVSLGERATINCKSS |
| | CKASGGTFSSYAISWVRQAPG | QSVLYSSNNKNYLAWYQQKPGQPP |
| | QGLEWMGGIIPIFGAPVYAQDF | KLLIYWASTRASGVPDRFSGSGSGT |
| | QGRVTITADESTSTAYMELSSL | DFTLTISSLQAEDVAVYYCQQYERT |
| | RSEDTAVYYCARDRGADAFDI | PLTFGGGTKVEIK (SEQ ID NO: 720) |
| | WGQGTTVTVSS (SEQ ID NO: | |
| | 719) | |
| 3D09 | EVQLVQSGAEVKKPGASVKVS | DIQMTQSPSSLSASVGDRVTITCRAS |
| | CKASGGTFSSYAISWVRQAPG | QNINRWLAWYQQKPGKAPKLLIYA |
| | QGLEWMGWINPNSGGTNYAQ | ASTLQSGVPSRFSGSGSGTDFTLTISS |
| | KFQGRVTMTRDTSTSTVYMEL | LQPEDFATYYCQQSYSPPYTFGQGT |
| | SSLRSEDTAVYYCARANWLDY | KLEIK (SEQ ID NO: 499) |
| | WGQGTLVTVSS (SEQ ID NO: | |
| | 634) | |
| 3E08 | EVQLVQSGAEVKKPGASVKVS | DIQMTQSPSSLSASVGDRVTITCRAS |
| | CKASGGTFSSYAISWVRQAPG | QSISRWLAWYQQKPGKAPKLLIYAA |
| | QGLEWIGWINPNSGGTNYAQK | SSLQSGVPSRFSGSGSGTDFTLTISSL |
| | FQGRVTMTRDTSTSTVYMELS | QPEDFATYYCQQSYSPPYTFGQGTK |
| | SLRSEDTAVYYCARANWLDY | LEIK (SEQ ID NO: 722) |
| | WGQGTLVTVSS (SEQ ID NO: | |
| | 721) | |
| 3E09 | QVQLVQSGAEVKKPGASVKVS | DIQMTQSPSSLSASVGDRVTITCRAS |
| | CKASGGTFSSYAISWVRQAPG | QSISNWLAWYQQKPGKAPKLLIYGA |
| | QGLEWMGWINPNSGGTKYAQ | SSLQSGVPSRFSGSGSGTDFTLTISSL |
| | KFQGRVTMTRDTSTSTVYMEL | QPEDFATYYCQQSYTTPYTFGQGTK |
| | SSLRSEDTAVYYCAREISFLDY | LEIK (SEQ ID NO: 724) |
| | WGQGTLVTVSS (SEQ ID NO: | |
| | 723) | |
| 3B05 | QVQLVQSGAEVKKPGASVKVS | DIQMTQSPSSLSASVGDRVTITCRAS |
| | CKASGGTFSSYAVSWVRQAPG | QSISNWLAWYQQKPGKAPKLLIYGA |
| | QGLEWMGWIIPDSGGTNYAQK | STLQSGVPSRFSGSGSGTDFTLTISSL |
| | LQGRVTMTTDTSTGTAYMELR | QPEDFATYYCQQSYSKPTFGQGTKV |
| | SLRSDDTAVYYCTRGSGWYDY | EIK (SEQ ID NO: 726) |
| | WGQGTLVTVSS (SEQ ID NO: | |
| | 725) | |
| 3D04 | QVQLVQSGAEVKKPGASVKVS | DIQMTQSPSSLSASVGDRVTITCRAS |
| | CKASGYTFTSYDINWVRQAPG | QNINRWLAWYQQKPGKAPKLLIYA |
| | QGLEWMGWINPNSGGTNYAQ | ASSLQSGVPSRFSGSGSGTDFTLTISS |
| | KFQGRVTMTRDTSTSTVYMEL | LQPEDFATYYCQQSYSPPYTFGQGT |
| | SSLRSEDTAVYYCARANWLDY | KVEIK (SEQ ID NO: 728) |
| | WGQGTLVTISS (SEQ ID NO: | |
| | 727) | |
| 3D07 | QVQLVQSGAEVKKPGASVKVS | DIQMTQSPSSLSASVGDRVTITCRAS |
| | CKASGYTFTSYDINWVRQAPG | QSIRSWLAWYQQKPGKAPKLLIYAA |
| | QGLEWMGWINPNSGGTNYAQ | SSLQSGVPSRFSGSGSGTDFTLTISSL |
| | KFQGRVTMTRDTSTSTVYMEL | QPEDFATYYCQQSYSTPYTFGQGTK |
| | SSLRSEDTAVYYCARGEWLDY | VEIK (SEQ ID NO: 729) |
| | WGQGTLVTVSS (SEQ ID NO: | |
| | 637) | |
| 3F04 | EVQLLESGAEVKKPGASVKVS | DIQMTQSPSSLSASVGDRVTITCRAS |
| | CKASGYTFSSYGIFWVRQAPG | QDIGSWLAWYQQKPGKAPKLLIYA |
| | QGLEWMGWINPNSGGTNYAQ | ASSLQSGVPSRFSGSGSGTDFTLTISS |
| | KFQGRVTMTRDTSTSTVYMEL | LQPEDFATYYCQQSYSMITFGQGTK |
| | SSLRSEDTAVYYCARANWLDY | VEIK (SEQ ID NO: 731) |
| | WGQGTLVTVSS (SEQ ID NO: | |
| | 730) | |
| 3B02 | QVQLVQSGAEVKKPGASVKVS | DIQMTQSPSSLSASVGDRVTITCRAS |
| | CKASGYTFTSYGISWVRQAPG | EAIGTWLAWYQQKPGKAPKLLIYA |
| | QGLEWMGWINPNSGGTNYAQ | ASSLQSGVPSRFSGSGSGTDFTLTISS |
| | KFQGRVTMTRDTSTSTVYMEL | LQPEDFATYYCQQSYSPPYTFGQGT |
| | SSLRSEDTAVYYCARDFDAFDI | KLEIK (SEQ ID NO: 733) |
| | WGQGTMVTVSS (SEQ ID NO: | |
| | 732) | |
| 3B04 | QVQLVQSGAEVKKPGASVKVS | DIQMTQSPSSLSASVGDRVTITCRAS |
| | CKASGYTFTSYGISWVRQAPG | QNIRGYLNWYQQKPGKAPKLLIYA |
| | QGLEWMGWINPNSGGTNYAQ | ASSLQSGVPSRFSGSGSGTDFTLTISS |
| | KFQGRVTMTRDTSTSTVYMEL | LQPEDFATYYCQQSYSNPWTFGQGT |
| | SSLRSEDTAVYYCARSTDYYY | KVEIK (SEQ ID NO: 735) |
| | MDVWGKGTTVTVSS (SEQ ID | |
| | NO: 734) | |
| 3C01 | QVQLVQSGAEVKKPGASVKVS | DIQMTQSPSSLXASVGDRVTITCRAS |
| | CKASGYTFTSYGISWVRQAPG | QSVGSWLAWYQQKPGKAPKLLIYG |
| | QGLEWMGWINPNSGGTNYAQ | ASTLQDGVPSRFSGSGSGTDFTLTIS |
| | NFQGRVTMTRDTSTSTVYMEL | SLQPEDFATYYCQQSYNTPYTFGRG |
| | SSLRSEDTAVYYCARNYDSYD | TKVEIK (SEQ ID NO: 737) |
| | YWGQGTLVTVSS (SEQ ID NO: | |
| | 736) | |
| 3C06 | EVQLLESGAEVKKPGASVKVS | DIQMTQSPSSLSASVGDRVTITCRAS |
| | CKASGYTFTSYGISWVRQAPG | QRIGNWLAWYQQKPGKAPKLLIYA |
| | QGLEWMGWLNPNSGGTNYAQ | ASSLQSGVPSRFSGSGSGTDFTLTISS |
| | QFQGRVTMTRDTSTSTVYMEL | LQPEDFATYYCLQDYNYPYTFGQGT |
| | SSLRSEDTAVYYCARANWLDY | KLEIK (SEQ ID NO: 739) |
| | WGQGTLVTVSS (SEQ ID NO: | |
| | 738) | |
| 3F03 | QVQLVQSGAEVKKPGASVKVS | DIQMTQSPSSLSASVGDRVTITCRAS |
| | CKASGYTFTSYGISWVRQAPG | QAIGSWLAWYQQKPGKAPKLLIYA |
| | QGLEWMGWINPNSGGTNYAQ | ASSLQSGVPSRFSGSGSGTDFTLTISS |
| | KLQGRVTMTRDTSTSTVYMEL | LQPEDFATYYCQQSYSAPYTFGPGT |
| | SSLRSEDTAVYYCARDYGALD | KVDIK (SEQ ID NO: 741) |
| | YWGQGTLVTVSS (SEQ ID NO: | |
| | 740) | |
| 3G04 | QVQLVQSGAEVKKPGASVKVS | DIQMTQSPSSLSASVGDRVTITCRAS |
| | CKASGDTFTSYGISWVRQAPG | QSISSWLAWYQQKPGKAPKLLIYAA |
| | QGLEWMGWIIPNSGGTNYAQK | SSLQSGVPSRFSGSGSGTDFTLTISSL |
| | FQGRVTMTRDTSTSTVYMELS | QPEDFATYYCQQGYSTPYTFGQGTK |
| | SLRSEDTAVYYCARGSGWSDY | LEIK (SEQ ID NO: 743) |
| | WGQGTLVTVSS (SEQ ID NO: | |
| | 742) | |
| G307 | EVQLLESGAEVKKPGASVKVS | DIQMTQSPSSLSASVGDRVTITCRAS |
| | CKASGYTLTSYGISWVRQAPG | QNIGRWLAWYQQKPGKAPKLLIYA |
| | QGLEWMGWINPNSGGTNYAQ | ASSLQSGVPSRFSGSGSGTDFTLTISS |
| | NFQGRVTMTRDTSTSTVYMEL | LQPEDFATYYCRQSYSPPYTFGQGT |
| | SSLRSEDTAVYYCARANWLDY | KLEIK (SEQ ID NO: 745) |
| | WGQGTLVTVSS (SEQ ID NO: | |
| | 744) | |
| 3E01 | EVQLLESGAEVKKPGSSVKVSC | DIQMTQSPSSLSASVGDRVTITCRAS |
| | KASGYTFSSYNMHWVRQAPG | QSISSWLAWYQQKPGKAPKLLIYGA |
| | QGLEWMGWINPNSGGTNYAQ | SNLQTGVPSRFSGSGSGTDFTLTISSL |
| | KFQGRVTMTRDTSTSTVYMEL | QPEDFATYYCQQSYSPPYTFGQGTK |
| | SSLRSEDTAVYYCARANWLDY | VEIK (SEQ ID NO: 747) |
| | WGQGTLVTVSS (SEQ ID NO: | |
| | 746) | |
| 3F08 | EVQLLESGAEVKKPGSSVKVSC | DIQMTQSPSSLSASVGDRVTITCRAS |
| | KASGGTFSSYPISWVRQAPGQG | QSISRWLAWYQQKPGKAPKLLIYAT |
| | LEWMGWINPNSGGTNYAQNF | STLQSGVPSRFSGSGSGTDFTLTISSL |
| | QGRVTITADESTSTAYMELSSL | QPEDFATYYCQQSYSIPYAFGQGTK |
| | RSEDTAVYYCARANWLDYWG | VEIK (SEQ ID NO: 749) |
| | QGTLVTVSS (SEQ ID NO: 748) | |
| 3C09 | EVQLLESGAEVKKPGASVKVS | DIQMTQSPSSLSASVGDRVTITCRAS |
| | CKASGYTFSSYTISWVRQAPGQ | QTISRWLAWYQQKPGKAPKLLIYAA |
| | GLEWMGWINPNSGGTNYAQK | STLQSGVPSRFSGSGSGTDFTLTISSL |
| | FQGRVTMTRDTSTSTVYMELS | QPEDFATYYCQQSYSTPYTFGQGTK |
| | SLRSEDTAVYYCARANWLDY | LEIK (SEQ ID NO: 751) |
| | WGQGTLVTVSS (SEQ ID NO: | |
| | 750) | |
| 3A02 | QVQLVQSGAEVKKPGASVKVS | DIQMTQSPSSLSASVGDRVTITCRAS |
| | CKASGGTFSSYVISWVRQAPG | QSISKWLAWYQQKPGKAPKLLIYAA |
| | QGLEWMGWINPNNGGTNYAQ | SSLQSGVPSRFSGSGSGTDFTLTISSL |
| | KFQGRVTMTRDTSTSTVYMEL | QPEDFATYYCQQSYSAPYTFGQGTK |
| | SSLRSEDTAVYYCARDSGVGM | VEIK (SEQ ID NO: 753) |
| | DVWGQGTTVTVSS (SEQ ID | |
| | NO: 752) | |
| 3F05 | QVQLVQSGAEVKKPGASVKVS | DIQMTQSPSSLSASVGDRVTITCRAS |
| | CKASGGTFSSYVISWVRQAPG | QSISSWLAWYQQKPGKAPKLLIYAA |
| | QGLEWMGWINPNSGGTNYAQ | SSLQSGVPSRFSGSGSGTDFTLTISSL |
| | KFQGRVTMTRDTSTSTVYMEL | QPEDFATYYCQQSYSSPYTFGQGTK |
| | SSLRSEDTAVYYCARDSAGMD | VEIK (SEQ ID NO: 755) |
| | VWGQGTTVTVSS (SEQ ID NO: | |
| | 754) | |
| 3E07 | QVQLVQSGAEVKKPGASVKVS | DIQMTQSPSSLSASVGDRVTITCRAS |
| | CKASGGTFSSYVITWVRQAPG | QNINTWLAWYQQKPGKAPKLLIYA |
| | QGLEWMGWINPNSGGTNYAQ | ASTLQSGVPSRFSGSGSGTDFTLTISS |
| | EFQGRVTMTRDTSTSTVYMEL | LQPEDFATYYCQQSYIAPYTFGQGT |
| | SSLRSEDTAVYYCARGHMALD | KLEIK (SEQ ID NO: 757) |
| | YWGQGTLVTVSS (SEQ ID NO: | |
| | 756) | |
| 3E05 | EVQLLESGAEVKKPGASVKVS | DIQMTQSPSSLSASVGDRVTITCRAS |
| | CKASGYTFTSYVMNWVRQAP | QGISSWLAWYQQKPGKAPKLLIYAA |
| | GQGLEWMGWINPNSGGTSYA | SSLQSGVPSRFSGSGSGTDFTLTISSL |
| | QKFQGRVTMTRDTSTSTVYME | QPEDFATYYCQQSYSPPYTFGQGTK |
| | LSSLRSEDTAVYYCARANWLD | LEIK (SEQ ID NO: 759) |
| | YWGQGTTVTVSS (SEQ ID NO: | |
| | 758) | |
| 3F09 | QVQLVQSGAEVKKPGASVKVS | DIQMTQSPSSLSASVGDRVTITCRAS |
| | CKASGYTFTSYYLHWVRQAPG | QGISSWLAWYQQKPGKAPKLLIYAA |
| | QGLEWMGIINPSGGSTSYAQKF | SSLQSGVPSRFSGSGSGTDFTLTISSL |
| | QGRVTMTRDTSTSTVYMELSS | QPEDFATYYCQQAYSFPYTFGQGTK |
| | LRSEDTAVYYCAKDTEGYGSG | VEIK (SEQ ID NO: 761) |
| | SRPYGMDVWGQGTLVTVSS | |
| | (SEQ ID NO: 760) | |
| 3C07 | QVQLVQSGAEVKKPGASVKVS | DIQMTQSPSSLSASVGDRVTITCRAS |
| | CKASGYTFTSYYMHWVRQAP | QSIGTWLAWYQQKPGKAPKLLIYA |
| | GQGLEWMGWINPNSGGTNYA | ASTLQSGVPSRFSGSGSGTDFTLTISS |
| | QKFQGRVTMTRDTSTSTVYME | LQPEDFATYYCQQSYSTPYTFGQGT |
| | LSSLRSEDTAVYYCARGEWFD | KLEIK (SEQ ID NO: 763) |
| | PWGQGTLVTVSS (SEQ ID NO: | |
| | 762) | |
| 3C08 | QVQLVQSGAEVKKPGASVKVS | DIQMTQSPSSLSASVGDRVTITCRAS |
| | CKASGYTFTSYYMHWVRQAP | QSISSWLAWYQQKPGKAPKLLIYAA |
| | GQGLEWMGWINPNSGGTNYA | SSLQSGVPSRFSGSGSGTDFTLTISSL |
| | QKFQGRVTMTRDTSTSTVYME | QPEDFATYYCQQSYRTPYTFGQGTK |
| | LSSLRSEDTAVYYCARGEWLD | LEIK (SEQ ID NO: 764) |
| | YWGQGTLVTVSS (SEQ ID NO: | |
| | 554) | |
| 3G10 | QVQLVQSGAEVKKPGASVKVS | DIQMTQSPSSLSASVGDRVTITCRAS |
| | CKASGYTFTSYYMHWVRQAP | QSIGTWLAWYQQKPGKAPKLLIYA |
| | GQGLEWMGWINPNSGGTNYA | ASTLQSGVPSRFSGSGSGTDFTLTISS |
| | QKFQGRVTMTRDTSTSTVYME | LQPEDFATYYCQQSYSTPYTFGQGT |
| | LSSLRSEDTAVYYCARGEWFD | KLEIK (SEQ ID NO: 763) |
| | PWGQGTLVTVSS (SEQ ID NO: | |
| | 762) | |
| 3H01 | QVQLVQSGAEVKKPGASVKVS | DIQMTQSPSSLSASVGDRVTITCQAS |
| | CKASGYTFTTYDISWVRQAPG | QDISNYLNWYQQKPGKAPKLLIYAA |
| | QGLEWMGWMSPNSGGTNYAQ | SSLRGGVPSRFSGSGSGTDFTLTISSL |
| | NFQGRVTMTRDTSTSTVYMEL | QPEDFATYYCQQSYNTPYSFGQGTK |
| | SSLRSEDTAVYYCARSLHPYSN | LEIK (SEQ ID NO: 766) |
| | YWVYWGPGTLVTVSS (SEQ ID | |
| | NO: 765) | |
| 3G03 | QVQLVQSGAEVKKPGASVKVS | DIQMTQSPSSLSASVGDRVTITCRAS |
| | CKASGYSFTTYLIHWVRQAPG | QSISTWLAWYQQKPGKAPKLLIYAA |
| | QGLEWMGWINPNSGGTNYAQ | SSLQSGVPSRFSGSGSGTDFTLTISSL |
| | KFQGRVTMTRDTSTSTVYMEL | QPEDFATYYCQQSYSAPYTFGQGTK |
| | SSLRSEDTAVYYCARANWLDY | LEIK (SEQ ID NO: 768) |
| | WGQGTLVTVSS (SEQ ID NO: | |
| | 767) | |
| 3D10 | QVQLVQSGAEVKKPGASVKVS | DIQMTQSPSSLSASVGDRVTITCRAS |
| | CKASGGTLSTYTISWVRQAPG | QSISRWLAWYQQKPGKAPKLLIYAA |
| | QGLEWMGWMNPNSGGTNYA | STLQSGVPSRFSGSGSGTDFTLTISSL |
| | QKFQGRVTMTRDTSTSTVYME | QPEDFATYYCQQSYSTPYTFGQGTK |
| | LSSLRSEDTAVYYCARGDWQD | LEIK (SEQ ID NO: 529) |
| | IWGQGTTVTVSS (SEQ ID NO: | |
| | 769) | |
| 3B10 | QVQLVQSGAEVKKPGASVKVS | DIQMTQSPSSLSASVGDRVTITCRAS |
| | CKASGYTFSTYTITWVRQAPG | QSIGTWLAWYQQKPGKAPKLLIYA |
| | QGLEWMGWINPKSGGTNYAQ | ASTLQSGVPSRFSGSGSGTDFTLTISS |
| | KFQGRVTMTRDTSTSTVYMEL | LQPEDFATYYCQQSYGTPWTFGQGT |
| | SSLRSEDTAVYYCARGEWLDY | KVEIK (SEQ ID NO: 771) |
| | WGQGTLVTVSS (SEQ ID NO: | |
| | 770) | |
| 3D08 | QVQLVQSGAEVKKPGASVKVS | DIQMTQSPSSLSASVGDRVTITCQAS |
| | CKASGYTFTTYYVHWVRQAPG | QDISNYLNWYQQKPGKAPKLMIYA |
| | QGLEWMGRISPNSGGTNYAQK | ASTLQSGVPSRFSGSGSGTDFTLTISS |
| | FQGRVTMTRDTSTSTVYMELS | LQPEDFATYYCQQSYSTWTFGQGTK |
| | SLRSEDTAVYYCARDWSMDV | VEIK (SEQ ID NO: 773) |
| | WGQGTLVTVSS (SEQ ID NO: | |
| | 772) | |
| 3B11 | QVQLVQSGAEVKKPGASVKVS | DIQMTQSPSSLSASVGDRVTITCRAS |
| | CKASGYTLTXHLIHWVRQAPG | QXVNRWLAWYQQKPGKAPKLLIYA |
| | QGLEWMGWINPNSGGTNYAQ | ASSLQSGVPSRFSGSGSGTDFTLTISS |
| | KLQGRVTMTRDTSTSTVYMEL | LQPEDFATYYCQQSYITPYTFGQGT |
| | SSLRSEDTAVYYCARDYGMDV | KLEIK (SEQ ID NO: 775) |
| | WGXGTTXTVSS (SEQ ID NO: | |
| | 774) | |

*X refers to an unidentified amino acid residue

1. An isolated antigen binding protein (ABP) that specifically binds to a human leukocyte antigen (HLA)-PEPTIDE target, wherein the HLA-PEPTIDE target comprises an HLA-restricted peptide complexed with an HLA Class I molecule, wherein the HLA-restricted peptide is located in the peptide binding groove of an α1/α2 heterodimer portion of the HLA Class I molecule, wherein the HLA Class I molecule is HLA subtype HLA-A*01:01 and the HLA-restricted peptide comprises the sequence NTDNNLAVY (SEQ ID NO: 1), and wherein the ABP binds the HLA-PEPTIDE target with greater affinity as compared to an off-target HLA-PEPTIDE comprising an off-target restricted peptide complexed with an HLA Class I molecule, wherein the off-target restricted peptide is located in the peptide binding groove of an α1/α2 heterodimer portion of the HLA Class I molecule, and wherein the off-target HLA-PEPTIDE is selected from HLA-A*01:01_ETDNNIVVY (SEQ ID NO: 2), HLA-A*01:01_YTDNWLAVY (SEQ ID NO: 3), HLA-A*01:01_GTDNWLAQY (SEQ ID NO: 4), HLA-A*01:01_PTDENLARY (SEQ ID NO: 5), and HLA-A*01:01_NTDNLLTEY (SEQ ID NO: 6).

2. (canceled)

3. The isolated ABP of claim 1, wherein the ABP binds to the HLA-PEPTIDE target with 100-10,000 stronger affinity as compared to the off-target HLA-PEPTIDE A*01:01_ETDNNIVVY (SEQ ID NO: 2), or for which binding to HLA-PEPTIDE A*01:01_ETDNNIVVY (SEQ ID NO: 2) is not detectable

4. (canceled)

5. (canceled)

6. The isolated ABP of claim 1, wherein the ABP binds to the off-target HLA-PEPTIDE A*01:01_ETDNNIVVY (SEQ ID NO: 2) with a Kd that is at least 1 uM or higher, or for which binding is undetectable.

7. (canceled)

8. (canceled)

9. The isolated ABP of claim 1, wherein the ABP comprises the heavy chain CDR3 (HCDR3) and the light chain CDR3 (LCDR3) from the clone designated 1C08, 1A03, 1A07, 1B04, 1C11, 1H06, 1D11, 1G01, 1H04, 1G06, 1B05, 1A08, 1F11, 1A04, 1F08, 1G09, 1F02, 1F04, 1C10, 1E04, 1B07, 1E01, 1F07, 1A10, 1E10, 1D10, 1D07, 1E05, 1F01, 1E08, 1C06, 1H03, 1H08, 1C04, 1D02, 1D05, 1E11, 1F05, 1G11, 1A02, 1A06, 1C05, 1D04, 1E03, 1A11, 1E09, 1C01, 1D09, 1B03, 1H02, 1D03, 1B02, 1F03, 1C09, 1C07, 2G03, 2H05, 2C03, 2F05, 2F04, 2G04, 2C01, 2E01, 2A10, 2G06, 2H07, 2G07, 2B01, 2B10, 2D11, 2G02, 2H08, 2G01, 2E06, 2B02, 2D04, 2A07, 2H04, 2B11, 2F11, 2D08, 2F03, 2A05, 2D07, 2E09, 2G09, 2A04, 2H03, 2E04, 2A09, 2D09, 2B08, 2G11, 2C08, 2C05, 2C07, 2F01, 2H01, 2B07, 2C06, 2A11, 2E10, 2A06, 2C04, 2D02, 2E05, 2F02, 2F07, 2G10, 2E03, 2H06, 2F09, 2E08, 2F08, 2C09, 2B06, 2F10, 2E07, 3F02, 3H02, 3A09, 3G05, 3F07, 3A06, 3G01, 3D02, 3F01, 3B07, 3F06, 3A10, 3C02, 3E02, 3D11, 3A05, 3A08, 3F11, 3E11, 3A03, 3D03, 3E03, 3D06, 3G08, 3A01, 3B09, 3E04, 3B06, 3C04, 3D01, 3D09, 3E08, 3E09, 3B05, 3D04, 3D07, 3F04, 3B02, 3B04, 3C01, 3C06, 3F03, 3G04, G307, 3E01, 3F08, 3C09, 3A02, 3F05, 3E07, 3E05, 3F09, 3C07, 3C08, 3G10, 3H01, 3G03, 3D10, 3B10, 3D08, or 3B11.

10. The isolated ABP of claim 1, wherein the ABP comprises all three heavy chain CDRs (HCDR1, HCDR2, HCDR3) and all three light chain CDRs (LCDR1, LCDR2, LCDR3) from the clone designated 1C08, 1A03, 1A07, 1B04, 1C11, 1H06, 1D11, 1G01, 1H04, 1G06, 1B05, 1A08, 1F11, 1A04, 1F08, 1G09, 1F02, 1F04, 1C10, 1E04, 1B07, 1E01, 1F07, 1A10, 1E10, 1D10, 1D07, 1E05, 1F01, 1E08, 1C06, 1H03, 1H08, 1C04, 1D02, 1D05, 1E11, 1F05, 1G11, 1A02, 1A06, 1C05, 1D04, 1E03, 1A11, 1E09, 1C01, 1D09, 1B03, 1H02, 1D03, 1B02, 1F03, 1C09, 1C07, 2G03, 2H05, 2C03, 2F05, 2F04, 2G04, 2C01, 2E01, 2A10, 2G06, 2H07, 2G07, 2B01, 2B10, 2D11, 2G02, 2H08, 2G01, 2E06, 2B02, 2D04, 2A07, 2H04, 2B11, 2F11, 2D08, 2F03, 2A05, 2D07, 2E09, 2G09, 2A04, 2H03, 2E04, 2A09, 2D09, 2B08, 2G11, 2C08, 2C05, 2C07, 2F01, 2H01, 2B07, 2C06, 2A11, 2E10, 2A06, 2C04, 2D02, 2E05, 2F02, 2F07, 2G10, 2E03, 2H06, 2F09, 2E08, 2F08, 2C09, 2B06, 2F10, 2E07, 3F02, 3H02, 3A09, 3G05, 3F07, 3A06, 3G01, 3D02, 3F01, 3B07, 3F06, 3A10, 3C02, 3E02, 3D11, 3A05, 3A08, 3F11, 3E11, 3A03, 3D03, 3E03, 3D06, 3G08, 3A01, 3B09, 3E04, 3B06, 3C04, 3D01, 3D09, 3E08, 3E09, 3B05, 3D04, 3D07, 3F04, 3B02, 3B04, 3C01, 3C06, 3F03, 3G04, G307, 3E01, 3F08, 3C09, 3A02, 3F05, 3E07, 3E05, 3F09, 3C07, 3C08, 3G10, 3H01, 3G03, 3D10, 3B10, 3D08, or 3B11.

11. The isolated ABP of claim 1, wherein the ABP comprises a variable heavy chain (VH) sequence from the clone designated 1C08, 1A03, 1A07, 1B04, 1C11, 1H06, 1D11, 1G01, 1H04, 1G06, 1B05, 1A08, 1F11, 1A04, 1F08, 1G09, 1F02, 1F04, 1C10, 1E04, 1B07, 1E01, 1F07, 1A10, 1E10, 1D10, 1D07, 1E05, 1F01, 1E08, 1C06, 1H03, 1H08, 1C04, 1D02, 1D05, 1E11, 1F05, 1G11, 1A02, 1A06, 1C05, 1D04, 1E03, 1A11, 1E09, 1C01, 1D09, 1B03, 1H02, 1D03, 1B02, 1F03, 1C09, 1C07, 2G03, 2H05, 2C03, 2F05, 2F04, 2G04, 2C01, 2E01, 2A10, 2G06, 2H07, 2G07, 2B01, 2B10, 2D11, 2G02, 2H08, 2G01, 2E06, 2B02, 2D04, 2A07, 2H04, 2B11, 2F11, 2D08, 2F03, 2A05, 2D07, 2E09, 2G09, 2A04, 2H03, 2E04, 2A09, 2D09, 2B08, 2G11, 2C08, 2C05, 2C07, 2F01, 2H01, 2B07, 2C06, 2A11, 2E10, 2A06, 2C04, 2D02, 2E05, 2F02, 2F07, 2G10, 2E03, 2H06, 2F09, 2E08, 2F08, 2C09, 2B06, 2F10, 2E07, 3F02, 3H02, 3A09, 3G05, 3F07, 3A06, 3G01, 3D02, 3F01, 3B07, 3F06, 3A10, 3C02, 3E02, 3D11, 3A05, 3A08, 3F11, 3E11, 3A03, 3D03, 3E03, 3D06, 3G08, 3A01, 3B09, 3E04, 3B06, 3C04, 3D01, 3D09, 3E08, 3E09, 3B05, 3D04, 3D07, 3F04, 3B02, 3B04, 3C01, 3C06, 3F03, 3G04, G307, 3E01, 3F08, 3C09, 3A02, 3F05, 3E07, 3E05, 3F09, 3C07, 3C08, 3G10, 3H01, 3G03, 3D10, 3B10, 3D08, or 3B11.

12. The isolated ABP of claim 1, wherein the ABP comprises a variable light chain (VL) sequence from the clone designated 1C08, 1A03, 1A07, 1B04, 1C11, 1H06, 1D11, 1G01, 1H04, 1G06, 1B05, 1A08, 1F11, 1A04, 1F08, 1G09, 1F02, 1F04, 1C10, 1E04, 1B07, 1E01, 1F07, 1A10, 1E10, 1D10, 1D07, 1E05, 1F01, 1E08, 1C06, 1H03, 1H08, 1C04, 1D02, 1D05, 1E11, 1F05, 1G11, 1A02, 1A06, 1C05, 1D04, 1E03, 1A11, 1E09, 1C01, 1D09, 1B03, 1H02, 1D03, 1B02, 1F03, 1C09, 1C07, 2G03, 2H05, 2C03, 2F05, 2F04, 2G04, 2C01, 2E01, 2A10, 2G06, 2H07, 2G07, 2B01, 2B10, 2D11, 2G02, 2H08, 2G01, 2E06, 2B02, 2D04, 2A07, 2H04, 2B11, 2F11, 2D08, 2F03, 2A05, 2D07, 2E09, 2G09, 2A04, 2H03, 2E04, 2A09, 2D09, 2B08, 2G11, 2C08, 2C05, 2C07, 2F01, 2H01, 2B07, 2C06, 2A11, 2E10, 2A06, 2C04, 2D02, 2E05, 2F02, 2F07, 2G10, 2E03, 2H06, 2F09, 2E08, 2F08, 2C09, 2B06, 2F10, 2E07, 3F02, 3H02, 3A09, 3G05, 3F07, 3A06, 3G01, 3D02, 3F01, 3B07, 3F06, 3A10, 3C02, 3E02, 3D11, 3A05, 3A08, 3F11, 3E11, 3A03, 3D03, 3E03, 3D06, 3G08, 3A01, 3B09, 3E04, 3B06, 3C04, 3D01, 3D09, 3E08, 3E09, 3B05, 3D04, 3D07, 3F04, 3B02, 3B04, 3C01, 3C06, 3F03, 3G04, G307, 3E01, 3F08, 3C09, 3A02, 3F05, 3E07, 3E05, 3F09, 3C07, 3C08, 3G10, 3H01, 3G03, 3D10, 3B10, 3D08, or 3B11.

13. The isolated ABP of claim 1, wherein the ABP comprises the VH sequence and the VL sequence from the clone designated 1C08, 1A03, 1A07, 1B04, 1C11, 1H06, 1D11, 1G01, 1H04, 1G06, 1B05, 1A08, 1F11, 1A04, 1F08, 1G09, 1F02, 1F04, 1C10, 1E04, 1B07, 1E01, 1F07, 1A10, 1E10, 1D10, 1D07, 1E05, 1F01, 1E08, 1C06, 1H03, 1H08, 1C04, 1D02, 1D05, 1E11, 1F05, 1G11, 1A02, 1A06, 1C05, 1D04, 1E03, 1A11, 1E09, 1C01, 1D09, 1B03, 1H02, 1D03, 1B02, 1F03, 1C09, 1C07, 2G03, 2H05, 2C03, 2F05, 2F04, 2G04, 2C01, 2E01, 2A10, 2G06, 2H07, 2G07, 2B01, 2B10, 2D11, 2G02, 2H08, 2G01, 2E06, 2B02, 2D04, 2A07, 2H04, 2B11, 2F11, 2D08, 2F03, 2A05, 2D07, 2E09, 2G09, 2A04, 2H03, 2E04, 2A09, 2D09, 2B08, 2G11, 2C08, 2C05, 2C07, 2F01, 2H01, 2B07, 2C06, 2A11, 2E10, 2A06, 2C04, 2D02, 2E05, 2F02, 2F07, 2G10, 2E03, 2H06, 2F09, 2E08, 2F08, 2C09, 2B06, 2F10, 2E07, 3F02, 3H02, 3A09, 3G05, 3F07, 3A06, 3G01, 3D02, 3F01, 3B07, 3F06, 3A10, 3C02, 3E02, 3D11, 3A05, 3A08, 3F11, 3E11, 3A03, 3D03, 3E03, 3D06, 3G08, 3A01, 3B09, 3E04, 3B06, 3C04, 3D01, 3D09, 3E08, 3E09, 3B05, 3D04, 3D07, 3F04, 3B02, 3B04, 3C01, 3C06, 3F03, 3G04, G307, 3E01, 3F08, 3C09, 3A02, 3F05, 3E07, 3E05, 3F09, 3C07, 3C08, 3G10, 3H01, 3G03, 3D10, 3B10, 3D08, or 3B11.

14. The isolated ABP of claim 1, wherein the ABP comprises an antibody or antigen-binding fragment thereof.

15. The isolated ABP of claim 1, wherein the antigen binding protein is linked to a scaffold, optionally wherein the scaffold comprises serum albumin or Fc, optionally wherein Fc is human Fc and is an IgG (IgG1, IgG2, IgG3, IgG4), an IgA (IgA1, IgA2), an IgD, an IgE, or an IgM.

16. (canceled)

17. The isolated ABP of claim 1, wherein the antigen binding protein comprises an Fv fragment, a Fab fragment, a F(ab′)₂ fragment, a Fab′ fragment, an scFv fragment, an scFv-Fc fragment, and/or a single-domain antibody or antigen binding fragment thereof.

18.-31. (canceled)

32. The isolated ABP of claim 1, wherein the antigen binding protein is a portion of a chimeric antigen receptor (CAR) comprising: an extracellular portion comprising the antigen binding protein; and an intracellular signaling domain.

33. An isolated polynucleotide or set of polynucleotides encoding the antigen binding protein of claim 1 or an antigen-binding portion thereof.

34. A vector or set of vectors comprising the polynucleotide or set of polynucleotides of claim 33.

35. A host cell comprising the polynucleotide or set of polynucleotides of claim 33, optionally wherein the host cell is CHO or HEK293, or optionally wherein the host cell is a T cell.

36. A method of producing an antigen binding protein comprising expressing the antigen binding protein with the host cell of claim 35 and isolating the expressed antigen binding protein.

37. A pharmaceutical composition comprising the antigen binding protein of claim 1 and a pharmaceutically acceptable excipient.

38. A method of increasing an immune response in a subject, comprising administering to the subject the ABP of claim 1, optionally wherein the subject has cancer, optionally wherein the cancer is selected from a solid tumor and a hematological tumor.

39. A method of treating cancer in a subject, comprising administering to the subject an effective amount of the antigen binding protein of claim 1, optionally wherein the cancer is selected from a solid tumor and a hematological tumor.

40.-44. (canceled)

45. A kit comprising the antigen binding protein of claim 1 and instructions for use.

46. (canceled)
